(19) United States
(12) Patent Application Publication
(10) Pub. No.: US 2013/0326734 A1
(43) Pub. Date: Dec. 5, 2013

(54) COMPOSITIONS AND METHODS FOR CONTROLLING CARBON DIOXIDE- (CO2-) REGULATED STOMATAL APERTURES, WATER TRANSPIRATION AND WATER USE EFFICIENCY IN PLANTS

(71) Applicants: Julian I. Schroeder, La Jolla, CA (US); Honghong Hu, San Diego, CA (US); Shaowu Xue, Taiyuan (CN)

(72) Inventors: Julian I. Schroeder, La Jolla, CA (US); Honghong Hu, San Diego, CA (US); Shaowu Xue, Taiyuan (CN)

(73) Assignee: THE REGENTS OF THE UNIVERSITY OF CALIFORNIA, Oakland, CA (US)

(21) Appl. No.: 13/982,439

(22) PCT Filed: Jan. 24, 2012
(86) PCT No.: PCT/US2012/022331

§ 371 Date: Aug. 21, 2013

Related U.S. Application Data
(60) Provisional application No. 61/438,618, filed on Feb. 1, 2011.

(51) Int. Cl. C12N 15/82

(52) U.S. Cl. CPC C12N 15/8261; C12N 15/8243
USPC 800/290; 800/278; 800/298; 435/419; 435/468

(57) ABSTRACT

In alternative embodiments, the invention provides compositions and methods for manipulating the exchange of water and/or carbon dioxide (CO₂) through plant stomata by combining the control of expression of CO₂ sensor genes with the control of expression of OST1 protein kinase and the related protein kinases SnRK2.2 and SnRK2.3, and their genes. In alternative embodiments, the invention provides plants having increased water use efficiency, and drought-resistant plants; and methods for engineering of water transpiration and water use efficiency in plants, and engineering plants with increased water use efficiency and drought-resistant plants.

### TECHNICAL FIELD

This invention generally relates to plant molecular and cellular biology. In alternative embodiments, the invention provides compositions and methods for manipulating the exchange of water and/or carbon dioxide (CO₂) through plant stomata by combining the control of expression of CO₂ sensor genes with the control of expression of OST1 (Open Stomata 1) protein kinase and related protein kinases SnRK2.2 and SnRK2.3, and their genes. In alternative embodiments, the invention provides plants, plant tissues and cells, having increased water use efficiency, and drought-resistant plants, plant tissues and cells; and methods for engineering of water transpiration and water use efficiency in plants, and engineering plants with increased water use efficiency and drought-resistant plants, plant tissues and cells.

### BACKGROUND

Stomatal pores in the epidermis of plant leaves enable the control of plant water loss and the influx of CO₂ into plants from the atmosphere. Carbon dioxide is taken up for photosynthetic carbon fixation and water is lost through the process of transpiration through the stomatal pores. Each stomate is made up of a specialized pair of cells named guard cells, which can modify the size of the stomatal pore by controlling guard cell turgor status.

An important trait in agriculture, in biotechnological applications and the production of biofuels is the water use efficiency of plants. The water use efficiency defines how well a plant can balance the loss of water through stomata with the net CO2 uptake into leaves for photosynthesis and hence its biomass accumulation. Several biotic and abiotic factors influence the state of stomatal opening thereby optimizing the water use efficiency of a plant in a given condition.

The concentration of CO₂ regulates stomatal movements, where high levels of CO₂ will lead to stomatal closing and low levels of CO₂ will induce stomatal opening. Thus CO₂ regulates CO₂ influx into plants and plant water loss on a global scale.

### SUMMARY

In alternative embodiments, the invention provides methods for increasing the water use efficiency of a guard cell, a plant, plant leaf, plant organ or plant part; or increasing the rate of growth or biomass production in a plant, plant leaf, plant organ or plant part (e.g., under conditions of drought or increased atmospheric carbon dioxide); or enhancing the carbon dioxide (CO₂) sensitivity of a plant, plant leaf, plant organ or plant part; or down-regulating or decreasing carbon dioxide (CO₂) and/or water exchange in a guard cell of a plant, plant leaf, plant organ or plant part; comprising:

(a) in a cell of the plant, plant leaf, plant organ or plant part, or in a plant guard cell, increasing the expression and/or activity of:
(1) an OST1 (Open Stomata 1, also known as SnRK2.6) protein kinase-expressing nucleic acid or an OST1 protein kinase gene or mRNA (message) encoding a polypeptide with OST1 protein kinase activity; or
(2) a protein kinase SnRK2.2- or SnRK2.3-expressing nucleic acid or an SnRK2.2- or SnRK2.3 protein kinase gene or mRNA (message) encoding a polypeptide with SnRK2.2- or SnRK2.3 protein kinase activity (SnRK2 genes are SNF1 Related Protein Kinase Subfamily 2 genes) (SNF1 is “Sucrose non-fermenting 1”);

(b) the method of (a), wherein the increasing of expression and/or activity of the OST1, SnRK2.2- or SnRK2.3 protein kinase is by: (1) providing a heterologous OST1-, SnRK2.2- or SnRK2.3-expressing nucleic acid (e.g., a gene or message) and expressing the gene, message and/or protein in the guard cell, plant, plant leaf, plant organ or plant part; (2) increasing of expression and/or activity of a homologous OST1 -, SnRK2.2- or SnRK2.3-expressing nucleic acid (e.g., a gene or message); or, (3) a combination of (1) and (2);

(b) the method of (a), further comprising in the cell of the plant, plant leaf, plant organ or plant part, or in the plant guard cell, increasing the expression and/or activity of a CO₂ a sensor protein or a carbonic anhydrase by: (1) providing a heterologous CO₂ sensor protein-expressing nucleic acid (e.g., a gene or message), or a carbonic anhydrase-expressing nucleic acid (e.g., a gene or message) and expressing the gene, message and/or protein in the guard cell, plant, plant leaf, plant organ or plant part; (2) increasing of expression and/or activity of a homologous CO₂ sensor protein-expressing nucleic acid (e.g., a gene or message), or a homologous CO₂ sensor protein-expressing nucleic acid (e.g., a gene or message), or a homologous OST1 carbonic anhydrase-expressing nucleic acid (e.g., a gene or message); or, (3) a combination of (1) and (2); or

(c) the method of (b), wherein the carbonic anhydrase is a β-carbonic anhydrase;

thereby increasing the water use efficiency of the guard cell, plant, plant leaf, plant organ or plant part; or increasing the rate of growth or biomass production in the plant, plant leaf, plant organ or plant part; or enhancing the carbon dioxide (CO₂) sensitivity of the plant, plant leaf, plant organ or plant part; or down-regulating or decreasing carbon dioxide (CO₂) and/or water exchange in the guard cell of the plant, plant leaf, plant organ or plant part.

In alternative embodiments, the invention provides methods for up-regulating or increasing carbon dioxide (CO₂) and/or water exchange in a guard cell, a plant, plant leaf, plant organ or plant part; decreasing the water use efficiency of a guard cell, a plant, plant leaf, plant organ or plant part; or decreasing (desensitizing) the carbon dioxide (CO₂) sensitivity of a plant, plant leaf, plant organ or plant part; or upregulating or increasing carbon dioxide (CO₂) and/or water exchange in a guard cell of a plant, plant leaf, plant organ or plant part; comprising:

(a) in a cell of the plant, plant leaf, plant organ or plant part, or in a plant guard cell, decreasing the expression and/or activity of:
(1) an OST1 protein kinase-expressing nucleic acid or an OST1 protein kinase gene or mRNA (message) encoding a polypeptide with OST1 protein kinase activity; or
(2) a protein kinase SnRK2.2- or SnRK2.3-expressing nucleic acid or an SnRK2.2- or SnRK2.3 protein kinase gene or mRNA (message) encoding a polypeptide with SnRK2.2- or SnRK2.3 protein kinase activity;

(b) the method of (a), wherein the decreasing of expression and/or activity of the OST1, SnRK2.2 or SnRK2.3 protein kinase is by: (1) providing a heterologous antisense or iRNA OST1, SnRK2.2 or SnRK2.3 protein kinase nucleic acid (e.g., to decrease the expression or activity of a gene or message), or any nucleic acid inhibitory to the expression or the OST1, SnRK2.6 or SnRK2.6 protein kinase; and, expressing the inhibitory nucleic acid, the antisense or the iRNA in the guard cell, plant, plant leaf, plant organ or plant part; (2) decreasing of expression and/or activity of a homologous OST1 , SnRK2.2- or SnRK2.3 kinase-expressing nucleic acid (e.g., a gene or message); or, (3) a combination of (1) and (2);

(b) the method of (a), further comprising in the cell of the plant, plant leaf, plant organ or plant part, or in the plant guard cell, decreasing the expression and/or activity of a CO₂ a sensor protein or a carbonic anhydrase by: (1) providing a heterologous antisense or iRNA to a CO₂ sensor protein- or a carbonic anhydrase-expressing nucleic acid (e.g., a gene or message), or any nucleic acid inhibitory to the expression of the CO₂ sensor protein or the carbonic anhydrase, and expressing the inhibitory nucleic acid, the antisense or the iRNA in the guard cell, plant, plant leaf, plant organ or plant part; (2) decreasing of expression and/or activity of a homologous CO₂ sensor protein-expressing nucleic acid (e.g., a gene or message) or a homologous carbonic anhydrase-expressing nucleic acid (e.g., a gene or message); or, (3) a combination of (1) and (2); or

(c) the method of (b), wherein the carbonic anhydrase is a β-carbonic anhydrase;

thereby up-regulating or increasing carbon dioxide (CO₂) and/or water exchange in the guard cell, plant, plant leaf, plant organ or plant part; decreasing the water use efficiency of the guar cell, plant, plant leaf, plant organ or plant part; or increasing the rate of growth or biomass production in the plant, plant leaf, plant organ or plant part; or decreasing (desensitizing) the carbon dioxide (CO₂) sensitivity of the plant, plant leaf, plant organ or plant part; or up-regulating or increasing carbon dioxide (CO₂) and/or water exchange in the guard cell of the plant, plant leaf, plant organ or plant part.

In alternative embodiments of the methods, the polypeptide having carbonic anhydrase activity comprises an amino acid sequence having between about 75% to 100% sequence identity with an amino acid sequence of (comprising) SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46.

In alternative embodiments of the methods, the polypeptide having carbonic anhydrase activity is encoded by a nucleotide sequence of (comprising) SEQ ID NO:1, SEQ ID NO:2, SEQ ID NO:3, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, or SEQ ID NO:45. In alternative embodiments of the methods, the polypeptide having OST1 protein kinase activity comprises an amino acid sequence having between 75% to 100% sequence identity with an amino acid sequence of (comprising) SEQ ID NO:12 or SEQ ID NO:14; or the polypeptide having OST1 protein kinase activity is encoded by a nucleotide sequence of (comprising) SEQ ID NO:11 or SEQ ID NO:13.

In alternative embodiments of the methods, the plant is characterized by controlled CO₂ exchange under ambient 365 ppm CO₂, elevated ppm CO₂ or reduced ppm CO₂, or the plant is characterized by controlled water exchange under ambient 365 ppm CO₂, elevated ppm CO₂ or reduced ppm CO₂.

In alternative embodiments of the methods, the CO₂ sensor protein-expressing nucleic acid or gene, carbonic anhydrase-expressing nucleic acid, message or gene, and/or the protein kinase-expressing nucleic acid, message or gene, is oeprably linked to a plant expressible promoter, an inducible promoter, a constitutive promoter, a guard cell specific promoter, a drought-inducible promoter, a stress-inducible promoter or a guard cell active promoter.

In alternative embodiments of the methods, the up-regulating or increasing carbon dioxide (CO₂) and/or water exchange in a guard cell of a plant, plant cell, plant leaf, plant organ or plant part; decreasing the water use efficiency of a guard cell, a plant, plant leaf, plant organ or plant part; or decreasing (desensitizing) the carbon dioxide (CO₂) sensitivity of a plant, plant leaf, plant organ or plant part; or upregulating or increasing carbon dioxide (CO₂) and/or water exchange in a guard cell or a plant, plant leaf, plant organ or plant part; comprises:

(a) providing (i) a nucleic acid inhibitory to the expression of a CO₂ sensor protein-expressing nucleic acid or a CO₂ sensor gene or transcript (mRNA), each encoding a polypeptide having a carbonic anhydrase (CA) activity or a β-carbonic anhydrase activity; and/or (ii) a nucleic acid inhibitory (e.g., antisense, iRNA) to the expression an and OST1, SnRK2.2- or SnRK2.3 protein kinase-expressing nucleic acid or an OST1 , SnRK2.2- or SnRK2.3 protein kinase gene or transcript;

(b) expressing the nucleic acid inhibitory to the expression of the CO₂ sensor protein-expressing nucleic acid, gene or transcript (e.g., expressing an antisense, iRNA or inhibitory nucleic acid) in a guard cell; and/or, expressing a nucleic acid inhibitory to the expression of the protein kinase-expressing nucleic acid, gene or transcript,

thereby up-regulating or increasing carbon dioxide (CO₂) and/or water exchange in a guard cell; decreasing the water use efficiency of a guard cell, a plant, plant leaf, plant organ or plant part; or decreasing (desensitizing) the carbon dioxide (CO₂) sensitivity of a plant, plant leaf, plant organ or plant part; or upregulating or increasing carbon dioxide (CO₂) and/or water exchange in a guard cell or a plant, plant leaf, plant organ or plant part.

In alternative embodiments of the methods, the nucleic acid inhibitory to the expression of a CO₂ sensor protein-expressing nucleic acid comprises:

(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence encoding a polypeptide having carbonic anhydrase activity,

the polypeptide optionally comprising an amino acid sequence having between about 75% and 100% sequence identity with an amino acid sequence of: SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46, or

(b) a partial or complete complementary sequence of the nucleotide sequence (a).

In alternative embodiments of the methods, the nucleic acid inhibitory to the expression of a CO₂ sensor protein-expressing nucleic acid comprises:

(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, or SEQ ID NO:45; or

(b) a partial or complete complementary sequence of the nucleotide sequence (a).

In alternative embodiments of the methods, the nucleic acid inhibitory to the expression of the polypeptide having OST1 protein kinase activity comprises:

(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence encoding an amino acid sequence having between 75% and 100% sequence identity with amino acid sequence of SEQ ID NO:12 or SEQ ID NO: 14; or

(b) a partial or complete complementary sequence of the nucleotide sequence (a).

In alternative embodiments of the methods, the nucleic acid inhibitory to the expression of the polypeptide having OST1 protein kinase activity comprises:

(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence of SEQ ID No.11 or SEQ ID NO:13; or

(b) a partial or complete complementary sequence of the nucleotide sequence (a).

In alternative embodiments of the methods, the nucleic acid inhibitory to the expression of a CO₂ sensor protein-expressing nucleic acid comprises the nucleotide sequence of at least about 11, 12, 13, 14, 15, 16, 17, 18 or 19 or more nucleotides and a complementary sequence to the nucleotide sequence of at least about 11, 12, 13, 14, 15, 16, 17, 18, or 19 or more nucleotides.

In alternative embodiments of the methods, the nucleotide sequence comprising the at least about 11, 12, 13, 14, 15, 16, 17, 18, or 19 or more nucleotides is a nucleotide sequence comprising at least 50 or 100 or 300 nucleotides having between 75 to 100% sequence identity to the nucleotide sequence encoding a polypeptide having carbonic anhydrase activity and/or nucleotide sequence encoding a polypeptide having OST1 protein kinase activity.

In alternative embodiments of the methods, the plant is characterized by controlled CO₂ exchange under ambient 365 ppm CO₂, elevated ppm CO₂ or reduced ppm CO₂, or the plant is characterized by controlled water exchange under ambient 365 ppm CO₂, elevated ppm CO₂ or reduced ppm CO₂.

In alternative embodiments of the methods, the CO₂ sensor protein-inhibitory nucleic acid an/or the OST1 protein kinase-inhibitory nucleic acid is operably linked to a plant expressible promoter an inducible promoter, a constitutive promoter, a guard cell specific promoter, a drought-inducible promoter, a stress-inducible promoter or a guard cell active promoter.

In alternative embodiments, the invention provides methods for regulating water exchange in a cell of a plant, plant leaf, plant organ or plant part comprising:

(a) expressing or increasing the expression of a CO₂ sensor protein-encoding or a carbonic anhydrase-encoding gene or transcript, and an OST1, SnRK2.2- or SnRK2.3 protein kinase-encoding gene or transcript, by providing a CO₂ sensor protein expressing and an OST1, SnRK2.2- or SnRK2.3 protein kinase nucleic acid, gene or transcript, as set forth in a composition or method of this invention, in the plant, guard cell, plant cell, plant leaf, plant organ or plant part; or

(b) decreasing the expression of a CO₂ sensor protein encoding gene or transcript or a carbonic anhydrase gene or transcript and an OST1, SnRK2.2- or SnRK2.3 protein kinase-encoding gene or transcript in the plant, guard cell, plant cell, plant leaf, plant organ or plant part, by expressing a nucleic acid inhibitory to the expression of the CO₂ sensor protein-expressing or carbonic anhydrase-expressing nucleic acid, gene or transcript and the OST1, SnRK2.2- or SnRK2.3 protein kinase-expressing nucleic acid, gene or transcript, as set forth in a method of the invention, in the plant, guard cell, plant cell, plant leaf, plant organ, or plant part;

thereby regulating water exchange, wherein down-regulating or decreasing water exchange is achieved by expression or increased expression of the carbonic anhydrase or CO₂ sensor protein and the protein kinase and wherein up-regulating or increasing water exchange is achieved by reduction of expression of the carbonic anhydrase or CO₂ sensor protein and the protein kinase in the plant, guard cell, plant cell, plant leaf, plant organ, or plant part.

In alternative embodiments of the methods, the increasing or decreasing of the expression is in the plant guard cell.

In alternative embodiments, the invention provides methods for regulating water uptake or water loss in a plant, plant cell, plant leaf, plant organ or plant part comprising:
(a) expressing or increasing the expression of a CO₂ sensor protein-encoding or a carbonic anhydrase-encoding gene or transcript, and an OST1, SnRK2.2- or SnRK2.3 protein kinase-encoding gene or transcript, by providing a CO₂ sensor protein expressing and a OST1, SnRK2.2- or SnRK2.3 protein kinase nucleic acid, gene or transcript, as set forth in a composition or method of this invention, in the plant, guard cell, plant cell, plant leaf, plant organ, or plant part; or

(b) decreasing the expression of a CO₂ sensor protein encoding gene or transcript or a carbonic anhydrase gene or transcript and an OST1, SnRK2.2- or SnRK2.3 protein kinase-encoding gene or transcript in the plant, guard cell, plant cell, plant leaf, plant organ, or plant part, by expressing a nucleic acid inhibitory to the expression of the CO₂ sensor protein-expressing or carbonic anhydrase-expressing nucleic acid, gene or transcript and the OST1, SnRK2.2- or SnRK2.3 protein kinase-expressing nucleic acid, gene or transcript, as set forth in a method of this invention, in the plant, guard cell, plant cell, plant leaf, plant organ, or plant part;

thereby regulating water uptake or water loss, wherein down-regulating water uptake or causing water conservation is achieved by expression or increased expression of the carbonic anhydrase or CO₂ sensor protein and the OST1, SnRK2.2- or SnRK2.3 protein kinase and wherein up-regulating water exchange or increasing water loss is achieved by reduction of expression of the carbonic anhydrase or CO₂ sensor protein and the OST1, SnRK2.2- or SnRK2.3 protein kinase in the plant, plant cell, plant leaf, plant organ, or plant part. The increasing or decreasing of the expression can occur in the plant guard cell.

In alternative embodiments, the invention provides methods for making a plant with enhanced water use efficiency (WUE), or drought-resistant plant, plant cell, plant leaf, plant organ or plant part, comprising:

expressing or increasing the expression of a CO₂ sensor protein-encoding or a carbonic anhydrase-encoding gene or transcript, and an OST1, SnRK2.2- or SnRK2.3 protein kinase-encoding gene or transcript, by providing a CO₂ sensor protein expressing and an OST1, SnRK2.2- or SnRK2.3 protein kinase nucleic acid, gene or transcript, as set forth in a composition or method of this invention, in the plant, guard cell, plant cell, plant leaf, plant organ, or plant part

thereby regulating water uptake or water loss and increasing the WUE in the plant, plant cell, plant leaf, plant organ, or plant part.

The increasing of the expression can occur in the plant guard cell.

In alternative embodiments, the invention provides methods for making a heat-resistant plant, guard cell, plant cell, plant leaf, plant organ, or plant part, comprising:

decreasing the expression of a CO₂ sensor protein encoding gene or transcript or a carbonic anhydrase gene or transcript and an OST1, SnRK2.2- or SnRK2.3 protein kinase-encoding gene or transcript in the plant, guard cell, plant cell, plant leaf, plant organ, or plant part, by expressing a nucleic acid inhibitory to the expression of the CO₂ sensor protein-expressing or carbonic anhydrase-expressing nucleic acid, gene or transcript and the OST1, SnRK2.2- or SnRK2.3 protein kinase-expressing nucleic acid, gene or transcript, as set forth in a method of the invention, in the plant, guard cell, plant cell, plant leaf, plant organ, or plant part,

thereby making a heat-resistant plant, guard cell, plant cell, plant leaf, plant organ, or plant part.

The decreasing of the expression can occur in the plant guard cell.

In alternative embodiments, the invention provides methods for opening a stomatal pore in a guard cell, plant, plant part, a plant organ, a plant leaf, or a plant cell, comprising:

decreasing the expression of a CO₂ sensor protein encoding gene or transcript or a carbonic anhydrase gene or transcript and an OST1, SnRK2.2- or SnRK2.3 protein kinase-encoding gene or transcript in the plant, guard cell, plant cell, plant leaf, plant organ, or plant part, by expressing a nucleic acid inhibitory to the expression of the CO₂ sensor protein-expressing or carbonic anhydrase-expressing nucleic acid, gene or transcript and the OST1, SnRK2.2- or SnRK2.3 protein kinase-expressing nucleic acid, gene or transcript, as set forth in a method of the invention, in the plant, guard cell, plant cell, plant leaf, plant organ, or plant part,

thereby opening a stomatal pore in the guard cell, plant, plant part, plant organ, plant leaf, or plant cell.

The decreasing of the expression can occur in the plant guard cell.

In alternative embodiments, the invention provides methods for closing a stomatal pore on a guard cell in the epidermis or a plant, a plant leaf, plant organ or a plant cell, comprising:

expressing or increasing the expression of a CO₂ sensor protein-encoding or a carbonic anhydrase-encoding gene or transcript, and an OST1, SnRK2.2- or SnRK2.3 protein kinase-encoding gene or transcript, by providing a CO₂ sensor protein expressing and an OST1, SnRK2.2- or SnRK2.3 protein kinase nucleic acid, gene or transcript, as set forth in a composition or method of this invention, in the plant, guard cell, plant cell, plant leaf, plant organ, or plant part

thereby closing a stomatal pore on the guard cell in the epidermis of the plant, plant leaf, plant organ or plant cell.

The expression or increase in expression can occur in the plant guard cell.

In alternative embodiments, the invention provides methods for enhancing or optimizing biomass accumulation in a plant, a plant leaf, a plant organ, a plant part, a plant cell, or seed by balancing the loss of water through stomata with the net CO₂ uptake for photosynthesis, and hence enhancing or optimizing biomass accumulation in the plant, plant leaf, plant part, plant organ, plant cell, or seed, comprising opening or closing stomatal pores using a method of the invention.

In alternative embodiments, the invention provides methods for reducing leaf temperature and enhancing transpiration in a plant, a plant leave, or a plant cell, comprising opening a stomatal pore a cell or cells of the plant using a method of the invention.

In alternative embodiments, the plant is, or the guard cell, plant cell, plant part or plant organ, is isolated and/or derived from: (i) a dicotyledonous or monocotyledonous plant; (ii) wheat, oat, rye, barley, rice sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Curcurbita, Daucus, Elaeis, Fragaria, Glycine, Gossypium, Hellanthus, Heterocallis, Hordeum, Hyascyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, or Zea.

In alternative embodiments, the invention provides transgenic guard cells, plants, plant cells, plant tissues, plant seeds or fruits, plant parts or plant organs, comprising:

(a) (1) a heterologus OST1 protein kinase-expressing nucleic acid or an OST1 protein kinase gene or mRNA (message) encoding a polypeptide with OST1 protein kinase activity; or

(2) a heterologous protein kinase SnRK2.2- or SnRK2.3-expressing nucleic acid or an SnRK2.2- or SnRK2.2 protein kinase gene or mRNA (message) encoding a polypeptide with SnRK2.2- or SnRK2.2 protein kinase activity; or

(b) the transgenic plant cell, plant, plant part or plant organ of (a), further comprising a heterologous nucleic acid, gene or transcript encoding a protein having a carbonic anhydrase (CA) activity of a β-carbonic anhydrase activity, or encoding a CO₂ sensor protein,

wherein optionally the nucleic acid, gene or transcript is operably linked to a plant expressible promoter, an inducible promoter, a constitutive promoter, a guard cell specific promoter, a drought-inducible promoter, a stress-inducible promoter or a guard cell active promoter;

and optionally the nucleic acid, gene or transcript is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ.

In alternative embodiments, the invention provides transgenic guard cells, plants, plant cells, plat tissues, plant seeds or fruits, plant parts or plant organs, comprising:

(a)(1) a heterologus nucleic acid that is inhibitory to an OST1 protein kinase-expressing nucleic acid or an OST1 protein kinase gene or mRNA (message) encoding a polypeptide with OST1 protein kinase activity, or is inhibitory to the activity or the kinase; or

(2) a heterologus nucleic acid that is inhibitory to a protein kinase SnRK2.2 - or SnRK2.3-expressing nucleic acid or an SnKR2.2- or SnRK2.3 protein kinase gene or mRNA (message) encoding a polypeptide with SnRK2.2 - or SnRK2.3 protein kinase activity, or is inhibitory to the activity or the kinase; or

(b) the transgenic plant cell, plant, plant part or plant organ of (a), further comprising a heterologous nucleic acid that is inhibitory to a gene or transcript encoding a protein having a carbonic anhydrase (CA) activity or a β-carbonic anhydrase activity, or is inhibitory to a gene or transcript encoding a CO₂ sensor protein,

wherein optionally the inhibitory nucleic acid is operably linked to a plant expressible promoter, an inducible promoter, a constitutive promoter, a guard cell specific promoter, a drought-inducible promoter, a stress-inducible promoter or a guard cell active promoter;

and optionally the inhibitory nucleic acid is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ,

and optionally the inhibitory nucleic acid comprises an antisense RNA or an iRNA.

In alternative embodiments, the invention provides transgenic guard cells, plants, plant cells, plant tissues, plant seeds or fruits, plant parts or plant organs, comprising:

(a) a first and second recombinant gene, wherein the first recombinant gene comprises an expression-increasing recombinant first gene or an expression-inhibiting first recombinant gene, and wherein the second recombinant gene comprises an expression-increasing second recombinant gene or an expression-inhibiting second recombinant gene;

wherein the expression increasing first recombinant gene comprises:
i. a plant, plant cell or guard cell expressible promoter; and
ii. a heterologus nucleic acid encoding; a polypeptide having a carbonic anhydrase (CA) activity or a β-carbonic anhydrase activity, or, a CO₂ sensor protein; and

optionally further comprising a transcription termination and polyadenylation signal;

wherein the expression-inhibiting first recombinant gene comprises the following operably linked DNA fragments:
i. a plant, plant cell or guard cell expressible promoter; and
ii. a heterologus nucleic acid, which when transcribed produces a nucleic acid (e.g., a ribonucleic acid) inhibitory to the expression of a CO₂ sensor protein-expressing nucleic acid or a CO₂ sensor gene or transcript (mRNA), each optionally encoding a polypeptide having a carbonic anhydrase (CA) activity or a β-carbonic anhydrase activity,

optionally further comprising a transcription termination and polyadenylation signal;

wherein the expression-increasing second recombinant gene comprises:
i. a plant, plant cell or guard cell expressible promoter; and
ii. a heterologus nucleic acid encoding a polypeptide with OST1, SnRK2.2- or SnRK2.3 protein kinase activity;

optionally further comprising a transcription termination and polyadenylation signal;

wherein the expression inhibiting second recombinant gene:
i. a plant, plant cell or guard cell expressible promoter; and
ii. a heterologus nucleic acid, which when transcribed produces a nucleic acid (e.g., a ribonucleic acid) inhibitory to the expression of OST1, SnRK2.2- or SnRK2.3 protein kinase encoding gene;

optionally further comprising a transcription termination and polyadenylation signal.

In alternative embodiments, the nucleic acid (e.g., a DNA fragment) encoding a polypeptide having a carbonic anhydrase (CA) activity or a β-carbonic anhydrase activity encodes a polypeptide comprising an amino acid sequence having between 75% and 100% sequence identity with an amino acid sequence of SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46. In alternative embodiments, the polypeptide having carbonic anhydrase activity is encoded by a nucleotide sequence of (comprising) SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, or SEQ ID NO:45. In alternative embodiments, nucleic acid (e.g., DNA fragment) encoding the polypeptide with OST1, SnRK2.2- or SnRK2.3 protein kinase activity encodes a polypeptide comprising an amino acid sequence having between 75% and 100% sequence identity with an amino acid sequence of (comprising) SEQ ID NO:12 or SEQ ID NO:14. In alternative embodiments, the polypeptide having OST1 protein kinase activity is encoded by a nucleotide sequence selected from the nucleotide sequence of (comprising) SEQ ID NO:11 or SEQ ID NO:13.

In alternative embodiments, the nucleic acid (e.g., DNA fragment), which when transcribed yield an inhibitory nucleic acid (e.g., an inhibitory ribonucleic acid) to the expression of a CO₂ sensor protein-expressing nucleic acid comprises a nucleotide sequence of at least 11, 12, 13, 14, 15, 16, 17, 18, or 19 or more nucelotides having at least 90%, 91%, 92%, 93%, 94%, 95%, 96%, 97%, 98% or 99% or more sequence identity with a nucleotide sequence encoding a polypeptide having carbonic anhydrase activity comprising an amino acid sequence having between 75% and 100% sequence identity with an amino acid sequence selected from the amino acid sequence of (comprising) SEQ ID NO:1, SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46, or a complete or partial complement thereof.

In alternative embodiments, the nucleic acid (e.g., DNA fragment), which when transcribed yield a ribonucleic acid inhibitory to the expression of a CO₂ sensor protein-expressing nucleic acid comprises a nucleotide sequence of at least 11, 12, 13, 14, 15, 16, 17, 18, or 19 or more nucleotides having at least 94% sequence identity with a nucleotide sequence selected from the nucleotide sequence of (comprising) SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, or SEQ ID NO:45, or a complete or partial complement thereof.

In alternative embodiments, the ribonucleic acid inhibitory to the expression of a CO₂ sensor protein-expressing nucleic acid comprises the nucleotide sequence at least 11, 12, 13, 14, 15, 16, 17, 18, or 19 or more nucelotides and a complementary sequence to the nucleotide sequence at least 11, 12, 13, 14, 15, 16, 17, 18, or 19 or more nucelotides.

In alternative embodiments, the nucleic acid (e.g., DNA fragment), which when transcribed yield a ribonucleic acid inhibitory to the expression of a OST1 kinase protein-expressing nucleic acid comprises a nucleotide sequence of at least 11, 12, 13, 14, 15, 16, 17, 18, or 19 or more nucleotides having at least 90%, 91%, 92%, 93%, 94%, 95%, 96%, 97%, 98% or 99% or more sequence identity with a nucleotide sequence encoding a polypeptide having OST1 protein kinase activity comprising an amino acid sequence having between 75% and 100% sequence identity with an amino acid sequence selected from the amino acid sequence of (comprising) SEQ ID NO:12 or SEQ ID NO:14, or a complete or partial complement thereof.

In alternative embodiments, the nucleic acid (e.g., DNA fragment), which when transcribed yield a ribonucleic acid inhibitory to the expression of a OST1 protein kinase encoding nucleic acid comprises a nucleotide sequence of at least 11, 12, 13, 14, 15, 16, 17, 18, or 19 or more nucleotides having at least 90%, 91%, 92%, 93%, 94%, 95%, 96%, 97%, 98% or 99% or more sequence identity with a nucleotide sequence selected from the nucleotide sequence of (comprising) SEQ ID NO:11 or SEQ ID NO:13, or a complete or partial complement thereof.

In alternative embodiments, the ribonucleic acid inhibitory to the expression of a CO₂ sensor protein-expressing nucleic acid comprises the nucleotide sequence of at least 19 nucleotides and a complementary sequence to the nucleotide sequence of at least 19 nucleotides.

In alternative embodiments, the first recombinant gene is an expression increasing first recombinant gene, and the second recombinant gene is an expression increasing second recombinant gene. The first recombinant gene can be an expression inhibiting first recombinant gene, and the second recombinant gene is an expression inhibiting second recombinant gene. The first recombinant gene can be an expression increasing first recombinant gene, and the second recombinant gene is an expression inhibiting second recombinant gene. The first recombinant gene can be an expression inhibiting first recombinant gene, and the second recombinant gene is an expression increasing second recombinant gene.

In alternative embodiments, the plant is or the guard cell, plant, plant cell, plant tissue, plant seed or fruit, plant part or plant organ is isolated and/or derived from: (i) a dicotyledonous or monocotyledonous plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Curcurbita, Daucus, Elaeis, Fragaria, Glycine, Gossypium, Hellanthus, Heterocallis, Hordeum, Hyascyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, or Zea.

In alternative embodiments, the invention provides methods for altering the opening or closing of stomatal cells in a plant, plant part or plant organ, comprising providing cells of a guard cell, plant, plant cell, plant tissue, plant seed or fruit, plant part or plant organ with a first and second recombinant gene, wherein the first recombinant gene is selected from an expression increasing recombinant first gene or an expression inhibiting first recombinant gene, and wherein the second recombinant gene is selected from an expression increasing second recombinant gene or an expression inhibiting second recombinant gene as set forth in a composition or method of this invention, for
a. regulating carbon dioxide and water exchange in a plant;
b. regulating water uptake or water loss in a plant;
c. regulating water use efficiency or drought tolerance in a plant;
d. regulating biomass accumulation in a plant; or
e. regulating leaf temperature and transpiration in a plant.

In alternative embodiments, the first recombinant gene is an expression increasing first recombinant gene, and the second recombinant gene is an expression increasing second recombinant gene. The first recombinant gene can be an expression inhibiting first recombinant gene, and the second recombinant gene is an expression inhibiting second recombinant gene. The first recombinant gene can be an expression increasing first recombinant gene, and the second recombinant gene is an expression inhibiting second recombinant gene.

In alternative embodiments, the invention provides kits comprising a compound or compounds used to practice the methods of the invention, and optionally instructions to practice a method invention.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of embodiments of the invention and are not meant to limit the scope of the invention as encompassed by the claims.

Figures are described in detail herein.

Like reference symbols in the various drawings indicate like elements.

FIG. 1 illustrates data showing that high intracellular [CO₂] and [HCO3—] activate S-type anion channel currents in Arabidopsis ca1:ca4 double mutant guard cells but do not activate S-type anion currents in slac1 mutant guard cells with 2 μM [Ca²⁺]i. FIG. 1(A) Whole-cell currents without HCO3—/CO₂ and FIG. 1(B) with 11.5 mM free [HCO3—]i/2 mM free CO₂ in the pipette solution (pH 7.1) in ca1;ca4 double mutant guard cells. FIG. 1(C) Steady-state current-voltage relationships of the whole-cell currents recorded in ca1;ca4 mutant guard cells as in FIG. 1(A) (open circles, n=4 guard cells) and FIG. 1(B) (filled circles, n=9 guard cells). FIG. 1(D) Steady-state current-voltage relationships of whole-cell currents recorded in slac1-1 mutant guard cells (open circles: 0 mM added [HCO3—]i, n=6; filled circles; 11.5 mM free [HCO3—]i and 2 mM free [CO₂], n=6) and FIG. 1(E) in slac1-3 mutant guard cells (open circles; 0 mM added [HCO3—]i, n=4; filled circles: 11.5 mM free [HCO3—]i and 2 mM free [CO₂], n=8). Liquid junction potential was +1 mV. Data are mean±s.e.

FIG. 2 illustrates data showing that elevate [H+] (pH 6.1) together with 2 mM intracellular free [CO₂] did not activate S-type anion channel currents in wild type Col-0 guard cells when bicarbonate levels are lower. FIG. 2(A) Steady-state current-voltage relationships or whole-cell currents recorded in guard cells at 2 μM [Ca²⁺]i without bicarbonate in the pipette solution at pH 7.1 (open circles, n=6) and pH 6.1 (filled circles, n=5). FIG. 2(B) Steady-state current-voltage relationships of whole-cell currents at pH 6.1 without bicarbonate (open circles, n=5) and with 2 mM intracellular free [CO₂] and 1.1 mM free [HCO3—]i (filled circles, n=7) in the pipette solution. Liquid junction potential was +1 mV. FIG. 2(C) illustrates an example image of ratiometric pH sensitive Pt-GFP expressed guard cells. FIG. 2(D) Fluorescence ratio time series of guard cells expressing pH sensitive reporter Pt-GFP during extracellular perfusion with buffers of different pH as indicated by the top bar (n=6), FIG. 2(E) with MES buffer (10 mM MES, 10 mM KCl, 50 μM CaCl2, pH 5.6) and supplemented with sodium butyrate at mM-concentrations as indicated by the top bar of the graph and FIG. 2(F) with extracellular buffers bubbled with 0 ppm CO₂ and 800 ppm CO₂. GC denotes ratiometric fluorescence of guard cells and the ratio of non-guard cell background fluorescence (bg) is shown for the same experiments in (D, E, and F). Data are mean±s.e.

FIG. 3 illustrates data showing that high intracellular [HCO3—] at low [H+] and low free [CO₂] activate S-type anion channel currents in wild type Col-0 guard cells with 2 μM [Ca2+]i. FIG. 3(A) Typical recording of whole-cell currents in guard cell protoplasts without bicarbonate and FIG. 3(B) with 13.5 mM total bicarbonate (equivalent to 13.04 mM free [HCO3—]i/0.46 mM free [CO₂]) added to the pipette solution at pH 7.8. FIG. 3(C) Average steady-state current-voltage relationships of whole-cell currents recorded as in FIG. 3(A) (open circles, n=3) and FIG. 3(B) (filled circles, n=5). Liquid junction potential was +1 mV. Data are mean±s.e.

FIG. 4 illustrates data showing the requirement of both [Ca2+]i and elevated bicarbonate for activation of S-type anion channel currents in wild type (Col-0) guard cells. FIG. 4(A) Whole-cell currents in guard cell protoplasts at 2 μM [Ca²⁺]i without bicarbonate, FIG. 4(B) with 5.75 mM intracellular free [HCO3—]i/1 mM free [CO₂] (6.75 mM total bicarbonate added) and FIG. 4(C) with 11.5 mM intracellular free [HCO3—]i/2 mM free [CO₂] (13.5 mM total bicarbonate added) in the pipette solution at pH 7.1. FIG. 4(D) Whole-cell currents in guard cell protoplasts wit 0.15 μM [Ca2+]i without bicarbonate and FIG. 4(E) with 11.5 mM free [HCO3—]i/2 mM free [CO₂] (13.5 mM total bicarbonate in the pipette solution at pH 7.1. FIG. 4(F) Whole-cell currents in guard cell protoplasts with 0.6 μm [Ca²⁺]i and 11.5 mM intracellular free [HCO3—]i/2 mM free [CO₂] in the pipette solution at pH 7.1. FIG. 4(G) Steady-state current-voltage relationships of whole-cell currents as recorded in FIG. 4(A) (open triangles, n=6), FIG. 4(B) (open square, n=7), FIG. 4(C) (filled triangles, n=10), FIG. 4(D) (open circles, n=5). FIG. 4(E) (filled circles, n=7), and FIG. 4(F) (filled squares, n=7). Average data shown by dashed lines in FIG. 4(G) with or without of 5.75 mM and 11.5 mM free [HCO3—]i at 2 μM [Ca2+]i correspond to data reported in Hu et al (2010) and are included for comparison to 0.15 μM and 0.6 μM [Ca2+]i data. Liquid junction potential was +1 mV. Data are mean±s.e.

FIG. 5 illustrates data showing that enhanced bicarbonate sensitivity of S-type anion channel activation in ht1-2 mutant guard cells only at elevated [Ca²⁺]i. FIG. 5(A) Whole-cell currents in wild type Col-0 guard cells at 2 μM [Ca²⁺]i without bicarbonate and FIG. 5(B) with 6.75 mM total bicarbonate (equivalent to 5.75 mM free [HCO3—]i/1 mM free [Ca2]) added to the pipette solution. FIG. 5(C) Whole-cell currents in ht1-2 mutant guard cells at 2 μM [Ca²⁺]i without bicarbonate and FIG. 5(D) with 6.75 mM bicarbonate (equivalent to 5.75 mM free [HCO3—]i/1 mM free [CO₂]) in the pipette solution. FIG. 5(E) Average steady-state current-voltage relationships of whole-cell currents as recorded in FIG. 5(A) (open triangles, n=6), FIG. 5(B) (filled triangles, n=7), FIG. 5(C) (open circles, n=5) and FIG. 5(D) (filled circles, n=9). Average data for wild type Col-0 controls (WT) shown by dashed lines in FIG. 5(E) with 0 and 6.75 mM total bicarbonate (5.75 mM free [HCO3—]) with 2 μM [Ca2+]i correspond to data reported in Hu et al (2010) and are included for comparison to ht1-2 mutant data. FIG. 5(F) Whole-cell currents in ht1-2 mutant guard cell protoplasts at low 0.15 μM [Ca2+]i without bicarbonate and FIG. 5(G) with 6.75 mM bicarbonate (equivalent to 5.75 mM free [HCO3—]i/1 mM free [CO₂]) added to the pipette solution. FIG. 5(H) Average steady-state current-voltage relationships of whole-cell currents as recorded in FIG. 5(F) (open circles, n=5) and FIG. 5(G) (filled circles, n=5). Liquid junction potential was +1 mV. Data are mean±s.e.

FIG. 6 illustrates data showing that HCO3—/CO2 activation S-type anion channel currents is disrupted to ost1-2 and ost1-3 mutant guard cells with 2 μM [Ca2+]i. FIG. 6(A) Whole-cell recording without bicarbonate and FIG. 6(B) with 13.5 mM total bicarbonate (11.5 mM free [HCO3—]i+2 mM free [CO2])added to the pipette solution in ost1-2 mutant guard cells. FIG. 6(C) Whole-cell recording with 13.5 mM total bicarbonate in the pipette solution in ost1-3 mutant guard cells. FIG. 6(D) Whole-cell currents with 13.5 mM total bicarbonate and FIG. 6(E) without bicarbonate added to the pipette solution in wild type Ler guard cell protoplasts. FIG. 6(F) Steady-state current-voltage relationships of recordings as in FIG. 6(A) (open squares: ost1-2, —[HCO3—]i, n=5), FIG. 6(B) (filled squares: ost1-2, +[HCO3—]i, n=6), FIG. 6(C) (filled triangles; ost1-3, +[HCO3—]i, n=6), FIG. 6(D) (filled circles: wild type Ler, +[HCO3—]i, n=7) and FIG. 6(E) (open circles: wild type Ler, −[HCO3—]i, n=5). The pipette solution was adjusted to pH 7.1 in all the recordings. Liquid junction potential was +1 mV. Data are mean±s.e.

FIG. 7 illustrates data showing that CO₂-induced stomatal closure is strongly impaired in ost1 mutants. FIG. 7(A) Stomatal closure is impaired in ost1-3 mutant leaves in response to elevated [CO₂], *P<0.05, student's test. FIG. 7(B) Time-resolved relative stomatal conductance responses to [CO₂] in ost1-3 mutant and wild type Col-0 intact leaves (n=4 for each genotype). FIG. 7(C) Patterns of relative stomatal conductance in responses to changes in [CO₂] in intact ost1-3 and wild type Col plants (n=8 for ost1-3, n=6 for Col) and FIG. 7(D) in intact ost1-1, ost1-2 and wild type Ler plants (n=4 for each genotype). Data shown in (B, C, and D) were normalized in FIGS. 13A, B, and C (or Supplementary FIGS. 4A, B and C), respectively. Imposed CO₂ concentrations are shown at the bottom. Data are mean±s.e.

FIG. 8 illustrates data showing that [CO₂]-induced stomatal closure is not strongly affected in ABA receptor pyr1;pyl1; pyl2;pyl4 quadruple mutant and PP2C abi1-1 and abi2-1 mutant plants. FIG. 8(A) ABA receptor pyr1;pyl1; pyl2;pyl4 quadruple mutant does not abrogate CO2-regulation of stomatal conductance in intact leaves (n=4 for each genotype). Data shown were normalized in FIG. 13D (or Supplementary FIG. 4D). FIG. 8(B) Time-resolved stomatal conductance responses to [CO₂] in abi1-1, abi2-1 mutants and wild type Col-0 leaves (n=4 for wild type, n=6 for abi1-1 and abi2-1 mutants). FIG. 8(C,D) Normalized data of FIG. 8(B). Data are mean±s.e.

FIG. 9 illustrates a model for mechanisms of alternative embodiments of the invention showing the sequence of events that mediate CO₂ regulation of S-type anion channels and stomatal closing. [Ca2+]i sensitivity priming and [Ca2+]i-independent mechanisms are proposed to regulate SLAC1-dependent S-type anion currents in parallel via an “AND”-like gate.

FIG. 10 (or Supplementary FIG. 1, or FIG. S1) illustrates data showing that no large S-type anion currents were activated by extracellular application of with bicarbonate. FIG. 10(A) Whole-cell currents recording in Col-0 wild type guard cells (n=6). The bath solution contained 30 mM CsCl, 2 mM MgCl₂, 1 mM CaCl₂ and 10 mM Mes/Tris, pH 5.6. The pipette solution contained 150 mM CsCl, 2 mM MgCl₂, 6.7 mM EGTA, 6.03 mM CaCl₂ (2 μM[Ca²+]i), 5 mM Mg-ATP, 5 mM Tris-GTP, 1 mM HEPES/Tris, pH 7.1. Liquid junction potential was −1 mV. FIG. 10(B) Whole-cell recording of guard cells perfused with total 13.5 mM bicarbonate-containing solution (11.5 mM free HCO₃⁻ and 2 mM CO₂) at pH 7.1. The other components of the bath were 30 mM CsCl, 2 mM MgCl₂, 1 mM CaCl₂ and 10 mM HEPES/Tris, pH 7.1. Bath volume was 200 μl and perfused for 2 min at 1 ml/min, n=6. Liquid junction potential was −2 mV. FIG. 10(C) Steady-state current-voltage relationships of whole-cell currents as shown in FIG. 10(A) and FIG. 10(B). At a voltage of −144 mV, the control (background) current was −13±5 pA (n=6), and the current was −17±5 pA in a bicarbonate-containing solution (n=6), P>0.05.

FIG. 11 (or Supplementary FIG. 2, or FIG. S2) illustrates data showing that reversal potential of S-type anion currents activated by 50 mM total bicarbonate added to the pipette solution. FIG. 11(A) Typical recording of S-type anion currents activated by intracellular 50 mM total bicarbonate, 50 mM total bicarbonate at pH 7.1 equivalent to 43.4 mM free [HCO₃⁻], and 6.6 mM [CO₂] was calculated using the Henderson-Hasselbalch equation as described in the Methods. FIG. 11(B) Steady-state current-voltage relationship showed reversal potential of S-type anion currents at +26.0±0.9 mV (n=4). Data are mean±s.e. Liquid junction potential was +3 mV.

FIG. 12 (or Supplementary FIG. 3, or FIG. S3) illustrates data showing that extracellular pH shifts cause measurable intracellular pH changes in guard cells. Fluorescence ratio time series of guard cells from another transformed line expressing pH sensitive reporter Pt-GFP during extracellular perfusion with buffers of different pH as indicated by the top bar (See also FIG. 2D). GC denotes ratiometric fluorescence in guard cells and the ratio of non-guard cell background fluorescence (bg) is shown for the same experiments.

FIG. 13 (or Supplementary FIG. 4, FIG. S4) illustrates data shown CO₂-induced stomatal closure in ost1 and pyr1;pyl1; pyl2;pyl4 quadruple mutant mutants. FIG. 13(A) Stomatal conductance responses to [CO₂] in ost1-3 mutant and Col-0 wild type intact leaves (n=4 for each genotype). FIG. 13(B) Stomatal conductance in responses to [CO₂] changes in intact ost1-3 and Col-0 wild type plants (n=8 for ost1-3, n=6 for WT). FIG. 13(C) Stomatal conductance in responses to [CO₂] changes in intact ost1-1, ost1-2 and Ler wild type plants (n=4 for each genotype). Data shown in FIGS. 7B, C and D were normalized in (A), (B) and (C), respectively. FIG. 13(D) Stomatal conductance in responses to [CO₂] changes in pyr1;pyl1; pyl2;pyl4 quadruple mutant and Col-0 wild type intact leaves (n=4 for each genotype). Data shown in FIG. 8A were normalized in FIG. 13(D). Imposed CO₂ concentrations are shown at the bottom. Data are mean±s.e.

### DETAILED DESCRIPTION

In alternative embodiments, the invention provides compositions and methods for manipulating the exchange of water and carbon dioxide (CO₂) through plant stomata by controlling both CO₂ sensor genes, which can be designated “CO₂ Sen genes” and OST1 (Open Stomata 1, also known as SnRK2.6), SnRK2.2 or SnRK2.3 protein kinase genes (SnRK2 genes are SNF1 Related Protein Kinase Subfamily 2 genes) SNF1 is “Sucrose non-fermenting 1”). The invention provides compositions and methods for over or under-expressing CO₂ sensor nucleic acids and CO₂ sensor polypeptides and OST1, SnRK2.2 or SnRK2.3 protein kinase genes. The invention provides compositions and methods for over-expressing CO₂ sensor nucleic acids and CO₂ sensor polypeptides and OST1, SnRK2.2 or SnRK2.3 protein kinase genes, to engineer and improved CO₂ response in a plant, plant part, plant organ, a leaf, and the like.

While the invention is not based on any particular mechanism of action, embodiments of the invention are based on the elucidation of the mechanism for CO₂ control of gas exchange in plants. The inventors demonstrated that bicarbonate, but not elevated CO₂, acts as intracellular signaling molecule to activate SLAC1-mediated anion channels. Elevated bicarbonate enhances (primes) the [Ca2+]i sensitivity of SLAC1 channel activation. The ht1-2 kinase mutant is found to enhance the HCO₃_{^{−}} sensitivity of anion channel activation but also requires cytosolic Ca2+ for S-type anion channel activation, further defining the placement of HT1 effects on the CO₂ signaling cascade.

The inventors' analysis of OST1 on CO₂ regulation of stomatal movements and anion channels demonstrate that the OST1 protein kinase is a major regulator of CO₂-induced stomatal closing and CO₂ activation of anion channels in guard cells, leading to a new model for CO₂ control of gas exchange in plants and further possibilities to modulate the exchange of water and/or carbon dioxide (CO₂) through plant stomata.

Over-expression of one or several CO₂ sensor genes, including the CO₂ sensor nucleic acids (e.g., as genes or messages or transcripts), or CO₂ sensor polypeptides, and overexpression of OST1 protein kinase encoding nucleic acids (such as genes, messages or transcripts) evokes an improved CO₂ response. Thus, overexpression of both CO₂ sensor proteins and OST1, SnRK2.2 or SnRK2.3 protein kinase enhances WUE and produces a more efficient and drought resistant plant, particularly in light of the continuously rising atmospheric CO₂ concentrations.

In alternative embodiments, the invention provides transgenic plants (including crop plants, such as a field row plants), cells, plant tissues, seeds and organs, and the like, (which in alternative embodiments express one or more recombinant nucleic acids encoding all or one of the CO₂Sen proteins, and all or one of the OST1, SnRK2.2- or SnRK2.3 protein kinases) which can close their stomata to a greater extent that wild-type plants, thereby preserving their water usage. Because water use efficiency defines how well a plant can balance the loss of water through stomata with the net CO₂ uptake for photosynthesis, and hence its biomass accumulation, the compositions and methods of the invention can also be used to increase a plant's biomass, and thus the compositions and methods of the invention have applications in the biofuels/alternative energy area.

In alternative embodiments, the invention also provides compositions and methods for inhibiting the expression of CO2Sens genes, transcripts and CO2Sensor proteins and of OST1, SnRK2.2- or SnRK2.3 protein kinase genes, transcripts and CO2Sensor proteins using e.g. inhibitory RNA mediated repression (including antisense RNA, co-suppression RNA, siRNA, microRNA, double-stranded RNA, hairpin RNA and/or RNAi) of the expression of CO2 sensors and OST1, SnRK2.2- or SnRK2.3 protein kinase in cells, such as guard cells, in any plant including agricultural crops.

In alternative embodiments, the invention provides transgenic plants which have a lower expression of CO2sens proteins and OST1, SnRK2.2- or SnRK2.3 protein kinases (CO2sensor and OST1, SnRK2.2- or SnRK2.3-under-expressing plants) and can open their stomata to a greater extent than wild-type plants.

In alternative embodiments, the invention provides plants, plant cells, plant organs and the like, e.g., agriculture crops, that can withstand increased temperatures—thus preventing a “breakdown” of metabolism, photosynthesis and growth. Thus, compositions and methods of this invention, by inhibiting both the expression of CO2Sensor nucleic acids and/or CO2Sens proteins as well as expression of OST1, SnRK2.2- or SnRK2.3 protein kinase, help crops that otherwise would be sensitive to elevated temperatures to cope with the increased atmospheric CO2 concentrations, also reducing or ameliorating an accelerated increase in leaf temperatures.

In alternative embodiments, the invention provides compositions and methods comprising inhibitory RNA (including antisense and RNAi) for repression of CO2 sensors and OST1, SnRK2.2- or SnRK2.3 protein kinase expression in guard cells to reduce leaf temperature though enhancing transpiration in these crops and also to maximize crop yields.

In alternative embodiments, the invention provides compositions and methods for down-regulating/decreasing or alternatively increasing carbon dioxide (CO2) and/or water exchange in a plant, e.g., through the guard cell of a plant, plant cell, plant leaf, plant organ or plant part comprising inter alia use of a polypeptide having carbonic anhydrase, and an OST1, SnRK2.2- or SnRK2.3 protein kinase.

While the invention is not based on any particular mechanism of action, embodiments of compositions and methods of the invention are based on regulation of the opening or closing of stomata, including regulation of the efficiency of the exchange of water and CO2 through stomata can further be modulate or balanced in a more controlled way by controlling CO2 sensor and OST1, SnRK2.2- or SnRK2.3 protein kinase genes and/or transcripts thereby expressing or increasing the expression of CO2 sensor genes and/or transcripts and simultaneously decreasing the expression of OST1, SnRK2.2- or SnRK2.3 protein kinase genes and/or transcripts or inversely by decreasing the expression of CO2 sensor genes and/or transcripts and simultaneously expressing or increasing the expression of OST1, SnRK2.2- or SnRK2.3 protein kinase genes and/or transcripts.

In alternative embodiments, the invention provides methods for down-regulating or decreasing carbon dioxide (CO2) and/or water exchange in a guard cell of a plant, plant cell, plant leaf, plant organ or plant part comprising expressing in a cell a polypeptide having a carbonic anhydrase (carbonate dehydratase) activity, or a β-carbonic anhydrase activity in combination with a polypeptide having OST1, SnRK2.2- or SnRK2.3 protein kinase activity.

In alternative embodiments, any carbonic anhydrase (carbonate dehydratase) can be used, e.g., including plant or bacterial carbonic anhydrase (carbonate dehydratase) enzymes. Exemplary carbonic anhydrase (carbonate dehydratase) enzymes that can be used to practice this invention include carbonic anhydrase (carbonate dehydratase) enzymes isolated or derived from:

Rice (Oryza sativa)
NM_001072713 (= Genbank accession number)
Oryza sativa (japonica cultivar-group) Osl2g0153500 (Osl2g0153500) mRNA, complete
cds
gi|115487387|ref|NM_001072713.1|[115487387]
NM_001072308 (= Genbank accession number)
Oryza sativa (japonica cultivar-group) Os1lgO153200 (Os1lgO153200) mRNA,
complete cds
gi|115484228|ref|NM_001072308.1|[115484228]
NM_001069944 (= Genbank accession number)
Oryza sativa (japonica cultivar-group) Os09g0464000 (Os09g0464000) mRNA, complete
cds
gi|115479630|ref|NM_001069944.1|[115479630]
NM_001069887 (= Genbank accession number)
Oryza sativa (japonica cultivar-group) Os09g0454000 (Os09g0454500) mRNA, complete
cds
gi|115479516|ref|NM_001069887.1|[115479516]
NM_001068550 (= Genbank accession number)
Oryza sativa (japonica cultivar-group) Os08g0470200 (Os08g0470200) mRNA, complete
cds
gi|115476837|ref|NM_001068550.1|[115476837]
NM_001068366 (= Genbank accession number)
Oryza sativa (japonica cultivar-group) Os08g0423500 (Os08g0423500) mRNA, complete
cds
gi|115476469|ref|NM_001068366.1|[115476469]
NM_001064586 (= Genbank accession number)
Oryza sativa (japonica cultivar-group) Os06g0610100 (Os06g0610100) mRNA, complete
cds
gi|115468903|ref|NM_001064586.1|[115468903]
NM_001053565 (= Genbank accession number)
Oryza sativa (japonica cultivar-group) Os02g0533300 (Os02g0533300) mRNA, complete
cds
gi|115446500|ref|NM_001053565.1|[115446500]
NM00_1050212 (= Genbank accession number)
Oryza sativa (japonica cultivar-group) Os01g0640000 (Os01g0640000) mRNA, complete
cds
gi|115438794|ref|NM_001050212.1|[115438794]
NM_001050211 (= Genbank accession number)
Oryza sativa (japonica cultivar-group) Os01g0639900 (OsO1g0639900) mRNA, partial
cds
gi|115438792|ref|NM_001050211.11[115438792]
EF576561
Oryza sativa (indica cultivar-group) clone OSS-385-480-G10 carbonic anhydrase mRNA,
partial cds
gi|149392692|gb|EF576561.1|[149392692]
AF182806
Oryza sativa carbonic anhydrase 3 mRNA, complete cds
gi|5917782|gb|AF182806.1|AF182806[5917782]
U08404
Oryza sativa chloroplast carbonic anhydrase mRNA, complete cds
gi|606816|gb|U08404.1|OSU08404[606816]
Corn: (Zea mays)
NM_001111889
Zea mays carbonic anhydrase (LOC542302), mRNA
gi|162459146|ref|NM_001111889.1|[162459146]
U08403
Zea mays Golden Bantam carbonic anhydrase mRNA, complete cds
gi|606814|gb|U08403.1|ZMU08403 [606814]
U08401
Zea mays carbonic anhydrase mRNA, complete cds
gi|606810|gb|U08401.1|ZMU08401[606810]
M95073
Zea mays putative carbonic anhydrase homolog mRNA, partial cds gi|168561|
gb|M95073.1|MZEORFN[168561
Soybean: (Glycine max)
J239132
Glycine max mRNA for carbonic anhydrase
gi|4902524|emb|AJ239132.1|[4902524]
Tomato (Lycopersicon)
AJ849376
Lycopersicon esculentum mRNA for chloroplast carbonic anhydrase (ca2 gene)
gi|56562176]emb|AJ849376.1|[56562176]
AJ849375
Lycopersicon esculentum mRNA for carbonic anhydrase (ca1 gene)
gi|56562174|emb|AJ849375.1|[56562174]
Tobacco (Nicotiana)
AF492468
Nicotiana langsdorffu × Nicotiana sanderae neclarin III (NEC3) mRNA,
complete cds
gi|29468279|gb|AF492468.1|[29468279]
AF4554759
Nicotiana tabacum beta-carbonic anhydrase (CA) mRNA, complete cds; nuclear gene for
chloroplast product
gi|22550385|gb|AF454759.2|[22550385]
AB009887
Nicotiana tabacum mRNA for carbonic anhydrase, partial cds
gi|8096276|dbj|AB009887.1|[8096276]
AB012863
Nicotiana paniculata mRNA for NPCA1, complete cds
gi|3061270|dbj|AB012863.1|[3061270]
L19255
Nicotiana tabacum chloroplastic carbonic anhydrase mRNA, 3′ end
gi|310920|gb|L19255.1|TOBCARANHY[310920]
M94135
Nicotiana tabacum chloroplast carbonic anhydrase gene, complete cds
gi|170218|gb|M94135.1|TOBCLCAA[170218]
AY974608
Nicotiana benthamiana clone 30F62 chloroplast carbonic anhydrase mRNA, partial cds;
nuclear gene for chloroplast product
gi|62865756|gb|AY974608.1|[62865756]
AY974607
Nicotiana benthamiana clone 30C84 chloroplast carbonic anhydrase mRNA, partial cds;
nuclear gene for chloroplast product
gi|62865754|gb|AY974607.1|[62865754]
AY974606
Nicotiana benthamiana clone 3 OB 10 chloroplast carbonic anhydrase mRNA, partial cds;
nuclear gene for chloroplast product
gi|62865752|gb|AY974606.1|[62865752]
Barley (Hordeum)
L36959
Hordeum vulgare carbonic anhydrase mRNA, complete cds
gi|558498|gb|L36959.1|BLYCA[558498]
Cotton (Gossypium)
AF132855
Gossypium hirsutum carbonic anhydrase isoform 2 (CA2)
mRNA, partial cds; nuclear gene for plastid product
gi|4754914|gb|AF132855.1|AF132855[4754914]
AF132854
Gossypium hirsutum carbonic anhydrase isoform 1 (CA1) mRNA, partial cds; nuclear
gene for plastid product
gi|4754912|gb|AF132854.1|AF132854[4754912]
Poplar (Populus)
U55837
Populus tremula × Populus tremuloides carbonic anhydrase (CA1a) mRNA, nuclear gene
encoding chloroplast protein, complete cds
gi|1354514|gb|U55837.1|PTU55837[1354514]
U55838
Populus tremula × Populus tremuloides carbonic anhydrase (CA1b) mRNA, nuclear gene
encoding chloroplast protein, complete cds
gi|354516|gb|U55838.1|PTU55838[1354516]
Cucumis
DQ641132
Cucumis sativus clone CU8F3 carbonic anhydrase mRNA, partial cds
gi|117663159|gb|DQ641132.1|[117663159]
Medicago
X93312
M. sativa mRNA for carbonic anhydrase
gi|1938226|emb|X93312.1|[1938226]
Phaseolus
AJ547634
Phaseolus vulgaris partial mRNA for carbonic anhydrase (ca gene)
gi|28556429|emb|AJ547634.1|[28556429]
Pisum
X52558
Pea cap mRNA for carbonic anhydrase (EC 4.2.1.1)
gi|20672|emb|X52558.11[20672]
M63627
P. sativum carbonic anhydrase mRNA, complete cds
gi|169056|gb|M63627.1|PEACAMRA[169056]
Pyrus
AF195204
Pyrus pyrifolia strain Whangkeumbae carbonic anhydrase isoform 1 (Cal1)
mRNA, complete cds
gi|8698882|gb|AF195204.1|AF195204[8698882]
Prunus
EF640698
Prunus dulcis clone Pdbes-E45 putative carbonic anhydrase mRNA, partial cds
gi|148807206|gb|EF640698.1|[148807206]
Vigna
AF139464
Vigna radiata carbonic anhydrase (CipCal) mRNA, complete cds; nuclear gene for
chloroplast product
gi|8954288|gb|AF139464.2|AF139464[8954288]

In alternative embodiments, carbonic anhydrase encoding nucleic acids from any carbonic anhydrase gene, e.g., including plant and bacterial genes, can be used to practice this invention; for example, a nucleic acid from any carbonic anhydrase gene of any plant can be used, including any carbonic anhydrase-encoding nucleic acid sequence from any gene family of Arabidopsis, e.g., any carbonic anhydrase-encoding nucleic acid sequence from an Arabidopsis family, e.g., from Arabidopsis thaliana, can be used to practice the compositions and methods of this invention, such as the nucleic acid sequences encoding a polypeptide having the amino acid sequence of SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46. Such nucleotide sequences include the nucleotide sequence of SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, or SEQ ID NO:45.

In alternative embodiments, carbonic anhydrases encoding nucleic acids may be used having between 75% and 100% sequence identity to any of the nucleotide sequences above, which include those having at least about 71%, 72%, 73%, 74%, 75%, 76%, 77%, 78% or 79%, 80%, 81%, 82%, 83%, 84%, 85%, 86%, 87%, 88% or 89%, 90%, 91%, 92%, 93%, 94%, 95%, 96%, 97%, 98%, 99% or 100 % sequence identity to a nucleotide sequence encoding an amino acid sequence of any of SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46, such as a nucleotide sequence having 71%, 72%, 73%, 74%, 75%, 76%, 77%, 78% or 79%, 80%, 81%, 82%, 83%, 84%, 85%, 86%, 87%, 88% or 89%, 90%, 91%, 92%, 93%, 94%, 95%, 96%, 97%, 98%, 99% or 100% sequence identity to any nucleotide sequence of SEQ ID NO:1, SEQ ID NO:2, SEQ ID NO:3, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, or SEQ ID NO:45.

In alternative embodiments, OST1, SnRK2.2- or SnRK2.3 protein kinase encoding genes include genes encoding a polypeptide with OST1 protein kinase activity having between 75% and 100% sequence identity to the amino acid sequence of SEQ ID 12 or SEQ ID 14 including those having 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99% or 100% sequence identity to the amino acid sequence of SEQ ID NO:12 or SEQ ID NO:14. such nucleotide sequences may have 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleotide sequence of SEQ ID 11 or 13.

In alternative embodiments, compositions and methods of the invention comprise combinations, wherein the carbonic anhydrase can be either a β carbonic anhydrase 4 or a β carbonic anhydrase 1. In alternative embodiments, alternative (exemplary) combinations are:

i) Expressing, increasing the expression, upregulating a polypeptide with β carbonic anhydrase activity having an amino acid sequence sharing between 75% and 100% sequence identity to an amino acid of SEQ ID 8 (CA1) and expressing, increasing the expression or upregulating a polypeptide with OST1 protein kinase activity sharing between 75% and 100% sequence identity to the amino acid sequence of SEQ ID 12 (OST1.1)

ii) Expressing, increasing the expression, upregulating a polypeptide with β carbonic anhydrase activity having an amino acid sequence sharing between 75% and 100% sequence identity to an amino acid of SEQ ID 8 (CA1) and expressing, increasing the expression or upregulating a polypeptide with OST1 protein kinase activity sharing between 75% and 100% sequence identity to the amino acid sequence of SEQ ID 14 (OST1.2)

iii) Expressing, increasing the expression, upregulating a polypeptide with β carbonic anhydrase activity having an amino acid sequence sharing between 75% and 100% sequence identity to an amino acid of SEQ ID 3 (CA4) and expressing, increasing the expression or upregulating a polypeptide with OST1 protein kinase activity sharing between 75% and 100% sequence identity to the amino acid sequence of SEQ ID 12 (OST1.1)

iv) Expressing, increasing the expression, upregulating a polypeptide with β carbonic anhydrase activity having an amino acid sequence sharing between 75% and 100% sequence identity to an amino acid of SEQ ID 3 (CA4) and expressing, increasing the expression or upregulating a polypeptide with OST1 protein kinase activity sharing between 75% and 100% sequence identity to the amino acid sequence of SEQ ID 14 (OST1.1)

v) Expressing, increasing the expression, upregulating the expression of CA1 nucleotide sequence having between 75% and 100% sequence identity to an nucleotide sequence of SEQ ID 7 (CA1) and expressing, increasing the expression or upregulating the expression of OST1 protein kinase nucleotide sequence sharing between 75% and 100% sequence identity to an nucleotide sequence of SEQ ID 11 (OST1.1)

vi) Expressing, increasing the expression, upregulating the expression of CA1 nucleotide sequence having between 75% and 100% sequence identity to an nucleotide sequence of SEQ ID 7 (CA1) and expressing, increasing the expression or upregulating the expression of OST1 protein kinase nucleotide sequence sharing between 75% and 100% sequence identity to an nucleotide sequence of SEQ ID 13 (OST1.2)

vii) Expressing, increasing the expression, upregulating the expression of CA4 nucleotide sequence having between 75% and 100% sequence identity to an nucleotide sequence of SEQ ID 1 (CA4) and expressing, increasing the expression or upregulating the expression of OST1 protein kinase nucleotide sequence sharing between 75% and 100% sequence identity to an nucleotide sequence of SEQ ID 11 (OST1.1)

viii) Expressing, increasing the expression, upregulating the expression of CA4 nucleotide sequence having between 75% and 100% sequence identity to an nucleotide sequence of SEQ ID 1 (CA4) and expressing, increasing the expression or upregulating the expression of OST1 protein kinase nucleotide sequence sharing between 75% and 100% sequence identity to an nucleotide sequence of SEQ ID 13 (OST1.2)

ix) Expressing, increasing the expression, upregulating the expression of CA4 nucleotide sequence having between 75% and 100% sequence identity to an nucleotide sequence of SEQ ID 2 (CA4) and expressing, increasing the expression or upregulating the expression of OST1 protein kinase nucleotide sequence sharing between 75% and 100% sequence identity to an nucleotide sequence of SEQ ID 11 (OST1.1)

x) Expressing, increasing the expression, upregulating the expression of CA4 nucleotide sequence having between 75% and 100% sequence identity to an nucleotide sequence of SEQ ID 2 (CA4) and expressing, increasing the expression or upregulating the expression of OST1 protein kinase nucleotide sequence sharing between 75% and 100% sequence identity to an nucleotide sequence of SEQ ID 13 (OST1.2)

xi) Reducing or downregulating the expression of a polypeptide with β carbonic anhydrase activity having an amino acid sequence sharing between 75% and 100% sequence identity to an amino acid of SEQ ID 8 (CA1) and expressing, increasing the expression or upregulating a polypeptide with OST1 protein kinase activity sharing between 75% and 100% sequence identity to the amino acid sequence of SEQ ID 12 (OST1.1)

xii) Reducing or downregulating the expression of a polypeptide with β carbonic anhydrase activity having an amino acid sequence sharing between 75% and 100% sequence identity to an amino acid of SEQ ID 8 (CA1) and expressing, increasing the expression or upregulating a polypeptide with OST1 protein kinase activity sharing between 75% and 100% sequence identity to the amino acid sequence of SEQ ID 14 (OST1.2)

xiii) Reducing or downregulating the expression of a polypeptide with β carbonic anhydrase activity having an amino acid sequence sharing between 75% and 100% sequence identity to an amino acid of SEQ ID 3 (CA4) and expressing, increasing the expression or upregulating a polypeptide with OST1 protein kinase activity sharing between 75% and 100% sequence identity to the amino acid sequence of SEQ ID 12 (OST1.1)

xiv) Reducing or downregulating the expression of a polypeptide with β carbonic anhydrase activity having an amino acid sequence sharing between 75% and 100% sequence identity to an amino acid of SEQ ID 3 (CA4) and expressing, increasing the expression or upregulating a polypeptide with OST1 protein kinase activity sharing between 75% and 100% sequence identity to the amino acid sequence of SEQ ID 14 (OST1.2)

xv) Reducing or downregulating the expression of a CA1 nucleotide sequence having between 75% and 100% sequence identity to an nucleotide sequence of SEQ ID 7 (CA1) and expressing, increasing the expression or upregulating the expression of OST1 protein kinase nucleotide sequence sharing between 75% and 100% sequence identity to an nucleotide sequence of SEQ ID 11 (OST1.1)

xvi) Reducing or downregulating the expression of a CA1 nucleotide sequence having between 75% and 100% sequence identity to an nucleotide sequence of SEQ ID 7 (CA1) and expressing, increasing the expression or upregulating the expression of OST1 protein kinase nucleotide sequence sharing between 75% and 100% sequence identity to an nucleotide sequence of SEQ ID 13 (OST1.2)

xvii) Reducing or downregulating the expression of a CA4 nucleotide sequence having between 75% and 100% sequence identity to an nucleotide sequence of SEQ ID 1 (CA4) and expressing, increasing the expression or upregulating the expression of OST1 protein kinase nucleotide sequence sharing between 75% and 100% sequence identity to an nucleotide sequence of SEQ ID 11 (OST1.1)

xviii) Reducing or downregulating the expression of a CA4 nucleotide sequence having between 75% and 100% sequence identity to an nucleotide sequence of SEQ ID 1 (CA4) and expressing, increasing the expression or upregulating the expression of OST1 protein kinase nucleotide sequence sharing between 75% and 100% sequence identity to an nucleotide sequence of SEQ ID 13 (OST1.2)

xix) Reducing or downregulating the expression of a CA4 nucleotide sequence having between 75% and 100% sequence identity to an nucleotide sequence of SEQ ID 2 (CA4) and expressing, increasing the expression or upregulating the expression of OST1 protein kinase nucleotide sequence sharing between 75% and 100% sequence identity to an nucleotide sequence of SEQ ID 11 (OST1.1)

xx) Reducing or downregulating the expression of a CA4 nucleotide sequence having between 75% and 100% sequence identity to an nucleotide sequence of SEQ ID 2 (CA4) and expressing, increasing the expression or upregulating the expression of OST1 protein kinase nucleotide sequence sharing between 75% and 100% sequence identity to an nucleotide sequence of SEQ ID 13 (OST1.2)

In alternative embodiments, the invention provides combinations between upregulating one protein and downregulating the expression of another protein, e.g., as set forth in the above paragraphs i) to xx), which can be made as described herein.

In alternative embodiments, expression or upregulating of the expression of a protein can be achieved by introduction (e.g., through transformation or crossing with a transgenic plant) or a recombinant gene comprising one, several or all of the following operably linked fragments
i. a plant expressible promoter;
ii. an, optionally heterologus, DNA fragment encoding a polypeptide having a carbonic anhydrase (CA) activity or a β-carbonic anhydrase activity and
iii. optionally, a transcription termination and polyadenylation signal; or
i. a plant expressible promoter;
ii. an, optionally heterologus, DNA fragment encoding a polypeptide with OST1 protein kinase activity;
iii. optionally, a transcription termination and polyadenylation signal.

### Plant (Expressible) Promoters

In alternative embodiments, nucleic acids, protein coding sequences or genes used to practice the invention is oeprably linked to a plant expressible promoter, an inducible promoter, a constitutive promoter, a guard cell specific promoter, a drought-inducible promoter, a stress-inducible promoter or a guard cell active promoter. Promoters used to practice the invention include a strong promoter, particularly in plant guard cells, and in some embodiments is guard cell specific, e.g., the promoters described in WO2008/134571.

In alternative embodiments, nucleic acids, protein coding sequences or genes also can be operatively linked to any constitutive and/or plant specific, or plant cell specific promoter, e.g., a cauliflower mosaic virus (CaMV) 35S promoter, a mannopine synthase (MAS) promoter a 1′ or 2′ promoter derived from T-DNA of Agrobacterium tumefaciens, a figwort mosaic virus 34S promoter, an actin promoter, a rice actin promoter, a ubiquitin promoter, e.g., a maize ubiquitin-1 promoter, and the like.

Examples of constitutive plant promoters which can be useful for expressing the sequences in accordance with the invention include: the cauliflower mosaic virus (CaMV) 35S promoter, which confers constitutive, high-level expression in most plant tissues (see, e.g., Odell et al. (1985) Nature 313:810-812); the nopaline synthase promoter (An et al. (1988) Plant Physiol. 88: 547-552); and the octopine synthase promoter (Fromm et al. (1989) Plant Cell 1:977-984).

A variety of plant gene promoters that regulate gene expression in response to environmental, hormonal, chemical, developmental signals, and in a tissue-active manner can be used for expression of a sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like.

Numerous known promoters have been characterized and can be employed to promote expression of a polynucleotide used to practice the invention, e.g., in a trangenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A1 1 promoter (e.g., see U.S. Pat. No. 4,943,674) and the tomato polygalacturonase promoter (e.g., see Bird et al (1988) Plant Mol. Biol. 11:651-662), root-specific promoters, such as those disclosed in U.S. Pat. Nos. 5,618,988, 5,837,848 and 5,905,186, pollen-active promoters such as PTA29, PTA26 and PTA13 (e.g., see U.S. Pat. No. 5,792,929), promoters active in vascular tissue (e.g., see Ringli and Keller (1998) Plant Mol. Biol. 37:977-988), flower-specific (e.g., see Kaiser et al. (1995) Plant Mol. Biol. 28:231-243), pollen (e.g., see Baerson et al. (1994) Plant Mol. Biol. 26:1947-1959), carpels (e.g., see Ohl et al. (1990) Plant Cell 2:, pollen and ovules (e.g., see Baerson et al. (1993) Plant Mol. Biol. 22:255-267), auxin-inducible promoters (such as that described in van der Kop et al. (1999) Plant Mol. Biol. 39: 979-990 or Baumann et al., (1999) Plant Cell 11:323-334), cytokinin-inducible promoter (e.g., see Guevara-Garcia (1998) Plant Mol. Biol. 38:743-753), promoters responsive to gibberellin (e.g., see Shi et al. (1998) Plant Mol. Biol. 38:1053-1060, Willmott et al. (1998) Plant Molec. Biol. 38:817-825) and the like.

Additional promoters that can be used to practice this invention are those that elicit expression in response to heat (e.g., see Ainley et al. (1993) Plant Mol. Biol. 22:13-23), light (e.g., the pea rbcS-3A promoter, Kuhlemeier et al. (1989) Plant Cell 1:471-478, and the maize rbcS promoter, Schaffher and Sheen (1991) Plant Cell 3:997-1012); wounding (e.g., wunl, Siebertz (1989) Plant Cell 1:961-968); pathogens (such as the PR-I promoter described in Buchel et al. (1999) Plant Mol. Biol. 40: 387-396, and the PDF 1.2 promoter described in Manners et al. (1998) Plant Mol. Biol. 38: 1071-1080), and chemicals such as methyl jasmonate or salicylic acid (e.g., see Gatz (1997) Annu. Rev. Plant Physiol. Plant Mol. Biol. 48: 89-108). In addition, the timing of the expression can be controlled by using promoters such as those acting at senescence (e.g., see Gan and Amasino (1995) Science 270: 1986-1988); or late seed development (e.g., see Odell et al. (1994) Plant Physiol. 106: 447-458).

In alternative embodiments, tissue-specific and/or developmental stage-specific promoters are used, e.g., promoter that can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene AP1; and Mandel (1995) Plant Molecular Biology, Vol. 29, pp 995-1004, describing the meristem promoter elF4. Tissue specific promoters which are active throughout the life cycle of a particular tissue can be used. In one aspect, the nucleic acids of the invention are operably linked to a promoter active primarily only in cotton fiber cells, in one aspect, the nucleic acids of the invention are operably linked to a promoter active primarily during the stages of cotton fiber cell elongation, e.g., as described by Rinehart (1996) supra. The nucleic acids can be operably linked to the Fb12A gene promoter to be preferentially expressed in cotton fiber cells (Ibid). See also, John (1997) Proc. Natl. Acad. Sci. USA 89:5769-5775; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60). Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF 13 promoter from Agrobacterium rhizogenes (which exhibits high activity in roots, see. e.g., Hansen (1997) supra); a maize pollen specific promoter (see, e.g., Guerrero (1990) Mol. Gen. Genet. 224:161 168); a tomato promoter active during fruit ripening, senescence and abscission of leaves and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

In alternative embodiments, plant promoters which are inducible upon exposure to plant hormones, such as auxims, are used to express the nucleic acids used to practice the invention. For example, the invention can use the auxin-response elements E1 promoter fragment (AuxREs) in the soybean (Glycine max L.) (Liu (1997) Plant Physiol. 115:397-407); the auxim-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274: 1900-1902).

In alternative embodiments, nucleic acids used to practice the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- (e.g., hormone- or pesticide-) induced promoters, i.e., promoter responsive to a chemical which can be applied to the transgenic plant in the field, expression of a polypeptide of the invention can be induced at a particular stage of development of the plant.

In alternative embodiments, the invention also provides for transgenic plants containing an inducible gene encoding for polypeptides used to practice the invention whose host range is limited to target plant species, such as corn, rice, barley, wheat, potato or other crops, inducible at any stage of development of the crop.

In alternative embodiments, a tissue-specific plant promoter may drive expression of operably linked sequences in tissues other than the target tissue. In alternative embodiments, a tissue-specific promoter that drives expression preferentially in the target tissue or cell type, but may also lead to some expression in other tissues as well, is used.

In alternative embodiments, proper polypeptide expression may require polyadenylation region at the 3′-end of the coding region. The polyadenylation region can be derived from the natural gene, from a variety of other plant (or animal or other) genes, or from genes in the Agrobacterial T-DNA.

### Antisense Inhibitory Molecules

In alternative embodiments, downregulation of CO₂sensor genes or OST1, SnRK2.2 or SnRK2.3 genes or transcripts can be achieved by introduction of a recombinant gene expressing inhibitory RNA targeted towards CO₂sensor genes or OST1, either separately or together.

In alternative embodiments, the invention provides an antisense inhibitory molecules comprising a sequence used to practice this invention (which include both sense and antisense strands), e.g., which target CO₂sensor genes or OST1, SnRK2.2 or SnRK2.3 genes or transcripts. Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl)glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J. 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

### RNA Interference (RNAi)

In one aspect, the invention provides an RNA inhibitory molecule, a so-called “RNAi” molecule, comprising a sequence used to practice this invention. In alternative embodiments, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA)molecules. The RNAi molecule, e.g., siRNA (small inhibitory RNA) can inhibit expression of a CO2Sen genes or OST1 genes, and/or miRNA (micro RNA) to inhibit translation of a CO2Sen genes or OST1 genes.

In alternative aspects, the RNAi is about 11, 12, 13, 14, 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's of the invention. The process may be practiced in vitro, ex vivo or in vivo. In one aspect, the RNAi molecules of the invention can be used to generate a loss-of-function mutation in a cell, an plant tissue or organ or seed, or a plant.

In alternative embodiments, intracellular introduction of the RNAi (e.g., miRNA or siRNA) is by internalization of a target cell specific ligand bonded to an RNA binding protein comprising and RNAi (e.g., microRNA) is adsorbed. The ligand is specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, the invention provides lipid-based formulations for delivering, e.g., introducing nucleic acids of the invention as nucleic acid-lipid particles comprising and RNAi molecule to a cell, see e.g., U.S. Patent App. Pub. No. 20060008910.

In alternative embodiments, methods for making and using RNAi molecules, e.g., siRNA and/or miRNA, for selectively degrade RNA include, e.g., U.S. Pat. No. 6,506,559; 6,511,824; 6,515,109; 6,489,127.

In alternative embodiments, known and routine methods for making expression constructs, e.g., vectors or plasmids, from which an inhibitory polynucleotide (e.g., a duplex siRNA of the invention) is transcribed are used. A regulatory region (e.g., promoter, enhancer, silencer, splice donor, acceptor, etc.) can be used to transcribe an RNA strand or RNA strands of an inhibitory polynucleotide from an expression construct. When making a duplex siRNA (e.g., to a CO₂Sen gene, or OST1, SnRK2.2 or SnRK2.3 gene) inhibitory molecule, the sense and antisense strands of the targeted portion of the targeted IRES can be transcribed as two separate RNA strands that will anneal together, or as a single RNA strand that will form a hairpin loop and anneal with itself.

For example, in alternative embodiments, a construct targeting a portion of a CO2Sen gene or OST1, SnRK2.2 or SnRK2.3 gene is inserted between two promoters (e.g., two plant, viral, bacteriophage T7 or other promoters) such that transcription occurs bidirectionally and will result in complementary RNA strands that may subsequently anneal to form an inhibitory siRNA of the invention. Alternatively, a targeted portion of a CO₂Sen gene or OST1, SnRK2.2 or SnRK2.3 can be designed as a first and second coding region together on a single expression vector, wherein the first coding region of the targeted gene is in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene or inhibitory siRNA, e.g., a CO₂Sen gene-or OST1, SnRK2.2 or SnRK2.3 gene inhibitory siRNA used to practice the invention.

In alternative embodiments, transcription of the sense and antisense targeted portion of the targeted nucleic acid, e.g., a CO2Sen gene, or OST1, SnRK2.2 or SnRK2.3 gene, is controlled by a single promoter, and the resulting transcript will be a single hairpin RNA strand that is self-complementary, e.g., forms a duplex by folding back on itself to create a (e.g., CO2Sen gene, or OST1, SnRK2.2 or SnRK2.3 gene)-inhibitory siRNA molecule. In this configuration, a spacer, e.g., of nucleotides, between the sense and antisense coding regions of the targeted portion of the targeted (e.g., CO2Sen gene-or OST1, SnRK2.2 or SnRK2.3) gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

### Inhibitory Ribozymes

In alternative embodiments, the invention provides ribozymes capable of binding CO2 sensor and/or OST1, SnRK2.2 or SnRK2.3 coding sequence, gene or message. These ribozymes can inhibit gene activity by e.g., targeting mRNA.

Strategies for designing ribozymes and selecting the gene specific antisense sequence for targeting are well described in the scientific and patent literature, and the skilled artisan can design such ribozymes using the reagents and sequences used to practice this invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly

### Plants Comprising Nucleic Acids of This Invention

In alternative embodiments, the invention provides transgenic plants, plant parts, plant organs or tissue, and seeds comprising nucleic acids, polypeptides, expression cassettes or vectors or a transfected or transformed cell of the invention. The invention also provides plant products, e.g., seeds, leaves, extracts and the like, comprising a nucleic acid and/or a polypeptide according to the invention. In alternative embodiments, the transgenic plant can be dicotyledonous (a dicot) or monocotyledonous (a monocot). The invention also provides methods of making and using these transgenic plants and seeds. The transgenic plant or plant cell expressing a polypeptide of the present invention may be constructed in accordance with any method known in the art. See, for example, U.S. Pat. No. 6,309,872.

Nucleic acids and expression constructs used to practice the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's CO2Sen protein production is regulated by endogenous transcriptional or translational control elements, or by a heterologous promoter, e.g., a promoter of this invention. The invention also provides “knockout plants” where insertion of gene sequence by, e.g., homologous recombination, has disrupted the expression of the endogenous gene. Means to generate “knockout” plants are well-known in the art.

The nucleic acids and polypeptides used to practice the invention can be expressed in or inserted in any plant, plant part, plant cell or seed. Transgenic plants of the invention, or a plant or plant cell comprising a nucleic acid used to practice this invention (e.g., a transfected, infected or transformed cell) can be dicotyledonous or monocotyledonous. Examples of monocots comprising a nucleic acid of this invention, e.g., as monocot transgenic plants of the invention, are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicots comprising a nucleic acid of this invention, e.g., as dicot transgenic plants of the invention, are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, plant or plant cell comprising a nucleic acid of this invention, including the transgenic plants and seeds of the invention, include a broad range of plants, including but not limited to, species from the genera Anacardium, Arachis, Asparagus, Atropa, Avena, Brassica, Citrus, Citrullus, Capsicum, Carthamus, Cocos, Cojfea, Cucumis, Curcurbita, Daucus, Elaeis, Fragaria, Glycine, Gossypium, Hellanthus, Heterocallis, Hordeum, Hyascyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, or Zea.

The nucleic acids and polypeptides used to practice this invention can be expressed in or inserted in any plant cell, organ, seed or tissue, including differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

### Transgenic Plants

In alternative embodiments, the invention provides transgenic plants, plant cells, organs, seeds or tissues, comprising and expressing the nucleic acids used to practice this invention, e.g., CO2Sen gene and proteins and OST1, SnRK2.2 or SnRK2.3 genes; for example, the invention provides plants, e.g., transgenic plants, plant cells, organs, seeds or tissues that show improved growth under limiting water conditions; thus, the invention provides drought-tolerant plants, plant cells, organs, seeds or tissues (e.g., crops).

A transgenic plant of this invention can also include the machinery necessary for expressing or altering the activity of a polypeptide encoded by an endogenous gene, for example, by altering the phosphorylation state of the polypeptide to maintain it in an activated state.

Transgenic plants (or plant cells, or plant explants, or plant tissues) incorporating the polynucleotides of the invention and/or expressing the polypeptides of the invention can be produced by a variety of well-established techniques as described above.

Following construction of a vector, most typically an expression cassette, including a polynucleotide, e.g., encoding a transcription factor or transcription factor homolog, of the invention, standard techniques can be used to introduce the polynucleotide into a plant, a plant cell, a plant explant or a plant tissue of interest. In one aspect the plant cell, explant or tissue can be regenerated to produce a transgenic plant.

The plant can be any higher plant, including gymnosperms, monocotyledonous and dicotyledonous plants. Suitable protocols are available for Leguminosae (alfalfa, soybean, clover, etc.), Umbelliferae (carrot, celery, parsnip), Cruciferae (cabbage, radish, rapeseed, broccoli, etc.), curcurbitaceae (melons and cucumber), Gramineae (wheat, corn, rice, barley, millet, etc.), Solanaceae (potato, tomato, tobacco, peppers, etc.), and various other crops. See protocols described in Ammirato et al., eds., (1984) Handbook of Plant Cell Culture—Crop Species, Macmillan Publ. Co., New York, N.Y.; Shimamoto et al. (1989) Nature 338: 274-276; Fromm et al. (1990) Bio/Technol. 8:833-839; and Vasil et al. (1990) Bio/Technol. 8: 429-434.

Transformation and regeneration of both monocotyledonous and dictoyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and

In alternative embodiments, the invention uses Agrobacterium tumefaciens mediated transformation. Transformation means introducing nucleotide sequence into a plant in a manner to cause stable or transient expression of the sequence.

Successful examples of the modification of plant characteristics by transformation with cloned sequences which serve to illustrate the current knowledge in this field of technology, and include for example: U.S. Pat. Nos. 5,571,706; 5,677,175; 5,510,471; 5,750,386; 5,597,945; 5,589615; 5,750871; 5,268,526; 5,780,708, 5,538,880; 5,773,269; 5,736,369 and 5,619,042.

In alternative embodiments, following transformation, plants are selected using a dominant selectable marker incorporated into the transformation vector. Such a marker can confer antibiotic or herbicide resistance on the transformed plants, and selection of transformants can be accomplished by exposing the plants to appropriate concentrations of the antibiotic or herbicide.

In alternative embodiments, after transformed plants are selected and grown to maturity, those plants showing a modified trait are identified. The modified trait can by any of those traits described above. In alternative embodiments, to confirm that the modified trait is due to changes in expression levels or activity of the transgenic polypeptide or polynucleotide can be determined by analyzing mRNA expression using Northern blots, RT-PCR or microarrays, or protein expression using immunoblots or Western blots or gel shift assays.

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's CO2 sensor production is regulated by endogenous transcriptional or translational control elements.

In alternative embodiments, the invention also provides “knockout plants” where insertion of gene sequence by, e.g., homologous recombination, has disrupted the expression of the endogenous gene. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants below.

In alternative embodiments, making transgenic plants or seeds comprises incorporating sequences used to practice the invention and, in one aspect (optionally), marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, e.g., Christou (1997) Plant Mol. biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In alternative embodiments, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

In alternative embodiments, a third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

In alternative embodiments, after the expression cassette is stably incorporated in transgenic plants, it can be introduced into other plants by sexual crossing. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides, e.g., a CO₂ sensor and OST1, SnRK2.2 or SnRK2.3 gene of the invention. The desired effects can be passed to future plant generations by standard propagation means.

The invention will be further described with reference to the examples described herein; however, it is to be understood that the invention is not limited to such examples.

### EXAMPLES

### Example 1

The following non-limiting Example demonstrates that genes and proteins of a CO₂ signaling pathway and the use of CO₂ sensor genes and OST1, SnRK2.2 or SnRK2.3 protein kinase genes can modulate stomatal movement.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A laboratory Manual, Cold Spring Harbor Laboratory Press, and in McPherson et al. (2000) PCR—Basics: From Background to Bench, First Edition, Spring Verlag, Germany.

Throughout the description and Examples, reference is made to the following sequences:

SEQ ID NO:1: nucleotide sequence of β carbonic anhydrase 4 (CA4) from Arabidopsis thaliana (At1g70410)

SEQ ID NO:2: nucleotide sequence of β carbonic anhydrase 4 (CA4) from Arabidopsis thaliana—coding sequence.

SEQ ID NO:3: nucleotide sequence of β carbonic anhydrase 4 (CA4) from Arabidopsis thaliana.

SEQ ID NO:4: nucleotide sequence of β carbonic anhydrase 6 (CA6) from Arabidopsis thaliana (At1g58180)

SEQ ID NO:5: nucleotide sequence of β carbonic anhydrase 6 (CA6) from Arabidopsis thaliana—coding sequence.

SEQ ID NO:6: nucleotide sequence of β carbonic anhydrase 6 (CA6) from Arabidopsis thaliana.

SEQ ID NO:7: nucleotide sequence of β carbonic anhydrase 1 (CA1) from Arabidopsis thaliana—variant 1

SEQ ID NO:8: nucleotide sequence of β carbonic anhydrase 1 (CA1) from Arabidopsis thaliana—variant 1

SEQ ID NO:9: nucleotide sequence of β carbonic anhydrase 1 (CA1) from Arabidopsis thaliana—variant 2

SEQ ID NO:10: nucleotide sequence of β carbonic anhydrase 1 (CA1) from Arabidopsis thaliana—variant 2

SEQ ID NO:11: nucleotide sequence of OST1 protein kinase cDNA from Arabidopsis thaliana —variant 1

SEQ ID NO:12: amino acid sequence of OST1 protein kinase cDNA from Arabidopsis thaliana —variant 1

SEQ ID NO:13: nucleotide sequence of OST1 protein kinase cDNA from Arabidopsis thaliana —variant 2

SEQ ID NO:14: amino acid sequence of OST1 protein kinase cDNA from Arabidopsis thaliana —variant 2

SEQ ID NO:15: nucleotide sequence of A. thaliana β carbonic anhydrase 2 (CA2) cDNA (At5g14740)

SEQ ID NO:16: amino acid sequence of A. thaliana β carbonic anhydrase 2 (CA2) cDNA (At5g14740)

SEQ ID NO:17: nucleotide sequence of A. thaliana α carbonic anhydrase 1 (CA1) cDNA (At3g52720)

SEQ ID NO:18: nucleotide sequence of A. thaliana α carbonic anhydrase 1 (CA1) cDNA (At3g52720)

SEQ ID NO:19: nucleotide sequence of A. thaliana α carbonic anhydrase 2 (CA2) cDNA (At2g28210)

SEQ ID NO:20: amino acid sequence of A. thaliana α carbonic anhydrase 1 (CA1) cDNA (At3g52720)

SEQ ID NO:21: nucleotide sequence of A. thaliana α carbonic anhydrase 3 (CA3) cDNA (At5g04180)

SEQ ID NO:22: amino acid sequence of A. thaliana α carbonic anhydrase 3 (CA3) cDNA (At5g04180)

SEQ ID NO:23: nucleotide sequence of A. thaliana α carbonic anhydrase 4 (CA4) cDNA (At4g20990)

SEQ ID NO:24: amino acid sequence of A. thaliana α carbonic anhydrase 2 (CA4) cDNA (At4g20990)

SEQ ID NO:25: nucleotide sequence of A. thaliana α carbonic anhydrase 5 (CA5) cDNA (At1g08065)

SEQ ID NO:26: amino acid sequence of A. thaliana α carbonic anhydrase 5 (CA5) cDNA (At1g08065)

SEQ ID NO:27: nucleotide sequence of A. thaliana α carbonic anhydrase 6 (CA6) cDNA (At4g21000)

SEQ ID NO:28: amino acid sequence of A. thaliana α carbonic anhydrase 6 (CA6) cDNA (At4g21000)

SEQ ID NO:29: nucleotide sequence of A. thaliana α carbonic anhydrase 7 (CA7) cDNA (At1g08080)

SEQ ID NO:30: amino acid sequence of A. thaliana α carbonic anhydrase 7 (CA7) cDNA (At1g08080)

SEQ ID NO:31: nucleotide sequence of A. thaliana α carbonic anhydrase 8 (CA8) cDNA (At5g56330)

SEQ ID NO:32: amino acid sequence of A. thaliana α carbonic anhydrase 8 (CA8) cDNA (At5g56330)

SEQ ID NO:33: nucleotide sequence of A. thaliana β carbonic anhydrase 3 (CA3) cDNA (At1g23730)

SEQ ID NO:34: amino acid sequence of A. thaliana β carbonic anhydrase 3 (CA3) cDNA (At1g23730)

SEQ ID NO:35: nucleotide sequence of A. thaliana β carbonic anhydrase 5 (CA5) cDNA (At4g33580)

SEQ ID NO:36: amino acid sequence of A. thaliana β carbonic anhydrase 5 (CA5) cDNA (At4g33580)

SEQ ID NO:37: nucleotide sequence of A. thaliana γ carbonic anhydrase 1 (CA1) cDNA (At1g19580)

SEQ ID NO:38: amino acid sequence of A. thaliana γ carbonic anhydrase 1 (CA1) cDNA (At1g19580)

SEQ ID NO:39: nucleotide sequence of A. thaliana γ carbonic anhydrase 2 (CA2) cDNA (At1g47260)

SEQ ID NO:40: amino acid sequence of A. thaliana γ carbonic anhydrase 2 (CA2) cDNA (At1g47260)

SEQ ID NO:41: nucleotide sequence of A. thaliana γ carbonic anhydrase 3 (CA3) cDNA (At5g66510)

SEQ ID NO:42: amino acid sequence of A. thaliana γ carbonic anhydrase 3 (CA3) cDNA (At5g66510)

SEQ ID NO:43: nucleotide sequence of A. thaliana γ carbonic anhydrase like 1 (CAL1) cDNA (At5g63510)

SEQ ID NO:44: amino acid sequence of A. thaliana γ carbonic anhydrase like 1 (CAL1) cDNA (At5g63510)

SEQ ID NO:45: nucleotide sequence of A. thaliana γ carbonic anhydrase2 (CAL2) cDNA (At3g48680)

SEQ ID NO:46: amino acid sequence of A. thaliana γ carbonic anhydrase 2 (CAL2) (At3g48680)

### Materials and Methods

### Plant Growth

The Arabidopsis mutant lines analyzed in this study were ca1;ca4 (Hu et al, 2010), Slac1-1, slac1-3 (Vahisalu et al, 2008), ht1-2 (Hashimoto et al, 2006), ost1-1, ost1-2 (Mustilli et al, 2002), ost1-3 (Yoshida et al, 2002), abi1-1, abi2-1 and pyr1;pyl1; pyl2;pyl4 in the backcrossed Columbia background (Nishimura et al, 2010), Plants were grown in a plant growth chamber at 21° C. temperature, 65%-85% humidity, except that abi1-1 and abi2-1 were grown constantly at 75-85% humidity and a 16-h-light/8-h-dark photoperiod regime at ˜75 μmol m⁻²s⁻¹.

### Electrophysiology

Arabidopsis guard cell protoplasts were isolated as described previously (Siegel et al, 2009). Whole-cell patch-clamp experiments were performed as described previously (Pei et al, 1997). During recordings of S-type anion currents, the membrane voltage was stepped to potentials starting at±35 mV to −145 mV for 7 s with −30 mV decrements and the holding potential was +30 mV. The interpulse period was 5 s. Liquid junction potentials (LJP) were determined using Clampex 10.0. No leak subtraction was applied for all current-voltage curves. Steady-state currents were the average currents during the last 500 ms of pulses. Detail contents of solutions are discussed, below (see “supplementary data”). Bicarbonate (CsHCO₃) was freshly dissolved in the pipette solution before patch clamp experiments and pH was adjusted to the indicated values. The pipette solution was stored using air-tight precision glass syringes during patch clamp experiments to slow CO₂ equilibration with the surrounding air and was not stored overnight. The concentrations of free CO₂ and bicarbonate in solutions were calculated using the Henderson-Hasselbalch equation (pH=pK₁+log [HCO₃⁻]/[CO₂]) (Hauser et al, 1995). [HCO₃⁻] represents the free bicarbonate concentration; [CO₂] represents the free CO₂ concentration. A value, pK₁=6.352, was used for calculations (Speight, 2005). To independently measure CO₂ concentrations in the solutions at different pH values, an InPro 5000 CO₂ sensor (Mettler Tolego 400, Mettler-Toledo Inc, USA) was used for dissolved CO₂. The InPro 5000 sensor employs a gas permeable silicone membrane. The significance of differences between data sets was assessed by noncoupled double-tailed Student's t-test analysis. Values of P<0.05 were considered statistically significant.

### Expression of pH Sensor Pt-GFP in Arabidopsis Guard Cells

The Pt-GFP cDNA was amplified with the primers PGF (5′-AACCATGGCGCAGACCTTCCTCTAT-3′, with NcoI site) and PGR (5′-AACTGCAGAGGCGTCTCGCATATCTC-′, with PstI site) from the construct pART7-PrGFP (Schulte et al, 2006), kindly provided by Dr. Christoph Plieth. The sequenced PCR product was digested with NcoI and PstI and then subcloned into the binary expression vector pGreenII 0179-pGCP(D1)-terminator under the control of guard cell specific promoter pGC1 (Yang et al, 2008). The construct pGC1::PtGFP was transformed to the Agrobacterium strain GV3101 containing helper plasmid pSOUP and then was introduced into Arabidopsis (Col-0) by the floral dip method (Clough & Bent, 1998).

### Fluorescence Imaging of Guard Cells Expressing Pt-GFP

Fluorescence imaging was performed with a TE300 inverted microscope using a TE-FM Epi-Fluorescence attachment (Nikon) as previously described (Allen et al, 2000). Fluorescence images at excitation wavelengths of 470 nm and 440 nm were taken every 2 s using light from a 75-Watt xenon short arc lamp (Osram, Germany). 32° neutral density filters were used to reduce bleaching of fluorescent reporter. Metafluor software (MDS, Inc.) was used to control filter wheels, shutter and COOLSNAP™ (CoolSNAP) CCD camera from Photomerics when recording and also processing raw data. The fluorescence ratio F470/F440 of Pt-GFP was analyzed as a detection of pH shifts (Schulte et al, 2006). Intact epidermes from pGC1::PtGFP expressing leaves were prepared and affixed to glass coverslips using medical adhesive (Hollister Incorporated Libertyville, Ill. USA) and then adhered to a glass slide with a hole in the middle generating a well, as described (Hu et al, 2010; Siegel et al, 2009; Young et al, 2006).

For recording intracellular Pt-GFP fluorescence in response to changes in extracellular pH incubation buffers, the pH of incubation buffers containing 10 mM MES, 10 mM KCl and 50 μM CaCl₂ at 5.0 and 7.5 was adjusted by adding Tris-HCl. The well was perfused with incubation buffer at pH 5.0 for 15 min to obtain a background value and subsequently perfused with buffer at pH 7.5 for 15 min and returned to pH 5.0 again. For recording intracellular Pt-GFP fluorescence in response to constant extracellular pH and added weak acid, the perfusion buffers contained 10 mM MES, 10 mM KCl and 50 μM CaCl₂, pH 5.6 supplemented with the indicated concentrations of sodium butyrate. For recording the Pt-GFP fluorescence of guard cells in response to CO₂ changes, the incubation buffer (10 mM MES, 10 mM KCl and 50μM CaCl₂, pH 6.15) was continually bubbled with 800 ppm CO₂ or bubbled with air through soda lime, which was considered as nominal 0 ppm CO₂ inside the buffer. Note that the final CO₂ concentrations to which leaf epidermes were exposed were as reported previously using the same experimental set up and conditions (Young et al, 2006). The well was perfused with buffers shifting from 800 ppm to 0 ppm CO₂ via a peristaltic pump and teflon tubing. Background fluorescence intensities at 470 nm were measured in regions lacking guard cells and are also shown for the corresponding experiments.

Bicarbonate Activates S-Type Anion Currents in ca1;ca4 Double Mutant Guard Cell Protoplasts

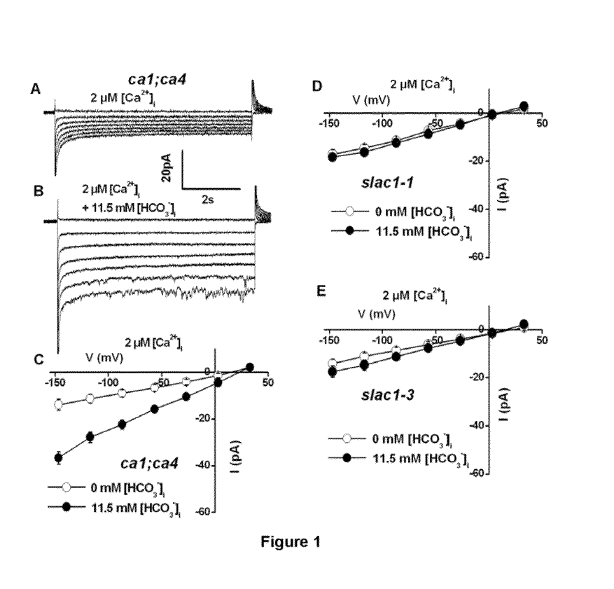

The βCA1 and βCA4 carbonic anhydrases act as upstream regulators in CO₂-induced stomatal movements in guard cells (Hu et al, 2010). Elevated CO₂ together with bicarbonate concentrations activate S-type anion channel currents in wild type Arabidopsis guard cells. Previous studies of CO₂ regulation of anion channels have only analyzed wild type guard cells (Brearley et al, 1997; Hu et al, 2010; Raschke et al, 2003). Therefore, we investigated whether elevated bicarbonate and intracellular CO₂ can by-pass the ca1;ca4 mutant and activate S-type anion currents in ca1;ca4 mutant guard cells. The addition of 13.5 mM total bicarbonate to the pipette solution (equivalent to 11.5 mM free bicarbonate ([HCO₃⁻]ᵢ)/2 mM free [CO₂] at pH 7.1) activated anion currents in patch clamped ca1;ca4 guard cell (FIGS. 1B and C), compared to control currents in the absence of added intracellular bicarbonate (FIG. 1A). Free [HCO₃^{-], and [CO}₂] were calculated using the Henderson-Hasselbalch equation as described in Methods. These findings are consistent with carbonic anhydrases acting as upstream regulators of CO₂ signaling and show that elevated bicarbonate and CO₂ together can activate S-type anion channel in ca1;ca4 double mutant guard cells.

Bicarbonate Activated S-Type Anion Currents are Greatly Impaired in slac1 Mutant Guard Cell Protoplasts

The reversal potential of CO₂+HCO₃⁻ activated whole-cell currents was +24.0±3.6 mV (n=8), which was close to the imposed chloride equilibrium potential of +31.1 mV, supports the hypothesis that CO₂+HCO₃⁻ activate guard cell anion channels. The bicarbonate and CO₂ concentrations used for anion current activation were very high (FIGS. 1B and C) (Hu et al, 2010), giving rise to the question whether these anion currents correspond to physiological guard cell anion channel currents, SLAC1 is required for Arabidopsis ABA- and CA²⁺-activation of guard cell S-type anion channel function (Negi et al, 2008; Vahisalu et al, 2008). To investigate whether high bicarbonate- and CO₂-activated anion currents are mediated by SLAC1, the recessive slac1-1 and slac1-3 mutants were analyzed. slac-1-1 mutant guard cell protoplasts displayed only small anion currents in the presence of 11.5 mM free [HCO₃⁻]i and 2 mM [CO₂] in the pipette solution, similar to control currents in the absence of added bicarbonate (FIG. 1D, P>0.05). Similar results were observed in slac1-3 mutant guard cells (FIG. 1E, P>0.05). These data suggest that the high intracellular [HCO₃⁻]+[CO₂]-mediated anion currents are mediated by the physiologically relevant SLAC1 anion channel (FIG. 1).

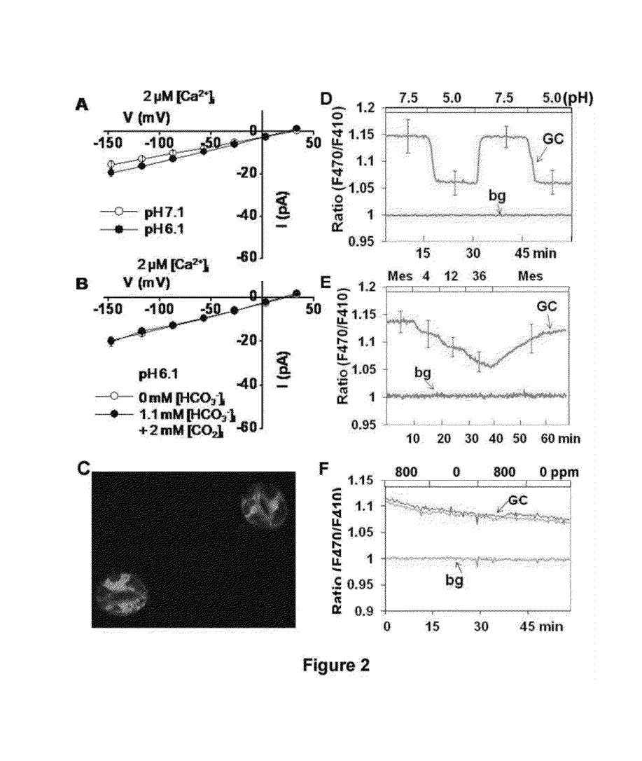

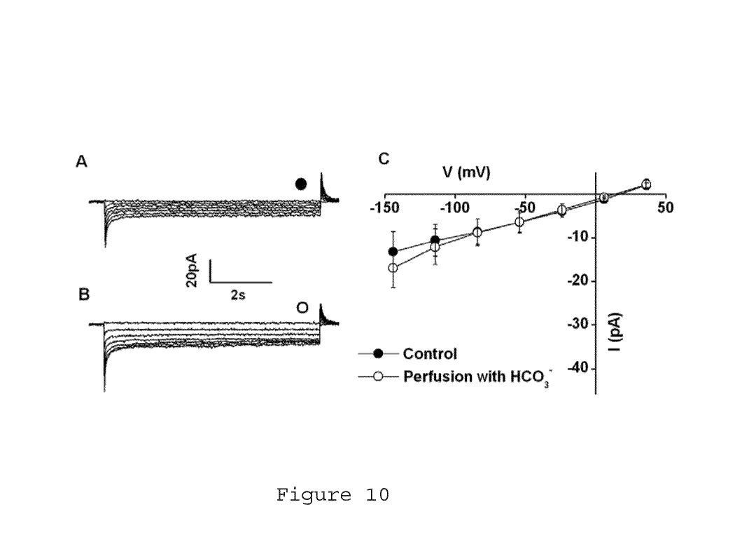

Next, we analyzed whether these anion currents show a clear HCO₃⁻ permeability in wild type guard cells. The total bicarbonate was elevated to 50 mM in the pipette solution at pH 7.1 (corresponds to 43.4 mM free [HCO₃⁻]ᵢ and 6.6 mM free [CO₂]). Under this high [HCO₃⁻] condition, the reversal potential of whole-cell currents was +26.0±0.9 mV (FIG. 10, or Supplementary FIG. 2, n=4). A relative permeability ratio of P_{HCO}3_{^{−}}/P_{Ci}_{^{−}}=0.06±0.01 was estimated using the Goldman equation. This CT over HCO₃⁻ selectivity of whole-cell anion currents is consistent with the anion selectivity of SLAC1 channels found in heterologous expression experiments in Xenopus laevis oocytes (Geiger et al, 2009).

### High [CO₂] and Protons Do Not Activate S-Type Anion Currents in the Absence of High Bicarbonate Levels in Guard Cells

Carbonic anhydrases reversibly catalyze the conversion of CO₂ into bicarbonate ions and free protons (Chandrashekar et al, 2009; Supuran, 2008). Whether high [CO₂], [HCO₃⁻], [H⁺] or a combination of these mediates activation of S-type anion channels in Arabidopsis guard cells remains to be investigated (Hu et al, 2010). We investigated whether intracellular acidification is capable of activating S-type anion currents in wild type guard cell protoplasts. Intracellular acidification at pH 6.1 alone did not significantly activate S-type anion channel currents compared with control recordings at pH 7.1 (FIG. 2A, P>0.05, Student's t-test). Interestingly, when the intracellular free [CO₂] was at a high concentration of 2 mM in the pipette solution (1.1 mM free [HCO₃⁻]ᵢ) at pH 6.1, S-type anion channel currents were not activated in wild type guard cell protoplasts, despite the high [CO₂] and high ([H⁺] applied (FIG. 2B, P>0.05, Student's t-test).

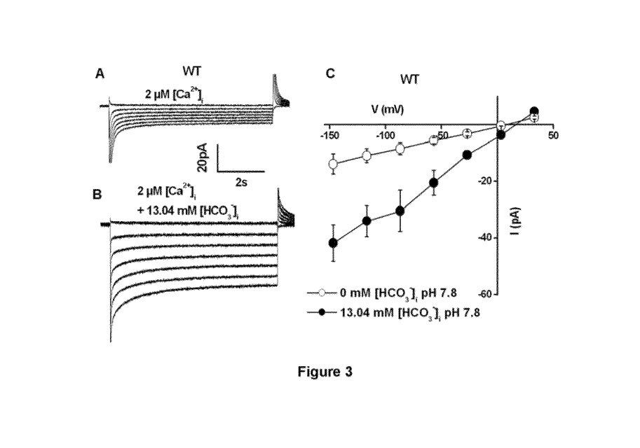

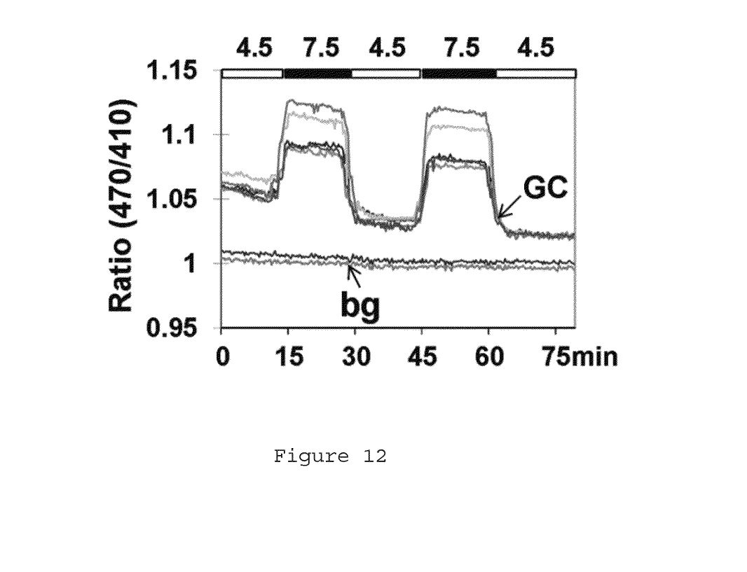

Previous research has shown no intracellular pH shift in Vicia faba guard cells in response to [CO₂] shifts (Brearly et al, 1997). To further investigate whether cytosolic pH is affected in Arabidopsis guard cells in response to [CO₂] shifts, a ratiometric pH indicator Pt-GFP (Schulte et al, 2006) under the control of a strong guard cell preferential promoter pGC1 (Yang et al, 2008) was transformed into Arabidopsis guard cells (FIG. 2C). In control experiments, in vivo recordings of pH in fluorescent pGC1::PtGFP transgenic guard cells showed clear reversible shifts in ratiometric intracellular pH fluorescence when the extracellular pH was repeatedly changed form pH 5.0 to pH 7.5 and back, see FIG. 2D and FIG. 12 (or Supplementary FIG. 3). Weak acids can control intracellular pH while maintaining a constant extracellular pH (Blatt & Armstrong, 1993; Grabov & Blatt, 1977). Therefore, the weak acid sodium butyrate was used to analyze whether Pt-GFP can report intracellular pH. Ratiometric fluorescence recordings of Pt-GFP-expressing guard cells showed clear shifts, when intact plant epidermes were perfused with defined concentrations of sodium butyrate-containing MES buffers (FIG. 2E), indicating intracellular pH changes were easily detected in guard cells (FIGS. 2D and E). However, no clear shifts in guard cell intracellular pH fluorescence were observed when the concentration of CO₂ bubbled in the extracellular perfusion buffers was repeatedly shifted from 0 ppm to 800 ppm (FIG. 2F), consistent with findings in Vicia faba guard cells using a pH sensitive dye (Brearley et al, 1997). In conclusion, protons alone or in combination with elevated CO₂ could not activate S-type anion channels (FIGS. 2A and B) and [CO₂] changes did not cause measurable changes in intracellular pH of Arabidopsis guard cells (FIG. 2F) (Brearley et al, 1997).

### Bicarbonate Activates S-Type Anion Currents at Low Free CO₂ in Guard Cells

To see whether elevated intracellular [HCO₃⁻] is sufficient to activate anion currents at low [H⁻] and low [CO₂], 13.5 mM total CsHCO₃ was added to the pipette solution and the free [HCO₃⁻] was calculated as 13.04 mM with 0.46 mM free [CO₂] at pH 7.8. These analyses clearly showed that compared with the control recordings (FIG. 3A), S-type anion currents were activated by the presence of high free HCO₃⁻ in the pipette solution (FIGS. 3B and C, P<0.05 at voltages from −146 mV to −26 mV, Student's t-test). Together the above analyses show that elevated intracellular HCO₃⁻ is the main molecule that mediates activation of S-type anion currents in guard cells.

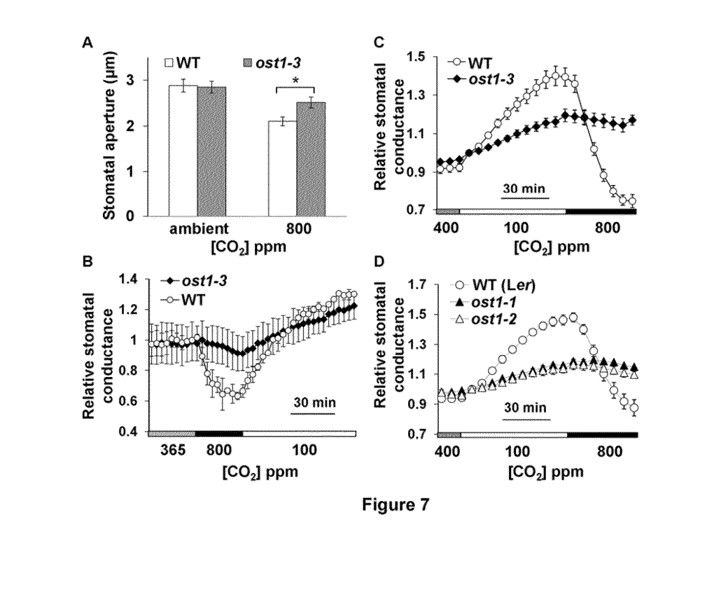

Extracellular bicarbonate was next tested on activation of S-type anion currents in wild type guard cells. After obtaining whole-cell recordings in wild type guard cells, the bath solution (200 μl) was perfused for 2 min at 1 ml min⁻¹ with a solution that contained 11.5 mM free [HCO₃⁻]ᵢ and 2 mM [CO₂] at pH 7.1; see FIG. 7.1; see FIG. 10A (or Supplementary FIG. 1A). No large S-type anion currents were activated; see FIGS. 10B and C (or Supplementary FIGS. 1B and C). A small increase in average anion current magnitude was not statistically significant and was not comparable to the clear activation of S-type anion currents by the same concentration of applied intracellular HCO₃⁻ (FIGS. 10B and C, or Supplementary FIGS. 1B and C).

### Elevated Intracellular [Ca²⁺] is Required for Bicarbonate Activation of S-Type Anion Channel Currents in Guard Cells

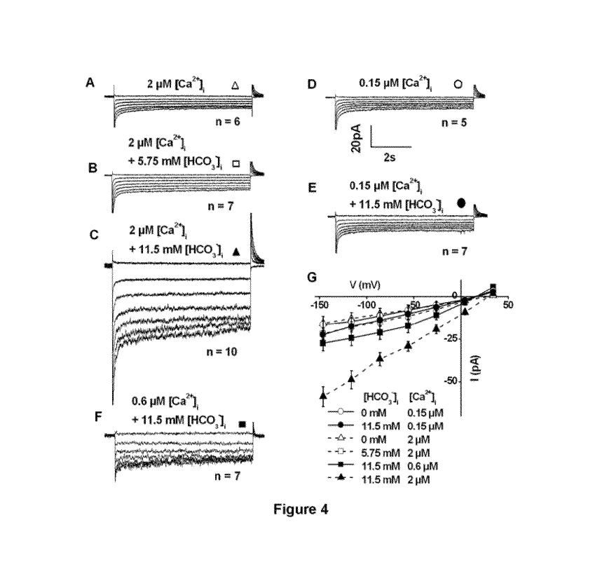

The above analyses of activation of S-type anion currents were all conducted at 2 μM cytosolic free Ca²⁺ ([Ca²⁺]ᵢ) (FIGS. 1-3). We investigated whether the elevated [Ca²⁺]ᵢ (2 μM) was necessary for bicarbonate activation of S-type anion channel currents in Arabidopsis guard cells. At 2 μM [Ca²⁺]ᵢ, anion currents were not strongly activated in the absence of added [HCO₃⁻]ᵢ (FIGS. 4A and G), consistent with previous studies (Allen et al, 2002; Siegel et al, 2009). In contrast, 11.5 mM free [HCO₃⁻]ᵢ activated strong S-type anion channels (FIGS. 4C and G, P<0.001), while an intermediate free [HCO₃⁻]ᵢ of 5.75 mM did not activate significant S-type anion currents (FIGS. 4B and G, P>0.05, Student's t-test). When [Ca²⁺]ᵢ was buffered to a baseline level of 0.15 μM even with high 11.5 mM free [HCO₃⁻]ᵢ and 2 mM free [CO₂] in the pipette solution (pH 7.1), S-type anion currents were not activate (FIGS. 4E and G). There was no significant difference between the average amplitudes of current recordings at 0.15 μM free [Ca⁺]ᵢ with or without added 11.5 mM free [HCO₃⁻]ᵢ (FIG. 4G, P>0.05, at voltages from −146 mV to +34 mV). In addition, an elevated cytosolic free [Ca²⁺]ᵢ of 0.6 μM together with high 11.5 mM free [HCO₃⁻]ᵢ and 2 mM free [CO₂] in the pipette solution (pH 7.1) activated anion currents of intermediate average amplitudes (FIGS. 4F and G).

A summary of cytosolic free Ca²⁺ and HCO₃⁻ activation of S-type anion channels are shown in Table I. These data demonstrate a requirement for an elevated [Ca²⁺]ᵢ in HCO⁻-mediated activation of guard cell anion channels and provide direct and mechanistic evidence for the model that CO₂-induced stomatal closing enhances the ability of [Ca²⁺]ᵢ to activate stomatal closing mechanisms (Young et al, 2006).

TABLE I
Cytosolic free [Ca²⁺]ᵢ and free [HCO₃]ᵢ activation
of anion currents at a voltage of −146 mV.

| [Ca²⁺]ᵢ | [HCO₃]ᵢ | I (pA) | | | |
|---|---|---|---|---|---|
| (μM) | (mM) | at −146 mV | P value | | Cell number |
| 0.15 | 0 | −16.4 ± 2.0^{a} | | | 5 |
| 2 | 0 | −15.6 ± 4.0^{b} | 0.76 | (b vs. a) | 6 |
| 0.15 | 11.5 | −22.3 ± 2.3^{c} | 0.071 | (c vs. a) | 7 |
| 2 | 5.75 | −21.8 ± 3.2^{d} | 0.054 | (d vs. b) | 7 |
| 2 | 11.5 | −58.7 ± 5.9^{e} | <0.001* | (e vs. b) | 10 |
| 0.6 | 11.5 | −27.3 ± 4.5^{f} | 0.056 | (f vs. a) | 7 |
| | | | 0.35 | (f vs. c) | |

^{a-f} Current values from FIG. 4G for comparision. Data are mean ± s.e.
*Stands for significant difference using Student's t-test.

Lower [Bicarbonate] is Sufficient for Activation of S-Type Anion Channel Currents in ht1-2 Guard Cells

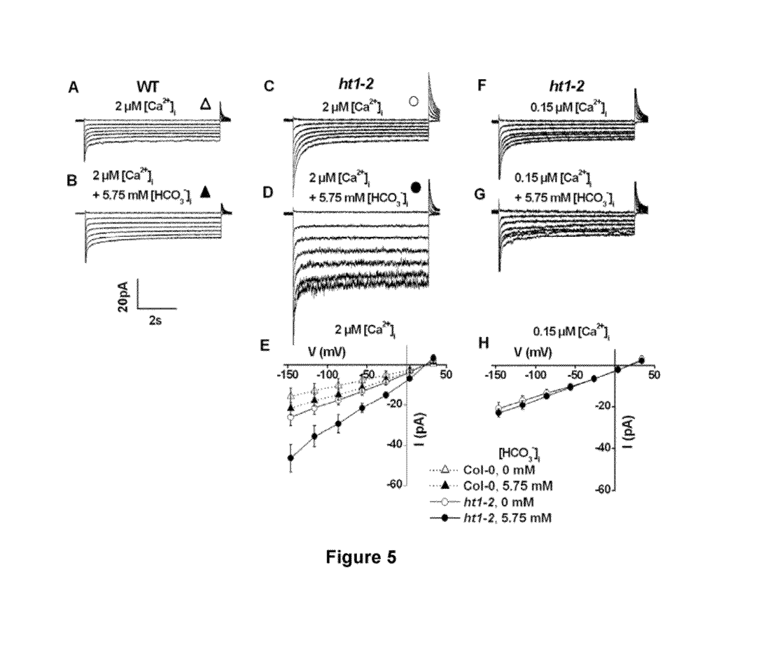

The Arabidopsis HT1 protein kinase functions as a negative regulator of CO₂-induced stomatal closing (Hashimoto et al, 2006). To test whether HT1 functions in the CO₂/HCO₃⁻ SLAC1 signaling pathway (FIGS. 1-3), the effects of bicarbonate on S-type anion currents in recessive ht1-2 mutant guard cells were analyzed. Whole-cell currents were recorded in guard cell protoplasts at lower intracellular [HCO₃⁻]ᵢ, 5.75 mM free [HCO₃⁻]ᵢ and 1 mM free [CO₂] at pH 7.1, compared to the above experiments (FIGS. 5A and B). In wild type control guard cells these intermediate [HCO₃⁻]ᵢ+[CO₂] together with 2 μM free [Ca²⁺]ᵢ showed small whole-cell current amplitudes that were slightly larger than wild type guard cells in the absence of added HCO₃⁻ (FIGS. 5A, B and E, P>0.05, Student's t-test) (Hu et al, 2010). However, significant activation of S-type anion currents by intracellular addition of 5.75 mM free [HCO₃⁻]ᵢ and 1 mM free [CO₂] (pH 7.1) was observed in ht1-2 guard cells (FIGS. 5D and E) compared to the control currents (FIGS. 5A-C and E, P<0.01 at voltages from −146 mV to −26 mV, Student's t-test). Note that 2 μM [Ca²⁺]ᵢ alone in ht1-2 guard cells was not sufficient to activate S-type anion currents (FIGS. 5C and E). While cytosolic [Ca²⁺]ᵢ was buffered to a typical resting level of 0.15 μM in ht1-2 guard cells, no significant S-type anion current activation was observed in the presence of 5.75 mM free [HCO₃⁻]ᵢ (FIG. 5F-H, P<0.05 at voltages from −146 mV to −26 mV, Student's t-test). Thus ht1-2 guard cells shown an enhanced sensitivity to intracellular HCO₃⁻, but this enhanced activation cannot by-pass the requirement for [Ca²⁺]ᵢ in HCO₃⁻ activation of S-type anion currents.

### The OST1 Kinase Functions in Bicarbonate Activation of S-Type Anion Currents in Guard Cell Protoplasts and Strongly Impairs CO₂-Induced Stomatal Closure

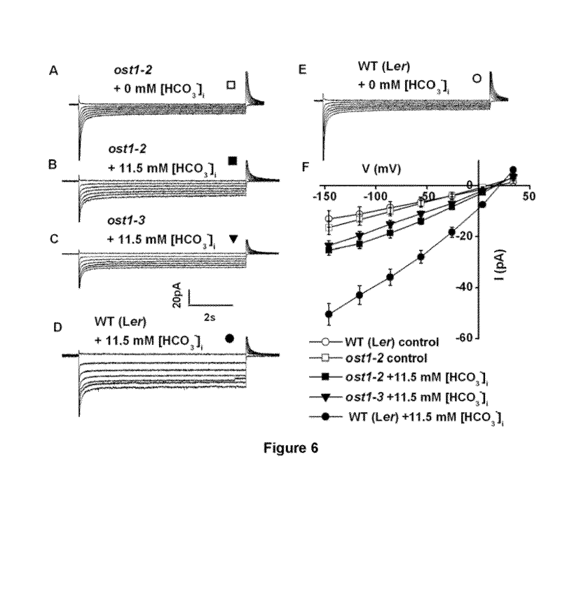

The OST1 protein kinase was previously demonstrated to mediate ABA-induced stomatal closing. Recessive ost1 mutants disrupt ABA-induced stomatal closure as well as ABA inhibition of light-induced stomatal opening, but low CO₂ induction of stomatal opening remained unaffected in the ost1-2 mutant, indicating that OST1 doesn't participate in CO₂ signaling (Mustilli et al, 23002; Yushida et al, 2002). Here, the effect of OST1 on bicarbonate activation of S-type anion channels was investigated. Using the same recording solutions in FIG. 1B, high [HCO₃⁻]ᵢ (11.5 mM) and [CO₂] (2 mM) activated only small S-type anion currents in Landsberg erecta (Ler) ost1-2 mutant guard cells (FIGS. 6A, B and F). Similar to Co1 wild-type guard cells (FIGS. 1, 3 and 4), high HCO₃⁻ activated S-type anion channel currents in Ler wild type guard cells (FIGS. 6D, E and F). While HCO₃⁻ activated S-type anion currents in Ler wild type guard cells were larger (I=−51±4.3 pA at a voltage of −146 mV, n=7) than that in ost1-2 mutant guard cells (I=−25.2±1.9 pA at a voltage of −146 mV, n=6) (FIG. 6F, P<0.001, Student's t-test). Moreover, bicarbonate activation of S-type anion channels was also strongly impaired in Co1 ost1-3 T-DNA insertion allele guard cells (FIGS. 6C and F) compared to Co1-0 wild type (FIGS. 4C and G). At a voltage of −146 mV, the current amplitude activated by bicarbonate in ost1-3 mutant guard cells was −24±1.9 pA (FIG. 6F, n=6), and in Co1-0 wild type, it was −59±5.9 pA (FIG. 4E, n=10, P<0.001, Student's t-test).

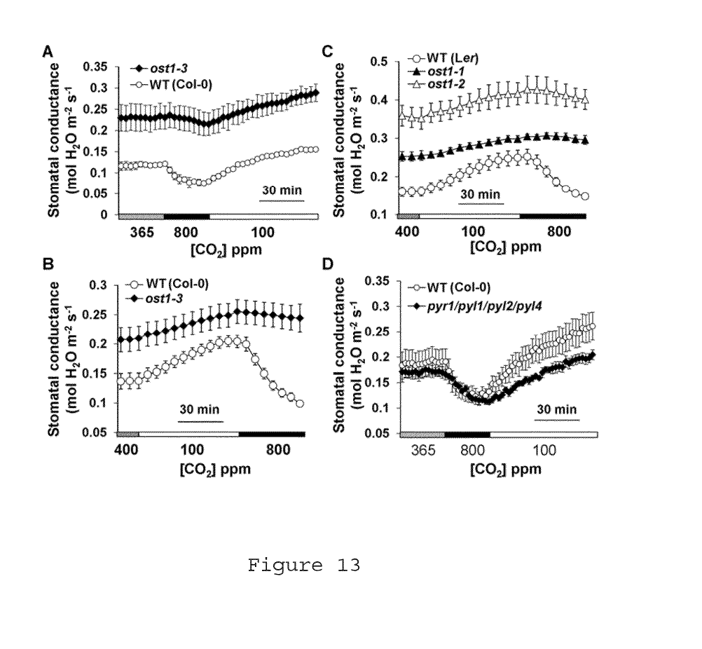

Elevated CO₂-induced stomatal closure was also impaired in ost1-3 mutant leaf epidermes compared to wild type controls in genotype-blind assays (FIG. 7A, P<0.05 at 800 ppm CO₂, Student's t-test). Stomatal conductance changes in intact ost1-3 mutant leaves were subsequently analyzed in response to [CO₂] shifts. Interestingly, stomatal conductance in ost1-3 mutant leaves showed a very strong CO₂ insensitivity when the [CO₂] was shifted to high concentrations; see FIG. 7B and FIG. 13A (or Supplementary FIG. 4A). To further investigate the unexpected strong CO₂ insensitivity of ost1, whole intact plant gas exchange experiments were pursued and the strong CO₂ insensitivity was observed in ost 1-1, ost1-2 and ost1-3 mutants, see FIG. 7C, D and FIGS. 13B and C (or Supplementary FIGS. 4B and C).

ABA Receptor pyr1;pyl1;pyl2;pyl4 Quadruple Mutant and Type 2C Protein Phosphatases abi1-1 and abi2-1 Mutants Maintain Functional CO₂ Response

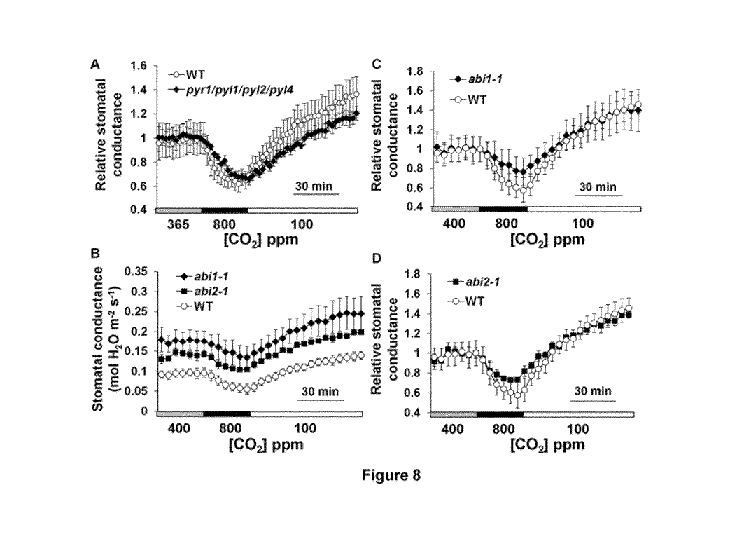

The PYR/RCAR ABA receptor family was recently identified in Arabidopsis as major ABA receptors (Ma et al, 2009; Park et al, 2009). Since these ABA receptors tightly regulate and form complexes with SnRK2 kinases including OST1 (Fujii et al, 2009; Ma et al, 2009; Nishimura et al, 2010; Park et al, 2009), CO₂ regulation of gas exchange in intact pyr1;pyl1;pyl2;pyl4 leaves was analyzed to see the requirement of ABA receptors for this CO₂ response. Intact leaves of the pyr1;pyl1;pyl2;pyl4 quadruple mutant showed clear CO₂ responses upon [CO₂] changes; see FIG. 8A and FIG. 13D (or supplementary FIG. 4D) and showed an average slight showing of the CO₂ response, observed in independent experimental sets but was not statistically significant (P=0.1, Student's t-test) at 18 min after 365 to 800 ppm CO₂ transition. Upon shifting [CO₂] from 365 to 800 ppm for 30 min, the initial rates of stomatal conductance changes were −0.038±0.014 mmol H₂O m⁻² s⁻¹ min⁻¹ for wild type plants and −0.035±0.008 mmol H₂O m⁻² s⁻¹ min⁻¹ for pyr1;pyl1;pyl2;pyl4 mutant plants (P=0.24, Student's t-test). During the first 30 min upon shifting [CO₂] from 800 to 100 ppm, the initial rates were 0.042±0.013 mmol H₂O m⁻² s⁻¹ min⁻¹ for wild type plants and 0.022±0.002 mmol H₂O m⁻² s⁻¹ min⁻¹ for pyr1;pyl1;pyl2;pyl4 mutant plants (P=0.06, Student's t-test).

ABI1 and ABI2 encode type 2C protein phosphatases (PP2Cs) (Leung et al, 1994; Leung et al, 1997, Meyer et al, 194; Rodriguez et al, 1998). The dominant mutants abi1-1 and abi2-1 exhibit ABA insensitivity in seed germination, root growth responses and guard cells signaling (Koornneef et al, 1984; Pei et al, 1997). ABI1, PYR1 and OST1 interact with each other in ABA signaling (Nishimura et all, 2010; Park et al, 2009), thereafter CO₂ regulation of gas exchange in abi1-1 and abi2-1 intact leaves were analyzed as well. Note that abi1-1 and abi2-1 leaves can wilt easily and therefore all gas exchange experiments were conducted on well-watered plants at ˜75-85% humidity, abi1-1 and abi2-1 mutants showed slightly impaired responses to changes of [CO₂] compared with wild type Co1-0 plants (FIGS. 8B, C and D). Average stomatal conductances of abi1-1 and abi2-1 were larger than that of wild type leaves (FIG. 8B). The initial rates of stomatal conductance changes were −0.041±0.01 mmol H₂O m⁻² s⁻¹ min⁻¹ for wild type plants, −0.035±0.007 mmol H₂O m⁻² s⁻² for abi1-1 and −0.037±0.007 mmol H₂O m⁻² s⁻² for abi2-1 mutant plants upon shifting [CO₂] from 400 to 800 ppm for 30 min. These data correlate with stomatal response assays in leaf epidermes suggesting that abi1-1 and abi2-1 may show a mild conditional effect on CO₂ responses (Leymarie et al, 1998a; Leymarie et al, 1998b; Webb & Hetherington, 1997).

### Discussion

Elevated [CO₂] in leaf intercellular spaces (Cᵢ) and elevated atmosphere [CO₂] cause closing of stomatal pores (Medlyn et al, 2001). Carbonic anhydrases have been identified that function early in CO₂ signal transduction (Hu et al, 2010). However, major questions in CO₂ signal transduction have arisen. Whether CO₂ or bicarbonate ion or a combination of these function in CO₂ signal transduction in guard cells remained unclear. The presented findings demonstrate that bicarbonate acts as an intracellular signaling molecule in CO₂ signal transduction, by activating SLAC1-mediated S-type anion channels in guard cells. We further found a synergistic action of intracellular HCO₃⁻ with cytosolic Ca²⁺, that requires both of these small molecules of CO₂ signaling to proceed. We also report the characterization of the cellular functions and relative positions within the CO₂ signal transduction cascade of mutants that strongly affect CO₂ control of stomatal movements, including ca1;ca4, slac1 and ht1, ht1-2 mutant guard cells show hypersensitivity to intracellularly applied HCO₃⁻, but continue to require cytosolic CA²⁺ for activation of SLAC1-dependent anion currents. In addition, we have unexpectedly found that loss-of-function mutations in the OST1 protein kinase cause a strong CO₂ insensitivity of stomatal regulation by analyses of S-type anion channel regulation, stomatal movements and gas exchange in intact leaves and in whole plants, which leads to a new model for early CO₂ signal transduction in guard cells.

### Control Function of the OST1 Protein Kinase in CO₂ Signal Transduction

Previous stomatal movement assays indicated that the OST1 protein kinase may not function in CO₂ inhibition of stomatal opening (Mustilli et al, 2002). Unexpectedly, we have found here in ost1 mutant guard cells in both Columbia and Landsberg accessions show a dramatic impairment in CO₂ regulation of stomatal conductance in intact leaves. Recent studies have shown that the OST1 kinase activates SLAC1 channels via phosphorylation (Geiger et al, 2009; Lee et al 2009; Vahisalu et al, 2010). Together our findings of impairment in bicarbonate activation of S-type anion currents in ost1-2 and ost1-3 mutant guard cells (FIGS. 6A, B and D) and the strong impairment in CO₂-induced stomatal closing and stomatal conductance changes in intact leaves and in intact plants (FIG. 7B-D) show that the OST1 protein kinase is a central transducer of CO₂ signal transduction in guard cells.

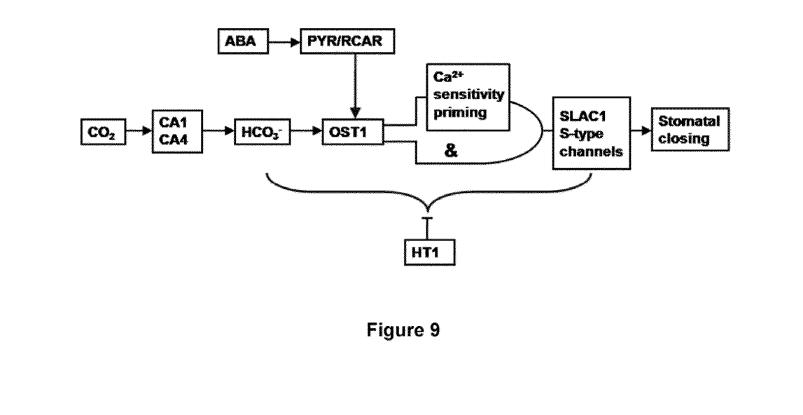

The PYR/RCAR abscisic acid receptors form a linear signal transduction module together with type 2C protein phosphatases and the OST1 protein kinase (Fujii et al, 2009; Ma et al, 2009; Nishimura et al, 2010; Park et al, 2009; Santiago et al, 2009; Umezawa et al, 2009). A quadruple mutant in four highly-expressed guard cell ABA receptors pyr1;pyl1;pyl2;pyl4 shows a strong impairment in ABA-induced stomatal closing (Nishimura et al, 2010). In contrast CO₂ regulation remained functional in intact leaves (FIG. 8). These data lead to an updated model for early CO₂ signal transduction in which the convergence point of CO₂ and ABA signal transduction occurs earlier than previously thought at the level of the OST1 protein kinase (FIG. 9). The CO₂ response of pyr1;pyl1;pyl2;pyl4 quadruple mutant plants exhibited an average slight showing compared to wild type plants (FIG. 8). This may be attributable to the convergence of CO₂ and ABA signaling at the level of the OST1 protein kinase as revealed here. Thus a degree of cross-talk between ABA and CO₂ signaling can be expected. Classical studies have shown that very low subthreshold concentrations of ABA do not cause an ABA response, but amplify CO₂-induced stomatal closing (Raschke, 1975). Our findings provide a mechanistic basis for this classical observation, with both CO₂ and ABA signal transduction occurring via the OST1 protein kinase (FIG. 9), as ost1 mutant alleles show both strong CO₂ (FIG. 7) and ABA insensitivities (Mustilli et al., 2002; Yoshida et al., 2002).

The dominant protein phosphatase 2C (PP2C) mutants, abi1-1 and abi2-1, have been reported to conditionally affect CO₂ signaling in guard cells (Leymarie et al 1998a; Leymarie et al, 1998b; Webb & Hetherington, 1997). ABI1 interacts with the OST1 protein kinase (Belin et al, 2006; Nishimura et al 2010; Umezawa et al. 2009; Vlad et al, 2009; Yoshida et al, 2006). The present study on CO₂ signaling and research indicating ABA-independent activation of the OST1 protein kinase (Yoshida et al, 2006; Zheng et al, 2010) indicates that the early ABA signaling module consisting of ABA receptors, PP2Cs and OST1/SnRK2 kinases (Ma et al, 2009; Park et al, 2009) may be more complex than present models (Fujii et al, 2009).

### Bicarbonate Activates S-Type Anion Channels

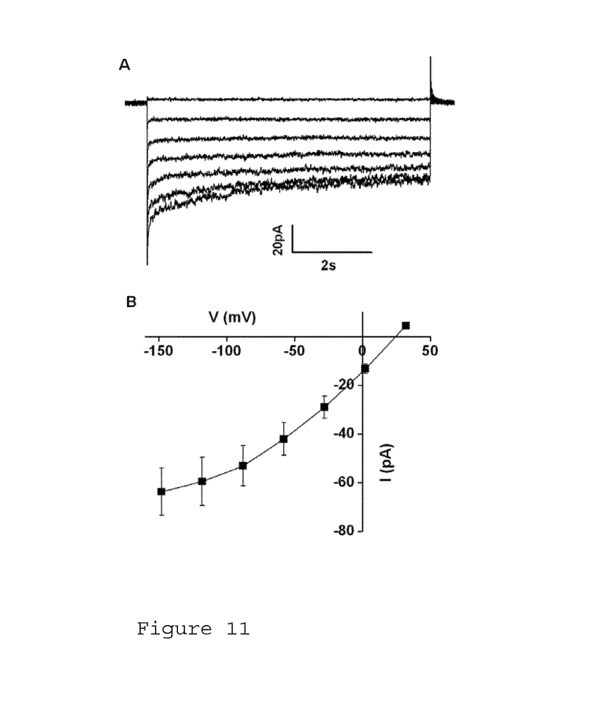

Elevated bicarbonate activation of S-type anion currents in ca1;ca4 double mutant guard cells (FIG. 1) is consistent with the model that βCA1 and βCA4 act very early in the guard cell CO₂ signal transduction pathway (FIG. 9). S-type anion channel activation by bicarbonate reported here (FIG. 3) shows similar properties to SLC26A9 channels in mammalian epithelial cells. SLC26A9, encoding a CT channel, is modulated by HCO₃⁻ (Loriol et al, 2008). Expression of SLC26A9 in Xenopus laevis oocytes, produced CT currents that increased in magnitude in the presence of 24 mM HCO₃⁻ compared to 2.4 mM HCO₃⁻. Furthermore, the SLC26A9 channel has no HCO₃⁻ permeability and is not regulated by intracellular pH (Loriol et al, 2008). In Arabidopsis hypocotyl cells, bicarbonate is permeable through voltage-dependent anion channels (R-type anion channels) with a relative permeability ratio P_{HCO3}_{^{−}}P_{CO}_{^{−}} of 0.8 (Frachisse et al, 1999). Different from that, the SLAC1 channel is impermeable to HCO₃⁻ (Geiger et al, 2009), and our analyses of S-type anion currents also support this; see FIG. 11 (or Supplementary FIG. 2). SLAC1 channels were not activated by bicarbonate when SLAC1 was heterologously expressed alone in Xenopus laevis oocytes (Geiger et al, 2009). This can be explained by our findings that bicarbonate activation of S-type anion channel in planta requires other essential components, in particular the OST1 protein kinase and elevated [Ca²⁺]ᵢ, with the HT1 protein kinase functioning as a negative regulator within this module of the CO₂ signal transduction cascade (FIGS. 4-6, and 9). Further research will be needed to identify the bicarbonate-binding proteins that mediate this response.

The intra cellular concentrations of bicarbonate and CO₂ used in patch clamp experiments in the present study for S-type anion channel activation were higher than physiological concentrations in planta. Note that patch clamping of guard cells includes dialysis of the cytoplasm (Hamill et al, 1981) and it is possible that additional diluted small molecules or proteins are required for full sensitivity of this HCO₃⁻ response. Furthermore, typically high CO₂ and HCO₃⁻ concentrations are used in electrophysiological studies, up to 72 mM HCO₃⁻ (Chandrashekar et al, 2009; Hu et al, 2010: Loriol et al, 2008; Yarmolinsky et al, 2009), although these experiments were conducted in different systems. The close correlation of high HCO₃⁻ regulation of S-type anion channels in the present study and the impaired CO₂ response phenotypes in intact leaves of the Arabidopsis cal1;cal4, slac1, ht1-2 and ost1 mutants (FIGS. 6 and 7) and the [Ca²⁺]ᵢ sensitivity of this response (FIG. 4) suggest that the analyzed intracellular HCO₃⁻ regulation responses are physiologically relevant (Hashimoto et al, 2006; Hu et al, 2010; Negi et al, 2008; Schwartz, 1985; Vahisalu et al, 2008; Webb et al, 1996; Young et al, 2006).

Intracellular acidification activates slow anion channel currents in the plasma membrane of Arabidopsis hypocotyl cells (Colcombet et al, 2005). However, intracellular acidification did not activate S-type anion currents in Arabidopsis guard cells, even in the presence of elevated 2 μM free [Ca²⁺]ᵢ (FIG. 2A). In animal chemosensitive neurons, intracellular pH was lowered in response to increasing CO₂ levels from 10% up to 50% [CO₂] (Putnam et al, 2004). Using the pH sensitive dye BCECP (2′,7′-bis-(2-carboxyethyl)-5,6-carboxyflourescein) and fluorescence microphotometry to measure cytosolic pH in Vicia faba guard cells, no significant pH change was observed during transition from 0 to 1000 ppm CO₂ (Brearley et al, 1997). Our findings correlate with the previous study as no detectable pH changes were observed in guard cells expressing the ratiometric pH sensor Pt-GFP when intact leaf epidermes were perfused with buffers bubbled with 0 ppm and 800 ppm CO₂ (FIG. 2F). These data are also compatible with models proposing a high pH buffering capacity of Vicia faba guard cells (Grabov & Blatt, 1997; Raschke et al, 1988).

### CO₂ Enhances the [Ca²⁺]ᵢ Sensitivity of S-type Anion Channel Activation

Calcium is a second messenger that transduces diverse stimuli in plants (Blatt, 2000; Hetherington & Brownlee, 2004; Kim et al, 2010; Kudla et al, 2010; Sanders et al, 1999). Elevated CO₂ caused an increase in [Ca²⁺]ᵢ in Commelina Communis guard cells (Webb et al, 1996). Furthermore, elevated CO₂ caused a dampening of spontaneous repetitive [Ca²⁺]ᵢ, transients whereas low CO₂ caused rapid [Ca²⁺]ᵢ transients in Arabidopsis guard cells (Young et al, 2006), which can be attributed to CO₂-induced depolarization of guard cells (Grabov & Blatt, 1998; Klusener et al, 2002; Staxen et al, 1999). In both plant species abolishment of [Ca²⁺]ᵢ elevations abolished CO₂-induced stomatal closing (Schwartz, 1985; Webb et al, 1996; Young et al. 2006). Time-resolved [Ca²⁺]ᵢ, imaging experiments led to the Ca²⁺ sensitivity priming hypothesis, in which CO₂ was hypothesized to enhance (prime) the Ca²⁺ sensitivity of signaling mechanisms that relay CO₂-induced stomatal closure (Young et al, 2006). However, additional and direct evidence for this CO₂ signaling hypothesis has been lacking. Recent studies showed that ABA enhances (primes) the [Ca²⁺]ᵢ, sensitivity of S-type anion channel and Kᵢₙ⁺ channel regulation, strongly supporting the hypothesis that ABA primes [Ca²⁺]ᵢ signal transduction (Siegel et al, 2009).

ABA increases cytosolic Ca²⁺ concentration by activating plasma membrane Ca²⁺ channels in Vicia faba and Arabidopsis guard cells (Grabov & Blatt, 1998; Hamilton et al, 2000; Murata et al, 2001; Pei et al., 2000; Schroeder & Hagiwara, 1990). Cytosolic [Ca²⁺]ᵢ interacts with other signaling molecules including nitric oxide (NO) (Garcia-Mata et al, 2003) and cytosolic pHᵢ (Grabov & Blatt, 1997) in ion channels regulation in guard cells. Recently, Chen et al (2010) showed that cytosolic free [Ca²⁺]ᵢ interacts with protein phosphorylation events during slow anion currents activation.

The present study shows that elevated bicarbonate enhances the [Ca²⁺]ᵢ sensitivity in S-type anion channels activation (FIG. 4). ABA- and Ca²⁺-activation of S-type anion channels and stomatal closing are mediated by Ca²⁺-dependent protein kinases (CDPKs) (Geiger et al, 2010; Mori et al, 2006; Zhu et al, 2007). Heterologous reconstitution analysis has proposed that ABA activates anion channels by the OST1 protein kinase, in parallel through a Ca²⁺-dependent CDPK pathway (Geiger et al, 2010). Together with previous studies (Allen et al, 2002; Hu et al, 2010; Israelsson et al, 2006; Siegel et al, 2009; Young et al, 2006), the present findings provide strong evidence that Ca²⁺ sensitivity priming is a mechanism that controls both CO₂ and ABA regulation on S-type anion channels (FIG. 9). Interestingly, here patch clamped guard cell protoplasts were exposed to elevated HCO₃⁻/CO₂ in the pipette solution for only ˜3 to 5 min prior to analyzing [Ca²⁺] activation of S-type anion currents (FIGS. 4C and G), whereas ABA signaling studies tested 30 min ABA pre-incubation (Siegel et al, 2009). This rapid 3 to 5 min HCO₃⁻/CO₂—[Ca²⁺]ᵢ response provides first evidence that Ca²⁺ sensitivity priming is a rapid modification and that transcriptional and translational mechanisms do not mediate Ca²⁺ sensitivity priming.

### Ht1 Kinase Mutant Enhances Bicarbonate Sensitivity but Requires [Ca²⁺]ᵢ

The HT1 protein kinase functions as a negative regulator of CO₂ signaling (Hashimoto et al, 2006) and our recent study showed that HT1 is epistatic to βCA1 and βCA4 in CO₂ responses pathway (Hu et al, 2010). However, the role of HT1 within the guard cell signaling network had not been further analyzed. The ht1-2 mutant exhibits a hypersensitive response in bicarbonate activation of S-type anion currents, demonstrating that the HT1 kinase functions as a negative regulator and affects CO₂ signaling downstream of HCO₃⁻ production and upstream of anion channel activation (FIG. 9). Cytosolic Ca²⁺ elevation is still required for S-type anion channel activation in ht1-2 mutant guard cells, showing that HT1 kinase-mediated CO₂ signaling does not by-pass Ca²⁺ sensitivity priming (FIGS. 5 and 9).

In conclusion, the present study identifies the OST1 protein kinase and the synergistic roles of the intercellular small molecules HCO₃⁻ and Ca²⁺ in guard cell CO₂ signal transduction and anion channel regulation. Furthermore, characterization of the positions and roles of OST1, the HT1 protein kinase, the βCA1 and βCA4 carbonic anhydrases, PYR/RCAR ABA receptors, ABI1 and ABI2 PP2Cs and SLAC1 in CO₂ regulation of S-type anion channels, leads to a revised model for CO₂ signal transduction (FIG. 9). During CO₂-induced stomatal closing, CO₂ is first catalyzed by CAs into bicarbonate. Elevated bicarbonate, but no protons or CO₂ activate S-type anion channels via an “AND”-like gate (FIG. 9). In the “AND”-like gate, one “input” occurs via the OST1 pathway, and the other “input” is mediated by the Ca²⁺ sensitivity priming pathway. The HT1 kinase acts as a negative regulator in the CO₂ signaling pathway downstream of HCO₃⁻ production and upstream of S-type anion channel activation, which continues to require [Ca²⁺]ᵢ, PYR/RCAR ABA receptors do not directly mediate guard cell CO₂ signaling and function upstream of the convergence point of CO₂ and ABA signaling (FIG. 8), whereas the OST1 protein kinase is an essential mediator of guard cell CO₂ signal transduction, providing evidence that mechanisms in addition to abscisic acid can activate OST1-dependent signaling (FIGS. 6 and 7).

### Supplementary Methods

### Solutions for Patch Clamp Experiments

For analyses of S-type anion currents, the pipette solution contained 150 mM CsCl, 2 mM MgCl₂, 6.7 mM EGTA, 2.61 mM CaCl₂ (150 mM [Ca²⁺]ᵢ), 4.84 mM CaCl₂ (0.6 μM [Ca²⁺]ᵢ), or 6.03 mM CaCl₂ (2 μM [Ca²⁺]ᵢ), 5 mM Mg-ATP, 5 mM Tris-GTP, 1 mM HEPES/Tris, pH 7.1. For experiments analyzing effects of protons on S-type anion currents, the pipette solution contained 150 mM CsCl, 2 mM MgCl₂, 6.7 mM EGTA, 0.6 mM CaCl₂ (2 μM [Ca²⁺]ᵢ), 5 mM Mg-ATP, 5 mM Tris-GTP, 1 mM Mes/Tris, pH 6.1. For experiments with pipette solution at pH 7.8, the pipette medium contained 150 mM CsCl, 2 mM MgCl₂, 2 μM free [Ca²⁺]ᵢ, 5 mM Mg-ATP, 5 mM Tris-GTP, 1 mM HEPES/Tris. Calcium affinities of EGTA and free Ca²⁺ concentrations were calculated using the WEBMAXC tool (http://www.stanford.edu/-epatton/webmaxc/webmaxcE.htm), which considers pH, [ATP] and ionic conditions. The bath solution contained 30 mM CsCl, 2 mM MgCl₂, 5 mM CaCl₂ and 10 mM Mes/Tris, pH 5.6. Osmolalities of all solutions were adjusted to 485 mmol1·kg⁻¹ for bath solutions and 500 mmol·kg⁻¹ for pipette solutions by addition of D-sorbitol.

### Stomatal Conductance Measurements

Stomatal conductance measurements of 5-week-old plants in response to the imposed [CO₂] at a light (PAR) fluence rate of 150 μmol m⁻²s⁻¹ were conducted with a Li-6400 gas exchange analyzer with a fluorometer chamber (Li-Cor Inc.) as described-previously (Hu at al, 2010). To reduce the wilting of abi1-1 and abi1-2 mutant leaves, all plants ware analyzed with a humidifier that humidified the air surrounding plants to ˜75-85%. Relative stomatal conductance values of intact leaves were calculated by normalization relative to 365 or 400 ppm just before transition to 800 ppm [CO₂]. Data shown are mean±s.e. of at least 3 leaves per genotype in the same experimental set.

For whole-plant gas-exchange experiments, 24 to 26-day-old plants were used. Plants were grown in pots as described previously (Kollist et al, 2007). For monitoring CO₂-induced changes in whole-plant stomatal conductance, a custom made device for Arabidopsis whole-plant gas-exchange measurements was used (Kollist et al, 2007). Before application of different CO₂ treatments, plants were acclimated in the measuring cuvettes for at least 1 h (Vahisalu et al, 2008). Experiments were performed at photosynthetic photon flux density of 150±3 μmol m⁻²s⁻¹, relative humidity of 60-70% (vapor pressure deficit=0.9-1.2 kPa) and air temperature of 24-25° C. Photographs of plants were taken before the experiment and rosette leaf area was calculated using ImageJ 1.37v (National Institutes of Health, USA). Stomatal conductance for water vapor was calculated us described previously (Kollist et al, 2007; Vahisalu et al. 2008). Data were normalized relative to the stomatal conductance at 400 ppm [CO₂] just before the transition to 100 ppm [CO₂].

### Stomatal Aperture Measurements

Three to 4-week-old plants grown in a plant-growth chamber were used for analyses of stomatal movements in response to ambient and elevated [CO₂]. Intact leaf epidermal layers with no mesophyll cells in the vicinity and ambient or high [CO₂] (800 ppm) incubation buffers were prepared as described (Hu et al., 2010; Young et al, 2006). Leaf epidermal layers were pre-incubated for 1.5 h in a buffer containing 10 mM MES, 10 mM KCl, 50 μM CaCl₂ at pH 6.15 and then perfused with incubation buffers continually bubbled with ambient air or 800 ppm CO₂ for 30 min. Stomatal apertures were measured using ImageJ software and analyzed. Data shown are from genotype blind analyses (n=3 experiments, 40 stomata per experiment and condition).

### REFERENCES

Allen G J, Chu S P, Schumacher K, Shimazaki C T, Vefeados D, Kemper A, Hawke S D, Tallman G, Tsien R Y, Harper J F, Chory J, Schroeder J I (2000) Alteration of stimulus-specific guard cell calcium oscillations and stomatal closing in Arabidopsis det 3 mutant. Science 289: 2338-2342
Allen G J, Murata Y, Chu SP, Nafisi M, Schroeder J I (2002) Hypersensitivity of abscisic acid-induced cytosolic calcium increases i the Arabidopsis farnesyltransferase mutant era1-2, Plant cell 14: 1649-1662
Belin C, de Franco P O, Bourbousse C, Chaignepain S, Schmittier J M, Vavasseur A, Giraudat J, Barbier-Brygoo H, Thomine S (2006) Identification of features regulating OST1 kinase activity and OST1 function in guard cells, Plant physiol 141: 1316-1327
Blatt M R (2000) Cellular signaling and volume control in stomatal movements in plants, Annu Rev Cell Dev Biol 16:221-241
Blatt M R, Armstrong F (1993) K⁺ channels of stomatal guard cells: abscisic acid-evoked control of the outward rectifier mediated by cytoplasmic pH. Planta 191: 330-341
Brearley J, Venis M A, Blatt M R (1997) The effect of elevated CO₂ concentrations in K⁺ and anion channels of Vicia faba L. guard cells. Planta 203: 145-154
Chandrashekar J, Yarmolinsky D, von Buchholtz L, Oka Y, Sly W, Ryba N J, Zuker C S (2009) The taste of carbonation. Science 326: 443-445
Chen Z H, Hills A, Lim C K, Blatt M R (2010) Dynamic regulation of guard cell anion channels by cytosolic free Ca²⁺ concentration and protein phosphorylation. Plant J 61: 816-825
Clough S J, Bent A F (1998) Floral dip: a simplified method for Agrobacterium-mediated transformation of Arabidopsis thaliana. Plant J 16: 735-743
Colcombet J, Lelievre F, Thomine S, Barbier-Brygoo H, Frachisse J M (2005) Distinct pH regulation of slow and rapid anion channels at the plasma membrane of Arabidopsis thaliana hypocotyl cells. J Exp Bot 56: 1897-1903
Evans N H, Hetherington A M (2001) Plant physiology: the ups and downs of guard cell signalling. Curr Biol 11: R92-94
Frachisse J M, Thomine S, Colcombet J, Guern J, Barbier-Brygoo H (1999) Sulfate is both a substrate and an activator of the voltage-dependent anion channel of Arabidopsis hypocotyl cells. Plant physiol 121: 253-262
Frommer W B (2010) CO₂mmon sense. Science 327: 275-276
Fujii H, Chinnusamy V, Rodrigues A, Rubio S, Antoni R, Park S Y, Cutler S R, Sheen J, Rodriguez P L, Zhu J K (2009) In vitro reconstitution of an abscisic acid signalling pathway. Nature 462: 660-664
Garcia-Mata C, Gay R, Sokolovski S, Hills A, Lamattina L, Blatt M R (2003) Nitric oxide regulates K⁺ and Cl⁻ channels in guard cells through a subset of abscisic acid-evoked signaling pathways. Proc Natl Acad Sci USA 100: 11116-11121
Geiger D, Scherzer S, Mumm P, Marten I, Ache P, Matschi S, Liese A, Wellmann C, Al-Rasheid K A S, Grill E, Romeis T, Hedrich R (2010) Guard cell anion channel SLAC1 is regulated by CDPK protein kinases with distinct Ca²⁺ affinities. Proc Natl Acad Sci USA 107: 8023-8028
Geiger D, Scherzer S, Mumm P, Stange A, Marten I, Bauer H, Ache P, Matschi S, Liese A, Al-Rasheid K A, Romeis T, Hedrich R (2009) Activity of guard cell anion channel SLAC1 is controlled by drought-stress signaling kinase-phosphatase pair. Proc Natl Acad Sci USA 106: 21425-21430
Grabov A, Blatt M R (1997) Parallel control of the inward-rectifier K⁺ channel by cytosolic free Ca²⁺ and pH in Vicia guard cells. Planta 201: 84-95
Grabov A, Blatt M R (1998) Membrane voltage initiates Ca²⁺ waves and potentiates Ca²⁺ increases with abscisic acid in stomatal guard cells. Proc Natl Acad Sci USA 95: 4778-4783
Grabov A, Blatt M R (1999) A steep dependence of inward-rectifying potassium channels on cytosolic free calcium concentration increase evoked by hyperpolarization in guard cells. Plant physiol 119: 277-288
Grabov A, Leung J, Giraudata J, Blatt M R (1997) Alternation of anion channel kinetics in wild type and abi1-1 transgenic Nicotiana benthamiana guard cells by abscisic acid. Plant J 12: 203-213
Hamill O P, Marty A, Neher E, Sakmann B, Sigworth F J (1981) Improved patch-clamp techniques for high-resolution current recording from cells and cell-free membrane patches. Pflugers Arch 391: 85-100
Hamilton D W, Hills A, Kohler B, Blatt M R (2000) Ca²⁺ channels at the plasma membrane of stomatal guard cells are activated by hyperpolarization and abscisic acid. Proc Natl Acad Sci USA 97: 4967-4972
Hanstein S M, Felle H H (2002) CO₂-triggered chloride release from guard cells in intact fava bean leaves. Kinetics of the onset of stomatal closure. Plant physiol 130: 940-950
Hashimoto M, Negi J, Young J, Israelsson M, Schroeder J I, Iba K (2006) Arabidopsis HT1 kinase controls stomatal movements in response to CO₂. Nat Cell Biol 8: 391-397
Hauser M, Eichelmann H, Heber U, Laisk A (1995) Chloroplast pH values and buffer capacities in darkened leaves as revealed by CO₂ solubilization in-vivo. Planta 196: 199-204
Hetherintgon A M, Brownlee C (2004) The generation of Ca²⁺ signals in plants. Annu Rev Plant Biol 55: 401-427
Hetherington A M, Woodward F I (2003) The role of stomata in sensing and driving environmental change. Nature 424: 901-908
Hu H, Boisson-Dernier A, Israelsson-Nordstrom M, Bohmer M, Xue s, Ries A, Godoski J, Kuhn J M, Schroeder J I (2010) Carbonic anhydrases are upstream regulators of CO₂-controlled stomatal movements in guard cells. Nat Cell Biol 12: 87-93
Hubbard K E, Nishimura N, Hitomi K. Getzoff E D, Schroeder J I (2010) Early abscisic acid signal transduction mechanisms: newly discovered components and newly emerging questions. Genes Dev 24: 1695-1708
Israelsson M, Siegel R S, Young J, Hashimoto M, Iba K, Schroeder J I (2006) Guard cell ABA and CO₂ signaling network updates and Ca²⁺ sensor priming hypothesis. Curr Opin Plant Biol 9: 654-663
Kelly W B, Esser J E, Schroeder J I (1995) Effects of cytosolic calcium and limited, possible dual, effects of G protein modulators on guard cell inward potassium channels. Plant J 8: 479-489
Kim T H, Bohmer M, Hu H, Nishimura N, Schroeder J I (2010) Guard cell signal transduction network: advances in understanding abscisic acid, CO₂ and Ca²⁺ signaling. Annu Rev Plant Biol 61: 561-591
Kinoshita T, Nishimura M, Shimazaki K (1995) Cytosolic concentration of Ca²⁺ regulates the plasma membrane H⁺-ATPase in guard cells of Fava Bean. Plant cell 7: 1333-1342
Klusener B, Young J J, Murata Y, Allen G J, Mori I C, Hugouvieux V, Schroeder J I (2002) Convergence of calcium signaling pathways of pathogenic elicitors and abscisic acid in Arabidopsis guard cells. Plant physiol 130: 2152-2163
Koomneef M, Reulling G, Karssen C (1984) The isolation and characterization of abscisic acid-insensitive mutants of Arabidopsis thaliana. Physiol Plant 74: 377-383
Kudla J, Batistic O, Hashimoto K (2010) Calcium signals: the lead currency of plant information processing. Plant cell 22: 541-563
Lee S C, Lan W, Buchanan B B, Luan S (2009) A protein kinase-phosphatase pair interacts with an ion channel to regulate ABA signaling in plant guard cells. Proc Natl Acad Sci USA 106: 21419-21424
Leonhardt N, Kwak J M, Robert N, Waner D, Leonhardt G, Schroeder J I (2004) Microarray expression analyses of Arabidopsis guard cells and isolation of a recessive abscisic acid hypersensitive protein phosphatase 2C mutant. Plant cell 16: 595-615
Leung J, Bouvier-Durand M, Morris P C Guerrier D, Chefdor F, Giraudat J (1994) Arabidopsis ABA response gene ABI1: features of a calcium-modulated protein phosphatase. Science 264: 1448-1452
Leung J, Merlot S, Giraudata J (1997) The Arabidopsis ABSCISIC ACID-INSENSITIVE2 (ABI2) and ABI1 genes encode homologous protein phosphatases 2C involved in abscisic acid signal transduction. Plant cell 9: 759-771
Leymarie J, Lasceve G, Vavassuer A (1998a) Interaction of stomatal responses to ABA and CO₂ in Arabidopsis thaliana. Aust J Plant Physiol 25: 785-791
Leymarie J, Vavassuer A, Lasceve G (1998b) CO₂ sensing in stomata abi1-1 and abi2-1 mutants of Arabidopsis thaliana. Plant Physiol Biochem 36: 539-543
Loriol C, Dulong S, Avella M, Gabillat N, Boulukos K, Borgese F, Ehrenfeld J (2008) Characterization of SLC26A9, facilitation of CT transport by bicarbonate. Cell Physiol Biochem 22: 15-30
Ma Y, Szostkiewicz I, Korte A, Moes D, Yang Y, Christmann A, Grill E (2009) Regulators of PP2C phosphatase activity function as abscisic acid sensors. Science 324: 1064-1068
MacRobbie E A (1998) signal transduction and ion channels in guard cells. Philosophical transactions of the Royal Society of London 353: 1475-1488.
Medlyn B E, Barton C V M, Broadmeadow M S J, Ceulemans R, De Angelis P, Forstreuter M, Freeman M, Jackson S B, Kellomaki S, Laitat E, Rey A, Roberntz P, Sigurdsson B D, Strassemeyer J, Wang K, Curtis P S, Jarvis P O (2001) Stomatal conductance of forest species after long-term exposure to elevated CO₂ concentration: a synthesiss. New Phytol 149: 247-264
Meyer K, Leube M P, Grill E (1994) A protein phosphatase 2C involved in ABA signal transduction in Arabidopsis thaliana. Science 264: 1452-1455
Mori I C, Murata Y, Yang Y, Munemasa S, Wang Y F, Andreoli S, Tiriac H, Alonso J M, Harper J F, Ecker J R, Kwak J M, Schroeder J I (2006) CDPKs CPK6 and CPK3 function in ABA regulation of guard cell S-type anion- and Ca²⁺-permeable channels and stomatal closure. PLoS Biol 4: 1749-1762.
Murata Y, Pei Z M, Mori I C, Schroeder J (2001) Abscisic acid activation of plasma membrane Ca²⁺ channels in guard cells requires cytosolic NAD(P)H and is differentially disrupted upstream and downstream of reactive oxygen species production in abi1-1 and abi2-1 protein phosphatase 2C mutants. The Plant cell 13: 2513-2523.
Mustilli A C, Merlot S, Vavassuer A, Fenzi F, Giraudat J (2002) Arabidopsis OST1 protein kinase mediates the regulation of stomatal aperture by abscisic acid and acts upstream of reactive oxygen species production. Plant cell 14: 3089-3099.
Negi J, Matsuda O, Nagasawa T, Oba Y, Takahashi H, Kawai-Yamada M, Uchimiya H, Hashimoto M, Iba K (2008) CO₂ regulator SLAC1 and its homologues are essential for anion homeostasis in plant cells. Nature 452: 483-486.
Nishimura N, Sarkeshik A, Nito K, Park S Y, Wang A, Carvalho P C, Lee S, Caddell D F, Cutler S R, Chory J, Yates J R, Schroeder J I (2010) PYR/PYL/RCAR family members are major in-vivo ABI1 protein phosphatase 2C-interacting in Arabidopsis. Plant J 61: 290-299
Park S Y, Fung P, Nishimura N, Jensen D R, Fujii H, Zhao Y, Lumba S, Santiago J, Rodrigues A, Chow T F, Alfred S E, Bonetta D, Finkelstein R, Provart N J, Desveaux D, Rodriguez P L, McCourt P, Zhu J K, Schroeder J I, Volkman B F, Cutler S R (2009) Abscisic acid inhibits type 2C protein phosphatases via the PYR/PYL family of START proteins. Science 324: 1068-1071
Pei Z M, Kuchitsu K, Ward J M, Schwarz M, Schroeder J I (1997) differential abscisic acid regulation of guard cell slow anion channels in Arabidopsis wild type and abi1 and abi2 mutants, Plant cell 9: 409-423
Pei Z M, Murata Y, Benning G, Thomine S, Klusener B, Allen G J, Grill E, Schroeder J I (2000) Calcium channels activated by hydrogen peroxide mediate absicisic acid signalling in guard cells. Nature 406: 731-734
Putnam R W, Filosa J A, Ritucci N A (2004) Cellular mechanisms involved in CO₂ and acid signaling in chemosensitive neurons. Am J Physiol Cell Physiol 287: C1493-1526
Raschke K (1975) Simultaneous requirement of carbon dioxide and abscisic acid for stomatal closing in Xanthium strumarium L. Planta 125: 243-259
Raschke K, Hedrich R, Reckmann U, Schroeder J I (1988) Exploring biophysical and biochemical components of the osmotic motor that drives stomatal movements. Bot Acta 101: 283-294
Raschke K, Shabahang M, Wolf R (2003) The slow and the quick anion conductance in whole guard cells: their voltage-dependent alternation, and the modulation of their activities by abscisic acid and CO₂. Planta 217: 639-650
Rodriguez P L, Benning G, Grill E (1998) ABI2, a second protein phosphatase 2C involved in abscisic acid signal transduction in Arabidopsis. FEBS Lett 421: 185-190
Roelfsema M R, Hanstein S, Felle H H, Hedrich R (2002) CO₂ provides an intermediate link in the red light response of guard cells. Plant J 32: 65-75
Roelfsema M R, Levchenko V, Hedrich R (2004) ABA depolarizes guard cells in intact plants, through a transient activation of R- and S-type anion channels. Plant J 37:578-588
Sanders D, Brownlee C, Harper J F (1999) Communicating with calcium. Plant cell 11: 691-706
Santiago J, Rodrigues A, Saez A, Rubio S, Antoni R, Dupeux F, Park S Y, Marquez J A, Cutler S R, Rodriguez P L (2009) Modulation of drought resistance by the abscisic acid receptor PYL5 through inhibition of clade A PP2Cs. Plant J 60: 575-588
Schmidt C, Schelle I, Liao Y J, Schroeder J I (1995) Strong regulation of slow anion channels and abscisic acid signaling in guard cells by phosphorylation and dephosphorylation events. Proc Natl Acad Sci USA 92: 9535-9539
Schmidt C, Schroeder J I (1994) Anion selectivity of slow anion channels in the plasma membrane of guard cells (large nitrate permeability). Plant physiol 106: 383-391
Schroeder J I, Allen G J, Hugouvieux V, Kwak J M, Waner D (2001) Guard cell signal transduction. Annu Rev Plant Physiol and Plant Mol Biol 52: 627-658
Schroeder J I, Hagiwara S (1989) Cytosolic calcium regulates ion channels in the plasma membrane of Vicia faba guard cells. Nature 338: 427-430
Schroeder J I, Hagiwara S (1990) Repetitive increases in cytosolic Ca²⁺ of guard cells by abscisic acid activation of nonselective Ca²⁺ permeable channels. Proc Natl Acad Sci USA 87: 9305-9309
Schroeder J I, Raschke K, Neher E (1987) Voltage dependence of K channels in guard-cell protoplasts. Proc Natl Acad Sci USA 84: 4108-4112
Schulte A, Lorenzen I, Bottcher M, Plieth C (2006) A novel fluorescent pH probe for expression in plants PI Methods 2:7
Schwartz A (1985) Role of Ca2+ and EGTA on stomatal movements in Commelina Communis L. Plant Physiol 79: 1003-1005
Siegel R S, Xue S, Murata Y, Yang Y, Nishimura N, Wang A, Schroeder J I (2009) Calcium elevation-dependent and attenuated resting calcium-dependent abscisic acid induction of stomatal closure and abscisic acid-induced enhancement of calcium sensitivities of S-type anion and inward-rectifying K⁺ channels in Arabidopsis guard cells. Plant J 59:207-220
Sirichandra C, Wasilewska A, Vlad F, Valon C, Leung J (2009) The guard cells as a single-cell model towards understanding drought tolerance and abscisic acid action. J Exp Bot 60: 1439-1463
Speight J G (2005) Lange's Handbook of Chemistry (16th Edition): McGraw-Hill Press, New York USA.
Staxen I, Pical C, Montgomery L T, Gray J E, Hetherington A M, McAinsh M R (1999) Abscisic acid induces oscillations in guard-cell cytosolic free calcium that involve phosphoinositide-specific phospholipase C. Proc Natl Acad Sci USA 96: 1779-1784
Supuran C T (2008) Carbonic anhydrases—an overview. Curr Pharm Des 14: 603-614
Tester M, Blatt M R (1989) Direct measurement of k channels in thylakoid membranes by incorporation of vesicles into planar lipid bilayers. Plant physiol 91: 249-252
Umezawa T, Sugiyama N, Mizoguchi M, Hayashi S, Myouga F, Yamaguchi-Shinozaki K, Ishihama Y, Hirayama T, Shinozaki K (2009) Type 2C protein phosphatases directly regulate abscisic acid-activated protein kinases in Arabidopsis. Proc Natl Acad Sci USA 106: 17588-17593
Vahisalu T, Kollist H, Wang Y F, Nishimura N, Chan W Y, Valerio G, Lamminmaki A, Brosche M, Moldau H, Desikan R, Schroeder J I, Kangasjarvi J (2008) SLAC1 is required for plant guard cell S-type anion channel function in stomatal signalling. Nature 452: 487-491
Vahisalu T, Puzorjova I, Brosche M, Valk E, Lepiku M, Moldau H, Pechter P, Wang Y S, Lindgren O, Salojarvi J, Loog M, Kangasjarvi J, Kollist H (2010) Ozone-triggered rapid stomatal response involves the production of reactive oxygen species, and is controlled by SLAC1 and OST1. Plant J 62: 442-453
Vlad F, Rubio S, Rodrigues A, Sirichandra C, Belin C, Robert N, Leung J, Rodriguez P L, Lauriere C, Merlot S (2009) Protein phosphatases 2C regulate the activation the Snf1-related Kinase OST1 by abscisic acid in Arabidopsis. Plant cell 21: 3170-3184
Webb A A, Hetherington A M (1997) Convergence of the abscisic acid, CO₂, and extracellular calcium signal transduction pathways in stomatal guard cells. Plant physiol 114: 1557-1560
Webb A A R, McAinsh M R, Mansfield T A, Hetherington A M (1996) Carbon dioxide induces increases in guard cell cytosolic free calcium. Plant J 9: 297-304
Yang Y, Costa A, Leonhardt N, Siegel R S, Schroeder J I (2008) Isolation of a strong Arabidopsis guard cell promoter and its potential as a research tool. Plant Methods 4: 1-15
Yarmolinsky D A, Zuker C S, Ryba N J (2009) Common sense about taste: from mammals to insects. Cell 139: 234-244
Yoshida R, Hobo T, Ichimura K, Mizoguchi T, Takahashi F, Aronso J, Ecker J R, Shinozaki K (2002) ABA-activated SnRK2 protein kinase is required for dehydration stress signaling in Arabidopsis. Plant Cell Physiol 43: 1743-1483
Yoshida R, Umezawa T, Mizoguchi T, Takahashi S, Takahashi F, Shinozaki K (2006) The regulatory domain of SRK2E/OST1/SnRK2.6 interacts with ABI1 and integrates abscisic acid (ABA) and osmotic stress signals controlling stomatal closure in Arabidopsis. J. Biol Chem 281: 5310-5318
Young J J, Mehta S, Israelsson M, Godoski J, Grill E, Schroeder J I (2006) CO₂ signaling in guard cells: calcium sensitivity response modulation, a Ca2+-independent phase, and CO₂ insensitivity of the gca2 mutant. Proc Natl Acad Sci USA 103: 7506-7511
Zheng Z, Xu X, Crosley R A, Greenwalt S A, Sun Y, Blakeslee B, Wang L, Ni W, Sopko M S, Yao C, Yau K, Burton S, Zhuang M, McCaskill D G, Gachotte D, Thompson M, Greene T W (2010) The protein kinase SnRK2.6 mediates the regulation of sucrose metabolism and plant growth in Arabidopsis. Plant physiol 153: 99-113
Zhu S Y, Yu X C, Wang X J, Zhao R, Li Y, Fan R C, Shang Y, Du S Y, Wang X F, Wu F Q, Xu Y H, Zhang X Y, Zhang D P (2007) Two calcium-dependent protein kinases, CPK4 and CPK11, regulate abscisic acid signal transduction in Arabidopsis. Plant cell 19: 3019-3036

### SUPPLEMENTARY REFERENCES

Hu H, Boisson-Dernier A, Israelsson-Nordstrom M, Bohmer M, Xue S, Ries A, Godoski J, Kuhn J M, Schroeder J I (2010) Carbonic anhydrases are upstream regulators of CO₂-controlled stomatal movements in guard cells. Nat Cell Biol 12: 87-93
Kollist T, Moldau H, Rasulov B, Oja V, Ramma H, Huve K, Jaspers P, Kangasjarvi J, Kollist H (2007) A novel device detects a rapid ozone-induced transient stomatal closure in intact Arabidopsis and its absence in abi2 mutant. Physiol Plantarium 129: 796-803
Vahisalu T, Kollist H, Wang Y F, Nishimura N, Chan W Y, Valerio G, Lamminmaki A, Brosche M, Moldau H, Desikan R, Schroeder J I, Kangasjarvi J (2008) SLAC1 is required for plant guard cell S-type anion channel function in stomatal signalling. Nature 452: 487-491
Young J J, Mehta S, Israelsson M, Godoski J, Grill E, Schroeder J I (2006) CO₂ signaling in guard cells: calcium sensitivity response modulation, a Ca²⁺-independent phase, and CO₂ insensitivity of the gca2 mutant. Proc Natl Acad Sci USA 103: 7506-7511.

| SEQ ID NO: 1 | |
|---|---|
| cgaacggtcg tcataattcc ttgaaacctc gaaaatccaa aaacccatat ccaatcttct | 60 |
| tcccatataa attaagattt ttatttattt atttgtttac ttatttcaat tcccaaaatc | 120 |
| ctctgcctca tcatcttcaa actgttacca cgtccatagg gttgtcgaag agctaggaag | 180 |
| agccttacca agagcttctt cttcccctaa catttaggtt ggtaggagaa gcaaaggaag | 240 |
| agatcattta taatggctcc tgcattcgga aaatgtttca tgttctgctg cgctaaaacc | 300 |
| tccccggaaa aagacgaaat ggcaacggaa tcgtacgaag ccgccattaa aggactcaat | 360 |
| gatcttctca gtacgaaagc ggatctcgga aacgtcgccg ccgcgaagat caaagcgttg | 420 |
| acggcggagc taaaggagct tgactcaagc aattcagacg caattgaacg aatcaagacc | 480 |
| ggttttactc aattcaaaac cgagaaatat ttgaagaata gtactttgtt caatcatctt | 540 |
| gccaagactc agaccccaaa gtttctggtg tttgcttgct ctgattctcg agtttgtcca | 600 |
| tctcacatct tgaatttcca acctggtgag gcttttgttg tcagaaacat agccaatatg | 660 |
| gttccacctt ttgaccagaa gagacactct ggagttggcg ccgccgttga atacgcagtt | 720 |
| gtacatctca aggtggagaa cattttggtg ataggccata gctgctgtgg tggtattaag | 780 |
| ggactcatgt ccattgaaga tgatgctgcc ccaactcaaa gtgacttcat tgaaaattgg | 840 |
| gtgaagatag gcgcatcagc gaggaacaag atcaaggagg aacataaaga cttgagctac | 900 |
| gatgatcaat gcaacaagtg tgagaaggaa gctgtgaacg tatcgcttgg aaacttgctt | 960 |
| tcgtacccat tcgtgagagc tgaggtggtg aagaacacac ttgcaataag aggaggtcac | 1020 |
| tacaatttcg tcaaaggaac gtttgatctc tgggagctcg atttcaagac cactcctgct | 1080 |
| tttgccttct cttaagaaag aaagctaccg gaacatataa aactcttttg agataaaaaa | 1140 |
| agacactttg actcatcttt cttcattctc tcatgttgat gattcctctc caacttcttt | 1200 |
| gatttctttt tgttaattca aaacttcaac tttgctgctt ctatttcaaa agctcaaaca | 1260 |
| ataaagctgt aaccaacgtt tgaaacttct atatttgtct aattgatgtt tgaacgaaga | 1320 |
| tttgaacttt ccttct | 1336 |
| SEQ ID NO: 2/SEQ ID NO: 3 | |
| atg gct cct gca ttc gga aaa tgt ttc atg ttc tgc tgc gct aaa acc | 48 |
| Met Ala Pro Ala Phe Gly Lys Cys Phe Met Phe Cys Cys Ala Lys Thr | |
| 1 5 10 15 | |
| tcc ccg gaa aaa gac gaa atg gca acg gaa tcg tac gaa gcc gcc att | 96 |
| Ser Pro Glu Lys Asp Glu Met Ala Thr Glu Ser Tyr Glu Ala Ala Ile | |
| 20 25 30 | |
| aaa gga ctc aat gat ctt ctc agt acg aaa gcg gat ctc gga aac gtc | 144 |
| Lys Gly Leu Asn Asp Leu Leu Ser Thr Lys Ala Asp Leu Gly Asn Val | |
| 35 40 45 | |
| gcc gcc gcg aag atc aaa gcg ttg acg gcg gag cta aag gag ctt gac | 192 |
| Ala Ala Ala Lys Ile Lys Ala Leu Thr Ala Glu Leu Lys Glu Leu Asp | |
| 50 55 60 | |
| tca agc aat tca gac gca att gaa cga atc aag acc ggt ttt act caa | 240 |
| Ser Ser Asn Ser Asp Ala Ile Glu Arg Ile Lys Thr Gly Phe Thr Gln | |
| 65 70 75 80 | |
| ttc aaa acc gag aaa tat ttg aag aat agt act ttg ttc aat cat ctt | 288 |
| Phe Lys Thr Glu Lys Tyr Leu Lys Asn Ser Thr Leu Phe Asn His Leu | |
| 85 90 95 | |
| gcc aag act cag acc cca aag ttt ctg gtg ttt gct tgc tct gat tct | 336 |
| Ala Lys Thr Gln Thr Pro Lys Phe Leu Val Phe Ala Cys Ser Asp Ser | |
| 100 105 110 | |
| cga gtt tgt cca tct cac atc ttg aat ttc caa cct ggt gag gct ttt | 384 |
| Arg Val Cys Pro Ser His Ile Leu Asn Phe Gln Pro Gly Glu Ala Phe | |
| 115 120 125 | |
| gtt gtc aga aac ata gcc aat atg gtt cca cct ttt gac cag aag aga | 432 |
| Val Val Arg Asn Ile Ala Asn Met Val Pro Pro Phe Asp Gln Lys Arg | |
| 130 135 140 | |
| cac tct gga gtt ggc gcc gcc gtt gaa tac gca gtt gta cat ctc aag | 480 |
| His Ser Gly Val Gly Ala Ala Val Glu Tyr Ala Val Val His Leu Lys | |
| 145 150 155 160 | |
| gtg gag aac att ttg gtg ata ggc cat agc tgc tgt ggt ggt att aag | 528 |
| Val Glu Asn Ile Leu Val Ile Gly His Ser Cys Cys Gly Gly Ile Lys | |
| 165 170 175 | |
| gga ctc atg tcc att gaa gat gat gct gcc cca act caa agt gac ttc | 576 |
| Gly Leu Met Ser Ile Glu Asp Asp Ala Ala Pro Thr Gln Ser Asp Phe | |
| 180 185 190 | |
| att gaa aat tgg gtg aag ata ggc gca tca gcg agg aac aag atc aag | 624 |
| Ile Glu Asn Trp Val Lys Ile Gly Ala Ser Ala Arg Asn Lys Ile Lys | |
| 195 200 205 | |
| gag gaa cat aaa gac ttg agc tac gat gat caa tgc aac aag tgt gag | 672 |
| Glu Glu His Lys Asp Leu Ser Tyr Asp Asp Gln Cys Asn Lys Cys Glu | |
| 210 215 220 | |
| aag gaa gct gtg aac gta tcg ctt gga aac ttg ctt tcg tac cca ttc | 720 |
| Lys Glu Ala Val Asn Val Ser Leu Gly Asn Leu Leu Ser Tyr Pro Phe | |
| 225 230 235 240 | |
| gtg aga gct gag gtg gtg aag aac aca ctt gca ata aga gga ggt cac | 768 |
| Val Arg Ala Glu Val Val Lys Asn Thr Leu Ala Ile Arg Gly Gly His | |
| 245 250 255 | |
| tac aat ttc gtc aaa gga acg ttt gat ctc tgg gag ctc gat ttc aag | 816 |
| Tyr Asn Phe Val Lys Gly Thr Phe Asp Leu Trp Glu Leu Asp Phe Lys | |
| 260 265 270 | |
| acc act cct gct ttt gcc ttc tct taa (SEQ ID NO: 2) | 843 |
| Thr Thr Pro Ala Phe Ala Phe Ser (SEQ ID NO: 3) | |
| 275 280 | |
| SEQ ID NO: 3 | |
| Met Ala Pro Ala Phe Gly Lys Cys Phe Met Phe Cys Cys Ala Lys Thr | |
| 1 5 10 15 | |
| Ser Pro Glu Lys Asp Glu Met Ala Thr Glu Ser Tyr Glu Ala Ala Ile | |
| 20 25 30 | |
| Lys Gly Leu Asn Asp Leu Leu Ser Thr Lys Ala Asp Leu Gly Asn Val | |
| 35 40 45 | |
| Ala Ala Ala Lys Ile Lys Ala Leu Thr Ala Glu Leu Lys Glu Leu Asp | |
| 50 55 60 | |
| Ser Ser Asn Ser Asp Ala Ile Glu Arg Ile Lys Thr Gly Phe Thr Gln | |
| 65 70 75 80 | |
| Phe Lys Thr Glu Lys Tyr Leu Lys Asn Ser Thr Leu Phe Asn His Leu | |
| 85 90 95 | |
| Ala Lys Thr Gln Thr Pro Lys Phe Leu Val Phe Ala Cys Ser Asp Ser | |
| 100 105 110 | |
| Arg Val Cys Pro Ser His Ile Leu Asn Phe Gln Pro Gly Glu Ala Phe | |
| 115 120 125 | |
| Val Val Arg Asn Ile Ala Asn Met Val Pro Pro Phe Asp Gln Lys Arg | |
| 130 135 140 | |
| His Ser Gly Val Gly Ala Ala Val Glu Tyr Ala Val Val His Leu Lys | |
| 145 150 155 160 | |
| Val Glu Asn Ile Leu Val Ile Gly His Ser Cys Cys Gly Gly Ile Lys | |
| 165 170 175 | |
| Gly Leu Met Ser Ile Glu Asp Asp Ala Ala Pro Thr Gln Ser Asp Phe | |
| 180 185 190 | |
| Ile Glu Asn Trp Val Lys Ile Gly Ala Ser Ala Arg Asn Lys Ile Lys | |
| 195 200 205 | |
| Glu Glu His Lys Asp Leu Ser Tyr Asp Asp Gln Cys Asn Lys Cys Glu | |
| 210 215 220 | |
| Lys Glu Ala Val Asn Val Ser Leu Gly Asn Leu Leu Ser Tyr Pro Phe | |
| 225 230 235 240 | |
| Val Arg Ala Glu Val Val Lys Asn Thr Leu Ala Ile Arg Gly Gly His | |
| 245 250 255 | |
| Tyr Asn Phe Val Lys Gly Thr Phe Asp Leu Trp Glu Leu Asp Phe Lys | |
| 260 265 270 | |
| Thr Thr Pro Ala Phe Ala Phe Ser | |
| 275 280 | |
| SEQ ID NO: 4 | |
| caaaattcat gtgttagttc ttcttcttta caaaattgag tttaaactgt tttattacta | 60 |
| atccaaatga ggaatcactt tgcactatta atagaaaata atacacaacc aaacatctaa | 120 |
| aagatactat aatagtagag atcaaagacc tgagcaaaaa ctgaaagaaa aaaaaaaaaa | 180 |
| aaaaaaaaga cttctcctca aaaatggcgt ttacactagg tggaagagct cgtcgtctag | 240 |
| tctctgcaac atcagttcat caaaatggtt gcttacacaa actgcaacaa attggatcgg | 300 |
| atcggtttca gcttggtgaa gcaaaagcaa taagattact acccaggaga acaaacatgg | 360 |
| ttcaagaatt aggaatcagg gaagaattta tggatctaaa cagagaaaca gagacaagtt | 420 |
| atgattttct ggatgaaatg agacacagat ttctgaaatt caagagacaa aagtatctac | 480 |
| cggagataga aaagtttaaa gctttggcca tagctcaatc accaaaggta atggtgatag | 540 |
| gatgtgcaga ttcaagggta tgtccatctt atgtactagg atttcaacct ggtgaagctt | 600 |
| ttactatccg aaatgtcgcc aatctcgtta ccccggttca gaatggacca acagaaacca | 660 |
| actcggctct tgagtttgcg gtcaccactc ttcaggttga gaacattata gttatgggtc | 720 |
| atagcaattg tggaggaatt gcagcactta tgagtcatca aaaccaccaa gggcaacact | 780 |
| ctagtttagt agaaaggtgg gttatgaatg ggaaagccgc taagttaaga acacaattag | 840 |
| cttcatcaca tttatccttt gatgaacaat gcagaaactg tgagaaggaa tctataaagg | 900 |
| attctgtgat gaatttgata acttattcat ggataagaga tagagtaaag agaggtgaag | 960 |
| tcaagattca tggatgttat tacaatttgt cagattgtag tcttgagaag tggagattaa | 1020 |
| gttcagacaa gactaactat ggattctata tttcagacag agagatatgg agttgagtaa | 1080 |
| atattgaaca atcctcagtt ctaatattca gatgtatctt tgtacatacg aaatgatatt | 1140 |
| tacacaattg g | 1151 |
| SEQ ID NO: 5/SEQ ID NO: 6 | |
| atg gcg ttt aca cta ggt gga aga gct cgt cgt cta gtc tct gca aca | 48 |
| Met Ala Phe Thr Leu Gly Gly Arg Ala Arg Arg Leu Val Ser Ala Thr | |
| 1 5 10 15 | |
| tca gtt cat caa aat ggt tgc tta cac aaa ctg caa caa att gga tcg | 96 |
| Ser Val His Gln Asn Gly Cys Leu His Lys Leu Gln Gln Ile Gly Ser | |
| 20 25 30 | |
| gat cgg ttt cag ctt ggt gaa gca aaa gca ata aga tta cta ccc agg | 144 |
| Asp Arg Phe Gln Leu Gly Glu Ala Lys Ala Ile Arg Leu Leu Pro Arg | |
| 35 40 45 | |
| aga aca aac atg gtt caa gaa tta gga atc agg gaa gaa ttt atg gat | 192 |
| Arg Thr Asn Met Val Gln Glu Leu Gly Ile Arg Glu Glu Phe Met Asp | |
| 50 55 60 | |
| cta aac aga gaa aca gag aca agt tat gat ttt ctg gat gaa atg aga | 240 |
| Leu Asn Arg Glu Thr Glu Thr Ser Tyr Asp Phe Leu Asp Glu Met Arg | |
| 65 70 75 80 | |
| cac aga ttt ctg aaa ttc aag aga caa aag tat cta ccg gag ata gaa | 288 |
| His Arg Phe Leu Lys Phe Lys Arg Gln Lys Tyr Leu Pro Glu Ile Glu | |
| 85 90 95 | |
| aag ttt aaa gct ttg gcc ata gct caa tca cca aag gta atg gtg ata | 336 |
| Lys Phe Lys Ala Leu Ala Ile Ala Gln Ser Pro Lys Val Met Val Ile | |
| 100 105 110 | |
| gga tgt gca gat tca agg gta tgt cca tct tat gta cta gga ttt caa | 384 |
| Gly Cys Ala Asp Ser Arg Val Cys Pro Ser Tyr Val Leu Gly Phe Gln | |
| 115 120 125 | |
| cct ggt gaa gct ttt act atc cga aat gtc gcc aat ctc gtt acc ccg | 432 |
| Pro Gly Glu Ala Phe Thr Ile Arg Asn Val Ala Asn Leu Val Thr Pro | |
| 130 135 140 | |
| gtt cag aat gga cca aca gaa acc aac tcg gct ctt gag ttt gcg gtc | 480 |
| Val Gln Asn Gly Pro Thr Glu Thr Asn Ser Ala Leu Glu Phe Ala Val | |
| 145 150 155 160 | |
| acc act ctt cag gtt gag aac att ata gtt atg ggt cat agc aat tgt | 528 |
| Thr Thr Leu Gln Val Glu Asn Ile Ile Val Met Gly His Ser Asn Cys | |
| 165 170 175 | |
| gga gga att gca gca ctt atg agt cat caa aac cac caa ggg caa cac | 576 |
| Gly Gly Ile Ala Ala Leu Met Ser His Gln Asn His Gln Gly Gln His | |
| 180 185 190 | |
| tct agt tta gta gaa agg tgg gtt atg aat ggg aaa gcc gct aag tta | 624 |
| Ser Ser Leu Val Glu Arg Trp Val Met Asn Gly Lys Ala Ala Lys Leu | |
| 195 200 205 | |
| aga aca caa tta gct tca tca cat tta tcc ttt gat gaa caa tgc aga | 672 |
| Arg Thr Gln Leu Ala Ser Ser His Leu Ser Phe Asp Glu Gln Cys Arg | |
| 210 215 220 | |
| aac tgt gag aag gaa tct ata aag gat tct gtg atg aat ttg ata act | 720 |
| Asn Cys Glu Lys Glu Ser Ile Lys Asp Ser Val Met Asn Leu Ile Thr | |
| 225 230 235 240 | |
| tat tca tgg ata aga gat aga gta aag aga ggt gaa gtc aag att cat | 768 |
| Tyr Ser Trp Ile Arg Asp Arg Val Lys Arg Gly Glu Val Lys Ile His | |
| 245 250 255 | |
| gga tgt tat tac aat ttg tca gat tgt agt ctt gag aag tgg aga tta | 816 |
| Gly Cys Tyr Tyr Asn Leu Ser Asp Cys Ser Leu Glu Lys Trp Arg Leu | |
| 260 265 270 | |
| agt tca gac aag act aac tat gga ttc tat att tca gac aga gag ata | 864 |
| Ser Ser Asp Lys Thr Asn Tyr Gly Phe Tyr Ile Ser Asp Arg Glu Ile | |
| 275 280 285 | |
| tgg agt tga (SEQ ID NO: 5) | 873 |
| Trp Ser (SEQ ID NO: 6) | 290 |
| SEQ ID NO: 6 | |
| Met Ala Phe Thr Leu Gly Gly Arg Ala Arg Arg Leu Val Ser Ala Thr | |
| 1 5 10 15 | |
| Ser Val His Gln Asn Gly Cys Leu His Lys Leu Gln Gln Ile Gly Ser | |
| 20 25 30 | |
| Asp Arg Phe Gln Leu Gly Glu Ala Lys Ala Ile Arg Leu Leu Pro Arg | |
| 35 40 45 | |
| Arg Thr Asn Met Val Gln Glu Leu Gly Ile Arg Glu Glu Phe Met Asp | |
| 50 55 60 | |
| Leu Asn Arg Glu Thr Glu Thr Ser Tyr Asp Phe Leu Asp Glu Met Arg | |
| 65 70 75 80 | |
| His Arg Phe Leu Lys Phe Lys Arg Gln Lys Tyr Leu Pro Glu Ile Glu | |
| 85 90 95 | |
| Lys Phe Lys Ala Leu Ala Ile Ala Gln Ser Pro Lys Val Met Val Ile | |
| 100 105 110 | |
| Gly Cys Ala Asp Ser Arg Val Cys Pro Ser Tyr Val Leu Gly Phe Gln | |
| 115 120 125 | |
| Pro Gly Glu Ala Phe Thr Ile Arg Asn Val Ala Asn Leu Val Thr Pro | |
| 130 135 140 | |
| Val Gln Asn Gly Pro Thr Glu Thr Asn Ser Ala Leu Glu Phe Ala Val | |
| 145 150 155 160 | |
| Thr Thr Leu Gl Val Glu Asn Ile Ile Val Met Gly His Ser Asn Cys | |
| 165 170 175 | |
| Gly Gly Ile Ala Ala Leu Met Ser His Gln Asn His Gln Gly Gln His | |
| 180 185 190 | |
| Ser Ser Leu Val Glu Arg Trp Val Met Asn Gly Lys Ala Ala Lys Leu | |
| 195 200 205 | |
| Arg Thr Gln Leu Ala Ser Ser His Leu Ser Phe Asp Glu Gln Cys Arg | |
| 210 215 220 | |
| Asn Cys Glu Lys Glu Ser Ile Lys Asp Ser Val Met Asn Leu Ile Thr | |
| 225 230 235 240 | |
| Tyr Ser Trp Ile Arg Asp Arg Val Lys Arg Gly Glu Val Lys Ile His | |
| 245 250 255 | |
| Gly Cys Tyr Tyr Asn Leu Ser Asp Cys Ser Leu Glu Lys Trp Arg Leu | |
| 260 265 270 | |
| Ser Ser Asp Lys Thr Asn Tyr Gly Phe Tyr Ile Ser Asp Arg Glu Ile | |
| 275 280 285 | |
| Trp Ser | |
| 290 | |
| SEQ ID NO: 7 | |
| atgagactcc gttcttttaa actcccaaat ctttcaacca atcccattat tcacttaagt | 60 |
| atatagtagc ttccataaga gtcttagttc taactataaa tacacatatc tcactctctc | 120 |
| tgatctccgc ttctcttcgc caacaaatgt cgaccgctcc tctctccggc ttctttctca | 180 |
| cttcactttc tccttctcaa tcttctctcc agaaactctc tcttcgtact tcttccaccg | 240 |
| tcgcttgcct cccacccgcc tcttcttctt cctcatcttc ctcctcctcg tcttcccgtt | 300 |
| ccgttccaac gcttatccgt aacgagccag tttttgccgc tcctgctcct atcattgccc | 360 |
| cttattggag tgaagagatg ggaaccgaag catacgacga ggctattgaa gctctcaaga | 420 |
| agcttctcat cgagaaggaa gagctaaaga cggttgcagc ggcaaaggtg gagcagatca | 480 |
| cagcggctct tcagacaggt acttcatccg acaagaaagc tttcgacccc gtcgaaacca | 540 |
| ttaagcaggg cttcatcaaa ttcaagaagg agaaatacga aaccaaccct gctttgtacg | 600 |
| gtgagctcgc aaagggtcaa agtcctaagt acatggtgtt tgcttgttca gactcacgtg | 660 |
| tgtgtccatc acacgttctg gactttcagc caggagatgc cttcgtggtc cgtaacatag | 720 |
| ccaacatggt tcctcctttc gacaaggtca aatacggtgg cgttggagca gccattgaat | 780 |
| acgcggtctt acaccttaag gtggagaaca ttgtggtgat aggacacagt gcatgtggtg | 840 |
| ggatcaaagg gcttatgtct ttccccttag atggaaacaa ctccactgac ttcatagagg | 900 |
| actgggtcaa aatctgttta ccagccaagt caaaggttat atcagaactt ggagattcag | 960 |
| cctttgaaga tcaatgtggc cgatgtgaaa gggaggcggt gaatgtttca ctagcaaacc | 1020 |
| tattgacata tccatttgtg agagaaggac ttgtgaaggg aacacttgct ttgaagggag | 1080 |
| gctactatga cttcgtcaag ggtgcttttg agctttgggg acttgaattt ggcctctccg | 1140 |
| aaactagctc tgttaaagat gtggctacca tactacattg gaagctgtag gaaactcttt | 1200 |
| gaagccttac ccgatttcac attgtcaatt caataacacc aagttgttgt ttacatgcag | 1260 |
| atcttgatga aactggtttt tgattttaca gaattaaaat cttgggggac agaaatttg | 1319 |
| SEQ ID NO: 8 | |
| Met Ser Thr Ala Pro Leu Ser Gly Phe Phe Leu Thr Ser Leu Ser Pro | |
| 1 5 10 15 | |
| Ser Gln Ser Ser Leu Gln Lys Leu Ser Leu Arg Thr Ser Ser Thr Val | |
| 20 25 30 | |
| Ala Cys Leu Pro Pro Ala Ser Ser Ser Ser Ser Ser Ser Ser Ser Ser | |
| 35 40 45 | |
| Ser Ser Arg Ser Val Pro Thr Leu Ile Arg Asn Glu Pro Val Phe Ala | |
| 50 55 60 | |
| Ala Pro Ala Pro Ile Ile Ala Pro Tyr Trp Ser Glu Glu Met Gly Thr | |
| 65 70 75 80 | |
| Glu Ala Tyr Asp Glu Ala Ile Glu Ala Leu Lys Lys Leu Leu Ile Glu | |
| 85 90 95 | |
| Lys Glu Glu Leu Lys Thr Val Ala Ala Ala Lys Val Glu Gln Ile Thr | |
| 100 105 110 | |
| Ala Ala Leu Gln Thr Gly Thr Ser Ser Asp Lys Lys Ala Phe Asp Pro | |
| 115 120 125 | |
| Val Glu Thr Ile Lys Gln Gly Phe Ile Lys Phe Lys Lys Glu Lys Tyr | |
| 130 135 140 | |
| Glu Thr Asn Pro Ala Leu Tyr Gly Glu Leu Ala Lys Gly Gln Ser Pro | |
| 145 150 155 160 | |
| Lys Tyr Met Val Phe Ala Cys Ser Asp Ser Arg Val Cys Pro Ser His | |
| 165 170 175 | |
| Val Leu Asp Phe Gln Pro Gly Asp Ala Phe Val Val Arg Asn Ile Ala | |
| 80 185 190 | |
| Asn Met Val Pro Pro Phe Asp Lys Val Lys Tyr Gly Gly Val Gly Ala | |
| 195 200 205 | |
| Ala Ile Glu Tyr Ala Val Leu His Leu Lys Val Glu Asn Ile Val Val | |
| 210 215 220 | |
| Ile Gly His Ser Ala Cys Gly Gly Ile Lys Gly Leu Met Ser Phe Pro | |
| 225 230 235 240 | |
| Leu Asp Gly Asn Asn Ser Thr Asp Phe Ile Glu Asp Trp Val Lys Ile | |
| 245 250 255 | |
| Cys Leu Pro Ala Lys Ser Lys Val Ile Ser Glu Leu Gly Asp Ser Ala | |
| 260 265 270 | |
| Phe Glu Asp Gln Cys Gly Arg Cys Glu Arg Glu Ala Val Asn Val Ser | |
| 275 280 285 | |
| Leu Ala Asn Leu Leu Thr Tyr Pro Phe Val Arg Glu Gly Leu Val Lys | |
| 290 295 300 | |
| Gly Thr Leu Ala Leu Lys Gly Gly Tyr Tyr Asp Phe Val Lys Gly Ala | |
| 305 310 315 320 | |
| Phe Glu Leu Trp Gly Leu Glu Phe Gly Leu Ser Glu Thr Ser Ser Val | |
| 325 330 335 | |
| Lys Asp Val Ala Thr Ile Leu His Trp Lys Leu | |
| 340 345 | |
| SEQ ID NO: 9 | |
| atgagactcc gttcttttaa actcccaaat ctttcaacca atcccattat tcacttaagt | 60 |
| atatagtagc ttccataaga gtcttagttc taactataaa tacacatatc tcactctctc | 120 |
| tgatctccgc ttctcttcgc caacaaatgt cgaccgctcc tctctccggc ttctttctca | 180 |
| cttcactttc tccttctcaa tcttctctcc agaaactctc tcttcgtact tcttccaccg | 240 |
| tcgcttgcct cccacccgcc tcttcttctt cctcatcttc ctcctcctcg tcttcccgtt | 300 |
| ccgttccaac gcttatccgt aacgagccag tttttgccgc tcctgctcct atcattgccc | 360 |
| cttattggag tgaagagatg ggaaccgaag catacgacga ggctattgaa gctctcaaga | 420 |
| agcttctcat cgagaaggaa gagctaaaga cggttgcagc ggcaaaggtg gagcagatca | 480 |
| cagcggctct tcagacaggt acttcatccg acaagaaagc tttcgacccc gtcgaaacca | 540 |
| ttaagcaggg cttcatcaaa ttcaagaagg agaaatacga aaccaaccct gctttgtacg | 600 |
| gtgagctcgc aaagggtcaa agtcctaagt acatggtgtt tgcttgttca gactcacgtg | 660 |
| tgtgtccatc acacgttctg gactttcagc caggagatgc cttcgtggtc cgtaacatag | 720 |
| ccaacatggt tcctcctttc gacaaggtca aatacggtgg cgttggagca gccattgaat | 780 |
| acgcggtctt acaccttaag gtggagaaca ttgtggtgat aggacacagt gcatgtggtg | 840 |
| ggatcaaagg gcttatgtct ttccccttag atggaaacaa ctccactgac ttcatagagg | 900 |
| actgggtcaa aatctgttta ccagccaagt caaaggttat atcagaactt ggagattcag | 960 |
| cctttgaaga tcaatgtggc cgatgtgaaa gggaggcggt gaatgtttca ctagcaaacc | 1020 |
| tattgacata tccatttgtg agagaaggac ttgtgaaggg aacacttgct ttgaagggag | 1080 |
| gctactatga cttcgtcaag ggtgcttttg agctttgggg acttgaattt ggcctctccg | 1140 |
| aaactagctc tgtatgaacc aatccatcat catcatcatc atcatgacca tccatcatca | 1200 |
| tcatcattat tatcatcgta tataatatat atctacccca tatgtaattt gtaatgtgcc | 1260 |
| tttgactgtg atgagttatc tctccctctc taccaacttt cttcatatat ataaaacaaa | 1320 |
| aaggaaaagc agatgatata gatctttcgt ggtttaatta tgaacaattg tctttattat | 1380 |
| ttgtgtatca aatcggttgt atttatggtt tgattttatt ttctatgttg tttggtaggt | 1440 |
| taaa | 1444 |
| SEQ ID NO: 10 | |
| Met Ser Thr Ala Pro Leu Ser Gly Phe Phe Leu Thr Ser Leu Ser Pro | |
| 1 5 10 15 | |
| Ser Gln Ser Ser Leu Gln Lys Leu Ser Leu Arg Thr Ser Ser Thr Val | |
| 20 25 30 | |
| Ala Cys Leu Pro Pro Ala Ser Ser Ser Ser Ser Ser Ser Ser Ser Ser | |
| 35 40 45 | |
| Ser Ser Arg Ser Val Pro Thr Leu Ile Arg Asn Glu Pro Val Phe Ala | |
| 50 55 60 | |
| Ala Pro Ala Pro Ile Ile Ala Pro Tyr Trp Ser Glu Glu Met Gly Thr | |
| 65 70 75 80 | |
| Glu Ala Tyr Asp Glu Ala Ile Glu Ala Leu Lys Lys Leu Leu Ile Glu | |
| 85 90 95 | |
| Lys Glu Glu Leu Lys Thr Val Ala Ala Ala Lys Val Glu Gln Ile Thr | |
| 100 105 110 | |
| Ala Ala Leu Gln Thr Gly Thr Ser Ser Asp Lys Lys Ala Phe Asp Pro | |
| 115 120 125 | |
| Val Glu Thr Ile Lys Gln Gly Phe Ile Lys Phe Lys Lys Glu Lys Tyr | |
| 130 135 140 | |
| Glu Thr Asn Pro Ala Leu Tyr Gly Glu Leu Ala Lys Gly Gln Ser Pro | |
| 145 150 155 160 | |
| Lys Tyr Met Val Phe Ala Cys Ser Asp Ser Arg Val Cys Pro Ser His | |
| 165 170 175 | |
| Val Leu Asp Phe Gln Pro Gly Asp Ala Phe Val Val Arg Asn Ile Ala | |
| 180 185 190 | |
| Asn Met Val Pro Pro Phe Asp Lys Val Lys Tyr Gly Gly Val Gly Ala | |
| 195 200 205 | |
| Ala Ile Glu Tyr Ala Val Leu His Leu Lys Val Glu Asn Ile Val Val | |
| 210 215 220 | |
| Ile Gly His Ser Ala Cys Gly Gly Ile Lys Gly Leu Met Ser Phe Pro | |
| 225 230 235 240 | |
| Leu Asp Gly Asn Asn Ser Thr Asp Phe Ile Glu Asp Trp Val Lys Ile | |
| 245 250 255 | |
| Cys Leu Pro Ala Lys Ser Lys Val Ile Ser Glu Leu Gly Asp Ser Ala | |
| 260 265 270 | |
| Phe Glu Asp Gln Cys Gly Arg Cys Glu Arg Glu Ala Val Asn Val Ser | |
| 275 280 285 | |
| Leu Ala Asn Leu Leu Thr Tyr Pro Phe Val Arg Glu Gly Leu Val Lys | |
| 290 295 300 | |
| Gly Thr Leu Ala Leu Lys Gly Gly Tyr Tyr Asp Phe Val Lys Gly Ala | |
| 305 310 315 320 | |
| Phe Glu Leu Trp Gly Leu Glu Phe Gly Leu Ser Glu Thr Ser Ser Val | |
| 325 330 335 | |
| SEQ ID NO: 11/SEQ ID NO: 12 | |
| ttgttcattt cctctgatgt cttggtgtcg ttagatattg tctcccaaaa aagaaatctt | 60 |
| cttgacacag agattgaagt cgcaaagaga cagaggaaag agggggagaa a atg gat | 117 |
| Met Asp | |
| 1 | |
| cga cca gca gtg agt ggt cca atg gat ttg ccg att atg cac gat agt | 165 |
| Arg Pro Ala Val Ser Gly Pro Met Asp Leu Pro Ile Met His Asp Ser | |
| 5 10 15 | |
| gat agg tat gaa ctc gtc aag gat att ggc tcc ggt aat ttt gga gtt | 213 |
| Asp Arg Tyr Glu Leu Val Lys Asp Ile Gly Ser Gly Asn Phe Gly Val | |
| 20 25 30 | |
| gcg aga ttg atg aga gac aag caa agt aat gag ctt gtt gct gtt aaa | 261 |
| Ala Arg Leu Met Arg Asp Lys Gln Ser Asn Glu Leu Val Ala Val Lys | |
| 35 40 45 50 | |
| tat atc gag aga ggt gag aag ata gat gaa aat gta aaa agg gag ata | 309 |
| Tyr Ile Glu Arg Gly Glu Lys Ile Asp Glu Asn Val Lys Arg Glu Ile | |
| 55 60 65 | |
| atc aac cac agg tcc tta aga cat ccc aat atc gtt aga ttc aaa gag | 357 |
| Ile Asn His Arg Ser Leu Arg His Pro Asn Ile Val Arg Phe Lys Glu | |
| 70 75 80 | |
| gtt ata tta aca cca acc cat tta gcc att gtt atg gaa tat gca tct | 405 |
| Val Ile Leu Thr Pro Thr His Leu Ala Ile Val Met Glu Tyr Ala Ser | |
| 85 90 95 | |
| gga gga gaa ctt ttc gag cga ata tgc aat gca ggc cgc ttc agc gaa | 453 |
| Gly Gly Glu Leu Phe Glu Arg Ile Cys Asn Ala Gly Arg Phe Ser Glu | |
| 100 105 110 | |
| gac gag gcg agg ttt ttc ttc cag caa ctc att tca gga gtt agt tac | 501 |
| Asp Glu Ala Arg Phe Phe Phe Gln Gln Leu Ile Ser Gly Val Ser Tyr | |
| 115 120 125 130 | |
| tgt cat gct atg caa gta tgt cac cga gac tta aag ctc gag aat acg | 549 |
| Cys His Ala Met Gln Val Cys His Arg Asp Leu Lys Leu Glu Asn Thr | |
| 135 140 145 | |
| tta tta gat ggt agc ccg gcc cct cgt cta aag ata tgt gat ttc gga | 597 |
| Leu Leu Asp Gly Ser Pro Ala Pro Arg Leu Lys Ile Cys Asp Phe Gly | |
| 150 155 160 | |
| tat tct aag tca tca gtg tta cat tcg caa cca aaa tca act gtt gga | 645 |
| Tyr Ser Lys Ser Ser Val Leu His Ser Gln Pro Lys Ser Thr Val Gly | |
| 165 170 175 | |
| act cct gct tac atc gct cct gag gtt tta cta aag aaa gaa tat gat | 693 |
| Thr Pro Ala Tyr Ile Ala Pro Glu Val Leu Leu Lys Lys Glu Tyr Asp | |
| 180 185 190 | |
| gga aag gtt gca gat gtt tgg tct tgt ggg gtt act ctg tat gtc atg | 741 |
| Gly Lys Val Ala Asp Val Trp Ser Cys Gly Val Thr Leu Tyr Val Met | |
| 195 200 205 210 | |
| ctg gtt gga gca tat cct ttc gaa gat ccc gag gaa cca aag aat ttc | 789 |
| Leu Val Gly Ala Tyr Pro Phe Glu Asp Pro Glu Glu Pro Lys Asn Phe | |
| 215 220 225 | |
| agg aaa act ata cat aga atc ctg aat gtt cag tat gct att ccg gat | 837 |
| Arg Lys Thr Ile His Arg Ile Leu Asn Val Gln Tyr Ala Ile Pro Asp | |
| 230 235 240 | |
| tat gtt cac ata tct cct gaa tgt cgc cat ttg atc tcc aga ata ttt | 885 |
| Tyr Val His Ile Ser Pro Glu Cys Arg His Leu Ile Ser Arg Ile Phe | |
| 245 250 255 | |
| gtt gct gac cct gca aag agg ata tca att cct gaa ata agg aac cat | 933 |
| Val Ala Asp Pro Ala Lys Arg Ile Ser Ile Pro Glu Ile Arg Asn His | |
| 260 265 270 | |
| gaa tgg ttt cta aag aat cta ccg gca gat cta atg aac gat aac acg | 981 |
| Glu Trp Phe Leu Lys Asn Leu Pro Ala Asp Leu Met Asn Asp Asn Thr | |
| 275 280 285 290 | |
| atg acc act cag ttt gat gaa tcg gat caa ccg ggc caa agc ata gaa | 1029 |
| Met Thr Thr Gln Phe Asp Glu Ser Asp Gln Pro Gly Gln Ser Ile Glu | |
| 295 300 305 | |
| gaa att atg cag atc att gca gaa gca act gtt cct cct gca ggc act | 1077 |
| Glu Ile Met Gln Ile Ile Ala Glu Ala Thr Val Pro Pro Ala Gly Thr | |
| 310 315 320 | |
| cag aat ctg aac cat tac ctc aca gga agc ttg gac ata gat gac gat | 1125 |
| Gln Asn Leu Asn His Tyr Leu Thr Gly Ser Leu Asp Ile Asp Asp Asp | |
| 325 330 335 | |
| atg gag gaa gac tta gag agc gac ctt gat gat ctt gac atc gac agt | 1173 |
| Met Glu Glu Asp Leu Glu Ser Asp Leu Asp Asp Leu Asp Ile Asp Ser | |
| 340 345 350 | |
| agc gga gag att gtg tac gca atg tga tactatatat ctatttgcat | 1220 |
| Ser Gly Glu Ile Val Tyr Ala Met (SEQ ID NO: 12) | |
| 355 360 | |
| ggtttctgct acaaaaatgt caaacaaaaa atgttgaaga ataagattaa gatgttttgc | 1280 |
| ttgctattga gttggcccaa ctttgtctca atgagtacac tttgaatctt tgatatgcaa | 1340 |
| aagactaaat ttc (SEQ ID NO: 11) | 1353 |
| SEQ ID NO: 12 | |
| Met Asp Arg Pro Ala Val Ser Gly Pro Met Asp Leu Pro Ile Met His | |
| 1 5 10 15 | |
| Asp Ser Asp Arg Tyr Glu Leu Val Lys Asp Ile Gly Ser Gly Asn Phe | |
| 20 25 30 | |
| Gly Val Ala Arg Leu Met Arg Asp Lys Gln Ser Asn Glu Leu Val Ala | |
| 35 40 45 | |
| Val Lys Tyr Ile Glu Arg Gly Glu Lys Ile Asp Glu Asn Val Lys Arg | |
| 50 55 60 | |
| Glu Ile Ile Asn His Arg Ser Leu Arg His Pro Asn Ile Val Arg Phe | |
| 65 70 75 80 | |
| Lys Glu Val Ile Leu Thr Pro Thr His Leu Ala Ile Val Met Glu Tyr | |
| 85 90 95 | |
| Ala Ser Gly Gly Glu Leu Phe Glu Arg Ile Cys Asn Ala Gly Arg Phe | |
| 100 105 110 | |
| Ser Glu Asp Glu Ala Arg Phe Phe Phe Gln Gln Leu Ile Ser Gly Val | |
| 115 120 125 | |
| Ser Tyr Cys His Ala Met Gln Val Cys His Arg Asp Leu Lys Leu Glu | |
| 130 135 140 | |
| Asn Thr Leu Leu Asp Gly Ser Pro Ala Pro Arg Leu Lys Ile Cys Asp | |
| 145 150 155 160 | |
| Phe Gly Tyr Ser Lys Ser Ser Val Leu His Ser Gln Pro Lys Ser Thr | |
| 165 170 175 | |
| Val Gly Thr Pro Ala Tyr Ile Ala Pro Glu Val Leu Leu Lys Lys Glu | |
| 180 185 190 | |
| Tyr Asp Gly Lys Val Ala Asp Val Trp Ser Cys Gly Val Thr Leu Tyr | |
| 195 200 205 | |
| Val Met Leu Val Gly Ala Tyr Pro Phe Glu Asp Pro Glu Glu Pro Lys | |
| 210 215 220 | |
| Asn Phe Arg Lys Thr Ile His Arg Ile Leu Asn Val Gln Tyr Ala Ile | |
| 225 230 235 240 | |
| Pro Asp Tyr Val His Ile Ser Pro Glu Cys Arg His Leu Ile Ser Arg | |
| 245 250 255 | |
| Ile Phe Val Ala Asp Pro Ala Lys Arg Ile Ser Ile Pro Glu Ile Arg | |
| 260 265 270 | |
| Asn His Glu Trp Phe Leu Lys Asn Leu Pro Ala Asp Leu Met Asn Asp | |
| 275 280 285 | |
| Asn Thr Met Thr Thr Gln Phe Asp Glu Ser Asp Gln Pro Gly Gln Ser | |
| 290 295 300 | |
| Ile Glu Glu Ile Met Gln Ile Ile Ala Glu Ala Thr Val Pro Pro Ala | |
| 305 310 315 320 | |
| Gly Thr Gln Asn Leu Asn His Tyr Leu Thr Gly Ser Leu Asp Ile Asp | |
| 325 330 335 | |
| Asp Asp Met Glu Glu Asp Leu Glu Ser Asp Leu Asp Asp Leu Asp Ile | |
| 340 345 350 | |
| Asp Ser Ser Gly Glu Ile Val Tyr Ala Met | |
| 355 360 | |
| SEQ ID NO: 13/SEQ ID NO: 14 | |
| agagaaagct gtttcctttt tatattgaca gagaaaagga aagctgatag agagagagac | 60 |
| agagagagag aaacagagtt caagatcacg agccttcctt cttcttcttc ttcttcatcg | 120 |
| agagcgatca aaggaacaaa aaggatctca agaaacccac ttgtgttgtt ggttagatac | 180 |
| ttcacgggtc tctgaaaacg tctctttctc acaaccataa cttgatcacc caatactcct | 240 |
| tttctcatct taaaggctca aattcatcca cgtcacaccg ttgttcattt cctctgatgt | 300 |
| cttggtgtcg ttagatattg tctcccaaaa aagaaatctt cttgacacag agattgaagt | 360 |
| cgcaaagaga cagaggaaag agggggagaa aatggatcga ccagcagtga gtggtccaat | 420 |
| ggatttgccg attatgcacg atagtgatag gtatgaactc gtcaaggata ttggctccgg | 480 |
| taattttgga gttgcgagat tgatgagaga caagcaaagt aatgagcttg ttgctgttaa | 540 |
| atatatcgag agagtgttgt tttaaaggct ctaggtgttt cttttgttat ggaacgtggt | 600 |
| atta atg gtg gga ctt ttt gta ttt gta cag ata gat gaa aat gta aaa | 649 |
| Met Val Gly Leu Phe Val Phe Val Gln Ile Asp Glu Asn Val Lys | |
| 1 5 10 15 | |
| agg gag ata atc aac cac agg tcc tta aga cat ccc aat atc gtt aga | 697 |
| Arg Glu Ile Ile Asn His Arg Ser Leu Arg His Pro Asn Ile Val Arg | |
| 20 25 30 | |
| ttc aaa gag gtt ata tta aca cca acc cat tta gcc att gtt atg gaa | 745 |
| Phe Lys Glu Val Ile Leu Thr Pro Thr His Leu Ala Ile Val Met Glu | |
| 35 40 45 | |
| tat gca tct gga gga gaa ctt ttc gag cga atc tgc aat gca ggc cgc | 793 |
| Tyr Ala Ser Gly Gly Glu Leu Phe Glu Arg Ile Cys Asn Ala Gly Arg | |
| 50 55 60 | |
| ttc agc gaa gac gag gcg agg ttt ttc ttc cag caa ctc att tca gga | 841 |
| Phe Ser Glu Asp Glu Ala Arg Phe Phe Phe Gln Gln Leu Ile Ser Gly | |
| 65 70 75 | |
| gtt agt tac tgt cat gct atg caa gta tgt cac cga gac tta aag ctc | 889 |
| Val Ser Tyr Cys His Ala Met Gln Val Cys His Arg Asp Leu Lys Leu | |
| 80 85 90 95 | |
| gag aat acg tta tta gat ggt agc ccg gcc cct cgt cta aag ata tgt | 937 |
| Glu Asn Thr Leu Leu Asp Gly Ser Pro Ala Pro Arg Leu Lys Ile Cys | |
| 100 105 110 | |
| gat ttc gga tat tct aag tca tca gtg tta cat tcg caa cca aaa tca | 985 |
| Asp Phe Gly Tyr Ser Lys Ser Ser Val Leu His Ser Gln Pro Lys Ser | |
| 115 120 125 | |
| act gtt gga act cct gct tac atc gct cct gag gtt tta cta aag aaa | 1033 |
| Thr Val Gly Thr Pro Ala Tyr Ile Ala Pro Glu Val Leu Leu Lys Lys | |
| 130 135 140 | |
| gaa tat gat gga aag gtt gca gat gtt tgg tct tgt ggg gtt act ctg | 1081 |
| Glu Tyr Asp Gly Lys Val Ala Asp Val Trp Ser Cys Gly Val Thr Leu | |
| 145 150 155 | |
| tat gtc atg ctg gtt gga gca tat cct ttc gaa gat ccc gag gaa cca | 1129 |
| Tyr Val Met Leu Val Gly Ala Tyr Pro Phe Glu Asp Pro Glu Glu Pro | |
| 160 165 170 175 | |
| aag aat ttc agg aaa act ata cat aga atc ctg aat gtt cag tat gct | 1177 |
| Lys Asn Phe Arg Lys Thr Ile His Arg Ile Leu Asn Val Gln Tyr Ala | |
| 180 185 190 | |
| att ccg gat tat gtt cac ata tct cct gaa tgt cgc cat ttg atc tcc | 1225 |
| Ile Pro Asp Tyr Val His Ile Ser Pro Glu Cys Arg His Leu Ile Ser | |
| 195 200 205 | |
| aga ata ttt gtt gct gac cct gca aag agg ata tca att cct gaa ata | 1273 |
| Arg Ile Phe Val Ala Asp Pro Ala Lys Arg Ile Ser Ile Pro Glu Ile | |
| 210 215 220 | |
| agg aac cat gaa tgg ttt cta aag aat cta ccg gca gat cta atg aac | 1321 |
| Arg Asn His Glu Trp Phe Leu Lys Asn Leu Pro Ala Asp Leu Met Asn | |
| 225 230 235 | |
| gat aac acg atg acc act cag ttt gat gaa tcg gat caa ccg ggc caa | 1369 |
| Asp Asn Thr Met Thr Thr Gln Phe Asp Glu Ser Asp Gln Pro Gly Gln | |
| 240 245 250 255 | |
| agc ata gaa gaa att atg cag atc att gca gaa gca act gtt cct cct | 1417 |
| Ser Ile Glu Glu Ile Met Gln Ile Ile Ala Glu Ala Thr Val Pro Pro | |
| 260 265 270 | |
| gca ggc act cag aat ctg aac cat tac ctc aca gga agc ttg gac ata | 1465 |
| Ala Gly Thr Gln Asn Leu Asn His Tyr Leu Thr Gly Ser Leu Asp Ile | |
| 275 280 285 | |
| gat gac gat atg gag gaa gac tta gag agc gac ctt gat gat ctt gac | 1513 |
| Asp Asp Asp Met Glu Glu Asp Leu Glu Ser Asp Leu Asp Asp Leu Asp | |
| 290 295 300 | |
| atc gac agt agc gga gag att gtg tac gca atg tga tactatatat | 1559 |
| Ile Asp Ser Ser Gly Glu Ile Val Tyr Ala Met (SEQ ID NO: 14) | |
| 305 310 | |
| ctatttgcat ggtttctgct acaaaaatgt caaacaaaaa atgttgaaga ataagattaa | 1619 |
| gatgttttgc ttgctattga gttggcccaa ctttgtctca atgagtacac tttgaatctt | 1679 |
| tgatatgcaa aagactaaat ttc (SEQ ID NO: 13) | 1702 |
| SEQ ID NO: 14 | |
| Met Val Gly Leu Phe Val Phe Val Gln Ile Asp Glu Asn Val Lys Arg | |
| 1 5 10 15 | |
| Glu Ile Ile Asn His Arg Ser Leu Arg His Pro Asn Ile Val Arg Phe | |
| 20 25 30 | |
| Lys Glu Val Ile Leu Thr Pro Thr His Leu Ala Ile Val Met Glu Tyr | |
| 35 40 45 | |
| Ala Ser Gly Gly Gln Leu Phe Glu Arg Ile Cys Asn Ala Gly Arg Phe | |
| 50 55 60 | |
| Ser Glu Asp Glu Ala Arg Phe Phe Phe Gln Gln Leu Ile Ser Gly Val | |
| 65 70 75 80 | |
| Ser Tyr Cys His Ala Met Glu Val Cys His Arg Asp Leu Lys Leu Glu | |
| 85 90 95 | |
| Asn Thr Leu Leu Asp Gly Ser Pro Ala Pro Arg Leu Lys Ile Cys Asp | |
| 100 105 110 | |
| Phe Gly Tyr Ser Lys Ser Ser Val Leu His Ser Gln Pro Lys Ser Thr | |
| 115 120 125 | |
| Val Gly Thr Pro Ala Tyr Ile Ala Pro Gln Val Leu Leu Lys Lys Glu | |
| 130 135 140 | |
| Tyr Asp Gly Lys Val Ala Asp Val Trp Ser Cys Gly Val Thr Leu Tyr | |
| 145 150 155 160 | |
| Val Met Leu Val Gly Ala Tyr Pro Phe Glu Asp Pro Glu Glu Pro Lys | |
| 165 170 175 | |
| Asn Phe Arg Lys Thr Ile His Arg Ile Leu Asn Val Gln Tyr Ala Ile | |
| 180 185 190 | |
| Pro Asp Tyr Val His Ile Ser Pro Glu Cys Arg His Leu Ile Ser Arg | |
| 195 200 205 | |
| Ile Phe Val Ala Asp Pro Ala Lys Arg Ile Ser Ile Pro Gln Ile Arg | |
| 210 215 220 | |
| Asn His Glu Trp Phe Leu Lys Asn Leu Pro Ala Asp Leu Met Asn Asp | |
| 225 230 235 240 | |
| Asn Thr Met Thr Thr Gln Phe Asp Glu Ser Asp Gln Pro Gly Gln Ser | |
| 245 250 255 | |
| Ile Glu Glu Ile Met Gln Ile Ile Ala Glu Ala Thr Val Pro Pro Ala | |
| 260 265 270 | |
| Gly Thr Gln Asn Leu Asn His Tyr Leu Thr Gly Ser Leu Asp Ile Asp | |
| 275 280 285 | |
| Asp Asp Met Glu Glu Asp Leu Glu Ser Asp Leu Asp Asp Leu Asp Ile | |
| 290 295 300 | |
| Asp Ser Ser Gly Glu Ile Val Tyr Ala Met | |
| 305 310 | |
| SEQ ID NO: 15/SEQ ID NO: 16 | |
| aaatagagaa gctcttcaag tatccgatgt ttttgtttaa tcaacaagag gcggagatac | 60 |
| gggagaaatt gcatgtgtaa tcataaaatg tagatgttag cttcgtcgtt tttactatag | 120 |
| tttagttctc ttcttcttct tttttcgtca ttacaatctc tttcttaatt tacttcttct | 180 |
| tgatagtata attaagttgt ttgtaataat ctgtacaaag atgttgtgtt ctcataaaaa | 240 |
| attcaatttt gtaaagaagc tctacatgtt ccttgctctg taaac atg gtc ccc ttt | 297 |
| Met Val Pro Phe | |
| 1 | |
| tgg act aca gtt tct cga aat ggc tca tca gac tca gag acg act ctc | 345 |
| Trp Thr Thr Val Ser Arg Asn Gly Ser Ser Asp Ser Glu Thr Thr Leu | |
| 5 10 15 20 | |
| caa tct gct tca aaa gcc aca aaa cag tat aaa tat cct tct ctt cgt | 393 |
| Gln Ser Ala Ser Lys Ala Thr Lys Gln Tyr Lys Tyr Pro Ser Leu Arg | |
| 25 30 35 | |
| ccc tct cat cgc ctg tct ctc ctc ttc ctc ttc ccg ttc cat tta tcc | 441 |
| Pro Ser His Arg Leu Ser Leu Leu Phe Leu Phe Pro Phe His Leu Ser | |
| 40 45 50 | |
| gca aac gga gct tgt ttt cgg tgc acc tgc ttc agc cac ttc aaa ctt | 489 |
| Ala Asn Gly Ala Cys Phe Arg Cys Thr Cys Phe Ser His Phe Lys Leu | |
| 55 60 65 | |
| gaa ctg aga agg atg gga aac gaa tca tat gaa gac gcc atc gaa gct | 537 |
| Glu Leu Arg Arg Met Gly Asn Glu Ser Tyr Glu Asp Ala Ile Glu Ala | |
| 70 75 80 | |
| ctc aag aag ctt ctc att gag aag gat gat ctg aag gat gta gct gcg | 585 |
| Leu Lys Lys Leu Leu Ile Glu Lys Asp Asp Leu Lys Asp Val Ala Ala | |
| 85 90 95 100 | |
| gcc aag gtg aag aag atc acg gcg gag ctt cag gca gcc tcg tca tcg | 633 |
| Ala Lys Val Lys Lys Ile Thr Ala Glu Leu Gln Ala Ala Ser Ser Ser | |
| 105 110 115 | |
| gac agc aaa tct ttt gat ccc gtc gaa cga att aag gaa ggc ttc gtc | 681 |
| Asp Ser Lys Ser Phe Asp Pro Val Glu Arg Ile Lys Glu Gly Phe Val | |
| 120 125 130 | |
| acc ttc aag aag gag aaa tac gag acc aat cct gct ttg tat ggt gag | 729 |
| Thr Phe Lys Lys Glu Lys Tyr Glu Thr Asn Pro Ala Leu Tyr Gly Glu | |
| 135 140 145 | |
| ctc gcc aaa ggt caa agc cca aag tac atg gtg ttt gct tgt tcg gac | 777 |
| Leu Ala Lys Gly Gln Ser Pro Lys Tyr Met Val Phe Ala Cys Ser Asp | |
| 150 155 160 | |
| tca cga gtg tgc cca tca cac gta cta gac ttc cat cct gga gat gcc | 825 |
| Ser Arg Val Cys Pro Ser His Val Leu Asp Phe His Pro Gly Asp Ala | |
| 165 170 175 180 | |
| ttc gtg gtt cgt aat atc gcc aat atg gtt cct cct ttt gac aag gtc | 873 |
| Phe Val Val Arg Asn Ile Ala Asn Met Val Pro Pro Phe Asp Lys Val | |
| 185 190 195 | |
| aaa tat gca gga gtt gga gcc gcc att gaa tac gct gtc ttg cac ctt | 921 |
| Lys Tyr Ala Gly Val Gly Ala Ala Ile Glu Tyr Ala Val Leu His Leu | |
| 200 205 210 | |
| aag gtg gaa aac att gtg gtg ata ggg cac agt gca tgt ggt ggc atc | 969 |
| Lys Val Glu Asn Ile Val Val Ile Gly His Ser Ala Cys Gly Gly Ile | |
| 215 220 225 | |
| aag ggg ctt atg tca ttt cct ctt gac gga aac aac tct act gac ttc | 1017 |
| Lys Gly Leu Met Ser Phe Pro Leu Asp Gly Asn Asn Ser Thr Asp Phe | |
| 230 235 240 | |
| ata gag gat tgg gtc aaa atc tgt tta cca gca aag tca aaa gtt ttg | 1065 |
| Ile Glu Asp Trp Val Lys Ile Cys Leu Pro Ala Lys Ser Lys Val Leu | |
| 245 250 255 260 | |
| gca gaa agt gaa agt tca gca ttt gaa gac caa tgt ggc cga tgc gaa | 1113 |
| Ala Glu Ser Glu Ser Ser Ala Phe Glu Asp Gln Cys Gly Arg Cys Glu | |
| 265 270 275 | |
| agg gag gca gtg aat gtg tca cta gca aac cta ttg aca tat cca ttt | 1161 |
| Arg Glu Ala Val Asn Val Ser Leu Ala Asn Leu Leu Thr Tyr Pro Phe | |
| 280 285 290 | |
| gtg aga gaa gga gtt gtg aaa gga aca ctt gct ttg aag gga ggc tac | 1209 |
| Val Arg Glu Gly Val Val Lys Gly Thr Leu Ala Leu Lys Gly Gly Tyr | |
| 295 300 305 | |
| tat gac ttt gtt aat ggc tcc ttt gag ctt tgg gag ctc cag ttt gga | 1257 |
| Tyr Asp Phe Val Asn Gly Ser Phe Glu Leu Trp Glu Leu Glu Phe Gly | |
| 310 315 320 | |
| att tcc ccc gtt cat tct ata tga actaacacat caccatcacc atcgctacca | 1311 |
| Ile Ser Pro Val His Ser Ile (SEQ ID NO: 16) | |
| 325 330 | |
| ccaccatcac aaacatcatc atcgtcgtca tcatcatgat cagcatcttc atatataaat | 1371 |
| gttttactct tatttaattg ctacttgtaa tggtatacat ttacttgcga tgagcttctt | 1431 |
| ttccttcatt atccagttat aaaataaata aataaatcat gtttactttc acagatatcg | 1491 |
| ttttgctgaa gttgctttga ttt (SEQ ID NO: 15) | 1514 |
| SEQ ID NO: 16 | |
| Met Val Pro Phe Trp Thr Thr Val Ser Arg Asn Gly Ser Ser Asp Ser | |
| 1 5 10 15 | |
| Glu Thr Thr Leu Gln Ser Ala Ser Lys Ala Thr Lys Gln Tyr Lys Tyr | |
| 20 25 30 | |
| Pro Ser Leu Arg Pro Ser His Arg Leu Ser Leu Leu Phe Leu Phe Pro | |
| 35 40 45 | |
| Phe His Leu Ser Ala Asn Gly Ala Cys Phe Arg Cys Thr Cys Phe Ser | |
| 50 55 60 | |
| His Phe Lys Leu Glu Leu Arg Arg Met Gly Asn Glu Ser Tyr Glu Asp | |
| 65 70 75 80 | |
| Ala Ile Glu Ala Leu Lys Lys Leu Leu Ile Glu Lys Asp Asp Leu Lys | |
| 85 90 95 | |
| Asp Val Ala Ala Ala Lys Val Lys Lys Ile Thr Ala Glu Leu Gln Ala | |
| 100 105 110 | |
| Ala Ser Ser Ser Asp Ser Lys Ser Phe Asp Pro Val Glu Arg Ile Lys | |
| 115 120 125 | |
| Glu Gly Phe Val Thr Phe Lys Lys Glu Lys Tyr Glu Thr Asn Pro Ala | |
| 130 135 140 | |
| Leu Tyr Gly Glu Leu Ala Lys Gly Gln Ser Pro Lys Tyr Met Val Phe | |
| 145 150 155 160 | |
| Ala Cys Ser Asp Ser Arg Val Cys Pro Ser His Val Leu Asp Phe His | |
| 165 170 175 | |
| Pro Gly Asp Ala Phe Val Val Arg Asn Ile Ala Asn Met Val Pro Pro | |
| 180 185 190 | |
| Phe Asp Lys Val Lys Tyr Ala Gly Val Gly Ala Ala Ile Glu Tyr Ala | |
| 195 200 205 | |
| Val Leu His Leu Lys Val Glu Asn Ile Val Val Ile Gly His Ser Ala | |
| 210 215 220 | |
| Cys Gly Gly Ile Lys Gly Leu Met Ser Phe Pro Leu Asp Gly Asn Asn | |
| 225 230 235 240 | |
| Ser Thr Asp Phe Ile Glu Asp Trp Val Lys Ile Cys Leu Pro Ala Lys | |
| 245 250 255 | |
| Ser Lys Val Leu Ala Glu Ser Glu Ser Ser Ala Phe Glu Asp Gln Cys | |
| 260 265 270 | |
| Gly Arg Cys Glu Arg Glu Ala Val Asn Val Ser Leu Ala Asn Leu Leu | |
| 275 280 285 | |
| Thr Tyr Pro Phe Val Arg Glu Gly Val Val Lys Gly Thr Leu Ala Leu | |
| 290 295 300 | |
| Lys Gly Gly Tyr Tyr Asp Phe Val Asn Gly Ser Phe Glu Leu Trp Glu | |
| 305 310 315 320 | |
| Leu Gln Phe Gly Ile Ser Pro Val His Ser Ile | |
| 325 330 | |
| SEQ ID NO: 17/SEQ ID NO: 18 | |
| atgcagtaat ctgataaaac cctccacaga gatttccaac aaaacaggaa ctaaaacaca | 60 |
| ag atg aag att atg atg atg att aag ctc tgc ttc ttc tcc atg tcc | 107 |
| Met Lys Ile Met Met Met Ile Lys Leu Cys Phe Phe Ser Met Ser | |
| 1 5 10 15 | |
| ctc atc tgc att gca cct gca gat gct cag aca gaa gga gta gtg ttt | 155 |
| Leu Ile Cys Ile Ala Pro Ala Asp Ala Gln Thr Glu Gly Val Val Phe | |
| 20 25 30 | |
| gga tat aaa ggc aaa aat gga cca aac caa tgg gga cac tta aac cct | 203 |
| Gly Tyr Lys Gly Lys Asn Gly Pro Asn Gln Trp Gly His Leu Asn Pro | |
| 35 40 45 | |
| cac ttc acc aca tgc gcg gtc ggt aaa ttg caa tct cca att gat att | 251 |
| His Phe Thr Thr Cys Ala Val Gly Lys Leu Gln Ser Pro Ile Asp Ile | |
| 50 55 60 | |
| caa agg agg caa ata ttt tac aac cac aaa ttg aat tca ata cac cgt | 299 |
| Gln Arg Arg Gln Ile Phe Tyr Asn His Lys Leu Asn Ser Ile His Arg | |
| 65 70 75 | |
| gaa tac tac ttc aca aac gca aca cta gtg aac cac gtc tgt aat gtt | 347 |
| Glu Tyr Tyr Phe Thr Asn Ala Thr Leu Val Asn His Val Cys Asn Val | |
| 80 85 90 95 | |
| gcc atg ttc ttc ggg gag gga gca gga gat gtg ata ata gaa aac aag | 395 |
| Ala Met Phe Phe Gly Glu Gly Ala Gly Asp Val Ile Ile Glu Asn Lys | |
| 100 105 110 | |
| aac tat acc tta ctg caa atg cat tgg cac act cct tct gaa cat cac | 443 |
| Asn Tyr Thr Leu Leu Gln Met His Trp His Thr Pro Ser Glu His His | |
| 115 120 125 | |
| ctc cat gga gtc caa tat gca gct gag ctg cac atg gta cac caa gca | 491 |
| Leu His Gly Val Gln Tyr Ala Ala Glu Leu His Met Val His Gln Ala | |
| 130 135 140 | |
| aaa gat gga agc ttt gct gtg gtg gca agt ctc ttc aaa atc ggc act | 539 |
| Lys Asp Gly Ser Phe Ala Val Val Ala Ser Leu Phe Lys Ile Gly Thr | |
| 145 150 155 | |
| gaa gag cct ttc ctc tct cag atg aag gag aaa ttg gtg aag cta aag | 587 |
| Glu Glu Pro Phe Leu Ser Gln Met Lys Glu Lys Leu Val Lys Leu Lys | |
| 160 165 170 175 | |
| gaa gag aga ctc aaa ggg aac cac aca gca caa gtg gaa gta gga aga | 635 |
| Glu Glu Arg Leu Lys Gly Asn His Thr Ala Gln Val Glu Val Gly Arg | |
| 180 185 190 | |
| atc gac aca aga cac att gaa cgt aag act cga aag tac tac aga tac | 683 |
| Ile Asp Thr Arg His Ile Glu Arg Lys Thr Arg Lys Tyr Tyr Arg Tyr | |
| 195 200 205 | |
| att ggt tca ctc act act cct cct tgc tcc gag aac gtt tct tgg acc | 731 |
| Ile Gly Ser Leu Thr Thr Pro Pro Cys Ser Glu Asn Val Ser Trp Thr | |
| 210 215 220 | |
| atc ctt ggc aag gtg agg tca atg tcaaaggaac aagtagaact actcagatct | 785 |
| Ile Leu Gly Lys Val Arg Ser Met (SEQ ID NO: 18) | |
| 225 230 | |
| ccattggaca cttctttcaa gaacaattca agaccgtgtc aacccctcaa cggccggaga | 845 |
| gttgagatgt tccacgacca cgagcgtgtc gataaaaaag aaaccggtaa caaaaagaaa | 905 |
| aaacccaatt aaaatagttt tacattgtct attggtttgt ttagaaccct aattagcttt | 965 |
| gtaaaactaa taatctctta tgtagtactg tgttgttgtt tacgacttga tatacgattt | 1025 |
| ccaaat (SEQ ID NO: 17) | 1031 |
| SEQ ID NO: 18 | |
| Met Lys Ile Met Met Met Ile Lys Leu Cys Phe Phe Ser Met Ser Leu | |
| 1 5 10 15 | |
| Ile Cys Ile Ala Pro Ala Asp Ala Gln Thr Glu Gly Val Val Phe Gly | |
| 20 25 30 | |
| Tyr Lys Gly Lys Asn Gly Pro Asn Gln Trp Gly His Leu Asn Pro His | |
| 35 40 45 | |
| Phe Thr Thr Cys Ala Val Gly Lys Leu Gln Ser Pro Ile Asp Ile Gln | |
| 50 55 60 | |
| Arg Arg Gln Ile Phe Tyr Asn His Lys Leu Asn Ser Ile His Arg Glu | |
| 65 70 75 80 | |
| Tyr Tyr Phe Thr Asn Ala Thr Leu Val Asn His Val Cys Asn Val Ala | |
| 85 90 95 | |
| Met Phe Phe Gly Glu Gly Ala Gly Asp Val Ile Ile Glu Asn Lys Asn | |
| 100 105 110 | |
| Tyr Thr Leu Leu Gln Met His Trp His Thr Pro Ser Glu His His Leu | |
| 115 120 125 | |
| His Gly Val Gln Tyr Ala Ala Glu Leu His Met Val His Gln Ala Lys | |
| 130 135 140 | |
| Asp Gly Ser Phe Ala Val Val Ala Ser Leu Phe Lys Ile Gly Thr Glu | |
| 145 150 155 160 | |
| Glu Pro Phe Leu Ser Gln Met Lys Glu Lys Leu Val Lys Leu Lys Glu | |
| 165 170 175 | |
| Glu Arg Leu Lys Gly Asn His Thr Ala Gln Val Glu Val Gly Arg Ile | |
| 180 185 190 | |
| Asp Thr Arg His Ile Glu Arg Lys Thr Arg Lys Tyr Tyr Arg Tyr Ile | |
| 195 200 205 | |
| Gly Ser Leu Thr Thr Pro Pro Cys Ser Glu Asn Val Ser Trp Thr Ile | |
| 210 215 220 | |
| Leu Gly Lys Val Arg Ser Met | |
| 225 230 | |
| SEQ ID NO: 19/SEQ ID NO: 20 | |
| atg gat gaa tat gta gag gat gaa cac gaa ttc agc tac gaa tgg aac | 48 |
| Met Asp Glu Tyr Val Glu Asp Glu His Glu Phe Ser Tyr Glu Trp Asn | |
| 1 5 10 15 | |
| caa gag aac ggg cca gcg aaa tgg gga aag cta aga ccg gaa tgg aaa | 96 |
| Gln Glu Asn Gly Pro Ala Lys Trp Gly Lys Leu Arg Pro Glu Trp Lys | |
| 20 25 30 | |
| atg tgc gga aaa gga gaa atg caa tcg cct att gat ctt atg aac aaa | 144 |
| Met Cys Gly Lys Gly Glu Met Gln Ser Pro Ile Asp Leu Met Asn Lys | |
| 35 40 45 | |
| aga gtt aga ctt gtt act cat ctt aaa aag ctt act aga cac tac aaa | 192 |
| Arg Val Arg Leu Val Thr His Leu Lys Lys Leu Thr Arg His Tyr Lys | |
| 50 55 60 | |
| cct tgt aac gcc act ctc aaa aat aga ggc cat gat atg atg ctg aaa | 240 |
| Pro Cys Asn Ala Thr Leu Lys Asn Arg Gly His Asp Met Met Leu Lys | |
| 65 70 75 80 | |
| ttt gga gaa gaa ggg tca ggg agt att acg gtc aat gga act gag tat | 288 |
| Phe Gly Glu Glu Gly Ser Gly Ser Ile Thr Val Asn Gly Thr Glu Tyr | |
| 85 90 95 | |
| aaa ctc tta cag ctt cat tgg cat tct ccc tct gaa cat act atg aat | 336 |
| Lys Leu Leu Gln Leu His Trp His Ser Pro Ser Glu His Thr Met Asn | |
| 100 105 110 | |
| gga aga agg ttt gct ctc gag cta cac atg gtt cac gaa aac att aac | 384 |
| Gly Arg Arg Phe Ala Leu Glu Leu His Met Val His Glu Asn Ile Asn | |
| 115 120 125 | |
| gga agt ttg gct gta gtc aca gtc ctc tac aaa atc gga agg cca gat | 432 |
| Gly Ser Leu Ala Val Val Thr Val Leu Tyr Lys Ile Gly Arg Pro Asp | |
| 130 135 140 | |
| tct ttt ctc gga ttg ctg gaa aat aaa ttg tcg gca att aca gat caa | 480 |
| Ser Phe Leu Gly Leu Leu Glu Asn Lys Leu Ser Ala Ile Thr Asp Gln | |
| 145 150 155 160 | |
| aat gag gcg gag aaa tat gta gat gtg att gac cca agg gat att aag | 528 |
| Asn Glu Ala Glu Lys Tyr Val Asp Val Ile Asp Pro Arg Asp Ile Lys | |
| 165 170 175 | |
| att ggg agc aga aaa ttt tat aga tac att gga tca ctt act act cct | 576 |
| Ile Gly Ser Arg Lys Phe Tyr Arg Tyr Ile Gly Ser Leu Thr Thr Pro | |
| 180 185 190 | |
| cct tgt acg caa aat gtt att tgg acc gtc gtt aaa aag gta aat act | 624 |
| Pro Cys Thr Gln Asn Val Ile Trp Thr Val Val Lys Lys Val Asn Thr | |
| 195 200 205 | |
| cat cgt tat ttt ctt ctc ttt ttt act taa tcaaacatag cattaataga | 674 |
| His Arg Tyr Phe Leu Leu Phe Phe Thr (SEQ ID NO: 20) | |
| 210 215 | |
| tcattacaag gtactaatag tgtgaatatc catatccaaa aggtttatcc atctacatgt | 734 |
| ta (SEQ ID NO: 19) | 736 |
| SEQ ID NO: 20 | |
| Met Asp Glu Tyr Val Glu Asp Glu His Glu Phe Ser Tyr Glu Trp Asn | |
| 1 5 10 15 | |
| Gln Glu Asn Gly Pro Ala Lys Trp Gly Lys Leu Arg Pro Glu Trp Lys | |
| 20 25 30 | |
| Met Cys Gly Lys Gly Glu Met Gln Ser Pro Ile Asp Leu Met Asn Lys | |
| 35 40 45 | |
| Arg Val Arg Leu Val Thr His Leu Lys Lys Leu Thr Arg His Tyr Lys | |
| 50 55 60 | |
| Pro Cys Asn Ala Thr Leu Lys Asn Arg Gly His Asp Met Met Leu Lys | |
| 65 70 75 80 | |
| Phe Gly Glu Glu Gly Ser Gly Ser Ile Thr Val Asn Gly Thr Glu Tyr | |
| 85 90 95 | |
| Lys Leu Leu Gln Leu His Trp His Ser Pro Ser Glu His Thr Met Asn | |
| 100 105 110 | |
| Gly Arg Arg Phe Ala Leu Glu Leu His Met Val His Glu Asn Ile Asn | |
| 115 120 125 | |
| Gly Ser Leu Ala Val Val Thr Val Leu Tyr Lys Ile Gly Arg Pro Asp | |
| 130 135 140 | |
| Ser Phe Leu Gly Leu Leu Glu Asn Lys Leu Ser Ala Ile Thr Asp Gln | |
| 145 150 155 160 | |
| Asn Glu Ala Glu Lys Tyr Val Asp Val Ile Asp Pro Arg Asp Ile Lys | |
| 165 170 175 | |
| Ile Gly Ser Arg Lys Phe Tyr Arg Tyr Ile Gly Ser Leu Thr Thr Pro | |
| 180 185 190 | |
| Pro Cys Thr Gln Asn Val Ile Trp Thr Val Val Lys Lys Val Asn Thr | |
| 195 200 205 | |
| His Arg Tyr Phe Leu Leu Phe Phe Thr | |
| 210 215 | |
| SEQ ID NO: 21/SEQ ID NO: 22 | |
| aaaacacatt ctgagaagaa gaagaagaaa ataagaaaaa acaaaag atg aaa acc | 56 |
| Met Lys Thr | |
| 1 | |
| att atc ctt ttt gta aca ttt ctt gct ctt tct tct tca tct cta gcc | 104 |
| Ile Ile Leu Phe Val Thr Phe Leu Ala Leu Ser Ser Ser Ser Leu Ala | |
| 5 10 15 | |
| gat gag aca gag act gaa ttt cat tac aaa ccc ggt gag ata gcc gat | 152 |
| Asp Glu Thr Glu Thr Glu Phe His Tyr Lys Pro Gly Glu Ile Ala Asp | |
| 20 25 30 35 | |
| ccc tcg aaa tgg agc agt atc aag gct gaa tgg aaa att tgc ggg aca | 200 |
| Pro Ser Lys Trp Ser Ser Ile Lys Ala Glu Trp Lys Ile Cys Gly Thr | |
| 40 45 50 | |
| ggg aag agg caa tcg cca atc aat ctt act cca aaa ata gct cgc att | 248 |
| Gly Lys Arg Gln Ser Pro Ile Asn Leu Thr Pro Lys Ile Ala Arg Ile | |
| 55 60 65 | |
| gtt cac aat tct aca gag att ctt cag aca tat tac aaa cct gta gag | 296 |
| Val His Asn Ser Thr Glu Ile Leu Gln Thr Tyr Tyr Lys Pro Val Glu | |
| 70 75 80 | |
| gct att ctt aag aac cgt gga ttc gac atg aag gtt aag tgg gaa gac | 344 |
| Ala Ile Leu Lys Asn Arg Gly Phe Asp Met Lys Val Lys Trp Glu Asp | |
| 85 90 95 | |
| gat gca ggg aag atc gtg atc aat gat acc gac tat aaa ttg gtt caa | 392 |
| Asp Ala Gly Lys Ile Val Ile Asn Asp Thr Asp Tyr Lys Leu Val Gln | |
| 100 105 110 115 | |
| agc cac tgg cac gca cct tca gag cat ttt ctc gat gga cag agg ttg | 440 |
| Ser His Trp His Ala Pro Ser Glu His Phe Leu Asp Gly Gln Arg Leu | |
| 120 125 130 | |
| gca atg gaa ctt cac atg gta cac aaa agt gta gaa ggg cac ttg gca | 488 |
| Ala Met Glu Leu His Met Val His Lys Ser Val Glu Gly His Leu Ala | |
| 135 140 145 | |
| gtg att gga gtt ctc ttc aga gaa gga gaa cca aat gct ttc att tcg | 536 |
| Val Ile Gly Val Leu Phe Arg Glu Gly Glu Pro Asn Ala Phe Ile Ser | |
| 150 155 160 | |
| cgg atc atg gac aag atc cat aag atc gca gac gta caa gat gga gag | 584 |
| Arg Ile Met Asp Lys Ile His Lys Ile Ala Asp Val Gln Asp Gly Glu | |
| 165 170 175 | |
| gtc agc atc gga aag ata gat cca aga gaa ttt gga tgg gat ctt aca | 632 |
| Val Ser Ile Gly Lys Ile Asp Pro Arg Glu Phe Gly Trp Asp Leu Thr | |
| 180 185 190 195 | |
| aag ttt tat gaa tac aga ggt tct ctc acg act cct cct tgc acg gaa | 680 |
| Lys Phe Tyr Glu Tyr Arg Gly Ser Leu Thr Thr Pro Pro Cys Thr Glu | |
| 200 205 210 | |
| gat gtc atg tgg acc atc atc aac aag gtg ggg act gtt tca cgt gag | 728 |
| Asp Val Met Trp Thr Ile Ile Asn Lys Val Gly Thr Val Ser Arg Glu | |
| 215 220 225 | |
| caa att gat gta ttg aca gat gct cgt cgc ggt ggt tat gag aag aac | 776 |
| Gln Ile Asp Val Leu Thr Asp Ala Arg Arg Gly Gly Tyr Glu Lys Asn | |
| 230 235 240 | |
| gcg aga cca gct caa cct ctg aac gga cgt ctg gtt tat tta aac gag | 824 |
| Ala Arg Pro Ala Gln Pro Leu Asn Gly Arg Leu Val Tyr Leu Asn Glu | |
| 245 250 255 | |
| cag tcc agt cca agt cca act cca cgg cta aga ata cca cga gtt ggt | 872 |
| Gln Ser Ser Pro Ser Pro Thr Pro Arg Leu Arg Ile Pro Arg Val Gly | |
| 260 265 270 275 | |
| ccg gtc taa gacagtctta taggacaagg caactccgag ccctaatttc | 921 |
| Pro Val (SEQ ID NO: 22) | |
| catacaaaga aaattcggaa aagaattttg aagatgtatg aaaattggga gccataacta | 981 |
| ttttttttta actattcttt tgattaaaag ataaaactac gcaatattat atgcataaag | 1041 |
| tttttctttt atacatgtat tccaataaac aagatgtaat aatatccaac cataatgagt | 1101 |
| tgtttgatta ttttataaca caagatctct cac (SEQ ID NO: 21) | 1134 |
| SEQ ID NO: 22 | |
| Met Lys Thr Ile Ile Leu Phe Val Thr Phe Leu Ala Leu Ser Ser Ser | |
| 1 5 10 15 | |
| Ser Leu Ala Asp Glu Thr Glu Thr Glu Phe His Tyr Lys Pro Gly Glu | |
| 20 25 30 | |
| Ile Ala Asp Pro Ser Lys Trp Ser Ser Ile Lys Ala Glu Trp Lys Ile | |
| 35 40 45 | |
| Cys Gly Thr Gly Lys Arg Gln Ser Pro Ile Asn Leu Thr Pro Lys Ile | |
| 50 55 60 | |
| Ala Arg Ile Val His Asn Ser Thr Glu Ile Leu Gln Thr Tyr Tyr Lys | |
| 65 70 75 80 | |
| Pro Val Glu Ala Ile Leu Lys Asn Arg Gly Phe Asp Met Lys Val Lys | |
| 85 90 95 | |
| Trp Glu Asp Asp Ala Gly Lys Ile Val Ile Asn Asp Thr Asp Tyr Lys | |
| 100 105 110 | |
| Leu Val Gln Ser His Trp His Ala Pro Ser Glu His Phe Leu Asp Gly | |
| 115 120 125 | |
| Gln Arg Leu Ala Met Glu Leu His Met Val His Lys Ser Val Glu Gly | |
| 130 135 140 | |
| His Leu Ala Val Ile Gly Val Leu Phe Arg Glu Gly Glu Pro Asn Ala | |
| 145 150 155 160 | |
| Phe Ile Ser Arg Ile Met Asp Lys Ile His Lys Ile Ala Asp Val Gln | |
| 165 170 175 | |
| Asp Gly Glu Val Ser Ile Gly Lys Ile Asp Pro Arg Glu Phe Gly Trp | |
| 180 185 190 | |
| Asp Leu Thr Lys Phe Tyr Glu Tyr Arg Gly Ser Leu Thr Thr Pro Pro | |
| 195 200 205 | |
| Cys Thr Glu Asp Val Met Trp Thr Ile Ile Asn Lys Val Gly Thr Val | |
| 210 215 220 | |
| Ser Arg Glu Gln Ile Asp Val Leu Thr Asp Ala Arg Arg Gly Gly Tyr | |
| 225 230 235 240 | |
| Glu Lys Asn Ala Arg Pro Ala Gln Pro Leu Asn Gly Arg Leu Val Tyr | |
| 245 250 255 | |
| Leu Asn Glu Gln Ser Ser Pro Ser Pro Thr Pro Arg Leu Arg Ile Pro | |
| 260 265 270 | |
| Arg Val Gly Pro Val | |
| 275 | |
| SEQ ID NO: 23/SEQ ID NO: 24 | |
| atg gat acc aac gca aaa aca att ttc ttc atg gct atg tgt ttc atc | 48 |
| Met Asp Thr Asn Ala Lys Thr Ile Phe Phe Met Ala Met Cys Phe Ile | |
| 1 5 10 15 | |
| tat cta tct ttc cct aat att tca cac gct cat tct gaa gtc gac gac | 96 |
| Tyr Leu Ser Phe Pro Asn Ile Ser His Ala His Ser Glu Val Asp Asp | |
| 20 25 30 | |
| gaa act cca ttt act tac gaa caa aaa acg gaa aag gga cca gag gga | 144 |
| Glu Thr Pro Phe Thr Tyr Glu Gln Lys Thr Glu Lys Gly Pro Glu Gly | |
| 35 40 45 | |
| tgg ggc aaa ata aat ccg cac tgg aaa gtt tgt aac acc gga aga tat | 192 |
| Trp Gly Lys Ile Asn Pro His Trp Lys Val Cys Asn Thr Gly Arg Tyr | |
| 50 55 60 | |
| caa tcc ccg atc gat ctt act aac gaa aga gtc agt ctt att cat gat | 240 |
| Gln Ser Pro Ile Asp Leu Thr Asn Glu Arg Val Ser Leu Ile His Asp | |
| 65 70 75 80 | |
| caa gca tgg aca aga caa tat aaa cca gct ccg gct gta att aca aac | 288 |
| Gln Ala Trp Thr Arg Gln Tyr Lys Pro Ala Pro Ala Val Ile Thr Asn | |
| 85 90 95 | |
| aga ggc cat gac att atg gta tca tgg aaa gga gat gct ggg aag atg | 336 |
| Arg Gly His Asp Ile Met Val Ser Trp Lys Gly Asp Ala Gly Lys Met | |
| 100 105 110 | |
| aca ata cgg aaa acg gat ttt aat ttg gtg caa tgc cat tgg cat tca | 384 |
| Thr Ile Arg Lys Thr Asp Phe Asn Leu Val Gln Cys His Trp His Ser | |
| 115 120 125 | |
| cct tct gag cat acc gtt aac gga act agg tac gac cta gag ctt cac | 432 |
| Pro Ser Glu His Thr Val Asn Gly Thr Arg Tyr Asp Leu Glu Leu His | |
| 130 135 140 | |
| atg gtt cac acg agt gca cga ggc aga act gcg gtt atc gga gtt ctt | 480 |
| Met Val His Thr Ser Ala Arg Gly Arg Thr Ala Val Ile Gly Val Leu | |
| 145 150 155 160 | |
| tac aaa tta ggc gaa cct aat gaa ttc ctc acc aag cta cta aat gga | 528 |
| Tyr Lys Leu Gly Glu Pro Asn Glu Phe Leu Thr Lys Leu Leu Asn Gly | |
| 165 170 175 | |
| ata aaa gca gtg gga aat aaa gag ata aat cta ggg atg att gat cca | 576 |
| Ile Lys Ala Val Gly Asn Lys Glu Ile Asn Leu Gly Met Ile Asp Pro | |
| 180 185 190 | |
| cga gag att agg ttt caa aca aga aaa ttc tat aga tac att ggc tct | 624 |
| Arg Glu Ile Arg Phe Gln Thr Arg Lys Phe Tyr Arg Tyr Ile Gly Ser | |
| 195 200 205 | |
| ctc act gtt cct cct tgc act gaa ggc gtc att tgg act gtc gtc aaa | 672 |
| Leu Thr Val Pro Pro Cys Thr Glu Gly Val Ile Trp Thr Val Val Lys | |
| 210 215 220 | |
| agg gtg aac aca ata tca atg gag caa att aca gct ctt agg caa gcc | 720 |
| Arg Val Asn Thr Ile Ser Met Glu Gln Ile Thr Ala Leu Arg Gln Ala | |
| 225 230 235 240 | |
| gtt gac gat gga ttt gag aca aat tca aga ccg gtt caa gac tca aag | 768 |
| Val Asp Asp Gly Phe Glu Thr Asn Ser Arg Pro Val Gln Asp Ser Lys | |
| 245 250 255 | |
| gga aga tca gtt tgg ttc tat gat cca aat gtt tga (SEQ ID NO: 23) | 804 |
| Gly Arg Ser Val Trp Phe Tyr Asp Pro Asn Val (SEQ ID NO: 24) | |
| 260 265 | |
| SEQ ID NO: 24 | |
| Met Asp Thr Asn Ala Lys Thr Ile Phe Phe Met Ala Met Cys Phe Ile | |
| 1 5 10 15 | |
| Tyr Leu Ser Phe Pro Asn Ile Ser His Ala His Ser Glu Val Asp Asp | |
| 20 25 30 | |
| Glu Thr Pro Phe Thr Tyr Glu Gln Lys Thr Glu Lys Gly Pro Glu Gly | |
| 35 40 45 | |
| Trp Gly Lys Ile Asn Pro His Trp Lys Val Cys Asn Thr Gly Arg Tyr | |
| 50 55 60 | |
| Gln Ser Pro Ile Asp Leu Thr Asn Glu Arg Val Ser Leu Ile His Asp | |
| 65 70 75 80 | |
| Gln Ala Trp Thr Arg Gln Tyr Lys Pro Ala Pro Ala Val Ile Thr Asn | |
| 85 90 95 | |
| Arg Gly His Asp Ile Met Val Ser Trp Lys Gly Asp Ala Gly Lys Met | |
| 100 105 110 | |
| Thr Ile Arg Lys Thr Asp Phe Asn Leu Val Gln Cys His Trp His Ser | |
| 115 120 125 | |
| Pro Ser Glu His Thr Val Asn Gly Thr Arg Tyr Asp Leu Glu Leu His | |
| 130 135 140 | |
| Met Val His Thr Ser Ala Arg Gly Arg Thr Ala Val Ile Gly Val Leu | |
| 145 150 155 160 | |
| Tyr Lys Leu Gly Glu Pro Asn Glu Phe Leu Thr Lys Leu Leu Asn Gly | |
| 165 170 175 | |
| Ile Lys Ala Val Gly Asn Lys Glu Ile Asn Leu Gly Met Ile Asp Pro | |
| 180 185 190 | |
| Arg Glu Ile Arg Phe Gln Thr Arg Lys Phe Tyr Arg Tyr Ile Gly Ser | |
| 195 200 205 | |
| Leu Thr Val Pro Pro Cys Thr Glu Gly Val Ile Trp Thr Val Val Lys | |
| 210 215 220 | |
| Arg Val Asn Thr Ile Ser Met Glu Gln Ile Thr Ala Leu Arg Gln Ala | |
| 225 230 235 240 | |
| Val Asp Asp Gly Phe Glu Thr Asn Ser Arg Pro Val Gln Asp Ser Lys | |
| 245 250 255 (SEQ ID NO: 23) | |
| Gly Arg Ser Val Trp Phe Tyr Asp Pro Asn Val (SEQ ID NO: 24) | |
| 260 265 | |
| SEQ ID NO: 25 /SEQ ID NO: 26 | |
| gatcaacatc tccttgaagt tgtttcataa gaataagagc tataaaagag gataaaacca | 60 |
| aaatttgaat ttttttcttc tatctctctc cccaagatat atagcacaag aaa atg | 116 |
| Met | |
| 1 | |
| aag ata cca tca att ggc tat gtc ttt ttc ctt atc ttc atc tct att | 164 |
| Lys Ile Pro Ser Ile Gly Tyr Val Phe Phe Leu Ile Phe Ile Ser Ile | |
| 5 10 15 | |
| aca att gtt tcg agt tca cca gat cat gga gaa gtt gag gac gaa acg | 212 |
| Thr Ile Val Ser Ser Ser Pro Asp His Gly Glu Val Glu Asp Glu Thr | |
| 20 25 30 | |
| cag ttt aac tac gag aag aaa gga gag aag ggg cca gag aac tgg gga | 260 |
| Gln Phe Asn Tyr Glu Lys Lys Gly Glu Lys Gly Pro Glu Asn Trp Gly | |
| 35 40 45 | |
| aga cta aag cca gag tgg gca atg tgt gga aaa ggc aac atg cag tct | 308 |
| Arg Leu Lys Pro Glu Trp Ala Met Cys Gly Lys Gly Asn Met Gln Ser | |
| 50 55 60 65 | |
| ccg att gat ctt acg gac aaa aga gtc ttg att gat cat aat ctt gga | 356 |
| Pro Ile Asp Leu Thr Asp Lys Arg Val Leu Ile Asp His Asn Leu Gly | |
| 70 75 80 | |
| tac ctt cgt agc cag tat tta cct tca aat gcc acc att aag aac aga | 404 |
| Tyr Len Arg Ser Gln Tyr Len Pro Ser Asn Ala Thr Ile Lys Asn Arg | |
| 85 90 95 | |
| ggc cat gat atc atg atg aaa ttt gaa gga gga aat gca ggt tta ggt | 452 |
| Gly His Asp Ile Met Met Lys Phe Glu Gly Gly Asn Ala Gly Leu Gly | |
| 100 105 110 | |
| atc act att aat ggt act gaa tat aaa ctt caa cag att cat tgg cac | 500 |
| Ile Thr Ile Asn Gly Thr Glu Tyr Lys Leu Gln Gln Ile His Trp His | |
| 115 120 125 | |
| tct cct tcc gaa cac aca ctc aat ggc aaa agg ttt gtt ctt gag gaa | 548 |
| Ser Pro Ser Glu His Thr Leu Asn Gly Lys Arg Phe Val Leu Glu Gln | |
| 130 135 140 145 | |
| cac atg gtt cat cag agc aaa gat gga cgc aac gct gtt gtc gct ttc | 596 |
| His Met Val His Gln Ser Lys Asp Gly Arg Asn Ala Val Val Ala Phe | |
| 150 155 160 | |
| ttt tac aaa ttg gga aaa cct gac tat ttt ctc ctc acg ttg gaa aga | 644 |
| Phe Tyr Lys Leu Gly Lys Pro Asp Tyr Phe Leu Leu Thr Leu Glu Arg | |
| 165 170 175 | |
| tac ttg aag agg ata act gat aca cac gaa tcc cag gaa ttt gtc gag | 692 |
| Tyr Leu Lys Arg Ile Thr Asp Thr His Glu Ser Gln Glu Phe Val Glu | |
| 180 185 190 | |
| atg gtt cat cct aga aca ttc ggt ttt gaa tca aaa cac tat tat aga | 740 |
| Met Val His Pro Arg Thr Phe Gly Phe Glu Ser Lys His Tyr Tyr Arg | |
| 195 200 205 | |
| ttt atc gga tca ctt aca act cca ccg tgt tct gaa aat gtg att tgg | 788 |
| Phe Ile Gly Ser Leu Thr Thr Pro Pro Cys Ser Glu Asn Val Ile Trp | |
| 210 215 220 225 | |
| acg att tcc aaa gag atg agg act gtg aca tta aaa caa ttg atc atg | 836 |
| Thr Ile Ser Lys Glu Met Arg Thr Val Thr Leu Lys Gln Leu Ile Met | |
| 230 235 240 | |
| ctt cga gtg act gta cac gat caa tct aac tca aat gct aga ccg ctt | 884 |
| Leu Arg Val Thr Val His Asp Gln Ser Asn Ser Asn Ala Arg Pro Leu | |
| 245 250 255 | |
| cag cgt aaa aat gag cgt ccg gtg gca ctt tac ata cca aca tgg cat | 932 |
| Gln Arg Lys Asn Glu Arg Pro Val Ala Leu Tyr Ile Pro Thr Trp His | |
| 260 265 270 | |
| agt aaa cta tat taa atatttaagt ttggtttata ttctttctag taatctttga | 987 |
| Ser Lys Leu Tyr (SEQ ID NO: 26) | |
| 275 | |
| aatattgtaa gagataatgc ttctaataaa taacattgga tttattggaa ttaatgtatt | 1047 |
| gaaaaaacta tgcaaatact acagtgtatt ttggaacgac c (SEQ ID NO: 25) | |
| SEQ ID NO: 26 | |
| Met Lys Ile Pro Ser Ile Gly Tyr Val Phe Phe Leu Ile Phe Ile Ser | |
| 1 5 10 15 | |
| Ile Thr Ile Val Ser Ser Ser Pro Asp His Gly Glu Val Glu Asp Glu | |
| 20 25 30 | |
| Thr Gln Phe Asn Tyr Glu Lys Lys Gly Glu Lys Gly Pro Glu Asn Trp | |
| 35 40 45 | |
| Gly Arg Leu Lys Pro Glu Trp Ala Met Cys Gly Lys Gly Asn Met Gln | |
| 50 55 60 | |
| Ser Pro Ile Asp Leu Thr Asp Lys Arg Val Leu Ile Asp His Asn Leu | |
| 65 70 75 80 | |
| Gly Tyr Leu Arg Ser Gln Tyr Leu Pro Ser Asn Ala Thr Ile Lys Asn | |
| 85 90 95 | |
| Arg Gly His Asp Ile Met Met Lys Phe Glu Gly Gly Asn Ala Gly Leu | |
| 100 105 110 | |
| Gly Ile Thr Ile Asn Gly Thr Glu Tyr Lys Leu Gln Gln Ile His Trp | |
| 115 120 125 | |
| His Ser Pro Ser Glu His Thr Leu Asn Gly Lys Arg Phe Val Leu Glu | |
| 130 135 140 | |
| Glu His Met Val His Gln Ser Lys Asp Gly Arg Asn Ala Val Val Ala | |
| 145 150 155 160 | |
| Phe Phe Tyr Lys Leu Gly Lys Pro Asp Tyr Phe Leu Leu Thr Leu Glu | |
| 165 170 175 | |
| Arg Tyr Leu Lys Arg Ile Thr Asp Thr His Glu Ser Gln Glu Phe Val | |
| 180 185 190 | |
| Glu Met Val His Pro Arg Thr Phe Gly Phe Glu Ser Lys His Tyr Tyr | |
| 195 200 205 | |
| Arg Phe Ile Gly Ser Leu Thr Thr Pro Pro Cys Ser Glu Asn Val Ile | |
| 210 215 220 | |
| Trp Thr Ile Ser Lys Glu Met Arg Thr Val Thr Leu Lys Gln Leu Ile | |
| 225 230 235 240 | |
| Met Leu Arg Val Thr Val His Asp Gln Ser Asn Ser Asn Ala Arg Pro | |
| 245 250 255 | |
| Leu Gln Arg Lys Asn Glu Arg Pro Val Ala Leu Tyr Ile Pro Thr Trp | |
| 260 265 270 | |
| His Ser Lys Len Tyr | |
| 275 | |
| SEQ ID NO: 27/SEQ ID NO: 28 | |
| atg gat gcc aac aca aaa aca att tta ttt ttt gta gtg ttc ttc atc | 48 |
| Met Asp Ala Asn Thr Lys Thr Ile Leu Phe Phe Val Val Phe Phe Ile | |
| 1 5 10 15 | |
| gat tta ttt tcc cct aat att tta ttc gtt tat gct cgt gaa atc ggc | 96 |
| Asp Leu Phe Ser Pro Asn Ile Leu Phe Val Tyr Ala Arg Glu Ile Gly | |
| 20 25 30 | |
| aac aaa ccg cta ttt aca tac aaa caa aaa aca gag aaa gga cca gcg | 144 |
| Asn Lys Pro Leu Phe Thr Tyr Lys Gln Lys Thr Glu Lys Gly Pro Ala | |
| 35 40 45 | |
| gaa tgg ggc aaa tta gac cct caa tgg aaa gtt tgt agc acc gga aaa | 192 |
| Glu Trp Gly Lys Leu Asp Pro Gln Trp Lys Val Cys Ser Thr Gly Lys | |
| 50 55 60 | |
| att caa tct ccg att gat ctc act gac gaa aga gtc agt ctt att cat | 240 |
| Ile Gln Ser Pro Ile Asp Leu Thr Asp Glu Arg Val Ser Leu Ile His | |
| 65 70 75 80 | |
| gat caa gcc ttg agt aaa cat tac aaa cca gct tcg gct gta att caa | 288 |
| Asp Gln Ala Leu Ser Lys His Tyr Lys Pro Ala Ser Ala Val Ile Gln | |
| 85 90 95 | |
| agt aga gga cat gac gtt atg gta tcg tgg aaa gga gat ggt ggg aaa | 336 |
| Ser Arg Gly His Asp Val Met Val Ser Trp Lys Gly Asp Gly Gly Lys | |
| 100 105 110 | |
| ata aca ata cat caa acg gat tat aaa ttg gtg cag tgc cat tgg cat | 384 |
| Ile Thr Ile His Gln Thr Asp Tyr Lys Leu Val Gln Cys His Trp His | |
| 115 120 125 | |
| tca ccg tct gag cat acc att aac gga act agc tat gac cta gag ctt | 432 |
| Ser Pro Ser Glu His Thr Ile Asn Gly Thr Ser Tyr Asp Leu Glu Leu | |
| 130 135 140 | |
| cac atg gtt cac acg agt gct agt ggc aaa acc act gtg gtt gga gtt | 480 |
| His Met Val His Thr Ser Ala Ser Gly Lys Thr Thr Val Val Gly Val | |
| 145 150 155 160 | |
| ctt tat aaa tta ggt gaa cct gat gaa ttc ctc aca aag ata cta aat | 528 |
| Leu Tyr Lys Leu Gly Glu Pro Asp Glu Phe Leu Thr Lys Ile Leu Asn | |
| 165 170 175 | |
| gga ata aaa gga gta ggg aaa aaa gag ata gat cta gga atc gtg gat | 576 |
| Gly Ile Lys Gly Val Gly Lys Lys Glu Ile Asp Leu Gly Ile Val Asp | |
| 180 185 190 | |
| cct cga gat att aga ttt gaa acc aac aat ttc tat aga tac att ggc | 624 |
| Pro Arg Asp Ile Arg Phe Glu Thr Asn Asn Phe Tyr Arg Tyr Ile Gly | |
| 195 200 205 | |
| tct ctc act att cct cca tgc acc gaa ggc gtt att tgg acc gtc cag | 672 |
| Ser Leu Thr Ile Pro Pro Cys Thr Glu Gly Val Ile Trp Thr Val Gln | |
| 210 215 220 | |
| aaa agg gta tta tat ttt ttt tgt ttc tgt tat aga tta att atc ttc | 720 |
| Lys Arg Val Leu Tyr Phe Phe Cys Phe Cys Tyr Arg Leu Ile Ile Phe | |
| 225 230 235 240 | |
| gtt aca cct tac ata aac att ttt tgg att ttt gtt ttt gta ttt tgg | 768 |
| Val Thr Pro Tyr Ile Asn Ile Phe Trp Ile Phe Val Phe Val Phe Trp | |
| 245 250 255 | |
| tgt atg cta atg taa (SEQ ID NO: 27) | 783 |
| Cys Met Leu Met (SEQ ID NO: 28) | |
| 260 | |
| SEQ ID NO: 28 | |
| Met Asp Ala Asn Thr Lys Thr Ile Leu Phe Phe Val Val Phe Phe Ile | |
| 1 5 10 15 | |
| Asp Leu Phe Ser Pro Asn Ile Leu Phe Val Tyr Ala Arg Glu Ile Gly | |
| 20 25 30 | |
| Asn Lys Pro Leu Phe Thr Tyr Lys Gln Lys Thr Glu Lys Gly Pro Ala | |
| 35 40 45 | |
| Glu Trp Gly Lys Leu Asp Pro Gln Trp Lys Val Cys Ser Thr Gly Lys | |
| 50 55 60 | |
| Ile Gln Ser Pro Ile Asp Leu Thr Asp Glu Arg Val Ser Leu Ile His | |
| 65 70 75 80 | |
| Asp Gln Ala Len Ser Lys His Tyr Lys Pro Ala Ser Ala Val Ile Gln | |
| 85 90 95 | |
| Ser Arg Gly His Asp Val Met Val Ser Trp Lys Gly Asp Gly Gly Lys | |
| 100 105 110 | |
| Ile Thr Ile His Gln Thr Asp Tyr Lys Leu Val Gln Cys His Trp His | |
| 115 120 125 | |
| Ser Pro Ser Glu His Thr Ile Asn Gly Thr Ser Tyr Asp Leu Glu Leu | |
| 130 135 140 | |
| His Met Val His Thr Ser Ala Ser Gly Lys Thr Thr Val Val Gly Val | |
| 145 150 155 160 | |
| Leu Tyr Lys Leu Gly Glu Pro Asp Glu Phe Leu Thr Lys Ile Leu Asn | |
| 165 170 175 | |
| Gly Ile Lys Gly Val Gly Lys Lys Glu Ile Asp Leu Gly Ile Val Asp | |
| 180 185 190 | |
| Pro Arg Asp Ile Arg Phe Glu Thr Asn Asn Phe Tyr Arg Tyr Ile Gly | |
| 195 200 205 | |
| Ser Leu Thr Ile Pro Pro Cys Thr Glu Gly Val Ile Trp Thr Val Gln | |
| 210 215 220 | |
| Lys Arg Val Leu Tyr Phe Phe Cys Phe Cys Tyr Arg Leu Ile Ile Phe | |
| 225 230 235 240 | |
| Val Thr Pro Tyr Ile Asn Ile Phe Trp Ile Phe Val Phe Val Phe Trp | |
| 245 250 255 | |
| Cys Met Leu Met | |
| 260 | |
| SEQ ID NO: 29/SEQ ID NO: 30 | |
| atg gtg aac tac tca tca atc agt tgc atc ttc ttt gtg gct ctg ttt | 48 |
| Met Val Asn Tyr Ser Ser Ile Ser Cys Ile Phe Phe Val Ala Leu Phe | |
| 1 5 10 15 | |
| agt att ttc aca att gtt tcg att tcg agt gct gct tca agt cac gga | 96 |
| Ser Ile Phe Thr Ile Val Ser Ile Ser Ser Ala Ala Ser Ser His Gly | |
| 20 25 30 | |
| gaa gtt gag gac gaa cgc gag ttt aac tac aag aag aac gat gag aag | 144 |
| Glu Val Glu Asp Glu Arg Glu Phe Asn Tyr Lys Lys Asn Asp Glu Lys | |
| 35 40 45 | |
| ggg cca gag aga tgg gga gaa ctt aaa ccg gaa tgg gaa atg tgt gga | 192 |
| Gly Pro Glu Arg Trp Gly Glu Leu Lys Pro Glu Trp Glu Met Cys Gly | |
| 50 55 60 | |
| aaa gga gag atg caa tct ccc ata gat ctt atg aac gag aga gtt aac | 240 |
| Lys Gly Glu Met Gln Ser Pro Ile Asp Leu Met Asn Glu Arg Val Asn | |
| 65 70 75 80 | |
| att gtt tct cat ctt gga agg ctt aat aga gac tat aat cct tca aat | 288 |
| Ile Val Ser His Leu Gly Arg Leu Asn Arg Asp Tyr Asn Pro Ser Asn | |
| 85 90 95 | |
| gca act ctt aag aac aga ggc cat gac atc atg tta aaa ttt gaa gat | 336 |
| Ala Thr Leu Lys Asn Arg Gly His Asp Ile Met Leu Lys Phe Glu Asp | |
| 100 105 110 | |
| gga gca gga act att aag atc aat ggt ttt gaa tat gaa ctt caa cag | 384 |
| Gly Ala Gly Thr Ile Lys Ile Asn Gly Phe Glu Tyr Glu Leu Gln Gln | |
| 115 120 125 | |
| ctt cac tgg cac tct ccg tct gaa cat act att aat gga aga agg ttt | 432 |
| Leu His Trp His Ser Pro Ser Glu His Thr Ile Asn Gly Arg Arg Phe | |
| 130 135 140 | |
| gca ctt gag ctg cat atg gtt cac gaa ggc agg aat aga aga atg gct | 480 |
| Ala Leu Glu Leu His Met Val His Glu Gly Arg Asn Arg Arg Met Ala | |
| 145 150 155 160 | |
| gtt gtg act gtg ttg tac aag atc gga aga gca gat act ttt atc aga | 528 |
| Val Val Thr Val Leu Tyr Lys Ile Gly Arg Ala Asp Thr Phe Ile Arg | |
| 165 170 175 | |
| tcg ttg gag aaa gaa tta gag ggc att gct gaa atg gag gag gct gag | 576 |
| Ser Leu Glu Lys Glu Leu Glu Gly Ile Ala Glu Met Glu Glu Ala Glu | |
| 180 185 190 | |
| aaa aat gta gga atg att gat ccc acc aaa att aag atc gga agc aga | 624 |
| Lys Asn Val Gly Met Ile Asp Pro Thr Lys Ile Lys Ile Gly Ser Arg | |
| 195 200 205 | |
| aaa tat tac aga tac act ggt tca ctt acc act cct cct tgc act caa | 672 |
| Lys Tyr Tyr Arg Tyr Thr Gly Ser Leu Thr Thr Pro Pro Cys Thr Gln | |
| 210 215 220 | |
| aac gtt act tgg agc gtc gtt aga aag gtt agg acc gtg aca aga aaa | 720 |
| Asn Val Thr Trp Ser Val Val Arg Lys Val Arg Thr Val Thr Arg Lys | |
| 225 230 235 240 | |
| caa gtg aag ctc ctc cgc gtg gca gtg cac gat gat gct aat tcg aat | 768 |
| Gln Val Lys Leu Leu Arg Val Ala Val His Asp Asp Ala Asn Ser Asn | |
| 245 250 255 | |
| gcg agg ccg gtt caa cca acc aac aag cgc ata gtg cac tta tac aga | 816 |
| Ala Arg Pro Val Gln Pro Thr Asn Lys Arg Ile Val His Len Tyr Arg | |
| 260 265 270 | |
| cca ata gtt taa tatatgaaga tactgaaagc ttttactaat c (SEQ ID NO: 29) | 859 |
| Pro Ile Val (SEQ ID NO: 30) | |
| 275 | |
| SEQ ID NO: 30 | |
| Met Val Asn Tyr Ser Ser Ile Ser Cys Ile Phe Phe Val Ala Leu Phe | |
| 1 5 10 15 | |
| Ser Ile Phe Thr Ile Val Ser Ile Ser Ser Ala Ala Ser Ser His Gly | |
| 20 25 30 | |
| Glu Val Glu Asp Glu Arg Glu Phe Asn Tyr Lys Lys Asn Asp Glu Lys | |
| 35 40 45 | |
| Gly Pro Glu Arg Trp Gly Glu Leu Lys Pro Glu Trp Glu Met Cys Gly | |
| 50 55 60 | |
| Lys Gly Glu Met Gln Ser Pro Ile Asp Leu Met Asn Glu Arg Val Asn | |
| 65 70 75 80 | |
| Ile Val Ser His Leu Gly Arg Leu Asn Arg Asp Tyr Asn Pro Ser Asn | |
| 85 90 95 | |
| Ala Thr Leu Lys Asn Arg Gly His Asp Ile Met Leu Lys Phe Glu Asp | |
| 100 105 110 | |
| Gly Ala Gly Thr Ile Lys Ile Asn Gly Phe Glu Tyr Glu Leu Gln Gln | |
| 115 120 125 | |
| Leu His Trp His Ser Pro Ser Glu His Thr Ile Asn Gly Arg Arg Phe | |
| 130 135 140 | |
| Ala Leu Glu Leu His Met Val His Glu Gly Arg Asn Arg Arg Met Ala | |
| 145 150 155 160 | |
| Val Val Thr Val Leu Tyr Lys Ile Gly Arg Ala Asp Thr Phe Ile Arg | |
| 165 170 175 | |
| Ser Leu Glu Lys Glu Len Glu Gly Ile Ala Glu Met Glu Glu Ala Glu | |
| 180 185 190 | |
| Lys Asn Val Gly Met Ile Asp Pro Thr Lys Ile Lys Ile Gly Ser Arg | |
| 195 200 205 | |
| Lys Tyr Tyr Arg Tyr Thr Gly Ser Leu Thr Thr Pro Pro Cys Thr Gln | |
| 210 215 220 | |
| Asn Val Thr Trp Ser Val Val Arg Lys Val Arg Thr Val Thr Arg Lys | |
| 225 230 235 240 | |
| Gln Val Lys Leu Leu Arg Val Ala Val His Asp Asp Ala Asn Ser Asn | |
| 245 250 255 | |
| Ala Arg Pro Val Gln Pro Thr Asn Lys Arg Ile Val His Leu Tyr Arg | |
| 260 265 270 | |
| Pro Ile Val | |
| 275 | |
| SEQ ID NO: 31/SEQ ID NO: 32 | |
| atg aag ata tca tca cta gga tgg gtc tta gtc ctt atc ttc atc tct | 48 |
| Met Lys Ile Ser Ser Leu Gly Trp Val Len Val Len Ile Phe Ile Ser | |
| 1 5 10 15 | |
| att acc att gtt tcg agt gca cca gca cct aaa cct cct aaa cct aag | 96 |
| Ile Thr Ile Val Ser Ser Ala Pro Ala Pro Lys Pro Pro Lys Pro Lys | |
| 20 25 30 | |
| cct gca cca gca cct aca cct cct aaa cct aag ccc aca cca gca cct | 144 |
| Pro Ala Pro Ala Pro Thr Pro Pro Lys Pro Lys Pro Thr Pro Ala Pro | |
| 35 40 45 | |
| aca cct cct aaa cct aag ccc aaa cca gca cct aca cct cct aaa cct | 192 |
| Thr Pro Pro Lys Pro Lys Pro Lys Pro Ala Pro Thr Pro Pro Lys Pro | |
| 50 55 60 | |
| aag cct gca cca gca cct aca cct cct aaa cct aag ccc gca cca gca | 240 |
| Lys Pro Ala Pro Ala Pro Thr Pro Pro Lys Pro Lys Pro Ala Pro Ala | |
| 65 70 75 80 | |
| cct aca cct cct aaa cct aag ccc aaa cca gca cct aca cct cct aat | 288 |
| Pro Thr Pro Pro Lys Pro Lys Pro Lys Pro Ala Pro Thr Pro Pro Asn | |
| 85 90 95 | |
| cct aag ccc aca cca gca cct aca cct cct aaa cct aag cct gca cca | 336 |
| Pro Lys Pro Thr Pro Ala Pro Thr Pro Pro Lys Pro Lys Pro Ala Pro | |
| 100 105 110 | |
| gca cca gca cca aca cca gca ccg aaa cct aaa cct gca cct aaa cca | 384 |
| Ala Pro Ala Pro Thr Pro Ala Pro Lys Pro Lys Pro Ala Pro Lys Pro | |
| 115 120 125 | |
| gca cca ggt gga gaa gtt gag gac gaa acc gag ttt agc tac gag acg | 432 |
| Ala Pro Gly Gly Glu Val Glu Asp Glu Thr Glu Phe Ser Tyr Glu Thr | |
| 130 135 140 | |
| aaa gga aac aag ggg cca gcg aaa tgg gga aca cta gat gca gag tgg | 480 |
| Lys Gly Asn Lys Gly Pro Ala Lys Trp Gly Thr Leu Asp Ala Glu Trp | |
| 145 150 155 160 | |
| aaa atg tgt gga ata ggc aaa atg caa tct cct att gat ctt cgg gac | 528 |
| Lys Met Cys Gly Ile Gly Lys Met Gln Ser Pro Ile Asp Leu Arg Asp | |
| 165 170 175 | |
| aaa aat gtg gta gtt agt aat aaa ttt gga ttg ctt cgt agc cag tat | 576 |
| Lys Asn Val Val Val Ser Asn Lys Phe Gly Leu Leu Arg Ser Gln Tyr | |
| 180 185 190 | |
| ctg cct tct aat acc acc att aag aac aga ggt cat gat atc atg ttg | 624 |
| Leu Pro Ser Asn Thr Thr Ile Lys Asn Arg Gly His Asp Ile Met Leu | |
| 195 200 205 | |
| aaa ttc aaa gga gga aat aaa ggt att ggt gtc act atc cgt ggt act | 672 |
| Lys Phe Lys Gly Gly Asn Lys Gly Ile Gly Val Thr Ile Arg Gly Thr | |
| 210 215 220 | |
| aga tat caa ctt caa caa ctt cat tgg cac tct cct tcc gaa cat aca | 720 |
| Arg Tyr Gln Leu Gln Gln Leu His Trp His Ser Pro Ser Glu His Thr | |
| 225 230 235 240 | |
| atc aat ggc aaa agg ttt gcg cta gag gaa cac ttg gtt cat gag agc | 768 |
| Ile Asn Gly Lys Arg Phe Ala Leu Glu Glu His Leu Val His Glu Ser | |
| 245 250 255 | |
| aaa gat aaa cgc tac gct gtt gtc gca ttc tta tac aat ctc gga gca | 816 |
| Lys Asp Lys Arg Tyr Ala Val Val Ala Phe Leu Tyr Asn Leu Gly Ala | |
| 260 265 270 | |
| tct gac cct ttt ctc ttt tcg ttg gaa aaa caa ttg aag aag ata act | 864 |
| Ser Asp Pro Phe Leu Phe Ser Leu Glu Lys Gln Leu Lys Lys Ile Thr | |
| 275 280 285 | |
| gat aca cat gcg tcc gag gaa cat att cgc act gtg tca agt aaa caa | 912 |
| Asp Thr His Ala Ser Glu Glu His Ile Arg Thr Val Ser Ser Lys Gln | |
| 290 295 300 | |
| gtg aag ctt ctc cgt gtg gct gta cac gat gct tca gat tca aat gcc | 960 |
| Val Lys Leu Leu Arg Val Ala Val His Asp Ala Ser Asp Ser Asn Ala | |
| 305 310 315 320 | |
| agg ccg ctt caa gca gtc aat aag cgc aag gta tat tta tac aaa cca | 1008 |
| Arg Pro Leu Gln Ala Val Asn Lys Arg Lys Val Tyr Leu Tyr Lys Pro | |
| 325 330 335 | |
| aag gtt aag tta atg aag aaa tac tgt aat ata agt tct tac tag (SEQ ID NO: 31) | 1053 |
| Lys Val Lys Leu Met Lys Lys Tyr Cys Asn Ile Ser Ser Tyr (SEQ ID NO: 32) | |
| 340 345 350 | |
| SEQ ID NO: 32 | |
| Met Lys Ile Ser Ser Leu Gly Trp Val Leu Val Leu Ile Phe Ile Ser | |
| 1 5 10 15 | |
| Ile Thr Ile Val Ser Ser Ala Pro Ala Pro Lys Pro Pro Lys Pro Lys | |
| 20 25 30 | |
| Pro Ala Pro Ala Pro Thr Pro Pro Lys Pro Lys Pro Thr Pro Ala Pro | |
| 35 40 45 | |
| Thr Pro Pro Lys Pro Lys Pro Lys Pro Ala Pro Thr Pro Pro Lys Pro | |
| 50 55 60 | |
| Lys Pro Ala Pro Ala Pro Thr Pro Pro Lys Pro Lys Pro Ala Pro Ala | |
| 65 70 75 80 | |
| Pro Thr Pro Pro Lys Pro Lys Pro Lys Pro Ala Pro Thr Pro Pro Asn | |
| 85 90 95 | |
| Pro Lys Pro Thr Pro Ala Pro Thr Pro Pro Lys Pro Lys Pro Ala Pro | |
| 100 105 110 | |
| Ala Pro Ala Pro Thr Pro Ala Pro Lys Pro Lys Pro Ala Pro Lys Pro | |
| 115 120 125 | |
| Ala Pro Gly Gly Glu Val Glu Asp Glu Thr Glu Phe Ser Tyr Glu Thr | |
| 130 135 140 | |
| Lys Gly Asn Lys Gly Pro Ala Lys Trp Gly Thr Leu Asp Ala Glu Trp | |
| 145 150 155 160 | |
| Lys Met Cys Gly Ile Gly Lys Met Gln Ser Pro Ile Asp Leu Arg Asp | |
| 165 170 175 | |
| Lys Asn Val Val Val Ser Asn Lys Phe Gly Leu Leu Arg Ser Gln Tyr | |
| 180 185 190 | |
| Leu Pro Ser Asn Thr Thr Ile Lys Asn Arg Gly His Asp Ile Met Leu | |
| 195 200 205 | |
| Lys Phe Lys Gly Gly Asn Lys Gly Ile Gly Val Thr Ile Arg Gly Thr | |
| 210 215 220 | |
| Arg Tyr Gln Leu Gln Gln Leu His Trp His Ser Pro Ser Glu His Thr | |
| 225 230 235 240 | |
| Ile Asn Gly Lys Arg Phe Ala Leu Glu Glu His Leu Val His Glu Ser | |
| 245 250 255 | |
| Lys Asp Lys Arg Tyr Ala Val Val Ala Phe Leu Tyr Asn Leu Gly Ala | |
| 260 265 270 | |
| Ser Asp Pro Phe Leu Phe Ser Leu Glu Lys Gln Leu Lys Lys Ile Thr | |
| 275 280 285 | |
| Asp Thr His Ala Ser Glu Glu His Ile Arg Thr Val Ser Ser Lys Gln | |
| 290 295 300 | |
| Val Lys Leu Leu Arg Val Ala Val His Asp Ala Ser Asp Ser Asn Ala | |
| 305 310 315 320 | |
| Arg Pro Leu Gln Ala Val Asn Lys Arg Lys Val Tyr Leu Tyr Lys Pro | |
| 325 330 335 | |
| Lys Val Lys Leu Met Lys Lys Tyr Cys Asn Ile Ser Ser Tyr | |
| 340 345 350 | |
| SEQ ID NO: 33/SEQ ID NO: 34 | |
| ctagagagca tcttcttata tcaactaaac tttgtattca tttccaagta tcactctaaa | 60 |
| tcatcttttt cgaattcgcc tcccaagat atg tcg aca gag tcg tac gaa gac | 113 |
| Met Ser Thr Glu Ser Tyr Glu Asp | |
| 1 5 | |
| gcc att aaa aga ctc gga gag ctt ctc agt aag aaa tcg gat ctc ggg | 161 |
| Ala Ile Lys Arg Leu Gly Glu Leu Leu Ser Lys Lys Ser Asp Leu Gly | |
| 10 15 20 | |
| aac gtg gca gcc gca aag atc aag aag tta acg gat gag tta gag gaa | 209 |
| Asn Val Ala Ala Ala Lys Ile Lys Lys Leu Thr Asp Glu Leu Glu Glu | |
| 25 30 35 40 | |
| ctt gat tcc aac aag tta gat gcc gta gaa cga atc aaa tcc gga ttt | 257 |
| Leu Asp Ser Asn Lys Leu Asp Ala Val Glu Arg Ile Lys Ser Gly Phe | |
| 45 50 55 | |
| ctc cat ttc aag act aat aat tat gag aag aat cct act ttg tac aat | 305 |
| Leu His Phe Lys Thr Asn Asn Tyr Glu Lys Asn Pro Thr Leu Tyr Asn | |
| 60 65 70 | |
| tca ctt gcc aag agc cag acc ccc aag ttt ttg gtg ttt gct tgt gcg | 353 |
| Ser Leu Ala Lys Ser Gln Thr Pro Lys Phe Leu Val Phe Ala Cys Ala | |
| 75 80 85 | |
| gat tca cga gtt agt cca tct cac atc ttg aat ttc caa ctt ggg gaa | 401 |
| Asp Ser Arg Val Ser Pro Ser His Ile Leu Asn Phe Gln Leu Gly Glu | |
| 90 95 100 | |
| gcc ttc atc gtt aga aac att gca aac atg gtg cca cct tat gac aag | 449 |
| Ala Phe Ile Val Arg Asn Ile Ala Asn Met Val Pro Pro Tyr Asp Lys | |
| 105 110 115 120 | |
| aca aag cac tct aat gtt ggt gcg gcc ctt gaa tat cca att aca gtc | 497 |
| Thr Lys His Ser Asn Val Gly Ala Ala Leu Glu Tyr Pro Ile Thr Val | |
| 125 130 135 | |
| ctc aac gtg gag aac att ctt gtt att gga cac agc tgt tgt ggt gga | 545 |
| Leu Asn Val Glu Asn Ile Leu Val Ile Gly His Ser Cys Cys Gly Gly | |
| 140 145 150 | |
| ata aag gga ctc atg gcc att gaa gat aat aca gct ccc act aag acc | 593 |
| Ile Lys Gly Leu Met Ala Ile Glu Asp Asn Thr Ala Pro Thr Lys Thr | |
| 155 160 165 | |
| gag ttc ata gaa aac tgg atc cag atc tgt gca ccg gcc aag aac agg | 641 |
| Glu Phe Ile Glu Asn Trp Ile Gln Ile Cys Ala Pro Ala Lys Asn Arg | |
| 170 175 180 | |
| atc aag cag gat tgt aaa gac cta agc ttt gaa gat cag tgc acc aac | 689 |
| Ile Lys Gln Asp Cys Lys Asp Leu Ser Phe Glu Asp Gln Cys Thr Asn | |
| 185 190 195 200 | |
| tgt gag aag gaa gcc gtg aac gtg tcc ttg ggg aat ctt ttg tct tac | 737 |
| Cys Glu Lys Glu Ala Val Asn Val Ser Leu Gly Asn Leu Leu Ser Tyr | |
| 205 210 215 | |
| cca ttc gtg aga gaa aga gtg gtg aag aac aag ctt gcc ata aga gga | 785 |
| Pro Phe Val Arg Glu Arg Val Val Lys Asn Lys Leu Ala Ile Arg Gly | |
| 220 225 230 | |
| gct cac tat gat ttc gta aaa gga acg ttt gat ctt tgg gaa ctt gac | 833 |
| Ala His Tyr Asp Phe Val Lys Gly Thr Phe Asp Leu Trp Glu Leu Asp | |
| 235 240 245 | |
| ttc aag act acc cct gcc ttt gcc ttg tct taa aagattcctc ctactcaaat | 886 |
| Phe Lys Thr Thr Pro Ala Phe Ala Leu Ser (SEQ ID NO: 34) | |
| 250 255 | |
| attttctcta tgttgtttct aattatgttc ttataatctt cttctgttgc ttctgtaatg | 946 |
| tcatctttgc tacttctatt ccaatagaaa tgaataaagc tttaaagagc (SEQ ID NO: 33) | 996 |
| SEQ ID NO: 34 | |
| Met Ser Thr Glu Ser Tyr Glu Asp Ala Ile Lys Arg Leu Gly Glu Leu | |
| 1 5 10 15 | |
| Leu Ser Lys Lys Ser Asp Leu Gly Asn Val Ala Ala Ala Lys Ile Lys | |
| 20 25 30 | |
| Lys Leu Thr Asp Glu Leu Glu Glu Leu Asp Ser Asn Lys Leu Asp Ala | |
| 35 40 45 | |
| Val Glu Arg Ile Lys Ser Gly Phe Leu His Phe Lys Thr Asn Asn Tyr | |
| 50 55 60 | |
| Glu Lys Asn Pro Thr Leu Tyr Asn Ser Leu Ala Lys Ser Gln Thr Pro | |
| 65 70 75 80 | |
| Lys Phe Leu Val Phe Ala Cys Ala Asp Ser Arg Val Ser Pro Ser His | |
| 85 90 95 | |
| Ile Leu Asn Phe Gln Leu Gly Glu Ala Phe Ile Val Arg Asn Ile Ala | |
| 100 105 110 | |
| Asn Met Val Pro Pro Tyr Asp Lys Thr Lys His Ser Asn Val Gly Ala | |
| 115 120 125 | |
| Ala Leu Glu Tyr Pro Ile Thr Val Leu Asn Val Glu Asn Ile Leu Val | |
| 130 135 140 | |
| Ile Gly His Ser Cys Cys Gly Gly Ile Lys Gly Leu Met Ala Ile Glu | |
| 145 150 155 160 | |
| Asp Asn Thr Ala Pro Thr Lys Thr Glu Phe Ile Glu Asn Trp Ile Gln | |
| 165 170 175 | |
| Ile Cys Ala Pro Ala Lys Asn Arg Ile Lys Gln Asp Cys Lys Asp Leu | |
| 180 185 190 | |
| Ser Phe Glu Asp Gln Cys Thr Asn Cys Glu Lys Glu Ala Val Asn Val | |
| 195 200 205 | |
| Ser Leu Gly Asn Leu Leu Ser Tyr Pro Phe Val Arg Glu Arg Val Val | |
| 210 215 220 | |
| Lys Asn Lys Leu Ala Ile Arg Gly Ala His Tyr Asp Phe Val Lys Gly | |
| 225 230 235 240 | |
| Thr Phe Asp Leu Trp Glu Leu Asp Phe Lys Thr Thr Pro Ala Phe Ala | |
| 245 250 255 | |
| Leu Ser | |
| SEQ ID NO: 35/SEQ ID NO: 36 | |
| attgttgtgt aaaactcttg ttcctcttcc tcttcaacgt gaacacttct atttctcaga | 60 |
| gaacattcac ctatatgtct ttcttcaagg agaagtcttc ctctttccag atttagatga | 120 |
| acactcttca gatgccttgt gccttattga tccagattcg aagtacccaa ctttactctc | 180 |
| tagacctttt tc atg gca gcc act ccc aca cac ttc tct gtc tec cat gat | 231 |
| Met Ala Ala Thr Pro Thr His Phe Ser Val Ser His Asp | |
| 1 5 10 | |
| cct ttt tct tcc acg tct ctc ctt aat ctc caa act caa gcg atc ttt | 279 |
| Pro Phe Ser Ser Thr Ser Leu Leu Asn Leu Gln Thr Gln Ala Ile Phe | |
| 15 20 25 | |
| ggt ccc aat cac agt tta aag aca acc cag ttg aga att cca gct tct | 327 |
| Gly Pro Asn His Ser Leu Lys Thr Thr Gln Leu Arg Ile Pro Ala Ser | |
| 30 35 40 45 | |
| ttc aga aga aaa gct aca aac ttg caa gtg atg gct tca gga aag aca | 375 |
| Phe Arg Arg Lys Ala Thr Asn Leu Gln Val Met Ala Ser Gly Lys Thr | |
| 50 55 60 | |
| cct gga ctg act cag gaa gct aat ggg gtt gca att gat aga caa aac | 423 |
| Pro Gly Leu Thr Gln Glu Ala Asn Gly Val Ala Ile Asp Arg Gln Asn | |
| 65 70 75 | |
| aac act gat gta ttt gac gac atg aaa cag cgg ttc ctg gcc ttc aag | 471 |
| Asn Thr Asp Val Phe Asp Asp Met Lys Gln Arg Phe Leu Ala Phe Lys | |
| 80 85 90 | |
| aag ctt aag tac atc agg gat gac ttt gaa cac tac aaa aat ctg gca | 519 |
| Lys Leu Lys Tyr Ile Arg Asp Asp Phe Glu His Tyr Lys Asn Leu Ala | |
| 95 100 105 | |
| gat gct caa gct cca aag ttt ctg gtg att gct tgt gca gac tct aga | 567 |
| Asp Ala Gln Ala Pro Lys Phe Leu Val Ile Ala Cys Ala Asp Ser Arg | |
| 110 115 120 125 | |
| gtt tgt cct tct gct gtc ctg gga ttc caa ccg ggt gac gca ttc act | 615 |
| Val Cys Pro Ser Ala Val Leu Gly Phe Gln Pro Gly Asp Ala Phe Thr | |
| 130 135 140 | |
| gtt cgt aac att gca aat tta gta cct cca tat gag tct gga cct act | 663 |
| Val Arg Asn Ile Ala Asn Leu Val Pro Pro Tyr Glu Ser Gly Pro Thr | |
| 145 150 155 | |
| gaa acc aaa gct gct cta gag ttc tct gtg aat act ctt aat gtg gaa | 711 |
| Glu Thr Lys Ala Ala Leu Glu Phe Ser Val Asn Thr Leu Asn Val Glu | |
| 160 165 170 | |
| aac atc tta gtc att ggt cat agc cgg tgt gga gga att caa gct tta | 759 |
| Asn Ile Leu Val Ile Gly His Ser Arg Cys Gly Gly Ile Gln Ala Leu | |
| 175 180 185 | |
| atg aaa atg gaa gac gaa gga gat tcc aga agt ttc ata cac aac tgg | 807 |
| Met Lys Met Glu Asp Glu Gly Asp Ser Arg Ser Phe Ile His Asn Trp | |
| 190 195 200 205 | |
| gta gtt gtg gga aag aag gca aag gaa agc aca aaa gct gtt gct tca | 855 |
| Val Val Val Gly Lys Lys Ala Lys Glu Ser Thr Lys Ala Val Ala Ser | |
| 210 215 220 | |
| aac ctc cat ttt gat cat cag tgc caa cat tgt gaa aag gca tcg ata | 903 |
| Asn Leu His Phe Asp His Gln Cys Gln His Cys Glu Lys Ala Ser Ile | |
| 225 230 235 | |
| aat cat tca tta gaa agg ctg ctt ggg tac ccg tgg ata gaa gag aaa | 951 |
| Asn His Ser Leu Glu Arg Leu Leu Gly Tyr Pro Trp Ile Glu Glu Lys | |
| 240 245 250 | |
| gtg cgg caa ggt tca ctg tct ctc cat ggt gga tac tat aat ttt gtt | 999 |
| Val Arg Gln Gly Ser Leu Ser Leu His Gly Gly Tyr Tyr Asn Phe Val | |
| 255 260 265 | |
| gat tgt acg ttc gag aaa tgg aca gtg gat tat gca gca agc aga ggt | 1047 |
| Asp Cys Thr Phe Glu Lys Trp Thr Val Asp Tyr Ala Ala Ser Arg Gly | |
| 270 275 280 285 | |
| aag aag aag gaa ggc agt gga atc gct gtt aaa gac cgg tca gtt tgg | 1095 |
| Lys Lys Lys Glu Gly Ser Gly Ile Ala Val Lys Asp Arg Ser Val Trp | |
| 290 295 300 | |
| tct tgacttacga ctatctcaat cttcatagag ttttttttca taatttatag | 1148 |
| Ser (SEQ ID NO: 36) | |
| agaaacatca aacccctttt ggttgggatt atcatgtgtt tgttccactt gtgtgttgaa | 1208 |
| gtcattttcc ttcttctgtc ttattgaggc agggactaat gtttgtttta tctttcagtt | 1268 |
| gtttcgttta aattccacat ttgtgcaatg aactggttgg tgtttcttta agatataatc | 1328 |
| attttgccac tgtagtgaga tcggaggcat gcat (SEQ ID NO: 35) | 1362 |
| SEQ ID NO: 36 | |
| Met Ala Ala Thr Pro Thr His Phe Ser Val Ser His Asp Pro Phe Ser | |
| 1 5 10 15 | |
| Ser Thr Ser Leu Leu Asn Leu Gln Thr Gln Ala Ile Phe Gly Pro Asn | |
| 20 25 30 | |
| His Ser Leu Lys Thr Thr Gln Leu Arg Ile Pro Ala Ser Phe Arg Arg | |
| 35 40 45 | |
| Lys Ala Thr Asn Leu Gln Val Met Ala Ser Gly Lys Thr Pro Gly Leu | |
| 50 55 60 | |
| Thr Gln Glu Ala Asn Gly Val Ala Ile Asp Arg Gln Asn Asn Thr Asp | |
| 65 70 75 80 | |
| Val Phe Asp Asp Met Lys Gln Arg Phe Leu Ala Phe Lys Lys Leu Lys | |
| 85 90 95 | |
| Tyr Ile Arg Asp Asp Phe Glu His Tyr Lys Asn Leu Ala Asp Ala Gln | |
| 100 105 110 | |
| Ala Pro Lys Phe Leu Val Ile Ala Cys Ala Asp Ser Arg Val Cys Pro | |
| 115 120 125 | |
| Ser Ala Val Leu Gly Phe Gln Pro Gly Asp Ala Phe Thr Val Arg Asn | |
| 130 135 140 | |
| Ile Ala Asn Leu Val Pro Pro Tyr Glu Ser Gly Pro Thr Glu Thr Lys | |
| 145 150 155 160 | |
| Ala Ala Leu Glu Phe Ser Val Asn Thr Leu Asn Val Glu Asn Ile Leu | |
| 165 170 175 | |
| Val Ile Gly His Ser Arg Cys Gly Gly Ile Gln Ala Leu Met Lys Met | |
| 180 185 190 | |
| Glu Asp Glu Gly Asp Ser Arg Ser Phe Ile His Asn Trp Val Val Val | |
| 195 200 205 | |
| Gly Lys Lys Ala Lys Glu Ser Thr Lys Ala Val Ala Ser Asn Leu His | |
| 210 215 220 | |
| Phe Asp His Gln Cys Gln His Cys Glu Lys Ala Ser Ile Asn His Ser | |
| 225 230 235 240 | |
| Leu Glu Arg Leu Leu Gly Tyr Pro Trp Ile Glu Glu Lys Val Arg Gln | |
| 245 250 255 | |
| Gly Ser Leu Ser Leu His Gly Gly Tyr Tyr Asn Phe Val Asp Cys Thr | |
| 260 265 270 | |
| Phe Glu Lys Trp Thr Val Asp Tyr Ala Ala Ser Arg Gly Lys Lys Lys | |
| 275 280 285 | |
| Glu Gly Ser Gly Ile Ala Val Lys Asp Arg Ser Val Trp Ser | |
| 290 295 300 | |
| SEQ ID NO: 37/SEQ ID NO: 38 | |
| atattaaacc actgtaactg taatttattg tttcgccgtc ccggaatgtt cctgttgaaa | 60 |
| tccattttcg ctgatttttt ttcttccgtc tcttcttcag cttcgaccat tttcgtcttc | 120 |
| ttcattcagt gttgagtcct cgtttacctg tgagctcgaa gaaagtgacg atca atg | 177 |
| Met | |
| 1 | |
| gga acc cta ggc aga gca ttt tac tcg gtc ggt ttt tgg atc cgt gag | 225 |
| Gly Thr Leu Gly Arg Ala Phe Tyr Ser Val Gly Phe Trp Ile Arg Glu | |
| 5 10 15 | |
| act ggt caa gct ctt gat cgc ctc ggt tgt cgc ctt caa ggc aaa aat | 273 |
| Thr Gly Gln Ala Leu Asp Arg Leu Gly Cys Arg Leu Gln Gly Lys Asn | |
| 20 25 30 | |
| tac ttc cga gaa caa ctg tca agg cat cgg aca ctg atg aat gta ttt | 321 |
| Tyr Phe Arg Glu Gln Leu Ser Arg His Arg Thr Leu Met Asn Val Phe | |
| 35 40 45 | |
| gat aag gct ccg att gtg gac aag gaa gct ttt gtg gca cca agc gcc | 369 |
| Asp Lys Ala Pro Ile Val Asp Lys Glu Ala Phe Val Ala Pro Ser Ala | |
| 50 55 60 65 | |
| tca gtt att ggg gac gtt cac att gga aga gga tcg tcc att tgg tat | 417 |
| Ser Val Ile Gly Asp Val His Ile Gly Arg Gly Ser Ser Ile Trp Tyr | |
| 70 75 80 | |
| gga tgc gta tta cga ggc gat gtg aac acc gta agt gtt ggg tca gga | 465 |
| Gly Cys Val Leu Arg Gly Asp Val Asn Thr Val Ser Val Gly Ser Gly | |
| 85 90 95 | |
| act aat att cag gac aac tca ctt gtg cat gtg gca aaa tca aac tta | 513 |
| Thr Asn Ile Gln Asp Asn Ser Leu Val His Val Ala Lys Ser Asn Leu | |
| 100 105 110 | |
| agc ggg aag gtg cac cca acc ata att gga gac aat gta acc att ggt | 561 |
| Ser Gly Lys Val His Pro Thr Ile Ile Gly Asp Asn Val Thr Ile Gly | |
| 115 120 125 | |
| cat agt gct gtt tta cat gga tgt act gtt gag gat gag acc ttt att | 609 |
| His Ser Ala Val Leu His Gly Cys Thr Val Glu Asp Glu Thr Phe Ile | |
| 130 135 140 145 | |
| ggg atg ggt gcg aca ctt ctt gat ggg gtc gtt gtt gaa aag cat ggg | 657 |
| Gly Met Gly Ala Thr Leu Leu Asp Gly Val Val Val Glu Lys His Gly | |
| 150 155 160 | |
| atg gtt gct gct ggt gca ctt gta cga caa aac acc aga att cct tct | 705 |
| Met Val Ala Ala Gly Ala Leu Val Arg Gln Asn Thr Arg Ile Pro Ser | |
| 165 170 175 | |
| gga gag gta tgg gga gga aac cca gca agg ttc ctc agg aag ctc act | 753 |
| Gly Glu Val Trp Gly Gly Asn Pro Ala Arg Phe Leu Arg Lys Leu Thr | |
| 180 185 190 | |
| gat gag gaa att gct ttt atc tct cag tca gca aca aac tac tca aac | 801 |
| Asp Glu Glu Ile Ala Phe Ile Ser Gln Ser Ala Thr Asn Tyr Ser Asn | |
| 195 200 205 | |
| ctc gca cag gct cac gct gca gag aat gca aag cca tta aat gtg att | 849 |
| Leu Ala Gln Ala His Ala Ala Glu Asn Ala Lys Pro Leu Asn Val Ile | |
| 210 215 220 225 | |
| gag ttc gag aag gtt cta cgc aag aag cat gct cta aag gac gag gag | 897 |
| Glu Phe Glu Lys Val Leu Arg Lys Lys His Ala Leu Lys Asp Glu Glu | |
| 230 235 240 | |
| tat gac tca atg ctc gga ata gtg aga gaa act cca cca gag ctt aac | 945 |
| Tyr Asp Ser Met Leu Gly Ile Val Arg Glu Thr Pro Pro Gln Leu Asn | |
| 245 250 255 | |
| ctc cct aac aac ata ctg cct gat aaa gaa acc aag cgt cct tct aat | 993 |
| Leu Pro Asn Asn Ile Leu Pro Asp Lys Glu Thr Lys Arg Pro Ser Asn | |
| 260 265 270 | |
| gtg aac tga tttttcaggg gtatgttttc tggccgaagc cctacagggt | 1042 |
| Val Asn (SEQ ID NO: 38) | |
| 275 | |
| gagatactca aggggattat gtttcggtct ctggtttgaa tatggcaggt agagtacatt | 1102 |
| agggtagacg gatttacagc ttttgaagaa gctatgttca acattttttc atggtttctt | 1162 |
| agggagtatt attgtctaat caaactttgt atgttatcac ttcggtcttt tgaacgtaag | 1222 |
| aatcaagttc atgaaacatg agtgaatatt agtctgatgc atgtgcgtat gcaaaaatcc | 1282 |
| atgtgcgcct atgttgctag gcaagcatga agaataaaga tccaaactgg atatatcata | 1342 |
| tatttatctt tttataatta ctgc (SEQ ID NO: 37) | 1366 |
| SEQ ID NO: 38 | |
| Met Gly Thr Leu Gly Arg Ala Phe Tyr Ser Val Gly Phe Trp Ile Arg | |
| 1 5 10 15 | |
| Glu Thr Gly Gln Ala Leu Asp Arg Leu Gly Cys Arg Leu Gln Gly Lys | |
| 20 25 30 | |
| Asn Tyr Phe Arg Glu Gln Leu Ser Arg His Arg Thr Leu Met Asn Val | |
| 35 40 45 | |
| Phe Asp Lys Ala Pro Ile Val Asp Lys Glu Ala Phe Val Ala Pro Ser | |
| 50 55 60 | |
| Ala Ser Val Ile Gly Asp Val His Ile Gly Arg Gly Ser Ser Ile Trp | |
| 65 70 75 80 | |
| Tyr Gly Cys Val Leu Arg Gly Asp Val Asn Thr Val Ser Val Gly Ser | |
| 85 90 95 | |
| Gly Thr Asn Ile Gln Asp Asn Ser Leu Val His Val Ala Lys Ser Asn | |
| 100 105 110 | |
| Leu Ser Gly Lys Val His Pro Thr Ile Ile Gly Asp Asn Val Thr Ile | |
| 115 120 125 | |
| Gly His Ser Ala Val Leu His Gly Cys Thr Val Glu Asp Glu Thr Phe | |
| 130 135 140 | |
| Ile Gly Met Gly Ala Thr Leu Leu Asp Gly Val Val Val Glu Lys His | |
| 145 150 155 160 | |
| Gly Met Val Ala Ala Gly Ala Leu Val Arg Gln Asn Thr Arg Ile Pro | |
| 165 170 175 | |
| Ser Gly Glu Val Trp Gly Gly Asn Pro Ala Arg Phe Leu Arg Lys Leu | |
| 180 185 190 | |
| Thr Asp Glu Glu Ile Ala Phe Ile Ser Gln Ser Ala Thr Asn Tyr Ser | |
| 195 200 205 | |
| Asn Leu Ala Gln Ala His Ala Ala Glu Asn Ala Lys Pro Leu Asn Val | |
| 210 215 220 | |
| Ile Glu Phe Glu Lys Val Leu Arg Lys Lys His Ala Leu Lys Asp Glu | |
| 225 230 235 240 | |
| Glu Tyr Asp Ser Met Leu Gly Ile Val Arg Glu Thr Pro Pro Glu Leu | |
| 245 250 255 | |
| Asn Leu Pro Asn Asn Ile Leu Pro Asp Lys Glu Thr Lys Arg Pro Ser | |
| 260 265 270 | |
| Asn Val Asn | |
| 275 | |
| SEQ ID NO: 39/SEQ ID NO: 40 | |
| cgaactcact cgagttaaaa aaaaaaatcc tcccatcaat acgcctccat aaacctctct | 60 |
| ctatctggtg gagcgacacc aaaaacaaca aagccttctc attttcacac tttgggtaat | 120 |
| cggagaatca caaaaaa atg gga acc cta gga cga gca att tac act gtg | 170 |
| Met Gly Thr Leu Gly Arg Ala Ile Tyr Thr Val | |
| 1 5 10 | |
| ggt aac tgg att cgt gga act ggt caa gct ctt gat cgc gtt ggt tct | 218 |
| Gly Asn Trp Ile Arg Gly Thr Gly Gln Ala Leu Asp Arg Val Gly Ser | |
| 15 20 25 | |
| ctt ctt caa gga agt cac cgt atc gag gaa cat ctg tcg agg cat cgg | 266 |
| Leu Leu Gln Gly Ser His Arg Ile Glu Glu His Leu Ser Arg His Arg | |
| 30 35 40 | |
| acg ttg atg aat gtg ttt gat aaa tca cca ttg gtg gat aaa gat gtg | 314 |
| Thr Leu Met Asn Val Phe Asp Lys Ser Pro Leu Val Asp Lys Asp Val | |
| 45 50 55 | |
| ttt gtg gct ccg agt gct tct gtt att ggt gat gtt cag atc gga aaa | 362 |
| Phe Val Ala Pro Ser Ala Ser Val Ile Gly Asp Val Gln Ile Gly Lys | |
| 60 65 70 75 | |
| ggc tcg tcg att tgg tat ggc tgt gtt ctt cga ggt gat gtg aat aac | 410 |
| Gly Ser Ser Ile Trp Tyr Gly Cys Val Leu Arg Gly Asp Val Asn Asn | |
| 80 85 90 | |
| atc agt gtt gga tct ggg acg aat atc caa gat aat acg ctt gta cat | 458 |
| Ile Ser Val Gly Ser Gly Thr Asn Ile Gln Asp Asn Thr Leu Val His | |
| 95 100 105 | |
| gtt gca aag acc aac ata agt ggc aag gtt cta cct act ctg att ggg | 506 |
| Val Ala Lys Thr Asn Ile Ser Gly Lys Val Leu Pro Thr Leu Ile Gly | |
| 110 115 120 | |
| gac aat gta aca gta ggt cac agt gct gtc att cat ggg tgt act gtt | 554 |
| Asp Asn Val Thr Val Gly His Ser Ala Val Ile His Gly Cys Thr Val | |
| 125 130 135 | |
| gag gat gat gct ttt gtt ggt atg gga gca aca cta ctt gat ggt gtg | 602 |
| Glu Asp Asp Ala Phe Val Gly Met Gly Ala Thr Leu Leu Asp Gly Val | |
| 140 145 150 155 | |
| gtg gtt gag aaa cat gcc atg gtt gct gct ggt tct ctt gtg aaa cag | 650 |
| Val Val Glu Lys His Ala Met Val Ala Ala Gly Ser Leu Val Lys Gln | |
| 160 165 170 | |
| aac acg cga atc cct tct gga gag gtg tgg gga gga aat cca gca aag | 698 |
| Asn Thr Arg Ile Pro Ser Gly Glu Val Trp Gly Gly Asn Pro Ala Lys | |
| 175 180 185 | |
| ttc atg aga aag tta aca gat gaa gag ata gta tac atc tca cag tca | 746 |
| Phe Met Arg Lys Leu Thr Asp Glu Glu Ile Val Tyr Ile Ser Gln Ser | |
| 190 195 200 | |
| gca aag aat tac atc aat ctc gca cag att cac gcc tca gag aat tca | 794 |
| Ala Lys Asn Tyr Ile Asn Leu Ala Gln Ile His Ala Ser Glu Asn Ser | |
| 205 210 215 | |
| aag tca ttt gag cag atc gag gtt gag aga gcg ctt agg aag aag tat | 842 |
| Lys Ser Phe Glu Gln Ile Glu Val Glu Arg Ala Leu Arg Lys Lys Tyr | |
| 220 225 230 235 | |
| gca cgc aag gac gag gat tac gat tca atg ctt ggg att acc cgt gaa | 890 |
| Ala Arg Lys Asp Glu Asp Tyr Asp Ser Met Leu Gly Ile Thr Arg Glu | |
| 240 245 250 | |
| act cca ccg gag ttg att ctt ccc gac aat gtc tta cca ggt ggt aaa | 938 |
| Thr Pro Pro Glu Leu Ile Leu Pro Asp Asn Val Leu Pro Gly Gly Lys | |
| 255 260 265 | |
| ccc gtc gcc aag gtt ccg tct act cag tac ttc taa ttccaatctc | 984 |
| Pro Val Ala Lys Val Pro Ser Thr Gln Tyr Phe (SEQ ID NO: 40) | |
| 270 275 | |
| aggttgtttt tgtgtgttga aatcatttca agacaggatt gattctctgg aaggtcaaga | 1044 |
| gagatattat tttggtttta acttttcttc cgagcaagca ggagatttat catccttgct | 1104 |
| caataatgta tggttgcatt atgaagtcat ttcttcgagg aacaatttgc agaaagagaa | 1164 |
| acaaagttgg attaatcttt c (SEQ ID NO: 39) | 1185 |
| SEQ ID NO: 40 | |
| Met Gly Thr Leu Gly Arg Ala Ile Tyr Thr Val Gly Asn Trp Ile Arg | |
| 1 5 10 15 | |
| Gly Thr Gly Gln Ala Leu Asp Arg Val Gly Ser Leu Leu Gln Gly Ser | |
| 20 25 30 | |
| His Arg Ile Glu Glu His Leu Ser Arg His Arg Thr Leu Met Asn Val | |
| 35 40 45 | |
| Phe Asp Lys Ser Pro Leu Val Asp Lys Asp Val Phe Val Ala Pro Ser | |
| 50 55 60 | |
| Ala Ser Val Ile Gly Asp Val Gln Ile Gly Lys Gly Ser Ser Ile Trp | |
| 65 70 75 80 | |
| Tyr Gly Cys Val Leu Arg Gly Asp Val Asn Asn Ile Ser Val Gly Ser | |
| 85 90 95 | |
| Gly Thr Asn Ile Gln Asp Asn Thr Leu Val His Val Ala Lys Thr Asn | |
| 100 105 110 | |
| Ile Ser Gly Lys Val Leu Pro Thr Leu Ile Gly Asp Asn Val Thr Val | |
| 115 120 125 | |
| Gly His Ser Ala Val Ile His Gly Cys Thr Val Glu Asp Asp Ala Phe | |
| 130 135 140 | |
| Val Gly Met Gly Ala Thr Leu Leu Asp Gly Val Val Val Glu Lys His | |
| 145 150 155 160 | |
| Ala Met Val Ala Ala Gly Ser Leu Val Lys Gln Asn Thr Arg Ile Pro | |
| 165 170 175 | |
| Ser Gly Glu Val Trp Gly Gly Asn Pro Ala Lys Phe Met Arg Lys Leu | |
| 180 185 190 | |
| Thr Asp Glu Glu Ile Val Tyr Ile Ser Gln Ser Ala Lys Asn Tyr Ile | |
| 195 200 205 | |
| Asn Leu Ala Gln Ile His Ala Ser Glu Asn Ser Lys Ser Phe Glu Gln | |
| 210 215 220 | |
| Ile Glu Val Glu Arg Ala Leu Arg Lys Lys Tyr Ala Arg Lys Asp Glu | |
| 225 230 235 240 | |
| Asp Tyr Asp Ser Met Leu Gly Ile Thr Arg Glu Thr Pro Pro Glu Leu | |
| 245 250 255 | |
| Ile Leu Pro Asp Asn Val Leu Pro Gly Gly Lys Pro Val Ala Lys Val | |
| 260 265 270 | |
| Pro Ser Thr Gln Tyr Phe | |
| 275 | |
| SEQ ID NO: 41/SEQ ID NO: 42 | |
| caaagactgc actctctcct cttcctctgg ctccggcgaa aaaccccttt tcgatttcat | 60 |
| tgataaaacg caaatcgatc tctcgtgtgg aagaagaaga agaacacg atg gga aca | 117 |
| Met Gly Thr | |
| 1 | |
| atg ggt aaa gca ttc tac agc gta gga ttc tgg atc cgt gaa act ggt | 165 |
| Met Gly Lys Ala Phe Tyr Ser Val Gly Phe Trp Ile Arg Glu Thr Gly | |
| 5 10 15 | |
| caa gca ctt gat cgg ctc ggt tgt cgc ctc caa ggg aaa aat cat ttc | 213 |
| Gln Ala Leu Asp Arg Leu Gly Cys Arg Leu Gln Gly Lys Asn His Phe | |
| 20 25 30 35 | |
| cga gaa cag cta tca agg cac cgc aca ctc atg aat gtt ttt gac aaa | 261 |
| Arg Glu Gln Leu Ser Arg His Arg Thr Leu Met Asn Val Phe Asp Lys | |
| 40 45 50 | |
| acc cct aat gtg gat aag ggg gct ttt gtg gct cct aac gct tct ctc | 309 |
| Thr Pro Asn Val Asp Lys Gly Ala Phe Val Ala Pro Asn Ala Ser Leu | |
| 55 60 65 | |
| tct ggt gat gtc cat gtg gga aga ggt tct tcc att tgg tat gga tgt | 357 |
| Ser Gly Asp Val His Val Gly Arg Gly Ser Ser Ile Trp Tyr Gly Cys | |
| 70 75 80 | |
| gtc ttg aga gac ata ccc ttt gat tta atg acc gac tct gca gga gat | 405 |
| Val Leu Arg Asp Ile Pro Phe Asp Leu Met Thr Asp Ser Ala Gly Asp | |
| 85 90 95 | |
| gct aac agc att agt gtt gga gct ggg acc aat att cag gac aac gct | 453 |
| Ala Asn Ser Ile Ser Val Gly Ala Gly Thr Asn Ile Gln Asp Asn Ala | |
| 100 105 110 115 | |
| ctt gtc cac gtt gct aag acc aac tta agt ggg aag gtc tta cct act | 501 |
| Leu Val His Val Ala Lys Thr Asn Leu Ser Gly Lys Val Leu Pro Thr | |
| 120 125 130 | |
| gtc att gga gac aat gtc acc att ggt cat agt gct gtt tta cat ggc | 549 |
| Val Ile Gly Asp Asn Val Thr Ile Gly His Ser Ala Val Leu His Gly | |
| 135 140 145 | |
| tgc act gtc gag gat gag gcc tat att ggt aca agt gca act gtc ttg | 597 |
| Cys Thr Val Glu Asp Glu Ala Tyr Ile Gly Thr Ser Ala Thr Val Leu | |
| 150 155 160 | |
| gat gga gct cat gtt gaa aaa cat gcc atg gtt gct tca gga gct ctt | 645 |
| Asp Gly Ala His Val Glu Lys His Ala Met Val Ala Ser Gly Ala Leu | |
| 165 170 175 | |
| gtt agg cag aac act aga att ccc tct ggc gag gtt tgg gga ggc aac | 693 |
| Val Arg Gln Asn Thr Arg Ile Pro Ser Gly Glu Val Trp Gly Gly Asn | |
| 180 185 190 195 | |
| cca gct aaa ttt ctg agg aag gtg aca gaa gaa gaa aga gtc ttc ttc | 741 |
| Pro Ala Lys Phe Leu Arg Lys Val Thr Glu Glu Glu Arg Val Phe Phe | |
| 200 205 210 | |
| tcc agt tcg gct gtg gag tac tcc aac tta gct caa gct cac gcc aca | 789 |
| Ser Ser Ser Ala Val Glu Tyr Ser Asn Leu Ala Gln Ala His Ala Thr | |
| 215 220 225 | |
| gag aac gca aag aac ttg gac gag gct gag ttc aag aag ctt cta aac | 837 |
| Glu Asn Ala Lys Asn Leu Asp Glu Ala Glu Phe Lys Lys Leu Leu Asn | |
| 230 235 240 | |
| aag aag aac gct cgc gat aca gaa tat gat tca gta ctc gat gat ctc | 885 |
| Lys Lys Asn Ala Arg Asp Thr Glu Tyr Asp Ser Val Leu Asp Asp Leu | |
| 245 250 255 | |
| acg ctc cct gag aat gta cca aaa gca gct tga ggcgtttaac ctgtgccgcc | 938 |
| Thr Leu Pro Glu Asn Val Pro Lys Ala Ala (SEQ ID NO: 42) | |
| 260 265 | |
| ttgcgaatct tgatttgttt ggatttgaaa agtaaaaaca aagaacttga tttcctgctt | 998 |
| ctccaataaa gttttcttgg gcgtaaaatc cattggccag tgctcactgg gaaagttttc | 1058 |
| ggcttaaagg cattcatttc tctgttaaag attgtgaggg gttttgttct cttgtaactt | 1118 |
| gagaaagaaa agttgtaacc ttttcttcct ttttatgtcg tctaataaat tgttgatcag | 1178 |
| acagacattt aggttgacct ttgcccataa aaagatagct ctgcttcaat aa (SEQ ID NO: 41) | 1230 |
| SEQ ID NO: 42 | |
| Met Gly Thr Met Gly Lys Ala Phe Tyr Ser Val Gly Phe Trp Ile Arg | |
| 1 5 10 15 | |
| Glu Thr Gly Gln Ala Leu Asp Arg Leu Gly Cys Arg Leu Gln Gly Lys | |
| 20 25 30 | |
| Asn His Phe Arg Glu Gln Leu Ser Arg His Arg Thr Leu Met Asn Val | |
| 35 40 45 | |
| Phe Asp Lys Thr Pro Asn Val Asp Lys Gly Ala Phe Val Ala Pro Asn | |
| 50 55 60 | |
| Ala Ser Leu Ser Gly Asp Val His Val Gly Arg Gly Ser Ser Ile Trp | |
| 65 70 75 80 | |
| Tyr Gly Cys Val Leu Arg Asp Ile Pro Phe Asp Leu Met Thr Asp Ser | |
| 85 90 95 | |
| Ala Gly Asp Ala Asn Ser Ile Ser Val Gly Ala Gly Thr Asn Ile Gln | |
| 100 105 110 | |
| Asp Asn Ala Leu Val His Val Ala Lys Thr Asn Leu Ser Gly Lys Val | |
| 115 120 125 | |
| Leu Pro Thr Val Ile Gly Asp Asn Val Thr Ile Gly His Ser Ala Val | |
| 130 135 140 | |
| Leu His Gly Cys Thr Val Glu Asp Glu Ala Tyr Ile Gly Thr Ser Ala | |
| 145 150 155 160 | |
| Thr Val Leu Asp Gly Ala His Val Glu Lys His Ala Met Val Ala Ser | |
| 165 170 175 | |
| Gly Ala Leu Val Arg Gln Asn Thr Arg Ile Pro Ser Gly Glu Val Trp | |
| 180 185 190 | |
| Gly Gly Asn Pro Ala Lys Phe Leu Arg Lys Val Thr Glu Glu Glu Arg | |
| 195 200 205 | |
| Val Phe Phe Ser Ser Ser Ala Val Glu Tyr Ser Asn Leu Ala Gln Ala | |
| 210 215 220 | |
| His Ala Thr Glu Asn Ala Lys Asn Leu Asp Glu Ala Glu Phe Lys Lys | |
| 225 230 235 240 | |
| Leu Leu Asn Lys Lys Asn Ala Arg Asp Thr Glu Tyr Asp Ser Val Leu | |
| 245 250 255 | |
| Asp Asp Leu Thr Leu Pro Glu Asn Val Pro Lys Ala Ala | |
| 260 265 | |
| SEQ ID NO: 43/SEQ ID NO: 44 | |
| actctctctc ttttcctctt tgcaaatcct tgaagaaatc caaaatccat agca atg | 57 |
| Met | |
| 1 | |
| gcg act tcg ata gct cga ttg tct cgg aga gga gtc act tct aac ctg | 105 |
| Ala Thr Ser Ile Ala Arg Leu Ser Arg Arg Gly Val Thr Ser Asn Leu | |
| 5 10 15 | |
| atc cgt cgt tgc ttc gct gcg gaa gcg gcg ttg gcg agg aag aca gag | 153 |
| Ile Arg Arg Cys Phe Ala Ala Glu Ala Ala Leu Ala Arg Lys Thr Glu | |
| 20 25 30 | |
| tta cct aaa ccg caa ttc acg gtg tcg ccg tcg acg gat cgt gtg aaa | 201 |
| Leu Pro Lys Pro Gln Phe Thr Val Ser Pro Ser Thr Asp Arg Val Lys | |
| 35 40 45 | |
| tgg gac tac aga ggc caa cga cag atc att cct ttg gga cag tgg ctt | 249 |
| Trp Asp Tyr Arg Gly Gln Arg Gln Ile Ile Pro Leu Gly Gln Trp Leu | |
| 50 55 60 65 | |
| ccg aag gta gcc gtt gat gct tac gtg gca ccc aac gtt gtg ctg gct | 297 |
| Pro Lys Val Ala Val Asp Ala Tyr Val Ala Pro Asn Val Val Leu Ala | |
| 70 75 80 | |
| ggt cag gtc aca gtc tgg gac ggc tcg tct gtt tgg aac ggt gcc gtt | 345 |
| Gly Gln Val Thr Val Trp Asp Gly Ser Ser Val Trp Asn Gly Ala Val | |
| 85 90 95 | |
| ttg cgc ggc gat ctc aac aaa atc act gtt gga ttc tgc tcg aat gta | 393 |
| Leu Arg Gly Asp Leu Asn Lys Ile Thr Val Gly Phe Cys Ser Asn Val | |
| 100 105 110 | |
| cag gaa cgg tgt gtt gtt cat gcc gcc tgg tct tcc cca aca gga tta | 441 |
| Gln Glu Arg Cys Val Val His Ala Ala Trp Ser Ser Pro Thr Gly Leu | |
| 115 120 125 | |
| cca gca gcg aca ata atc gac agg tat gtg aca gta ggt gcc tac agt | 489 |
| Pro Ala Ala Thr Ile Ile Asp Arg Tyr Val Thr Val Gly Ala Tyr Scr | |
| 130 135 140 145 | |
| ctt ctg aga tca tgt acc atc gaa cca gag tgc atc atc ggt caa cac | 537 |
| Leu Leu Arg Ser Cys Thr Ile Glu Pro Glu Cys Ile Ile Gly Gln His | |
| 150 155 160 | |
| tca ata cta atg gaa ggc tca ctg gtt gag acc cgg tca atc ttg gaa | 585 |
| Ser Ile Leu Met Glu Gly Ser Leu Val Glu Thr Arg Ser Ile Leu Glu | |
| 165 170 175 | |
| gcg ggt tca gtt gtg ccg cca gga aga agg atc cca tca ggt gaa cta | 633 |
| Ala Gly Ser Val Val Pro Pro Gly Arg Arg Ile Pro Ser Gly Glu Leu | |
| 180 185 190 | |
| tgg gga ggc aat cca gca aga ttc att aga acc cta acc aac gaa gaa | 681 |
| Trp Gly Gly Asn Pro Ala Arg Phe Ile Arg Thr Leu Thr Asn Glu Glu | |
| 195 200 205 | |
| acc cta gag atc cca aaa ctc gct gta gcc atc aac cac tta agc gga | 729 |
| Thr Leu Glu Ile Pro Lys Leu Ala Val Ala Ile Asn His Leu Ser Gly | |
| 210 215 220 225 | |
| gat tac ttc tct gag ttc cta cct tac tca act gtc tac tta gag gta | 777 |
| Asp Tyr Phe Ser Glu Phe Leu Pro Tyr Ser Thr Val Tyr Leu Glu Val | |
| 230 235 240 | |
| gag aag ttc aag aag tcc ctt ggg atc gcc gtt tag aag cttcatctt | 826 |
| Glu Lys Phe Lys Lys Ser Leu Gly Ile Ala Val Lys (SEQ ID NO : 44) | |
| 245 250 | |
| ttcgtgattc actttcatgt gtttatctat catatgaggt ctttctctct gcatattgca | 886 |
| ataagtagct gatgaacatc aaaacaagtc cggctctctt ttttggttct aaaacgtttg | 946 |
| tcatttcgtt ttttgggttc tttgtaaaat tccatttaaa actgattttg gctgaatatt | 1006 |
| gtctgaatga taatggcgac gacttctggt tttgtt (SEQ ID NO: 43) | 1042 |
| SEQ ID NO: 44 | |
| Met Ala Thr Ser Ile Ala Arg Leu Ser Arg Arg Gly Val Thr Ser Asn | |
| 1 5 10 15 | |
| Leu Ile Arg Arg Cys Phe Ala Ala Glu Ala Ala Leu Ala Arg Lys Thr | |
| 20 25 30 | |
| Glu Leu Pro Lys Pro Gln Phe Thr Val Ser Pro Ser Thr Asp Arg Val | |
| 35 40 45 | |
| Lys Trp Asp Tyr Arg Gly Gln Arg Gln Ile Ile Pro Leu Gly Gln Trp | |
| 50 55 60 | |
| Leu Pro Lys Val Ala Val Asp Ala Tyr Val Ala Pro Asn Val Val Leu | |
| 65 70 75 80 | |
| Ala Gly Gln Val Thr Val Trp Asp Gly Ser Ser Val Trp Asn Gly Ala | |
| 85 90 95 | |
| Val Leu Arg Gly Asp Leu Asn Lys Ile Thr Val Gly Phe Cys Ser Asn | |
| 100 105 110 | |
| Val Gln Glu Arg Cys Val Val His Ala Ala Trp Ser Ser Pro Thr Gly | |
| 115 120 125 | |
| Leu Pro Ala Ala Thr Ile Ile Asp Arg Tyr Val Thr Val Gly Ala Tyr | |
| 130 135 140 | |
| Ser Leu Leu Arg Ser Cys Thr Ile Glu Pro Glu Cys Ile Ile Gly Gln | |
| 145 150 155 160 | |
| His Ser Ile Leu Met Glu Gly Ser Leu Val Glu Thr Arg Ser Ile Leu | |
| 165 170 175 | |
| Glu Ala Gly Ser Val Val Pro Pro Gly Arg Arg Ile Pro Ser Gly Glu | |
| 180 185 190 | |
| Leu Trp Gly Gly Asn Pro Ala Arg Phe Ile Arg Thr Leu Thr Asn Glu | |
| 195 200 205 | |
| Glu Thr Leu Glu Ile Pro Lys Leu Ala Val Ala Ile Asn His Leu Ser | |
| 210 215 220 | |
| Gly Asp Tyr Phe Ser Glu Phe Leu Pro Tyr Ser Thr Val Tyr Leu Glu | |
| 225 230 235 240 | |
| Val Glu Lys Phe Lys Lys Ser Leu Gly Ile Ala Val | |
| 245 250 | |
| SEQ ID NO: 45/SEQ ID NO: 46 | |
| ctcccgacga ctcctctctg tctcctcctc cgggaagctt tctgtctctc tctctctctc | 60 |
| tctacacaag accttgaaga atccgattcc ataaca atg gcg act tcg tta gca | 114 |
| Met Ala Thr Ser Leu Ala | |
| 1 5 | |
| cga atc tct aaa aga agc ata aca tcg gct gtt tca tcg aat ctg att | 162 |
| Arg Ile Ser Lys Arg Ser Ile Thr Ser Ala Val Ser Ser Asn Leu Ile | |
| 10 15 20 | |
| cgg cgt tac ttc gcc gcg gaa gca gta gcg gtg gcg acg acg gaa aca | 210 |
| Arg Arg Tyr Phe Ala Ala Glu Ala Val Ala Val Ala Thr Thr Glu Thr | |
| 25 30 35 | |
| cct aaa ccg aaa tcg cag gtg acg ccg tcg ccg gat cgg gta aaa tgg | 258 |
| Pro Lys Pro Lys Ser Gln Val Thr Pro Ser Pro Asp Arg Val Lys Trp | |
| 40 45 50 | |
| gac tac aga ggc cag aga cag ata att cct ctg gga cag tgg cta ccg | 306 |
| Asp Tyr Arg Gly Gln Arg Gln Ile Ile Pro Leu Gly Gln Trp Leu Pro | |
| 55 60 65 70 | |
| aag gta gct gta gat gct tac gtg gca cct aac gtt gtg ttg gct ggt | 354 |
| Lys Val Ala Val Asp Ala Tyr Val Ala Pro Asn Val Val Leu Ala Gly | |
| 75 80 85 | |
| cag gtc acc gtc tgg gac ggc tcg tct gta tgg aac ggt gcc gtt ttg | 402 |
| Gln Val Thr Val Trp Asp Gly Ser Ser Val Trp Asn Gly Ala Val Leu | |
| 90 95 100 | |
| aga gga gat ctt aat aag atc acc gtt gga ttc tgc tca aat gtc cag | 450 |
| Arg Gly Asp Leu Asn Lys Ile Thr Val Gly Phe Cys Ser Asn Val Gln | |
| 105 110 115 | |
| gaa cgg tgt gtt gtt cat gct gcg tgg tcg tcg cct aca gga tta cca | 498 |
| Glu Arg Cys Val Val His Ala Ala Trp Ser Ser Pro Thr Gly Leu Pro | |
| 120 125 130 | |
| gca caa aca ttg atc gat agg tac gtg aca gtt ggt gca tac agt ctt | 546 |
| Ala Gln Thr Leu Ile Asp Arg Tyr Val Thr Val Gly Ala Tyr Ser Leu | |
| 135 140 145 150 | |
| tta aga tca tgc act atc gaa cca gaa tgc atc atc ggg caa cac tca | 594 |
| Leu Arg Ser Cys Thr Ile Glu Pro Glu Cys Ile Ile Gly Gln His Ser | |
| 155 160 165 | |
| atc cta atg gaa ggt tca ctg gtc gaa acc cgc tca atc cta gaa gct | 642 |
| Ile Leu Met Glu Gly Ser Leu Val Glu Thr Arg Ser Ile Leu Glu Ala | |
| 170 175 180 | |
| ggt tct gtt tta cca cct ggc aga aga atc cca tct ggt gaa cta tgg | 690 |
| Gly Ser Val Leu Pro Pro Gly Arg Arg Ile Pro Ser Gly Glu Leu Trp | |
| 185 190 195 | |
| gga ggc aat cca gca agg ttt att cga aca ctc acc aat gaa gaa acc | 738 |
| Gly Gly Asn Pro Ala Arg Phe Ile Arg Thr Leu Thr Asn Glu Glu Thr | |
| 200 205 210 | |
| tta gag atc ccg aaa ctt gct gtt gcc att aac cac cta agt gga gat | 786 |
| Leu Glu Ile Pro Lys Leu Ala Val Ala Ile Asn His Leu Ser Gly Asp | |
| 215 220 225 230 | |
| tac ttc tca gag ttc ttg cct tac tca act atc tat cta gag gtt gag | 834 |
| Tyr Phe Ser Glu Phe Leu Pro Tyr Ser Thr Ile Tyr Leu Glu Val Glu | |
| 235 240 245 | |
| aag ttc aag aaa tcc ctt gga atc gcc atc tag aaa gcttcttcca | 880 |
| Lys Phe Lys Lys Ser Leu Gly Ile Ala Ile Lys (SEQ ID NO: 46) | |
| 250 255 | |
| ggtttctggc tacttccctc attaagaaag cttcttcgtt ttcggaattt gatctgaata | 940 |
| agtagctgcg gaacaagaaa aagagcagag ctgtgtttca aatgttgtct tctctgtttg | 1000 |
| ttttgtttaa gttcatatcc ttgtgttcaa actttctatg aagatgataa tggtgaaaac | 1060 |
| tggaaagtgt aaaacttctt tcgtctcccc tcacaattgg aaaagctaat aatctcgtag | 1120 |
| tgttatagaa (SEQ ID NO: 45) | 1130 |
| SEQ ID NO: 46 | |
| Met Ala Thr Ser Leu Ala Arg Ile Ser Lys Arg Ser Ile Thr Ser Ala | |
| 1 5 10 15 | |
| Val Ser Ser Asn Leu Ile Arg Arg Tyr Phe Ala Ala Glu Ala Val Ala | |
| 20 25 30 | |
| Val Ala Thr Thr Glu Thr Pro Lys Pro Lys Ser Gln Val Thr Pro Ser | |
| 35 40 45 | |
| Pro Asp Arg Val Lys Trp Asp Tyr Arg Gly Gln Arg Gln Ile Ile Pro | |
| 50 55 60 | |
| Leu Gly Gln Trp Leu Pro Lys Val Ala Val Asp Ala Tyr Val Ala Pro | |
| 65 70 75 80 | |
| Asn Val Val Leu Ala Gly Gln Val Thr Val Trp Asp Gly Ser Ser Val | |
| 85 90 95 | |
| Trp Asn Gly Ala Val Leu Arg Gly Asp Leu Asn Lys Ile Thr Val Gly | |
| 100 105 110 | |
| Phe Cys Ser Asn Val Gln Glu Arg Cys Val Val His Ala Ala Trp Ser | |
| 115 120 125 | |
| Ser Pro Thr Gly Leu Pro Ala Gln Thr Leu Ile Asp Arg Tyr Val Thr | |
| 130 135 140 | |
| Val Gly Ala Tyr Ser Leu Leu Arg Ser Cys Thr Ile Glu Pro Glu Cys | |
| 145 150 155 160 | |
| Ile Ile Gly Gln His Ser Ile Leu Met Glu Gly Ser Leu Val Glu Thr | |
| 165 170 175 | |
| Arg Ser Ile Leu Glu Ala Gly Ser Val Leu Pro Pro Gly Arg Arg Ile | |
| 180 185 190 | |
| Pro Ser Gly Glu Leu Trp Gly Gly Asn Pro Ala Arg Phe Ile Arg Thr | |
| 195 200 205 | |
| Leu Thr Asn Glu Glu Thr Leu Glu Ile Pro Lys Leu Ala Val Ala Ile | |
| 210 215 220 | |
| Asn His Leu Ser Gly Asp Tyr Phe Ser Glu Phe Leu Pro Tyr Ser Thr | |
| 225 230 235 240 | |
| Ile Tyr Leu Glu Val Glu Lys Phe Lys Lys Ser Leu Gly Ile Ala Ile | |
| 245 250 255 | |

A number of embodiments of the invention have been described. Nevertheless, it can be understood that various modifications may be made without departing from the spirit and scope of the invention. Accordingly, other embodiments are within the scope of the following claims.

1. A method for:
increasing the water use efficiency of a guard cell, a plant, plant leaf, plant organ or plant part; or
increasing the rate of growth or biomass production in a plant, plant leaf, plant organ or plant part or under conditions of drought or increased atmospheric carbon dioxide; or
enhancing the carbon dioxide (CO₂) sensitivity of a plant, plant leaf, plant organ or plant part; or
down-regulating or decreasing carbon dioxide (CO₂) and/or water exchange in a guard cell of a plant, plant leaf, plant organ or plant part;
comprising:
(a) in a cell of the plant, plant leaf, plant organ or plant part, or in a plant guard cell, increasing the expression and/or activity of:
(1) an OST1 (Open Stomata 1, also known as SnRK2.6) protein kinase-expressing nucleic acid or an OST1 protein kinase gene or mRNA (message) encoding a polypeptide with OST1 protein kinase activity; or
(2) a protein kinase SnRK2.2- or SnRK2.3-expressing nucleic acid or an SnRK2.2- or SnRK2.3 protein kinase gene or mRNA or a message encoding a polypeptide with SnRK2.2- or SnRK2.3 protein kinase activity (SnRK2 genes are SNF1 Related Protein Kinase Subfamily 2 genes) (SNF1 is “Sucrose non-fermenting 1”);
(b) the method of (a), wherein the increasing of expression and/or activity of the OST1, SnRK2.2 or SnRK2.3 protein kinase is by:
(1) providing a heterologous OST1-, SnRK2.2- or SnRK2.3-expressing nucleic acid or a gene or a message and expressing the gene, message and/or protein in the guard cell, plant, plant leaf, plant organ or plant part;
(2) increasing of expression and/or activity of a homologous OST1-, SnRK2.2- or SnRK2.3-expressing nucleic acid or a gene or a message; or,
(3) a combination of (1) and (2);
(c) the method of (a), further comprising in the cell of the plant, plant leaf, plant organ or plant part, or in the plant guard cell, increasing the expression and/or activity of a CO₂ sensor protein or a carbonic anhydrase by:
(1) providing a heterologous CO₂ sensor protein-expressing nucleic acid or (e.g., a gene or a message, or a carbonic anhydrase-expressing nucleic acid (e.g., a gene or message) and expressing the gene, message and/or protein in the guard cell, plant, plant leaf, plant organ or plant part;
(2) increasing of expression and/or activity of a homologous CO₂ sensor protein-expressing nucleic acid or a gene or a message or a homologous OST1 carbonic anhydrase-expressing nucleic acid (e.g., a gene or message); or,
(3) a combination of (1) and (2); or
(d) the method of (c), wherein the carbonic anhydrase is a β-carbonic anhydrase;
thereby:
increasing the water use efficiency of the guard cell, plant, plant leaf, plant organ or plant part; or
increasing the rate of growth or biomass production in the plant, plant leaf, plant organ or plant part; or
enhancing the carbon dioxide (CO₂) sensitivity of the plant, plant leaf, plant organ or plant part; or
down-regulating or decreasing carbon dioxide (CO₂) and/or water exchange in the guard cell of the plant, plant leaf, plant organ or plant part.

2. A method for: up-regulating or increasing carbon dioxide (CO₂) and/or water exchange in a guard cell, a plant, plant leaf, plant organ or plant part;
decreasing the water use efficiency of a guard cell, a plant, plant leaf, plant organ or plant part; or
decreasing or desensitizing the carbon dioxide (CO₂) sensitivity of a plant, plant leaf, plant organ or plant part; or
upregulating or increasing carbon dioxide (CO₂) and/or water exchange in a guard cell of a plant, plant leaf, plant organ or plant part; comprising:
(1) (a) in a cell of the plant, plant leaf, plant organ or plant part, or in a plant guard cell, decreasing the expression and/or activity of:
(1) an OST1 protein kinase-expressing nucleic acid or an OST1 protein kinase gene or mRNA or message encoding a polypeptide with OST1 protein kinase activity; or
(2) a protein kinase SnRK2.2- or SnRK2.3-expressing nucleic acid or an SnRK2.2- or SnRK2.3 protein kinase gene or mRNA or message encoding a polypeptide with SnRK2.2 or SnRK2.3 protein kinase activity;
(b) the method of (a), wherein the decreasing of expression and/or activity of the OST1, SnRK2.2 or SnRK2.3 protein kinase is by:
(1) providing a heterologous antisense or iRNA OST1, SnRK2.2 or SnRK2.3 protein kinase nucleic acid or to decrease the expression or activity of a gene or a message, or any nucleic acid inhibitory to the expression of the OST1, SnRK2.2 or SnRK2.3 protein kinase; and,
expressing the inhibitory nucleic acid, the antisense or the iRNA in the guard cell, plant, plant leaf, plant organ or plant part;
(2) decreasing of expression and/or activity of a homologous OST1-, SnRK2.2- or SnRK2.3 kinase-expressing nucleic acid or a gene or a message; or, (3) a combination of (1) and (2);
(c) the method of (a), further comprising in the cell of the plant, plant leaf, plant organ or plant part, or in the plant guard cell, decreasing the expression and/or activity of a CO₂ sensor protein or a carbonic anhydrase by:
(1) providing a heterologous antisense or iRNA to a CO₂ sensor protein- or a carbonic anhydrase- expressing nucleic acid or a gene or a message, or any nucleic acid inhibitory to the expression of the CO₂ sensor protein or the carbonic anhydrase, and expressing the inhibitory nucleic acid, the antisense or the iRNA in the guard cell, plant, plant leaf, plant organ or plant part;
(2) decreasing of expression and/or activity of a homologous CO₂ sensor protein-expressing nucleic acid or a gene or a message or a homologous carbonic anhydrase-expressing nucleic acid (e.g., a gene or message); or,
(3) a combination of (1) and (2); or
(d) the method of (c), wherein the carbonic anhydrase is a β-carbonic anhydrase;
thereby:
up-regulating or increasing carbon dioxide (CO₂) and/or water exchange in the guard cell, plant, plant leaf, plant organ or plant part;
decreasing the water use efficiency of the guard cell, plant, plant leaf, plant organ or plant part;
increasing the rate of growth or biomass production in the plant, plant leaf, plant organ or plant part; or decreasing or desensitizing the carbon dioxide (CO₂) sensitivity of the plant, plant leaf, plant organ or plant part; or
up-regulating or increasing carbon dioxide (CO₂) and/or water exchange in the guard cell of the plant, plant leaf, plant organ or plant part; or
(2) the method of (1), wherein the polypeptide having carbonic anhydrase activity comprises an amino acid sequence having between about 75% to 100% sequence identity with a amino acid sequence comprising SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46; or
(3) the method of (1), wherein the polypeptide having carbonic anhydrase activity is encoded by a nucleotide sequence comprising SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45; or
(4) the method of (1), wherein the polypeptide having OST1 protein kinase activity comprises an amino acid sequence having between about 75% to 100% sequence identity with an amino acid sequence comprising SEQ ID NO:12 or SEQ ID NO:14; or
(5) the method of (1), wherein wherein the polypeptide having OST1 protein kinase activity is encoded by a nucleotide sequence comprising SEQ ID NO:11 or SEQ ID NO:13; or
(6) the method of (1), wherein the plant is characterized by controlled CO₂ exchange under ambient 365 ppm CO₂, elevated ppm CO₂ or reduced ppm CO₂, or the plant is characterized by controlled water exchange under ambient 365 ppm CO₂, elevated ppm CO₂ or reduced ppm CO₂; or
(7) the method of (1), wherein the CO₂ sensor protein-expressing nucleic acid or gene, carbonic anhydrase-expressing nucleic acid, message or gene, and/or the protein kinase-expressing nucleic acid, message or gene, is operably linked to a plant expressible promoter, an inducible promoter, a constitutive promoter, a guard cell specific promoter, a drought-inducible promoter, a stress-inducible promoter or a guard cell active promoter; or
(8) the method of (1), wherein the polypeptide having carbonic anhydrase activity comprises an amino acid sequence having between about 75% to 100% sequence identity with an amino acid sequence comprising SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46.

3. The method of claim 1, wherein:
(a) the polypeptide having carbonic anhydrase activity comprises an amino acid sequence having between about 75% to 100% sequence identity with an amino acid sequence comprising SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46; or
(b) the polypeptide having carbonic anhydrase activity is encoded by a nucleotide sequence comprising SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45.

4. (canceled)

5. The method of claim 1, wherein:
(a) the polypeptide having OST1 protein kinase activity comprises an amino acid sequence having between about 75% to 100% sequence identity with an amino acid sequence comprising SEQ ID NO:12 or SEQ ID NO:14;
(b) the polypeptide having OST1 protein kinase activity is encoded by a nucleotide sequence comprising SEQ ID NO:11 or SEQ ID NO:13;
(c) the plant is characterized by controlled CO₂ exchange under ambient 365 ppm CO₂, elevated ppm CO₂ or reduced ppm CO₂, or the plant is characterized by controlled water exchange under ambient 365 ppm CO₂, elevated ppm CO₂ or reduced ppm CO₂; or
(d) the CO₂ sensor protein-expressing nucleic acid or gene, carbonic anhydrase-expressing nucleic acid, message or gene, and/or the protein kinase-expressing nucleic acid, message or gene, is operably linked to a plant expressible promoter, an inducible promoter, a constitutive promoter, a guard cell specific promoter, a drought-inducible promoter, a stress-inducible promoter or a guard cell active promoter.

6-8. (canceled)

9. The method of claim 2, wherein the:
up-regulating or increasing carbon dioxide (CO₂) and/or water exchange in a guard cell of a plant, plant cell, plant leaf, plant organ or plant part;
decreasing the water use efficiency of a guard cell, a plant, plant leaf, plant organ or plant part; or
decreasing or desensitizing the carbon dioxide (CO₂) sensitivity of a plant, plant leaf, plant organ or plant part; or
upregulating or increasing carbon dioxide (CO₂) and/or water exchange in a guard cell of a plant, plant leaf, plant organ or plant part; comprises:
(a) providing:
(i) a nucleic acid inhibitory to the expression of a CO₂ sensor protein-expressing nucleic acid or a CO₂ sensor gene or transcript (mRNA), each encoding a polypeptide having a carbonic anhydrase (CA) activity or a β-carbonic anhydrase activity; and/or
(ii) a nucleic acid inhibitory or antisense to the expression of an OST1, SnRK2.2- or SnRK2.3 protein kinase-expressing nucleic acid or an OST1, SnRK2.2- or SnRK2.3 protein kinase gene or transcript; and
(b) expressing the nucleic acid inhibitory to the expression of the CO₂ sensor protein-expressing nucleic acid, gene or transcript or expressing an antisense, iRNA or inhibitory nucleic acid in a guard cell; and/or, expressing a nucleic acid inhibitory to the expression of the protein kinase-expressing nucleic acid, gene or transcript,
thereby up-regulating or increasing carbon dioxide (CO₂) and/or water exchange in a guard cell; decreasing the water use efficiency of a guard cell, a plant, plant leaf, plant organ or plant part; or decreasing or desensitizing the carbon dioxide (CO₂) sensitivity of a plant, plant leaf, plant organ or plant part; or upregulating or increasing carbon dioxide (CO₂) and/or water exchange in a guard cell of a plant, plant leaf, plant organ or plant part.

10. The method of claim 2, wherein the nucleic acid inhibitory to the expression of a CO₂ sensor protein-expressing nucleic acid comprises:
(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence encoding a polypeptide having carbonic anhydrase activity,
the polypeptide optionally comprising an amino acid sequence having between about 75% and 100% sequence identity with an amino acid sequence of: SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ or ID. No.46, or
(b) a partial or complete complementary sequence of the nucleotide sequence of (a).

11. The method of claim 2, wherein the nucleic acid inhibitory to the expression of a CO₂ sensor protein-expressing nucleic acid comprises:
(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence of SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45; or
(b) a partial or complete complementary sequence of the nucleotide sequence (a).

12. The method of claim 2, wherein the nucleic acid inhibitory to the expression of the polypeptide having OST1 protein kinase activity comprises:
(a) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence encoding an amino acid sequence having between 75% and 100% sequence identity with amino acid sequence of SEQ ID NO:12 or SEQ ID NO:14; or
(b) a partial or complete complementary sequence of the nucleotide sequence (a); or
(c) a nucleotide sequence of at least about 11, 12, 13, 14, 15, 16, 17, 18, or 19 or more nucleotides having at least about 90%, 91%, 92%, 93%, 94%, 95%, 96%, 97%, 98% or 99% or more sequence identity with a nucleotide sequence of SEQ ID No.11 or SEQ ID NO:13; or
(d) a partial or complete complementary sequence of the nucleotide sequence (c).

13. (canceled)

14. The method of claim 2, wherein:
(a) the nucleic acid inhibitory to the expression of a CO₂ sensor protein-expressing nucleic acid comprises the nucleotide sequence of at least about 11, 12, 13, 14, 15, 16, 17, 18, or 19 or more nucleotides and a complementary sequence to the nucleotide sequence of at least about 11, 12, 13, 14, 15, 16, 17, 18, or 19 or more nucleotides;
(b) the nucleotide sequence comprising the at least about 11, 12, 13, 14, 15, 16, 17, 18, or 19 or more nucleotides is a nucleotide sequence comprising at least 50 or 100 or 300 nucleotides having between 75 to 100% sequence identity to the nucleotide sequence encoding a polypeptide having carbonic anhydrase activity and/or nucleotide sequence encoding a polypeptide having OST1 protein kinase activity;
(c) the plant is characterized by controlled CO₂ exchange under ambient 365 ppm CO₂, elevated ppm CO₂ or reduced ppm CO₂, or the plant is characterized by controlled water exchange under ambient 365 ppm CO₂, elevated ppm CO₂ or reduced ppm CO₂; or
(d) the CO₂ sensor protein-inhibitory nucleic acid and/or the OST1 protein kinase-inhibitory nucleic acid is operably linked to a plant expressible promoter, an inducible promoter, a constitutive promoter, a guard cell specific promoter, a drought-inducible promoter, a stress-inducible promoter or a guard cell active promoter.

15-17. (canceled)

18. A method for regulating water exchange in a cell of a plant, plant cell, plant leaf, plant organ or plant part comprising:
(1) (a) expressing or increasing the expression of a CO₂ sensor protein-encoding or a carbonic anhydrase-encoding gene or transcript, and an OST1, SnRK2.2- or SnRK2.3 protein kinase-encoding gene or transcript, by providing and expressing a CO₂ sensor protein expressing and an OST1, SnRK2.2- or SnRK2.3 protein kinase nucleic acid, gene or transcript, in the plant, guard cell, plant cell, plant leaf, plant organ or plant part; or
(b) decreasing the expression of a CO₂ sensor protein encoding gene or transcript or a carbonic anhydrase gene or transcript and an OST1, SnRK2.2- or SnRK2.3 protein kinase-encoding gene or transcript in the plant, guard cell, plant cell, plant leaf, plant organ or plant part, by expressing a nucleic acid inhibitory to the expression of the CO₂ sensor protein-expressing or carbonic anhydrase-expressing nucleic acid, gene or transcript and the OST1, SnRK2.2- or SnRK2.3 protein kinase-expressing nucleic acid, gene or transcript, in the plant, guard cell, plant cell, plant leaf, plant organ, or plant part;
thereby regulating water exchange, wherein down-regulating or decreasing water exchange is achieved by expression or increased expression of the carbonic anhydrase or CO₂ sensor protein and the protein kinase and wherein up-regulating or increasing water exchange is achieved by reduction of expression of the carbonic anhydrase or CO₂ sensor protein and the protein kinase in the plant, guard cell, plant cell, plant leaf, plant organ or plant part; or
(2) the method of (1), wherein the increasing or decreasing of the expression is in the plant guard cell.

19-31. (canceled)

32. The method of claim 1, wherein the plant is, or the guard cell, plant cell, plant part or plant organ, is isolated and/or derived from: (i) a dicotyledonous or monocotyledonous plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solarium, Sorghum, Theobromus, Trigonella, Triticum, Vicia, Vitis, Vigna or Zea.

33. A transgenic guard cell, plant, plant cell, plant tissue, plant seed or fruit, plant part or plant organ, comprising:
(a) (1) a heterologous OST1 protein kinase-expressing nucleic acid or an OST1 protein kinase gene or mRNA (message) encoding a polypeptide with OST1 protein kinase activity; or
(2) a heterologous protein kinase SnRK2.2- or SnRK2.3-expressing nucleic acid or an SnRK2.2- or SnRK2.3 protein kinase gene or mRNA (message) encoding a polypeptide with SnRK2.2- or SnRK2.3 protein kinase activity;
(b) the transgenic plant cell, plant, plant part or plant organ of (a), further comprising a heterologous nucleic acid, gene or transcript encoding a protein having a carbonic anhydrase (CA) activity or a β-carbonic anhydrase activity, or encoding a CO₂ sensor protein,
wherein optionally the nucleic acid, gene or transcript is operably linked to a plant expressible promoter, an inducible promoter, a constitutive promoter, a guard cell specific promoter, a drought-inducible promoter, a stress-inducible promoter or a guard cell active promoter;
and optionally the nucleic acid, gene or transcript is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ;
(c)
(1) (1) a heterologous nucleic acid that is inhibitory to an OST1 protein kinase-expressing nucleic acid or an OST1 protein kinase gene or mRNA (message) encoding a polypeptide with OST1 protein kinase activity, or is inhibitory to the activity or the kinase; or
(2) a heterologous nucleic acid that is inhibitory to a protein kinase SnRK2.2- or SnRK2.3-expressing nucleic acid or an SnRK2.2- or SnRK2.3 protein kinase gene or mRNA (message) encoding a polypeptide with SnRK2.2- or SnRK2.3 protein kinase activity, or is inhibitory to the activity or the kinase; or
(b) the transgenic plant cell, plant, plant part or plant organ of (i), further comprising a heterologous nucleic acid that is inhibitory to a gene or transcript encoding a protein having a carbonic anhydrase (CA) activity or a β-carbonic anhydrase activity, or is inhibitory to a gene or transcript encoding a CO₂ sensor protein,
wherein optionally the inhibitory nucleic acid is operably linked to a plant expressible promoter, an inducible promoter, a constitutive promoter, a guard cell specific promoter, a drought-inducible promoter, a stress-inducible promoter or a guard cell active promoter;
and optionally the inhibitory nucleic acid is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ,
and optionally the inhibitory nucleic acid comprises an antisense RNA or an iRNA:
(d) a first and second recombinant gene, wherein the first recombinant gene comprises an expression-increasing recombinant first gene or an expression-inhibiting first recombinant gene, and wherein the second recombinant gene comprises an expression-increasing second recombinant gene or an expression-inhibiting second recombinant gene;
wherein the expression increasing first recombinant gene comprises:
i. a plant, plant cell or guard cell expressible promoter; and
ii. a heterologous nucleic acid encoding: a polypeptide having a carbonic anhydrase (CA) activity or a β-carbonic anhydrase activity, or, a CO₂ sensor protein; and
optionally further comprising a transcription termination and polyadenylation signal;
wherein the expression-inhibiting first recombinant gene comprises the following operably linked DNA fragments:
i. a plant, plant cell or guard cell expressible promoter; and
ii. a heterologous nucleic acid, which when transcribed produces a nucleic acid or a ribonucleic acid inhibitory to the expression of a CO₂ sensor protein-expressing nucleic acid or a CO₂ sensor gene or transcript, each optionally encoding a polypeptide having a carbonic anhydrase (CA) activity or a β-carbonic anhydrase activity,
optionally further comprising a transcription termination and polyadenylation signal;
wherein the expression-increasing second recombinant gene comprises:
i. a plant, plant cell or guard cell expressible promoter; and
ii. a heterologous nucleic acid encoding a polypeptide with OST1, SnRK2.2- or SnRK2.3 protein kinase activity;
optionally further comprising a transcription termination and polyadenylation signal;
wherein the expression inhibiting second recombinant gene:
i. a plant, plant cell or guard cell expressible promoter; and
ii. a heterologous nucleic acid, which when transcribed produces a nucleic acid (e.g., a ribonucleic acid) inhibitory to the expression of OST1, SnRK2.2- or SnRK2.3 protein kinase encoding gene;
optionally further comprising a transcription termination and polyadenylation signal;
(e) the guard cell, plant, plant cell, plant tissue, plant seed or fruit, plant part or plant organ of any of (a) to (d), wherein a nucleic acid or a DNA fragment encoding a polypeptide having a carbonic anhydrase (CA) activity or a β-carbonic anhydrase activity encodes a polypeptide comprising an amino acid sequence having between 75% and 100% sequence identity with a amino acid sequence of SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46;
(f) the guard cell, plant, plant cell, plant tissue, plant seed or fruit, plant part or plant organ of any of (a) to (e), wherein the polypeptide having carbonic anhydrase activity is encoded by a nucleotide sequence comprising SEQ ID NO:1, SEQ ID NO:2, SEQ ID NO:4, SEQ ID NO:5. SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45;
(g) the guard cell, plant, plant cell, plant tissue, plant seed or fruit, plant part or plant organ of any of (a) to (f), wherein the nucleic acid or the DNA fragment encoding the polypeptide with OST1, SnRK2.2- or SnRK2.3 protein kinase activity encodes a polypeptide comprising an amino acid sequence having between 75% and 100% sequence identity with an amino acid sequence comprising SEQ ID NO:12 or SEQ ID NO:14;
(h) the guard cell, plant, plant cell, plant tissue, plant seed or fruit, plant part or plant organ of any of (a) to (g), wherein the polypeptide having OST1 protein kinase activity is encoded by a nucleotide sequence selected from the nucleotide sequence comprising SEQ ID NO:11 or SEQ ID NO:13;
(i) the guard cell, plant, plant cell, plant tissue, plant seed or fruit, plant part or plant organ of any of (a) to (h), wherein the nucleic acid or the DNA fragment, which when transcribed yields an inhibitory nucleic acid or an inhibitory ribonucleic acid to the expression of a CO₂ sensor protein-expressing nucleic acid comprises a nucleotide sequence of at least 11, 12, 13, 14, 15, 16, 17, 18, or 19 or more nucleotides having at least 90%, 91%, 92%, 93%, 94%, 95%, 96%, 97%, 98% or 99% or more sequence identity with a nucleotide sequence encoding a polypeptide having carbonic anhydrase activity comprising an amino acid sequence having between 75% and 100% sequence identity with a amino acid sequence selected from the amino acid sequence comprising SEQ ID NO:1, SEQ ID NO:3, SEQ ID NO:6, SEQ ID NO:8, SEQ ID NO:10, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, or SEQ ID NO:46, or a complete or partial complement thereof;
(j) the guard cell, plant, plant cell, plant tissue, plant seed or fruit, plant part or plant organ of any of (a) to (i), wherein a nucleic acid or a DNA fragment, which when transcribed yield a ribonucleic acid inhibitory to the expression of a CO₂ sensor protein-expressing nucleic acid comprises a nucleotide sequence of at least 11, 12, 13, 14, 15, 16, 17, 18, or 19 or more nucleotides having at least 94% sequence identity with a nucleotide sequence selected from the nucleotide sequence comprising SEQ ID NO:1, SEQ ID NO:2, SEQ ID NO:4, SEQ ID NO:5, SEQ ID NO:7, SEQ ID NO:9, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45, or a complete or partial complement thereof;
(k) the guard cell, plant, plant cell, plant tissue, plant seed or fruit, plant part or plant organ of any of (a) to (j), wherein the ribonucleic acid inhibitory to the expression of a CO₂ sensor protein-expressing nucleic acid comprises the nucleotide sequence of at least 11, 12, 13, 14, 15, 16, 17, 18, or 19 or more nucleotides and a complementary sequence to the nucleotide sequence of at least 11, 12, 13, 14, 15, 16, 17, 18, or 19 or more nucleotides;
(l) the guard cell, plant, plant cell, plant tissue, plant seed or fruit, plant part or plant organ of any of (a) to (k), wherein a nucleic acid or a DNA fragment, which when transcribed yield a ribonucleic acid inhibitory to the expression of a OST1 kinase protein-expressing nucleic acid comprises a nucleotide sequence of at least 11, 12, 13, 14, 15, 16, 17, 18, or 19 or more nucleotides having at least 90%, 91%, 92%, 93%, 94%, 95%, 96%, 97%, 98% or 99% or more sequence identity with a nucleotide sequence encoding a polypeptide having OST1 protein kinase activity comprising an amino acid sequence having between 75% and 100% sequence identity with a amino acid sequence selected from the amino acid sequence of comprising SEQ ID NO:12 or SEQ ID NO:14, or a complete or partial complement thereof;
(m) the guard cell, plant, plant cell, plant tissue, plant seed or fruit, plant part or plant organ of any of (a) to (l), wherein a nucleic acid or a DNA fragment, which when transcribed yield a ribonucleic acid inhibitory to the expression of a OST1 protein kinase encoding nucleic acid comprises a nucleotide sequence of at least 11, 12, 13, 14, 15, 16, 17, 18, or 19 or more nucleotides having at least 90%, 91%, 92%, 93%, 94%, 95%, 96%, 97%, 98% or 99% or more sequence identity with a nucleotide sequence selected from the nucleotide sequence comprising SEQ ID NO:11 or SEQ ID NO:13, or a complete or partial complement thereof;
(n) the guard cell, plant, plant cell, plant tissue, plant seed or fruit, plant part or plant organ of any of (a) to (m), wherein the ribonucleic acid inhibitory to the expression of a CO₂ sensor protein-expressing nucleic acid comprises the nucleotide sequence of at least 19 nucleotides and a complementary sequence to the nucleotide sequence of at least 19 nucleotides;
(o) the guard cell, plant, plant cell, plant tissue, plant seed or fruit, plant part or plant organ of any of (a) to (n), wherein the first recombinant gene is an expression increasing first recombinant gene, and the second recombinant gene is an expression increasing second recombinant gene;
(p) the guard cell, plant, plant cell, plant tissue, plant seed or fruit, plant part or plant organ of any of (a) to (o), wherein the first recombinant gene is an expression inhibiting first recombinant gene, and the second recombinant gene is an expression inhibiting second recombinant gene;
(q) the guard cell, plant, plant cell, plant tissue, plant seed or fruit, plant part or plant organ of any of (a) to (p), wherein the first recombinant gene is an expression increasing first recombinant gene, and the second recombinant gene is an expression inhibiting second recombinant gene;
(r) the guard cell, plant, plant cell, plant tissue, plant seed or fruit, plant part or plant organ of any of (a) to (g), wherein the first recombinant gene is an expression inhibiting first recombinant gene, and the second recombinant gene is an expression increasing second recombinant gene; or
(s) the guard cell, plant, plant cell, plant tissue, plant seed or fruit, plant part or plant organ of any of (a) to (r), wherein the plant is or the guard cell, plant, plant cell, plant tissue, plant seed or fruit, plant part or plant organ is isolated and/or derived from: (i) a dicotyledonous or monocotyledonous plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solarium, Sorghum, Theobromus, Trigonella, Triticum, Vicia, Vitis, Vigna or Zea.

34-54. (canceled)


SEQUENCE LISTING (46 sequences)

SEQ ID NO: 1 - DNA, 1336 bp, Arabidopsis thaliana
cgaacggtcgtcataattccttgaaacctcgaaaatccaaaaacccatatccaatcttcttcccatataaattaagatttttatttatttatttgtttacttatttcaattcccaaaatcctctgcctcatcatcttcaaactgttaccacgtccatagggttgtcgaagagctaggaagagccttaccaagagcttcttcttcccctaacatttaggttggtaggagaagcaaaggaagagatcatttataatggctcctgcattcggaaaatgtttcatgttctgctgcgctaaaacctccccggaaaaagacgaaatggcaacggaatcgtacgaagccgccattaaaggactcaatgatcttctcagtacgaaagcggatctcggaaacgtcgccgccgcgaagatcaaagcgttgacggcggagctaaaggagcttgactcaagcaattcagacgcaattgaacgaatcaagaccggttttactcaattcaaaaccgagaaatatttgaagaatagtactttgttcaatcatcttgccaagactcagaccccaaagtttctggtgtttgcttgctctgattctcgagtttgtccatctcacatcttgaatttccaacctggtgaggcttttgttgtcagaaacatagccaatatggttccaccttttgaccagaagagacactctggagttggcgccgccgttgaatacgcagttgtacatctcaaggtggagaacattttggtgataggccatagctgctgtggtggtattaagggactcatgtccattgaagatgatgctgccccaactcaaagtgacttcattgaaaattgggtgaagataggcgcatcagcgaggaacaagatcaaggaggaacataaagacttgagctacgatgatcaatgcaacaagtgtgagaaggaagctgtgaacgtatcgcttggaaacttgctttcgtacccattcgtgagagctgaggtggtgaagaacacacttgcaataagaggaggtcactacaatttcgtcaaaggaacgtttgatctctgggagctcgatttcaagaccactcctgcttttgccttctcttaagaaagaaagctaccggaacatataaaactcttttgagataaaaaaagacactttgactcatctttcttcattctctcatgttgatgattcctctccaacttctttgatttctttttgttaattcaaaacttcaactttgctgcttctatttcaaaagctcaaacaataaagctgtaaccaacgtttgaaacttctatatttgtctaattgatgtttgaacgaagatttgaactttccttct

SEQ ID NO: 2 - DNA, 843 bp, Arabidopsis thaliana
  CDS (1)..(843)
atggctcctgcattcggaaaatgtttcatgttctgctgcgctaaaacctccccggaaaaagacgaaatggcaacggaatcgtacgaagccgccattaaaggactcaatgatcttctcagtacgaaagcggatctcggaaacgtcgccgccgcgaagatcaaagcgttgacggcggagctaaaggagcttgactcaagcaattcagacgcaattgaacgaatcaagaccggttttactcaattcaaaaccgagaaatatttgaagaatagtactttgttcaatcatcttgccaagactcagaccccaaagtttctggtgtttgcttgctctgattctcgagtttgtccatctcacatcttgaatttccaacctggtgaggcttttgttgtcagaaacatagccaatatggttccaccttttgaccagaagagacactctggagttggcgccgccgttgaatacgcagttgtacatctcaaggtggagaacattttggtgataggccatagctgctgtggtggtattaagggactcatgtccattgaagatgatgctgccccaactcaaagtgacttcattgaaaattgggtgaagataggcgcatcagcgaggaacaagatcaaggaggaacataaagacttgagctacgatgatcaatgcaacaagtgtgagaaggaagctgtgaacgtatcgcttggaaacttgctttcgtacccattcgtgagagctgaggtggtgaagaacacacttgcaataagaggaggtcactacaatttcgtcaaaggaacgtttgatctctgggagctcgatttcaagaccactcctgcttttgccttctcttaa

SEQ ID NO: 3 - protein, 280 aa, Arabidopsis thaliana
MAPAFGKCFMFCCAKTSPEKDEMATESYEAAIKGLNDLLSTKADLGNVAAAKIKALTAELKELDSSNSDAIERIKTGFTQFKTEKYLKNSTLFNHLAKTQTPKFLVFACSDSRVCPSHILNFQPGEAFVVRNIANMVPPFDQKRHSGVGAAVEYAVVHLKVENILVIGHSCCGGIKGLMSIEDDAAPTQSDFIENWVKIGASARNKIKEEHKDLSYDDQCNKCEKEAVNVSLGNLLSYPFVRAEVVKNTLAIRGGHYNFVKGTFDLWELDFKTTPAFAFS

SEQ ID NO: 4 - DNA, 1151 bp, Arabidopsis thaliana
caaaattcatgtgttagttcttcttctttacaaaattgagtttaaactgttttattactaatccaaatgaggaatcactttgcactattaatagaaaataatacacaaccaaacatctaaaagatactataatagtagagatcaaagacctgagcaaaaactgaaagaaaaaaaaaaaaaaaaaaaaagacttctcctcaaaaatggcgtttacactaggtggaagagctcgtcgtctagtctctgcaacatcagttcatcaaaatggttgcttacacaaactgcaacaaattggatcggatcggtttcagcttggtgaagcaaaagcaataagattactacccaggagaacaaacatggttcaagaattaggaatcagggaagaatttatggatctaaacagagaaacagagacaagttatgattttctggatgaaatgagacacagatttctgaaattcaagagacaaaagtatctaccggagatagaaaagtttaaagctttggccatagctcaatcaccaaaggtaatggtgataggatgtgcagattcaagggtatgtccatcttatgtactaggatttcaacctggtgaagcttttactatccgaaatgtcgccaatctcgttaccccggttcagaatggaccaacagaaaccaactcggctcttgagtttgcggtcaccactcttcaggttgagaacattatagttatgggtcatagcaattgtggaggaattgcagcacttatgagtcatcaaaaccaccaagggcaacactctagtttagtagaaaggtgggttatgaatgggaaagccgctaagttaagaacacaattagcttcatcacatttatcctttgatgaacaatgcagaaactgtgagaaggaatctataaaggattctgtgatgaatttgataacttattcatggataagagatagagtaaagagaggtgaagtcaagattcatggatgttattacaatttgtcagattgtagtcttgagaagtggagattaagttcagacaagactaactatggattctatatttcagacagagagatatggagttgagtaaatattgaacaatcctcagttctaatattcagatgtatctttgtacatacgaaatgatatttacacaattgg

SEQ ID NO: 5 - DNA, 873 bp, Arabidopsis thaliana
  CDS (1)..(873)
atggcgtttacactaggtggaagagctcgtcgtctagtctctgcaacatcagttcatcaaaatggttgcttacacaaactgcaacaaattggatcggatcggtttcagcttggtgaagcaaaagcaataagattactacccaggagaacaaacatggttcaagaattaggaatcagggaagaatttatggatctaaacagagaaacagagacaagttatgattttctggatgaaatgagacacagatttctgaaattcaagagacaaaagtatctaccggagatagaaaagtttaaagctttggccatagctcaatcaccaaaggtaatggtgataggatgtgcagattcaagggtatgtccatcttatgtactaggatttcaacctggtgaagcttttactatccgaaatgtcgccaatctcgttaccccggttcagaatggaccaacagaaaccaactcggctcttgagtttgcggtcaccactcttcaggttgagaacattatagttatgggtcatagcaattgtggaggaattgcagcacttatgagtcatcaaaaccaccaagggcaacactctagtttagtagaaaggtgggttatgaatgggaaagccgctaagttaagaacacaattagcttcatcacatttatcctttgatgaacaatgcagaaactgtgagaaggaatctataaaggattctgtgatgaatttgataacttattcatggataagagatagagtaaagagaggtgaagtcaagattcatggatgttattacaatttgtcagattgtagtcttgagaagtggagattaagttcagacaagactaactatggattctatatttcagacagagagatatggagttga

SEQ ID NO: 6 - protein, 290 aa, Arabidopsis thaliana
MAFTLGGRARRLVSATSVHQNGCLHKLQQIGSDRFQLGEAKAIRLLPRRTNMVQELGIREEFMDLNRETETSYDFLDEMRHRFLKFKRQKYLPEIEKFKALAIAQSPKVMVIGCADSRVCPSYVLGFQPGEAFTIRNVANLVTPVQNGPTETNSALEFAVTTLQVENIIVMGHSNCGGIAALMSHQNHQGQHSSLVERWVMNGKAAKLRTQLASSHLSFDEQCRNCEKESIKDSVMNLITYSWIRDRVKRGEVKIHGCYYNLSDCSLEKWRLSSDKTNYGFYISDREIWS

SEQ ID NO: 7 - DNA, 1319 bp, Arabidopsis thaliana
atgagactccgttcttttaaactcccaaatctttcaaccaatcccattattcacttaagtatatagtagcttccataagagtcttagttctaactataaatacacatatctcactctctctgatctccgcttctcttcgccaacaaatgtcgaccgctcctctctccggcttctttctcacttcactttctccttctcaatcttctctccagaaactctctcttcgtacttcttccaccgtcgcttgcctcccacccgcctcttcttcttcctcatcttcctcctcctcgtcttcccgttccgttccaacgcttatccgtaacgagccagtttttgccgctcctgctcctatcattgccccttattggagtgaagagatgggaaccgaagcatacgacgaggctattgaagctctcaagaagcttctcatcgagaaggaagagctaaagacggttgcagcggcaaaggtggagcagatcacagcggctcttcagacaggtacttcatccgacaagaaagctttcgaccccgtcgaaaccattaagcagggcttcatcaaattcaagaaggagaaatacgaaaccaaccctgctttgtacggtgagctcgcaaagggtcaaagtcctaagtacatggtgtttgcttgttcagactcacgtgtgtgtccatcacacgttctggactttcagccaggagatgccttcgtggtccgtaacatagccaacatggttcctcctttcgacaaggtcaaatacggtggcgttggagcagccattgaatacgcggtcttacaccttaaggtggagaacattgtggtgataggacacagtgcatgtggtgggatcaaagggcttatgtctttccccttagatggaaacaactccactgacttcatagaggactgggtcaaaatctgtttaccagccaagtcaaaggttatatcagaacttggagattcagcctttgaagatcaatgtggccgatgtgaaagggaggcggtgaatgtttcactagcaaacctattgacatatccatttgtgagagaaggacttgtgaagggaacacttgctttgaagggaggctactatgacttcgtcaagggtgcttttgagctttggggacttgaatttggcctctccgaaactagctctgttaaagatgtggctaccatactacattggaagctgtaggaaactctttgaagccttacccgatttcacattgtcaattcaataacaccaagttgttgtttacatgcagatcttgatgaaactggtttttgattttacagaattaaaatcttgggggacagaaatttg

SEQ ID NO: 8 - protein, 347 aa, Arabidopsis thaliana
MSTAPLSGFFLTSLSPSQSSLQKLSLRTSSTVACLPPASSSSSSSSSSSSRSVPTLIRNEPVFAAPAPIIAPYWSEEMGTEAYDEAIEALKKLLIEKEELKTVAAAKVEQITAALQTGTSSDKKAFDPVETIKQGFIKFKKEKYETNPALYGELAKGQSPKYMVFACSDSRVCPSHVLDFQPGDAFVVRNIANMVPPFDKVKYGGVGAAIEYAVLHLKVENIVVIGHSACGGIKGLMSFPLDGNNSTDFIEDWVKICLPAKSKVISELGDSAFEDQCGRCEREAVNVSLANLLTYPFVREGLVKGTLALKGGYYDFVKGAFELWGLEFGLSETSSVKDVATILHWKL

SEQ ID NO: 9 - DNA, 1444 bp, Arabidopsis thaliana
atgagactccgttcttttaaactcccaaatctttcaaccaatcccattattcacttaagtatatagtagcttccataagagtcttagttctaactataaatacacatatctcactctctctgatctccgcttctcttcgccaacaaatgtcgaccgctcctctctccggcttctttctcacttcactttctccttctcaatcttctctccagaaactctctcttcgtacttcttccaccgtcgcttgcctcccacccgcctcttcttcttcctcatcttcctcctcctcgtcttcccgttccgttccaacgcttatccgtaacgagccagtttttgccgctcctgctcctatcattgccccttattggagtgaagagatgggaaccgaagcatacgacgaggctattgaagctctcaagaagcttctcatcgagaaggaagagctaaagacggttgcagcggcaaaggtggagcagatcacagcggctcttcagacaggtacttcatccgacaagaaagctttcgaccccgtcgaaaccattaagcagggcttcatcaaattcaagaaggagaaatacgaaaccaaccctgctttgtacggtgagctcgcaaagggtcaaagtcctaagtacatggtgtttgcttgttcagactcacgtgtgtgtccatcacacgttctggactttcagccaggagatgccttcgtggtccgtaacatagccaacatggttcctcctttcgacaaggtcaaatacggtggcgttggagcagccattgaatacgcggtcttacaccttaaggtggagaacattgtggtgataggacacagtgcatgtggtgggatcaaagggcttatgtctttccccttagatggaaacaactccactgacttcatagaggactgggtcaaaatctgtttaccagccaagtcaaaggttatatcagaacttggagattcagcctttgaagatcaatgtggccgatgtgaaagggaggcggtgaatgtttcactagcaaacctattgacatatccatttgtgagagaaggacttgtgaagggaacacttgctttgaagggaggctactatgacttcgtcaagggtgcttttgagctttggggacttgaatttggcctctccgaaactagctctgtatgaaccaatccatcatcatcatcatcatcatgaccatccatcatcatcatcattattatcatcgtatataatatatatctaccccatatgtaatttgtaatgtgcctttgactgtgatgagttatctctccctctctaccaactttcttcatatatataaaacaaaaaggaaaagcagatgatatagatctttcgtggtttaattatgaacaattgtctttattatttgtgtatcaaatcggttgtatttatggtttgattttattttctatgttgtttggtaggttaaa

SEQ ID NO: 10 - protein, 336 aa, Arabidopsis thaliana
MSTAPLSGFFLTSLSPSQSSLQKLSLRTSSTVACLPPASSSSSSSSSSSSRSVPTLIRNEPVFAAPAPIIAPYWSEEMGTEAYDEAIEALKKLLIEKEELKTVAAAKVEQITAALQTGTSSDKKAFDPVETIKQGFIKFKKEKYETNPALYGELAKGQSPKYMVFACSDSRVCPSHVLDFQPGDAFVVRNIANMVPPFDKVKYGGVGAAIEYAVLHLKVENIVVIGHSACGGIKGLMSFPLDGNNSTDFIEDWVKICLPAKSKVISELGDSAFEDQCGRCEREAVNVSLANLLTYPFVREGLVKGTLALKGGYYDFVKGAFELWGLEFGLSETSSV

SEQ ID NO: 11 - DNA, 1353 bp, Arabidopsis thaliana
  CDS (112)..(1200)
ttgttcatttcctctgatgtcttggtgtcgttagatattgtctcccaaaaaagaaatcttcttgacacagagattgaagtcgcaaagagacagaggaaagagggggagaaaatggatcgaccagcagtgagtggtccaatggatttgccgattatgcacgatagtgataggtatgaactcgtcaaggatattggctccggtaattttggagttgcgagattgatgagagacaagcaaagtaatgagcttgttgctgttaaatatatcgagagaggtgagaagatagatgaaaatgtaaaaagggagataatcaaccacaggtccttaagacatcccaatatcgttagattcaaagaggttatattaacaccaacccatttagccattgttatggaatatgcatctggaggagaacttttcgagcgaatctgcaatgcaggccgcttcagcgaagacgaggcgaggtttttcttccagcaactcatttcaggagttagttactgtcatgctatgcaagtatgtcaccgagacttaaagctcgagaatacgttattagatggtagcccggcccctcgtctaaagatatgtgatttcggatattctaagtcatcagtgttacattcgcaaccaaaatcaactgttggaactcctgcttacatcgctcctgaggttttactaaagaaagaatatgatggaaaggttgcagatgtttggtcttgtggggttactctgtatgtcatgctggttggagcatatcctttcgaagatcccgaggaaccaaagaatttcaggaaaactatacatagaatcctgaatgttcagtatgctattccggattatgttcacatatctcctgaatgtcgccatttgatctccagaatatttgttgctgaccctgcaaagaggatatcaattcctgaaataaggaaccatgaatggtttctaaagaatctaccggcagatctaatgaacgataacacgatgaccactcagtttgatgaatcggatcaaccgggccaaagcatagaagaaattatgcagatcattgcagaagcaactgttcctcctgcaggcactcagaatctgaaccattacctcacaggaagcttggacatagatgacgatatggaggaagacttagagagcgaccttgatgatcttgacatcgacagtagcggagagattgtgtacgcaatgtgatactatatatctatttgcatggtttctgctacaaaaatgtcaaacaaaaaatgttgaagaataagattaagatgttttgcttgctattgagttggcccaactttgtctcaatgagtacactttgaatctttgatatgcaaaagactaaatttc

SEQ ID NO: 12 - protein, 362 aa, Arabidopsis thaliana
MDRPAVSGPMDLPIMHDSDRYELVKDIGSGNFGVARLMRDKQSNELVAVKYIERGEKIDENVKREIINHRSLRHPNIVRFKEVILTPTHLAIVMEYASGGELFERICNAGRFSEDEARFFFQQLISGVSYCHAMQVCHRDLKLENTLLDGSPAPRLKICDFGYSKSSVLHSQPKSTVGTPAYIAPEVLLKKEYDGKVADVWSCGVTLYVMLVGAYPFEDPEEPKNFRKTIHRILNVQYAIPDYVHISPECRHLISRIFVADPAKRISIPEIRNHEWFLKNLPADLMNDNTMTTQFDESDQPGQSIEEIMQIIAEATVPPAGTQNLNHYLTGSLDIDDDMEEDLESDLDDLDIDSSGEIVYAM

SEQ ID NO: 13 - DNA, 1702 bp, Arabidopsis thaliana
  CDS (605)..(1549)
agagaaagctgtttcctttttatattgacagagaaaaggaaagctgatagagagagagacagagagagagaaacagagttcaagatcacgagccttccttcttcttcttcttcttcatcgagagcgatcaaaggaacaaaaaggatctcaagaaacccacttgtgttgttggttagatacttcacgggtctctgaaaacgtctctttctcacaaccataacttgatcacccaatactccttttctcatcttaaaggctcaaattcatccacgtcacaccgttgttcatttcctctgatgtcttggtgtcgttagatattgtctcccaaaaaagaaatcttcttgacacagagattgaagtcgcaaagagacagaggaaagagggggagaaaatggatcgaccagcagtgagtggtccaatggatttgccgattatgcacgatagtgataggtatgaactcgtcaaggatattggctccggtaattttggagttgcgagattgatgagagacaagcaaagtaatgagcttgttgctgttaaatatatcgagagagtgttgttttaaaggctctaggtgtttcttttgttatggaacgtggtattaatggtgggactttttgtatttgtacagatagatgaaaatgtaaaaagggagataatcaaccacaggtccttaagacatcccaatatcgttagattcaaagaggttatattaacaccaacccatttagccattgttatggaatatgcatctggaggagaacttttcgagcgaatctgcaatgcaggccgcttcagcgaagacgaggcgaggtttttcttccagcaactcatttcaggagttagttactgtcatgctatgcaagtatgtcaccgagacttaaagctcgagaatacgttattagatggtagcccggcccctcgtctaaagatatgtgatttcggatattctaagtcatcagtgttacattcgcaaccaaaatcaactgttggaactcctgcttacatcgctcctgaggttttactaaagaaagaatatgatggaaaggttgcagatgtttggtcttgtggggttactctgtatgtcatgctggttggagcatatcctttcgaagatcccgaggaaccaaagaatttcaggaaaactatacatagaatcctgaatgttcagtatgctattccggattatgttcacatatctcctgaatgtcgccatttgatctccagaatatttgttgctgaccctgcaaagaggatatcaattcctgaaataaggaaccatgaatggtttctaaagaatctaccggcagatctaatgaacgataacacgatgaccactcagtttgatgaatcggatcaaccgggccaaagcatagaagaaattatgcagatcattgcagaagcaactgttcctcctgcaggcactcagaatctgaaccattacctcacaggaagcttggacatagatgacgatatggaggaagacttagagagcgaccttgatgatcttgacatcgacagtagcggagagattgtgtacgcaatgtgatactatatatctatttgcatggtttctgctacaaaaatgtcaaacaaaaaatgttgaagaataagattaagatgttttgcttgctattgagttggcccaactttgtctcaatgagtacactttgaatctttgatatgcaaaagactaaatttc

SEQ ID NO: 14 - protein, 314 aa, Arabidopsis thaliana
MVGLFVFVQIDENVKREIINHRSLRHPNIVRFKEVILTPTHLAIVMEYASGGELFERICNAGRFSEDEARFFFQQLISGVSYCHAMQVCHRDLKLENTLLDGSPAPRLKICDFGYSKSSVLHSQPKSTVGTPAYIAPEVLLKKEYDGKVADVWSCGVTLYVMLVGAYPFEDPEEPKNFRKTIHRILNVQYAIPDYVHISPECRHLISRIFVADPAKRISIPEIRNHEWFLKNLPADLMNDNTMTTQFDESDQPGQSIEEIMQIIAEATVPPAGTQNLNHYLTGSLDIDDDMEEDLESDLDDLDIDSSGEIVYAM

SEQ ID NO: 15 - DNA, 1514 bp, Arabidopsis thaliana
  CDS (286)..(1281)
aaatagagaagctcttcaagtatccgatgtttttgtttaatcaacaagaggcggagatacgggagaaattgcatgtgtaatcataaaatgtagatgttagcttcgtcgtttttactatagtttagttctcttcttcttcttttttcgtcattacaatctctttcttaatttacttcttcttgatagtataattaagttgtttgtaataatctgtacaaagatgttgtgttctcataaaaaattcaattttgtaaagaagctctacatgttccttgctctgtaaacatggtccccttttggactacagtttctcgaaatggctcatcagactcagagacgactctccaatctgcttcaaaagccacaaaacagtataaatatccttctcttcgtccctctcatcgcctgtctctcctcttcctcttcccgttccatttatccgcaaacggagcttgttttcggtgcacctgcttcagccacttcaaacttgaactgagaaggatgggaaacgaatcatatgaagacgccatcgaagctctcaagaagcttctcattgagaaggatgatctgaaggatgtagctgcggccaaggtgaagaagatcacggcggagcttcaggcagcctcgtcatcggacagcaaatcttttgatcccgtcgaacgaattaaggaaggcttcgtcaccttcaagaaggagaaatacgagaccaatcctgctttgtatggtgagctcgccaaaggtcaaagcccaaagtacatggtgtttgcttgttcggactcacgagtgtgcccatcacacgtactagacttccatcctggagatgccttcgtggttcgtaatatcgccaatatggttcctccttttgacaaggtcaaatatgcaggagttggagccgccattgaatacgctgtcttgcaccttaaggtggaaaacattgtggtgatagggcacagtgcatgtggtggcatcaaggggcttatgtcatttcctcttgacggaaacaactctactgacttcatagaggattgggtcaaaatctgtttaccagcaaagtcaaaagttttggcagaaagtgaaagttcagcatttgaagaccaatgtggccgatgcgaaagggaggcagtgaatgtgtcactagcaaacctattgacatatccatttgtgagagaaggagttgtgaaaggaacacttgctttgaagggaggctactatgactttgttaatggctcctttgagctttgggagctccagtttggaatttcccccgttcattctatatgaactaacacatcaccatcaccatcgctaccaccaccatcacaaacatcatcatcgtcgtcatcatcatgatcagcatcttcatatataaatgttttactcttatttaattgctacttgtaatggtatacatttacttgcgatgagcttcttttccttcattatccagttataaaataaataaataaatcatgtttactttcacagatatcgttttgctgaagttgctttgattt

SEQ ID NO: 16 - protein, 331 aa, Arabidopsis thaliana
MVPFWTTVSRNGSSDSETTLQSASKATKQYKYPSLRPSHRLSLLFLFPFHLSANGACFRCTCFSHFKLELRRMGNESYEDAIEALKKLLIEKDDLKDVAAAKVKKITAELQAASSSDSKSFDPVERIKEGFVTFKKEKYETNPALYGELAKGQSPKYMVFACSDSRVCPSHVLDFHPGDAFVVRNIANMVPPFDKVKYAGVGAAIEYAVLHLKVENIVVIGHSACGGIKGLMSFPLDGNNSTDFIEDWVKICLPAKSKVLAESESSAFEDQCGRCEREAVNVSLANLLTYPFVREGVVKGTLALKGGYYDFVNGSFELWELQFGISPVHSI

SEQ ID NO: 17 - DNA, 1031 bp, Arabidopsis thaliana
  CDS (63)..(755)
atgcagtaatctgataaaaccctccacagagatttccaacaaaacaggaactaaaacacaagatgaagattatgatgatgattaagctctgcttcttctccatgtccctcatctgcattgcacctgcagatgctcagacagaaggagtagtgtttggatataaaggcaaaaatggaccaaaccaatggggacacttaaaccctcacttcaccacatgcgcggtcggtaaattgcaatctccaattgatattcaaaggaggcaaatattttacaaccacaaattgaattcaatacaccgtgaatactacttcacaaacgcaacactagtgaaccacgtctgtaatgttgccatgttcttcggggagggagcaggagatgtgataatagaaaacaagaactataccttactgcaaatgcattggcacactccttctgaacatcacctccatggagtccaatatgcagctgagctgcacatggtacaccaagcaaaagatggaagctttgctgtggtggcaagtctcttcaaaatcggcactgaagagcctttcctctctcagatgaaggagaaattggtgaagctaaaggaagagagactcaaagggaaccacacagcacaagtggaagtaggaagaatcgacacaagacacattgaacgtaagactcgaaagtactacagatacattggttcactcactactcctccttgctccgagaacgtttcttggaccatccttggcaaggtgaggtcaatgtcaaaggaacaagtagaactactcagatctccattggacacttctttcaagaacaattcaagaccgtgtcaacccctcaacggccggagagttgagatgttccacgaccacgagcgtgtcgataaaaaagaaaccggtaacaaaaagaaaaaacccaattaaaatagttttacattgtctattggtttgtttagaaccctaattagctttgtaaaactaataatctcttatgtagtactgtgttgttgtttacgacttgatatacgatttccaaat

SEQ ID NO: 18 - protein, 231 aa, Arabidopsis thaliana
MKIMMMIKLCFFSMSLICIAPADAQTEGVVFGYKGKNGPNQWGHLNPHFTTCAVGKLQSPIDIQRRQIFYNHKLNSIHREYYFTNATLVNHVCNVAMFFGEGAGDVIIENKNYTLLQMHWHTPSEHHLHGVQYAAELHMVHQAKDGSFAVVASLFKIGTEEPFLSQMKEKLVKLKEERLKGNHTAQVEVGRIDTRHIERKTRKYYRYIGSLTTPPCSENVSWTILGKVRSM

SEQ ID NO: 19 - DNA, 736 bp, Arabidopsis thaliana
  CDS (1)..(654)
atggatgaatatgtagaggatgaacacgaattcagctacgaatggaaccaagagaacgggccagcgaaatggggaaagctaagaccggaatggaaaatgtgcggaaaaggagaaatgcaatcgcctattgatcttatgaacaaaagagttagacttgttactcatcttaaaaagcttactagacactacaaaccttgtaacgccactctcaaaaatagaggccatgatatgatgctgaaatttggagaagaagggtcagggagtattacggtcaatggaactgagtataaactcttacagcttcattggcattctccctctgaacatactatgaatggaagaaggtttgctctcgagctacacatggttcacgaaaacattaacggaagtttggctgtagtcacagtcctctacaaaatcggaaggccagattcttttctcggattgctggaaaataaattgtcggcaattacagatcaaaatgaggcggagaaatatgtagatgtgattgacccaagggatattaagattgggagcagaaaattttatagatacattggatcacttactactcctccttgtacgcaaaatgttatttggaccgtcgttaaaaaggtaaatactcatcgttattttcttctcttttttacttaatcaaacatagcattaatagatcattacaaggtactaatagtgtgaatatccatatccaaaaggtttatccatctacatgtta

SEQ ID NO: 20 - protein, 217 aa, Arabidopsis thaliana
MDEYVEDEHEFSYEWNQENGPAKWGKLRPEWKMCGKGEMQSPIDLMNKRVRLVTHLKKLTRHYKPCNATLKNRGHDMMLKFGEEGSGSITVNGTEYKLLQLHWHSPSEHTMNGRRFALELHMVHENINGSLAVVTVLYKIGRPDSFLGLLENKLSAITDQNEAEKYVDVIDPRDIKIGSRKFYRYIGSLTTPPCTQNVIWTVVKKVNTHRYFLLFFT

SEQ ID NO: 21 - DNA, 1134 bp, Arabidopsis thaliana
  CDS (48)..(881)
aaaacacattctgagaagaagaagaagaaaataagaaaaaacaaaagatgaaaaccattatcctttttgtaacatttcttgctctttcttcttcatctctagccgatgagacagagactgaatttcattacaaacccggtgagatagccgatccctcgaaatggagcagtatcaaggctgaatggaaaatttgcgggacagggaagaggcaatcgccaatcaatcttactccaaaaatagctcgcattgttcacaattctacagagattcttcagacatattacaaacctgtagaggctattcttaagaaccgtggattcgacatgaaggttaagtgggaagacgatgcagggaagatcgtgatcaatgataccgactataaattggttcaaagccactggcacgcaccttcagagcattttctcgatggacagaggttggcaatggaacttcacatggtacacaaaagtgtagaagggcacttggcagtgattggagttctcttcagagaaggagaaccaaatgctttcatttcgcggatcatggacaagatccataagatcgcagacgtacaagatggagaggtcagcatcggaaagatagatccaagagaatttggatgggatcttacaaagttttatgaatacagaggttctctcacgactcctccttgcacggaagatgtcatgtggaccatcatcaacaaggtggggactgtttcacgtgagcaaattgatgtattgacagatgctcgtcgcggtggttatgagaagaacgcgagaccagctcaacctctgaacggacgtctggtttatttaaacgagcagtccagtccaagtccaactccacggctaagaataccacgagttggtccggtctaagacagtcttataggacaaggcaactccgagccctaatttccatacaaagaaaattcggaaaagaattttgaagatgtatgaaaattgggagccataactatttttttttaactattcttttgattaaaagataaaactacgcaatattatatgcataaagtttttcttttatacatgtattccaataaacaagatgtaataatatccaaccataatgagttgtttgattattttataacacaagatctctcac

SEQ ID NO: 22 - protein, 277 aa, Arabidopsis thaliana
MKTIILFVTFLALSSSSLADETETEFHYKPGEIADPSKWSSIKAEWKICGTGKRQSPINLTPKIARIVHNSTEILQTYYKPVEAILKNRGFDMKVKWEDDAGKIVINDTDYKLVQSHWHAPSEHFLDGQRLAMELHMVHKSVEGHLAVIGVLFREGEPNAFISRIMDKIHKIADVQDGEVSIGKIDPREFGWDLTKFYEYRGSLTTPPCTEDVMWTIINKVGTVSREQIDVLTDARRGGYEKNARPAQPLNGRLVYLNEQSSPSPTPRLRIPRVGPV

SEQ ID NO: 23 - DNA, 804 bp, Arabidopsis thaliana
  CDS (1)..(804)
atggataccaacgcaaaaacaattttcttcatggctatgtgtttcatctatctatctttccctaatatttcacacgctcattctgaagtcgacgacgaaactccatttacttacgaacaaaaaacggaaaagggaccagagggatggggcaaaataaatccgcactggaaagtttgtaacaccggaagatatcaatccccgatcgatcttactaacgaaagagtcagtcttattcatgatcaagcatggacaagacaatataaaccagctccggctgtaattacaaacagaggccatgacattatggtatcatggaaaggagatgctgggaagatgacaatacggaaaacggattttaatttggtgcaatgccattggcattcaccttctgagcataccgttaacggaactaggtacgacctagagcttcacatggttcacacgagtgcacgaggcagaactgcggttatcggagttctttacaaattaggcgaacctaatgaattcctcaccaagctactaaatggaataaaagcagtgggaaataaagagataaatctagggatgattgatccacgagagattaggtttcaaacaagaaaattctatagatacattggctctctcactgttcctccttgcactgaaggcgtcatttggactgtcgtcaaaagggtgaacacaatatcaatggagcaaattacagctcttaggcaagccgttgacgatggatttgagacaaattcaagaccggttcaagactcaaagggaagatcagtttggttctatgatccaaatgtttga

SEQ ID NO: 24 - protein, 267 aa, Arabidopsis thaliana
MDTNAKTIFFMAMCFIYLSFPNISHAHSEVDDETPFTYEQKTEKGPEGWGKINPHWKVCNTGRYQSPIDLTNERVSLIHDQAWTRQYKPAPAVITNRGHDIMVSWKGDAGKMTIRKTDFNLVQCHWHSPSEHTVNGTRYDLELHMVHTSARGRTAVIGVLYKLGEPNEFLTKLLNGIKAVGNKEINLGMIDPREIRFQTRKFYRYIGSLTVPPCTEGVIWTVVKRVNTISMEQITALRQAVDDGFETNSRPVQDSKGRSVWFYDPNV

SEQ ID NO: 25 - DNA, 1088 bp, Arabidopsis thaliana
  CDS (114)..(947)
gatcaacatctccttgaagttgtttcataagaataagagctataaaagaggataaaaccaaaatttgaatttttttcttctatctctctccccaagatatatagcacaagaaaatgaagataccatcaattggctatgtctttttccttatcttcatctctattacaattgtttcgagttcaccagatcatggagaagttgaggacgaaacgcagtttaactacgagaagaaaggagagaaggggccagagaactggggaagactaaagccagagtgggcaatgtgtggaaaaggcaacatgcagtctccgattgatcttacggacaaaagagtcttgattgatcataatcttggataccttcgtagccagtatttaccttcaaatgccaccattaagaacagaggccatgatatcatgatgaaatttgaaggaggaaatgcaggtttaggtatcactattaatggtactgaatataaacttcaacagattcattggcactctccttccgaacacacactcaatggcaaaaggtttgttcttgaggaacacatggttcatcagagcaaagatggacgcaacgctgttgtcgctttcttttacaaattgggaaaacctgactattttctcctcacgttggaaagatacttgaagaggataactgatacacacgaatcccaggaatttgtcgagatggttcatcctagaacattcggttttgaatcaaaacactattatagatttatcggatcacttacaactccaccgtgttctgaaaatgtgatttggacgatttccaaagagatgaggactgtgacattaaaacaattgatcatgcttcgagtgactgtacacgatcaatctaactcaaatgctagaccgcttcagcgtaaaaatgagcgtccggtggcactttacataccaacatggcatagtaaactatattaaatatttaagtttggtttatattctttctagtaatctttgaaatattgtaagagataatgcttctaataaataacattggatttattggaattaatgtattgaaaaaactatgcaaatactacagtgtattttggaacgacc

SEQ ID NO: 26 - protein, 277 aa, Arabidopsis thaliana
MKIPSIGYVFFLIFISITIVSSSPDHGEVEDETQFNYEKKGEKGPENWGRLKPEWAMCGKGNMQSPIDLTDKRVLIDHNLGYLRSQYLPSNATIKNRGHDIMMKFEGGNAGLGITINGTEYKLQQIHWHSPSEHTLNGKRFVLEEHMVHQSKDGRNAVVAFFYKLGKPDYFLLTLERYLKRITDTHESQEFVEMVHPRTFGFESKHYYRFIGSLTTPPCSENVIWTISKEMRTVTLKQLIMLRVTVHDQSNSNARPLQRKNERPVALYIPTWHSKLY

SEQ ID NO: 27 - DNA, 783 bp, Arabidopsis thaliana
  CDS (1)..(783)
atggatgccaacacaaaaacaattttattttttgtagtgttcttcatcgatttattttcccctaatattttattcgtttatgctcgtgaaatcggcaacaaaccgctatttacatacaaacaaaaaacagagaaaggaccagcggaatggggcaaattagaccctcaatggaaagtttgtagcaccggaaaaattcaatctccgattgatctcactgacgaaagagtcagtcttattcatgatcaagccttgagtaaacattacaaaccagcttcggctgtaattcaaagtagaggacatgacgttatggtatcgtggaaaggagatggtgggaaaataacaatacatcaaacggattataaattggtgcagtgccattggcattcaccgtctgagcataccattaacggaactagctatgacctagagcttcacatggttcacacgagtgctagtggcaaaaccactgtggttggagttctttataaattaggtgaacctgatgaattcctcacaaagatactaaatggaataaaaggagtagggaaaaaagagatagatctaggaatcgtggatcctcgagatattagatttgaaaccaacaatttctatagatacattggctctctcactattcctccatgcaccgaaggcgttatttggaccgtccagaaaagggtattatattttttttgtttctgttatagattaattatcttcgttacaccttacataaacattttttggatttttgtttttgtattttggtgtatgctaatgtaa

SEQ ID NO: 28 - protein, 260 aa, Arabidopsis thaliana
MDANTKTILFFVVFFIDLFSPNILFVYAREIGNKPLFTYKQKTEKGPAEWGKLDPQWKVCSTGKIQSPIDLTDERVSLIHDQALSKHYKPASAVIQSRGHDVMVSWKGDGGKITIHQTDYKLVQCHWHSPSEHTINGTSYDLELHMVHTSASGKTTVVGVLYKLGEPDEFLTKILNGIKGVGKKEIDLGIVDPRDIRFETNNFYRYIGSLTIPPCTEGVIWTVQKRVLYFFCFCYRLIIFVTPYINIFWIFVFVFWCMLM

SEQ ID NO: 29 - DNA, 859 bp, Arabidopsis thaliana
  CDS (1)..(828)
atggtgaactactcatcaatcagttgcatcttctttgtggctctgtttagtattttcacaattgtttcgatttcgagtgctgcttcaagtcacggagaagttgaggacgaacgcgagtttaactacaagaagaacgatgagaaggggccagagagatggggagaacttaaaccggaatgggaaatgtgtggaaaaggagagatgcaatctcccatagatcttatgaacgagagagttaacattgtttctcatcttggaaggcttaatagagactataatccttcaaatgcaactcttaagaacagaggccatgacatcatgttaaaatttgaagatggagcaggaactattaagatcaatggttttgaatatgaacttcaacagcttcactggcactctccgtctgaacatactattaatggaagaaggtttgcacttgagctgcatatggttcacgaaggcaggaatagaagaatggctgttgtgactgtgttgtacaagatcggaagagcagatacttttatcagatcgttggagaaagaattagagggcattgctgaaatggaggaggctgagaaaaatgtaggaatgattgatcccaccaaaattaagatcggaagcagaaaatattacagatacactggttcacttaccactcctccttgcactcaaaacgttacttggagcgtcgttagaaaggttaggaccgtgacaagaaaacaagtgaagctcctccgcgtggcagtgcacgatgatgctaattcgaatgcgaggccggttcaaccaaccaacaagcgcatagtgcacttatacagaccaatagtttaatatatgaagatactgaaagcttttactaatc

SEQ ID NO: 30 - protein, 275 aa, Arabidopsis thaliana
MVNYSSISCIFFVALFSIFTIVSISSAASSHGEVEDEREFNYKKNDEKGPERWGELKPEWEMCGKGEMQSPIDLMNERVNIVSHLGRLNRDYNPSNATLKNRGHDIMLKFEDGAGTIKINGFEYELQQLHWHSPSEHTINGRRFALELHMVHEGRNRRMAVVTVLYKIGRADTFIRSLEKELEGIAEMEEAEKNVGMIDPTKIKIGSRKYYRYTGSLTTPPCTQNVTWSVVRKVRTVTRKQVKLLRVAVHDDANSNARPVQPTNKRIVHLYRPIV

SEQ ID NO: 31 - DNA, 1053 bp, Arabidopsis thaliana
  CDS (1)..(1053)
atgaagatatcatcactaggatgggtcttagtccttatcttcatctctattaccattgtttcgagtgcaccagcacctaaacctcctaaacctaagcctgcaccagcacctacacctcctaaacctaagcccacaccagcacctacacctcctaaacctaagcccaaaccagcacctacacctcctaaacctaagcctgcaccagcacctacacctcctaaacctaagcccgcaccagcacctacacctcctaaacctaagcccaaaccagcacctacacctcctaatcctaagcccacaccagcacctacacctcctaaacctaagcctgcaccagcaccagcaccaacaccagcaccgaaacctaaacctgcacctaaaccagcaccaggtggagaagttgaggacgaaaccgagtttagctacgagacgaaaggaaacaaggggccagcgaaatggggaacactagatgcagagtggaaaatgtgtggaataggcaaaatgcaatctcctattgatcttcgggacaaaaatgtggtagttagtaataaatttggattgcttcgtagccagtatctgccttctaataccaccattaagaacagaggtcatgatatcatgttgaaattcaaaggaggaaataaaggtattggtgtcactatccgtggtactagatatcaacttcaacaacttcattggcactctccttccgaacatacaatcaatggcaaaaggtttgcgctagaggaacacttggttcatgagagcaaagataaacgctacgctgttgtcgcattcttatacaatctcggagcatctgacccttttctcttttcgttggaaaaacaattgaagaagataactgatacacatgcgtccgaggaacatattcgcactgtgtcaagtaaacaagtgaagcttctccgtgtggctgtacacgatgcttcagattcaaatgccaggccgcttcaagcagtcaataagcgcaaggtatatttatacaaaccaaaggttaagttaatgaagaaatactgtaatataagttcttactag

SEQ ID NO: 32 - protein, 350 aa, Arabidopsis thaliana
MKISSLGWVLVLIFISITIVSSAPAPKPPKPKPAPAPTPPKPKPTPAPTPPKPKPKPAPTPPKPKPAPAPTPPKPKPAPAPTPPKPKPKPAPTPPNPKPTPAPTPPKPKPAPAPAPTPAPKPKPAPKPAPGGEVEDETEFSYETKGNKGPAKWGTLDAEWKMCGIGKMQSPIDLRDKNVVVSNKFGLLRSQYLPSNTTIKNRGHDIMLKFKGGNKGIGVTIRGTRYQLQQLHWHSPSEHTINGKRFALEEHLVHESKDKRYAVVAFLYNLGASDPFLFSLEKQLKKITDTHASEEHIRTVSSKQVKLLRVAVHDASDSNARPLQAVNKRKVYLYKPKVKLMKKYCNISSY

SEQ ID NO: 33 - DNA, 996 bp, Arabidopsis thaliana
  CDS (90)..(866)
ctagagagcatcttcttatatcaactaaactttgtattcatttccaagtatcactctaaatcatctttttcgaattcgcctcccaagatatgtcgacagagtcgtacgaagacgccattaaaagactcggagagcttctcagtaagaaatcggatctcgggaacgtggcagccgcaaagatcaagaagttaacggatgagttagaggaacttgattccaacaagttagatgccgtagaacgaatcaaatccggatttctccatttcaagactaataattatgagaagaatcctactttgtacaattcacttgccaagagccagacccccaagtttttggtgtttgcttgtgcggattcacgagttagtccatctcacatcttgaatttccaacttggggaagccttcatcgttagaaacattgcaaacatggtgccaccttatgacaagacaaagcactctaatgttggtgcggcccttgaatatccaattacagtcctcaacgtggagaacattcttgttattggacacagctgttgtggtggaataaagggactcatggccattgaagataatacagctcccactaagaccgagttcatagaaaactggatccagatctgtgcaccggccaagaacaggatcaagcaggattgtaaagacctaagctttgaagatcagtgcaccaactgtgagaaggaagccgtgaacgtgtccttggggaatcttttgtcttacccattcgtgagagaaagagtggtgaagaacaagcttgccataagaggagctcactatgatttcgtaaaaggaacgtttgatctttgggaacttgacttcaagactacccctgcctttgccttgtcttaaaagattcctcctactcaaatattttctctatgttgtttctaattatgttcttataatcttcttctgttgcttctgtaatgtcatctttgctacttctattccaatagaaatgaataaagctttaaagagc

SEQ ID NO: 34 - protein, 258 aa, Arabidopsis thaliana
MSTESYEDAIKRLGELLSKKSDLGNVAAAKIKKLTDELEELDSNKLDAVERIKSGFLHFKTNNYEKNPTLYNSLAKSQTPKFLVFACADSRVSPSHILNFQLGEAFIVRNIANMVPPYDKTKHSNVGAALEYPITVLNVENILVIGHSCCGGIKGLMAIEDNTAPTKTEFIENWIQICAPAKNRIKQDCKDLSFEDQCTNCEKEAVNVSLGNLLSYPFVRERVVKNKLAIRGAHYDFVKGTFDLWELDFKTTPAFALS

SEQ ID NO: 35 - DNA, 1362 bp, Arabidopsis thaliana
  CDS (193)..(1098)
attgttgtgtaaaactcttgttcctcttcctcttcaacgtgaacacttctatttctcagagaacattcacctatatgtctttcttcaaggagaagtcttcctctttccagatttagatgaacactcttcagatgccttgtgccttattgatccagattcgaagtacccaactttactctctagacctttttcatggcagccactcccacacacttctctgtctcccatgatcctttttcttccacgtctctccttaatctccaaactcaagcgatctttggtcccaatcacagtttaaagacaacccagttgagaattccagcttctttcagaagaaaagctacaaacttgcaagtgatggcttcaggaaagacacctggactgactcaggaagctaatggggttgcaattgatagacaaaacaacactgatgtatttgacgacatgaaacagcggttcctggccttcaagaagcttaagtacatcagggatgactttgaacactacaaaaatctggcagatgctcaagctccaaagtttctggtgattgcttgtgcagactctagagtttgtccttctgctgtcctgggattccaaccgggtgacgcattcactgttcgtaacattgcaaatttagtacctccatatgagtctggacctactgaaaccaaagctgctctagagttctctgtgaatactcttaatgtggaaaacatcttagtcattggtcatagccggtgtggaggaattcaagctttaatgaaaatggaagacgaaggagattccagaagtttcatacacaactgggtagttgtgggaaagaaggcaaaggaaagcacaaaagctgttgcttcaaacctccattttgatcatcagtgccaacattgtgaaaaggcatcgataaatcattcattagaaaggctgcttgggtacccgtggatagaagagaaagtgcggcaaggttcactgtctctccatggtggatactataattttgttgattgtacgttcgagaaatggacagtggattatgcagcaagcagaggtaagaagaaggaaggcagtggaatcgctgttaaagaccggtcagtttggtcttgacttacgactatctcaatcttcatagagttttttttcataatttatagagaaacatcaaaccccttttggttgggattatcatgtgtttgttccacttgtgtgttgaagtcattttccttcttctgtcttattgaggcagggactaatgtttgttttatctttcagttgtttcgtttaaattccacatttgtgcaatgaactggttggtgtttctttaagatataatcattttgccactgtagtgagatcggaggcatgcat

SEQ ID NO: 36 - protein, 302 aa, Arabidopsis thaliana
MAATPTHFSVSHDPFSSTSLLNLQTQAIFGPNHSLKTTQLRIPASFRRKATNLQVMASGKTPGLTQEANGVAIDRQNNTDVFDDMKQRFLAFKKLKYIRDDFEHYKNLADAQAPKFLVIACADSRVCPSAVLGFQPGDAFTVRNIANLVPPYESGPTETKAALEFSVNTLNVENILVIGHSRCGGIQALMKMEDEGDSRSFIHNWVVVGKKAKESTKAVASNLHFDHQCQHCEKASINHSLERLLGYPWIEEKVRQGSLSLHGGYYNFVDCTFEKWTVDYAASRGKKKEGSGIAVKDRSVWS

SEQ ID NO: 37 - DNA, 1366 bp, Arabidopsis thaliana
  CDS (175)..(1002)
atattaaaccactgtaactgtaatttattgtttcgccgtcccggaatgttcctgttgaaatccattttcgctgattttttttcttccgtctcttcttcagcttcgaccattttcgtcttcttcattcagtgttgagtcctcgtttacctgtgagctcgaagaaagtgacgatcaatgggaaccctaggcagagcattttactcggtcggtttttggatccgtgagactggtcaagctcttgatcgcctcggttgtcgccttcaaggcaaaaattacttccgagaacaactgtcaaggcatcggacactgatgaatgtatttgataaggctccgattgtggacaaggaagcttttgtggcaccaagcgcctcagttattggggacgttcacattggaagaggatcgtccatttggtatggatgcgtattacgaggcgatgtgaacaccgtaagtgttgggtcaggaactaatattcaggacaactcacttgtgcatgtggcaaaatcaaacttaagcgggaaggtgcacccaaccataattggagacaatgtaaccattggtcatagtgctgttttacatggatgtactgttgaggatgagacctttattgggatgggtgcgacacttcttgatggggtcgttgttgaaaagcatgggatggttgctgctggtgcacttgtacgacaaaacaccagaattccttctggagaggtatggggaggaaacccagcaaggttcctcaggaagctcactgatgaggaaattgcttttatctctcagtcagcaacaaactactcaaacctcgcacaggctcacgctgcagagaatgcaaagccattaaatgtgattgagttcgagaaggttctacgcaagaagcatgctctaaaggacgaggagtatgactcaatgctcggaatagtgagagaaactccaccagagcttaacctccctaacaacatactgcctgataaagaaaccaagcgtccttctaatgtgaactgatttttcaggggtatgttttctggccgaagccctacagggtgagatactcaaggggattatgtttcggtctctggtttgaatatggcaggtagagtacattagggtagacggatttacagcttttgaagaagctatgttcaacattttttcatggtttcttagggagtattattgtctaatcaaactttgtatgttatcacttcggtcttttgaacgtaagaatcaagttcatgaaacatgagtgaatattagtctgatgcatgtgcgtatgcaaaaatccatgtgcgcctatgttgctaggcaagcatgaagaataaagatccaaactggatatatcatatatttatctttttataattactgc

SEQ ID NO: 38 - protein, 275 aa, Arabidopsis thaliana
MGTLGRAFYSVGFWIRETGQALDRLGCRLQGKNYFREQLSRHRTLMNVFDKAPIVDKEAFVAPSASVIGDVHIGRGSSIWYGCVLRGDVNTVSVGSGTNIQDNSLVHVAKSNLSGKVHPTIIGDNVTIGHSAVLHGCTVEDETFIGMGATLLDGVVVEKHGMVAAGALVRQNTRIPSGEVWGGNPARFLRKLTDEEIAFISQSATNYSNLAQAHAAENAKPLNVIEFEKVLRKKHALKDEEYDSMLGIVRETPPELNLPNNILPDKETKRPSNVN

SEQ ID NO: 39 - DNA, 1185 bp, Arabidopsis thaliana
  CDS (138)..(974)
cgaactcactcgagttaaaaaaaaaaatcctcccatcaatacgcctccataaacctctctctatctggtggagcgacaccaaaaacaacaaagccttctcattttcacactttgggtaatcggagaatcacaaaaaaatgggaaccctaggacgagcaatttacactgtgggtaactggattcgtggaactggtcaagctcttgatcgcgttggttctcttcttcaaggaagtcaccgtatcgaggaacatctgtcgaggcatcggacgttgatgaatgtgtttgataaatcaccattggtggataaagatgtgtttgtggctccgagtgcttctgttattggtgatgttcagatcggaaaaggctcgtcgatttggtatggctgtgttcttcgaggtgatgtgaataacatcagtgttggatctgggacgaatatccaagataatacgcttgtacatgttgcaaagaccaacataagtggcaaggttctacctactctgattggggacaatgtaacagtaggtcacagtgctgtcattcatgggtgtactgttgaggatgatgcttttgttggtatgggagcaacactacttgatggtgtggtggttgagaaacatgccatggttgctgctggttctcttgtgaaacagaacacgcgaatcccttctggagaggtgtggggaggaaatccagcaaagttcatgagaaagttaacagatgaagagatagtatacatctcacagtcagcaaagaattacatcaatctcgcacagattcacgcctcagagaattcaaagtcatttgagcagatcgaggttgagagagcgcttaggaagaagtatgcacgcaaggacgaggattacgattcaatgcttgggattacccgtgaaactccaccggagttgattcttcccgacaatgtcttaccaggtggtaaacccgtcgccaaggttccgtctactcagtacttctaattccaatctcaggttgtttttgtgtgttgaaatcatttcaagacaggattgattctctggaaggtcaagagagatattattttggttttaacttttcttccgagcaagcaggagatttatcatccttgctcaataatgtatggttgcattatgaagtcatttcttcgaggaacaatttgcagaaagagaaacaaagttggattaatctttc

SEQ ID NO: 40 - protein, 278 aa, Arabidopsis thaliana
MGTLGRAIYTVGNWIRGTGQALDRVGSLLQGSHRIEEHLSRHRTLMNVFDKSPLVDKDVFVAPSASVIGDVQIGKGSSIWYGCVLRGDVNNISVGSGTNIQDNTLVHVAKTNISGKVLPTLIGDNVTVGHSAVIHGCTVEDDAFVGMGATLLDGVVVEKHAMVAAGSLVKQNTRIPSGEVWGGNPAKFMRKLTDEEIVYISQSAKNYINLAQIHASENSKSFEQIEVERALRKKYARKDEDYDSMLGITRETPPELILPDNVLPGGKPVAKVPSTQYF

SEQ ID NO: 41 - DNA, 1230 bp, Arabidopsis thaliana
  CDS (109)..(918)
caaagactgcactctctcctcttcctctggctccggcgaaaaaccccttttcgatttcattgataaaacgcaaatcgatctctcgtgtggaagaagaagaagaacacgatgggaacaatgggtaaagcattctacagcgtaggattctggatccgtgaaactggtcaagcacttgatcggctcggttgtcgcctccaagggaaaaatcatttccgagaacagctatcaaggcaccgcacactcatgaatgtttttgacaaaacccctaatgtggataagggggcttttgtggctcctaacgcttctctctctggtgatgtccatgtgggaagaggttcttccatttggtatggatgtgtcttgagagacataccctttgatttaatgaccgactctgcaggagatgctaacagcattagtgttggagctgggaccaatattcaggacaacgctcttgtccacgttgctaagaccaacttaagtgggaaggtcttacctactgtcattggagacaatgtcaccattggtcatagtgctgttttacatggctgcactgtcgaggatgaggcctatattggtacaagtgcaactgtcttggatggagctcatgttgaaaaacatgccatggttgcttcaggagctcttgttaggcagaacactagaattccctctggcgaggtttggggaggcaacccagctaaatttctgaggaaggtgacagaagaagaaagagtcttcttctccagttcggctgtggagtactccaacttagctcaagctcacgccacagagaacgcaaagaacttggacgaggctgagttcaagaagcttctaaacaagaagaacgctcgcgatacagaatatgattcagtactcgatgatctcacgctccctgagaatgtaccaaaagcagcttgaggcgtttaacctgtgccgccttgcgaatcttgatttgtttggatttgaaaagtaaaaacaaagaacttgatttcctgcttctccaataaagttttcttgggcgtaaaatccattggccagtgctcactgggaaagttttcggcttaaaggcattcatttctctgttaaagattgtgaggggttttgttctcttgtaacttgagaaagaaaagttgtaaccttttcttcctttttatgtcgtctaataaattgttgatcagacagacatttaggttgacctttgcccataaaaagatagctctgcttcaataa

SEQ ID NO: 42 - protein, 269 aa, Arabidopsis thaliana
MGTMGKAFYSVGFWIRETGQALDRLGCRLQGKNHFREQLSRHRTLMNVFDKTPNVDKGAFVAPNASLSGDVHVGRGSSIWYGCVLRDIPFDLMTDSAGDANSISVGAGTNIQDNALVHVAKTNLSGKVLPTVIGDNVTIGHSAVLHGCTVEDEAYIGTSATVLDGAHVEKHAMVASGALVRQNTRIPSGEVWGGNPAKFLRKVTEEERVFFSSSAVEYSNLAQAHATENAKNLDEAEFKKLLNKKNARDTEYDSVLDDLTLPENVPKAA

SEQ ID NO: 43 - DNA, 1042 bp, Arabidopsis thaliana
  CDS (55)..(816)
actctctctcttttcctctttgcaaatccttgaagaaatccaaaatccatagcaatggcgacttcgatagctcgattgtctcggagaggagtcacttctaacctgatccgtcgttgcttcgctgcggaagcggcgttggcgaggaagacagagttacctaaaccgcaattcacggtgtcgccgtcgacggatcgtgtgaaatgggactacagaggccaacgacagatcattcctttgggacagtggcttccgaaggtagccgttgatgcttacgtggcacccaacgttgtgctggctggtcaggtcacagtctgggacggctcgtctgtttggaacggtgccgttttgcgcggcgatctcaacaaaatcactgttggattctgctcgaatgtacaggaacggtgtgttgttcatgccgcctggtcttccccaacaggattaccagcagcgacaataatcgacaggtatgtgacagtaggtgcctacagtcttctgagatcatgtaccatcgaaccagagtgcatcatcggtcaacactcaatactaatggaaggctcactggttgagacccggtcaatcttggaagcgggttcagttgtgccgccaggaagaaggatcccatcaggtgaactatggggaggcaatccagcaagattcattagaaccctaaccaacgaagaaaccctagagatcccaaaactcgctgtagccatcaaccacttaagcggagattacttctctgagttcctaccttactcaactgtctacttagaggtagagaagttcaagaagtcccttgggatcgccgtttagaagcttcatctttttcgtgattcactttcatgtgtttatctatcatatgaggtctttctctctgcatattgcaataagtagctgatgaacatcaaaacaagtccggctctcttttttggttctaaaacgtttgtcatttcgttttttgggttctttgtaaaattccatttaaaactgattttggctgaatattgtctgaatgataatggcgacgacttctggttttgtt

SEQ ID NO: 44 - protein, 252 aa, Arabidopsis thaliana
MATSIARLSRRGVTSNLIRRCFAAEAALARKTELPKPQFTVSPSTDRVKWDYRGQRQIIPLGQWLPKVAVDAYVAPNVVLAGQVTVWDGSSVWNGAVLRGDLNKITVGFCSNVQERCVVHAAWSSPTGLPAATIIDRYVTVGAYSLLRSCTIEPECIIGQHSILMEGSLVETRSILEAGSVVPPGRRIPSGELWGGNPARFIRTLTNEETLEIPKLAVAINHLSGDYFSEFLPYSTVYLEVEKFKKSLGIAV

SEQ ID NO: 45 - DNA, 1130 bp, Arabidopsis thaliana
  CDS (97)..(870)
ctcccgacgactcctctctgtctcctcctccgggaagctttctgtctctctctctctctctctacacaagaccttgaagaatccgattccataacaatggcgacttcgttagcacgaatctctaaaagaagcataacatcggctgtttcatcgaatctgattcggcgttacttcgccgcggaagcagtagcggtggcgacgacggaaacacctaaaccgaaatcgcaggtgacgccgtcgccggatcgggtaaaatgggactacagaggccagagacagataattcctctgggacagtggctaccgaaggtagctgtagatgcttacgtggcacctaacgttgtgttggctggtcaggtcaccgtctgggacggctcgtctgtatggaacggtgccgttttgagaggagatcttaataagatcaccgttggattctgctcaaatgtccaggaacggtgtgttgttcatgctgcgtggtcgtcgcctacaggattaccagcacaaacattgatcgataggtacgtgacagttggtgcatacagtcttttaagatcatgcactatcgaaccagaatgcatcatcgggcaacactcaatcctaatggaaggttcactggtcgaaacccgctcaatcctagaagctggttctgttttaccacctggcagaagaatcccatctggtgaactatggggaggcaatccagcaaggtttattcgaacactcaccaatgaagaaaccttagagatcccgaaacttgctgttgccattaaccacctaagtggagattacttctcagagttcttgccttactcaactatctatctagaggttgagaagttcaagaaatcccttggaatcgccatctagaaagcttcttccaggtttctggctacttccctcattaagaaagcttcttcgttttcggaatttgatctgaataagtagctgcggaacaagaaaaagagcagagctgtgtttcaaatgttgtcttctctgtttgttttgtttaagttcatatccttgtgttcaaactttctatgaagatgataatggtgaaaactggaaagtgtaaaacttctttcgtctcccctcacaattggaaaagctaataatctcgtagtgttatagaa

SEQ ID NO: 46 - protein, 256 aa, Arabidopsis thaliana
MATSLARISKRSITSAVSSNLIRRYFAAEAVAVATTETPKPKSQVTPSPDRVKWDYRGQRQIIPLGQWLPKVAVDAYVAPNVVLAGQVTVWDGSSVWNGAVLRGDLNKITVGFCSNVQERCVVHAAWSSPTGLPAQTLIDRYVTVGAYSLLRSCTIEPECIIGQHSILMEGSLVETRSILEAGSVLPPGRRIPSGELWGGNPARFIRTLTNEETLEIPKLAVAINHLSGDYFSEFLPYSTIYLEVEKFKKSLGIAI